(19) United States
(12) Patent Application Publication
(10) Pub. No.: US 2008/0293701 A1
(43) Pub. Date: Nov. 27, 2008

(54) 1-Acylamino-2-Hydroxy-3-Amino- -Arylalkanes as Renin Inhibitors

(76) Inventors: John J. Baldwin, Gwynedd Valley, PA (US); David A. Claremon, Maple Glen, PA (US); Lawrence W. Dillard, Yardley, PA (US); Patrick T. Flaherty, Pittsburgh, PA (US); Alexey V. Ishchenko, Somerville, MA (US); Jing Yuan, Lansdale, PA (US); Zhenrong Xu, Horsham, PA (US); Gerard McGeehan, Garnet Valley, CA (US); Wenguang Zeng, Lawrenceville, NJ (US); Salvacion Cacatian, Blue Bell, PA (US); Colin Tice, Ambler, PA (US); Robert D. Simpson, Wilmington, DE (US); Wei Zhao, Eagleville, PA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/883,518

(22) PCT Filed: Feb. 1, 2006
(86) PCT No.: PCT/US2006/003489

§ 371 Date: Jul. 7, 2008

Related U.S. Application Data
(60) Provisional application No. 60/649,361, filed on Feb. 2, 2005.

(51) Int. Cl. A61K 31/546; C07C 233/62; A61K 31/164; A61K 31/4453; C07D 211/60; A61K 31/145; C07C 381/00; A61P 9/12; A61P 9/00

(52) U.S. Cl. 514/222.5; 564/123; 514/613; 546/226; 514/330; 514/600; 564/79

(57) ABSTRACT

1-Acylamino-2-hydroxy-3-amino-ω-arylalkanes of formula I. and the salts thereof, have renin-inhibiting properties and can be used as antihypertensive, medicinally active ingredients.

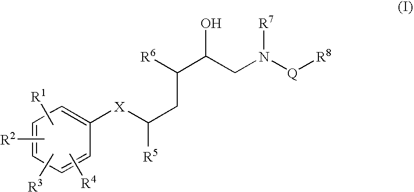

### RELATED APPLICATIONS

This application claims priority from U.S. application 60/649,361, filed Feb. 2, 2005, which is incorporated herein by reference.

### BACKGROUND OF THE INVENTION

In the renin-angiotensin-aldosterone system (RAAS) the biologically active peptide angiotensin II (Ang II) is generated by a two-step mechanism. The highly specific aspartic protease renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAAS represents a major advance in the treatment of cardiovascular diseases (Zaman, M. A. et al Nature Reviews Drug Discovery 2002, 1, 621-636). ACE inhibitors and AT₁ blockers have been accepted as treatments of hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N Engl. J: Med, 1992, 327, 669).

Interest in the development of renin inhibitors stems from the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be bypassed by chymase, a serine protease (Husain A., J. Hypertens., 1993, 11, 1155). In patients, inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the ATI receptor (e.g., by Iosartan) on the other hand overexposes other AT-receptor subtypes to Ang II, whose concentration is dramatically increased by the blockade of AT1 receptors. In summary, renin inhibitors are not only expected to be superior to ACE inhibitors and AT₁ blockers with regard to safety, but more importantly also with regard to their efficacy in blocking the RAAS.

Only limited clinical experience (Azizi M. et al., J. Hypertens., 1994, 12, 419; Neutel J. M. et al., Am. Heart, 1991, 122, 1094) has been generated with renin inhibitors because their peptidomimetic character imparts insufficient oral activity (Kleinert H. D., Cardiovasc. Drugs, 1995, 9, 645). The clinical development of several compounds has been stopped because of this problem together with the high cost of goods. Only one compound has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are not available. Recently, the first non-peptide renin inhibitors were described which show high in vitro activity (Oefner C. et al., Chem. Biol., 1999, 6, 127; Patent Application WO 97/09311; Maerki H. P. et al., Farmaco, 2001, 56, 21). The present invention relates to the unexpected identification of renin inhibitors of a non-peptidic nature and of low molecular weight. Orally active renin inhibitors which are active in indications beyond blood pressure regulation where the tissular renin-chymase system may be activated leading to pathophysiologically altered local functions such as renal, cardiac and vascular remodeling, atherosclerosis, and restenosis, are described.

All documents cited herein are incorporated by reference.

### SUMMARY OF THE INVENTION

It has now been found that 1-acylamino-2-hydroxy-3-amino-ω-arylalkanes of formula I

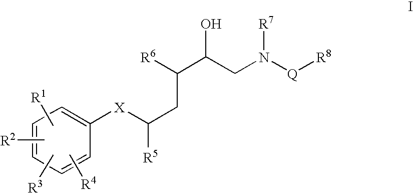

and the salts thereof have renin-inhibiting properties and can be used as antihypertensive, and renal, cardiac and vascular protecting medicinally active ingredients.

### DETAILED DESCRIPTION

An embodiment of the invention is a compound of formula I

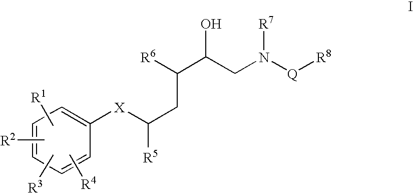

wherein
R¹ is hydrogen, halogen, hydroxy, lower alkoxy, cycloalkoxy, lower alkoxy-lower alkoxy, lower alkylthio-lower alkoxy, cyano-lower alkoxy, hydroxy-lower alkoxy, carboxy-lower alkoxy, lower alkoxycarbonyl-lower alkoxy, carbamoyl-lower alkoxy, N-mono- or N,N-di-lower alkylcarbamoyl-lower alkoxy, or aryl;
R² is hydrogen, halogen, cyano, carbamoyl, lower alkyl, lower haloalkyl, cycloalkyl, halocycloalkyl, cycloalkyl-lower alkyl, halocycloalkyl-loweralkyl, cyano-lower alkyl, hydroxy-lower alkyl, lower alkoxy-lower alkyl, lower haloalkoxy-lower alkyl, lower alkoxy-lower alkyl, lower haloalkoxy-lower alkyl, cycloalkoxy-lower alkyl, halocycloalkoxy-lower alkyl, hydroxy, lower alkanoyloxy-lower alkoxy, hydroxy-lower alkoxy, halo-(hydroxy)-lower alkoxy, lower alkanesulfonyl-(hydroxy)-lower alkoxy, amino-lower alkyl, lower alkylamino-lower alkyl, di-lower alkylamino-lower alkyl, lower alkanoylamino-lower alkyl, lower alkoxycarbonyl-amino-lower alkyl, aminocarbonylamino-lower alkyl, lower alkylaminocarbonylamino-lower alkyl, di(lower alkyl)aminocarbonylamino-lower alkyl, aminosulfonylamino-lower alkyl, lower alkylaminosulfonylamino-lower alkyl, di(lower alkyl)aminosulfonylamino-lower alkyl, amino-lower alkoxy, lower alkylamino-lower alkoxy, di-lower alkylamino-lower alkoxy, lower alkanoylamino-lower alkoxy, lower alkoxycarbonyl-amino-lower alkoxy, aminocarbonylamino-lower alkoxyl, lower alkylaminocarbonylamino-lower alkoxy, di(lower alkyl)aminocarbonylamino-lower alkoxy, aminosulfonylamino-lower alkoxy, lower alkylaminosulfonylamino-lower alkoxy, di(lower alkyl)aminosulfonylamino-lower alkoxy, oxo-lower alkoxy, lower alkoxy, lower haloalkoxy, cycloalkoxy, lower halocycloalkoxy, cycloalkyl-lower alkoxy, halocycloalkyl-lower alkoxy, lower alkenyloxy, cycloalkoxy-lower alkoxy, halocycloalkoxy-lower alkoxy, lower alkoxy-lower alkoxy, lower haloalkoxy-lower alkyl, lower alkoxy-lower alkenyl, lower alkenyloxy-lower alkoxy, lower alkoxy-lower alkenyloxy, lower alkenyloxy-lower alkyl, lower alkanoyl-lower alkoxy, lower alkylthio-lower alkoxy, lower alkanesulfonyl-lower alkoxy, lower alkylthio-(hydroxy)-lower alkoxy, aryl-lower alkoxy, optionally N-oxidized pyridyl-lower alkoxy, thiazolylthio-lower alkoxy or thiazolinylthio-lower alkoxy, imidazolylthio-lower alkoxy, optionally N-oxidized pyridylthio-lower alkoxy, pyrimidinylthio-lower alkoxy, cyano-lower alkoxy, carboxy-lower alkoxy, lower alkoxycarbonyl-lower alkoxy, carbamoyl-lower alkoxy, N-mono- or N,N-di-lower alkylcarbamoyl-lower alkoxy, carboxy-lower alkyl, lower alkoxycarbonyl-lower alkyl, carbamoyl-lower alkyl, or N-mono- or N,N-di-lower alkylcarbamoyl-lower alkyl;
R³ is hydrogen, halogen, cyano, carbamoyl, lower alkyl, lower haloalkyl, lower alkoxy-lower alkyl, cycloalkoxy-lower alkyl, hydroxy-lower alkyl, lower alkylthio-lower alkyl, lower alkanesulfonyl-lower alkyl, optionally partially hydrogenated or N-oxidized pyridyl-lower alkyl, thiazolyl-thio-lower alkyl or thiazolinylthio-lower alkyl, imidazolylthio-lower alkyl, optionally N-oxidized pyridylthio-lower alkyl, pyrimidinylthio-lower alkyl, amino-lower alkyl, lower alkylamino-lower alkyl, di-lower alkylamino-lower alkyl, lower alkanoyl-amino-lower alkyl, lower alkanesulfonylamino-lower alkyl, polyhalo-lower alkane-sulfonylamino-lower alkyl, pyrrolidino-lower alkyl, piperidino-lower alkyl, piperazino-lower alkyl, N′-lower alkylpiperazino-lower alkyl or N′-lower alkanoylpiperazino-lower alkyl, morpholino-lower alkyl, thiomorpholino-lower alkyl, S-oxothiomorpholino-lower alkyl or S,S-dioxothio-morpholino-lower alkyl, cyano-lower alkyl, carboxy-lower alkyl, lower alkoxy-carbonyl-lower alkyl, carbamoyl-lower alkyl, N-mono- or N,N-di-lower alkyl-carbamoyl-lower alkyl, cycloalkyl; phenyl or naphthyl that is unsubstituted or substituted with one to three groups independently selected from lower alkyl, lower alkoxy, hydroxy, lower alkylamino, di-lower alkylamino, halogen, trifluoromethyl, trifluoromethoxy, and cyano; hydroxy, lower alkoxy, cycloalkoxy, lower alkoxy-lower alkoxy, cycloalkoxy-lower alkoxy, hydroxy-lower alkoxy, aryl, lower haloalkoxy, lower alkylthio-lower alkoxy, lower haloalkylthio-lower alkoxy, lower alkanesulfonyl-lower alkoxy, lower haloalkanesulfonyl-lower alkoxy, optionally hydrogenated heteroaryl-lower alkoxy, heterocyclyl-lower alkoxy, optionally partially or fully hydrogenated heteroarylthio-lower alkoxy, such as thiazolylthio-lower alkoxy or thiazolinylthio-lower alkoxy, imidazolylthio-lower alkoxy, optionally N-oxidized pyridylthio-lower alkoxy, pyrimidinylthio-lower alkoxy, amino-lower alkoxy, lower alkylamino-lower alkoxy, di-lower alkylamino-lower alkoxy, lower alkanoylamino-lower alkoxy, lower alkanesulfonylamino-lower alkoxy, polyhalo-lower alkanesulfonylamino-lower alkoxy, pyrrolidino-lower alkoxy, piperidino-lower alkoxy, piperazino-lower alkoxy, N′-lower alkylpiperazino-lower alkoxy or N′-lower alkanoylpiperazino-lower alkoxy, morpholino-lower alkoxy, thiomorpholino-lower alkoxy, S-oxothiomorpholino-lower alkoxy or S,S-dioxothiomorpholino-lower alkoxy, cyano-lower alkoxy, carboxy-lower alkoxy, lower alkoxycarbonyl-lower alkoxy, carbamoyl-lower alkoxy, N-mono- or N,N-di-lower alkylcarbamoyl-lower alkoxy, carboxy-lower alkyl, lower alkoxycarbonyl-lower alkyl, carbamoyl-lower alkyl, or N-mono- or N,N-di-lower alkylcarbamoyl-lower alkyl; or
R² and R³ taken together with the atoms through which they are attached form a fused dioxolane, dioxane, benzene or cyclohexene ring, wherein said ring is substituted with up to 2 substituents independently selected from lower alkyl and lower alkoxy-lower alkyl;
R⁴ is hydrogen, lower alkyl, hydroxy, lower alkoxy, cycloalkoxy, lower alkoxy-lower alkoxy, or cycloalkyl-lower alkoxy; or
R³ and R⁴ taken together with the atoms through which they are attached form a fused dioxolane, dioxane, benzene or cyclohexene ring, wherein said ring is substituted with up to 2 substituents independently selected from lower alkyl and lower alkoxy-lower alkyl; provided that R³ does not form a ring with R²;
X is methylene or hydroxymethylene;
R⁵ is lower alkyl, lower haloalkyl, cycloalkyl, halocycloalkyl, lower alkyl-cycloalkyl, lower haloalkyl-cycloalkyl, cycloalkyl-lower alkyl, aryl, aryl-lower alkyl, heterocyclyl, heterocyclyl-lower alkyl;
R⁶ is amino, lower alkylamino, di-lower alkylamino, or lower alkanoylamino;
R⁷ is hydrogen, lower alkyl, lower haloalkyl, cycloalkyl, lower alkoxy-lower alkyl, or lower haloalkoxy-lower alkyl;
Q is carbonyl, thiocarbonyl, or sulfonyl;
R⁸ is lower alkyl, lower haloalkyl, C₈-C₁₅alkyl, C₈-C₁₅haloalkyl, cycloalkyl, halocycloalkyl, lower alkyl-cycloalkyl, cycloalkyl-lower alkyl, halocycloalkyl-lower alkyl, lower alkoxy-loweralkyl, lower haloalkoxy-lower alkyl, cycloalkoxy-lower alkyl, cycloalkoxy-cycloalkyl, lower alkylthio-lower alkyl, lower haloalkylthio-lower alkyl, lower alkanesulfonyl-lower alkyl, lower haloalkanesulfonyl-lower alkyl, lower alkylthio-cycloalkyl, lower haloalkylthio-cycloalkyl, lower alkanesulfonyl-cycloalkyl, lower haloalkanesulfonyl-cycloalkyl, aryl, aryl-lower alkyl, aryl-lower hydroxyalkyl, arylcycloalkyl, aryloxy-lower alkyl, aryloxy cycloalkyl, arylthio-lower alkyl, arylsulfonyl-lower alkyl, arylthio-cycloalkyl, arylsulfonyl-cycloalkyl, lower alkanoyl-lower alkyl, hydroxy-lower alkyl, amino-lower alkyl, lower alkanoylamino-lower alkyl, N-mono-lower alkylamino-lower alkyl, N,N-di-lower alkylamino-lower alkyl, piperidino-lower alkyl, hydroxypiperidino-lower alkyl, lower alkoxypiperidino-lower alkyl, morpholino-lower alkyl, dimethylmorpholino-lower alkyl, thiomorpholino-lower alkyl, S,S-dioxothiomorpholino-lower alkyl, carboxy-lower alkyl, lower alkoxycarbonyl-lower alkyl, carbamoyl-lower alkyl, N-mono-lower alkylcarbamoyl-lower alkyl, N,N-di-lower alkylcarbamoyl-lower alkyl, carboxy-(hydroxy)-lower alkyl, lower alkoxycarbonyl-(hydroxy)-lower alkyl, carbamoyl-(hydroxy)-lower alkyl, N-mono-lower alkylcarbamoyl-(hydroxy)-lower alkyl, N,N-di-lower alkylcarbamoyl-(hydroxy)-lower alkyl, 5- or 6-membered carboxycycloalkyl-lower alkyl, 5- or 6-membered lower alkoxycarbonyl-cycloalkyl-lower alkyl, 5- or 6-membered carbamoylcycloalkyl-lower alkyl, 5- or 6-membered N-mono-alkylcarbamoylcycloalkyl-lower alkyl, N,N-di-lower alkylcarbamoylcycloalkyl-lower alkyl, cyano-lower alkyl, sulfamoyl-lower alkyl, lower alkylsulfamoyl-lower alkyl, or di-lower alkylsulfamoyl-lower alkyl, imidazolyl-lower alkyl, oxopyrrolidinyl-lower alkyl, benzimidazolyl-lower alkyl, oxadiazolyl-lower alkyl, pyridyl-lower alkyl, oxopiperidinyl-lower alkyl or quinolinyl-lower alkyl, piperidin-4-yl-lower alkyl, or lower alkanoylpiperidin-4-yl-lower alkyl, wherein said aryl, imidazolyl, benzimidazolyl, oxadiazolyl, pyridyl, quinolinyl, aryloxy, arylthio and arylsulfonyl groups are optionally substituted with up to four groups independently selected from halo, cyano, nitro, optionally halogenated lower alkyl, optionally halogenated lower alkoxy, optionally halogenated lower alkylthio, optionally halogenated lower alkanesulfonyl, and lower alkoxycarbonyl;

### or R⁸ is OR⁹ or NR⁹R¹⁰

R⁹ is 1) hydrogen, lower alkyl, lower haloalkyl, lower alkenyl, (C₈-C₁₅)alkyl, (C₈-C₁₅)haloalkyl, cycloalkyl, halocycloalkyl, lower alkyl-cycloalkyl, cycloalkyl-lower alkyl, halocycloalkyl-lower alkyl, lower alkoxy-loweralkyl, lower haloalkoxy-lower alkyl, cycloalkoxy-lower alkyl, cycloalkoxy-cycloalkyl, lower alkylthio-lower alkyl, lower haloalkylthio-lower alkyl, lower alkanesulfonyl-lower alkyl, lower haloalkanesulfonyl-lower alkyl, lower alkylthio-cycloalkyl, lower haloalkylthio-cycloalkyl, lower alkanesulfonyl-cycloalkyl, lower haloalkanesulfonyl-cycloalkyl, aminocarbonyl-lower alkyl, lower alkyl-amonocarbonyl-lower alkyl, di(lower alkyl)-amonocarbonyl-lower alkyl, or 2) aryl, aryl-lower alkyl, aryloxy-lower alkyl, arylthio-lower alkyl, or arylsulfonyl-lower alkyl
wherein the aryl groups are optionally substituted with up to four groups independently selected from halo, cyano, optionally halogenated lower alkyl, optionally halogenated lower alkoxy, optionally halogenated lower alkylthio, and optionally halogenated lower alkanesulfonyl;
   R¹⁰ is 1) hydrogen, lower alkyl, lower haloalkyl, (C₈-C₁₅)alkyl, (C₈-C₁₅)haloalkyl, cycloalkyl, halocycloalkyl, cycloalkyl-lower alkyl, halocycloalkyl-lower alkyl, lower alkoxy-lower alkyl, lower haloalkoxy-lower alkyl, alkylthio-lower alkyl, lower haloalkylthio-lower alkyl, lower alkanesulfonyl-lower alkyl, lower haloalkanesulfonyl-lower alkyl, or 2) aryl or aryl-lower alkyl
wherein aryl is optionally substituted with up to four groups independently selected from halo, cyano, optionally halogenated lower alkyl, optionally halogenated lower alkoxy, optionally halogenated lower alkylthio, and optionally halogenated lower alkanesulfonyl;
   or R⁹ and R¹⁰ taken together with the nitrogen to which they are attached form a 4-, 5-, 6- or 7-membered heterocyclic ring composed of carbon atoms and 0 or 1 N, O, or S atoms in addition to the nitrogen atom to which R⁹ and R¹⁰ are attached, said ring atoms being substituted with the appropriate number of hydrogen atoms and optionally substituted with up to four groups independently selected from halogen, (C₁-C₆)alkyl, halo(C₁-C₆)alkyl, lower alkanoyl, lower alkoxycarbonyl, aryl, aryl-lower alkyl, and oxo, such that substitution of one oxo group on a carbon atom forms a carbonyl group and substitution of one or two oxo groups on sulfur forms sulfoxide or sulfone groups respectively; wherein the aryl and arylalkyl groups are substituted with up to four groups independently selected from halo, cyano, optionally halogenated lower alkyl, optionally halogenated lower alkoxy, optionally halogenated lower alkylthio, and optionally halogenated lower alkanesulfonyl;
   and the enantiomers, diastereomers, and salts thereof.

A preferred embodiment of the invention is a compound of the formula Ia

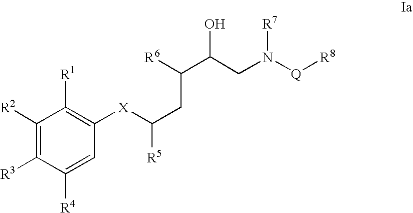

in which the substituents R¹-R⁸, X, and Q are defined as above for 1 and the enantiomers, diastereomers, and salts thereof.

Another embodiment of the invention is a compound of formula Ia, wherein R¹ is hydrogen or aryl;

R² is hydrogen, lower alkyl, cycloalkyl-lower alkyl, lower alkoxy-lower alkyl, lower haloalkoxy-lower alkyl, lower alkoxy-lower alkoxy, lower haloalkoxy-lower alkoxy, lower alkoxy-lower alkyl; cycloalkyl-lower alkoxy, phenyl-lower alkoxy that is unsubstituted or substituted by lower alkyl, lower alkoxy, hydroxy, halogen, nitro and/or by amino; optionally N-oxidized pyridyl-lower alkoxy, lower alkylthio-lower alkoxy, lower alkane-sulfonyl-lower alkoxy, lower alkanoyl-lower alkoxy, cyano-lower alkoxy, carboxy-lower alkoxy, lower alkoxycarbonyl-lower alkoxy, carbamoyl-lower alkoxy, lower alkylcarbamoyl-lower alkoxy, or di-lower alkylcarbamoyl-lower alkoxy;
R³ is hydrogen, halogen, cyano, lower alkyl, lower haloalkyl, aryl, hydroxy, lower alkoxy, or polyhalo-lower alkoxy; or
R² and R³ taken together with the atoms through which they are attached form a fused dioxolane ring, wherein said ring is substituted with up to 2 substituents independently selected from lower alkyl and lower alkoxy-lower alkyl;
R⁴ is hydrogen, lower alkoxy-lower alkoxy, lower alkoxy-lower alkyl, or cycloalkyl-lower alkoxy; or
R³ and R⁴ taken together with the atoms through which they are attached form a fused dioxolane ring, wherein said ring is substituted with up to 2 substituents independently selected from lower alkyl and lower alkoxy-lower alkyl; provided that R³ does not form a ring with R²;
X is methylene or hydroxymethylene;
R⁵ is lower alkyl or cycloalkyl;
R⁶ is amino, lower alkylamino, di-lower alkylamino, or lower alkanoylamino;
R⁷ is hydrogen or methyl;
Q is carbonyl, thiocarbonyl, or sulfonyl;
R⁸ is lower alkyl, lower haloalkyl, C₈-C₁₅ alkyl, C₈-C₁₅ haloalkyl, cycloalkyl, halocycloalkyl, lower alkyl-cycloalkyl, cycloalkyl-lower alkyl, halocycloalkyl-lower alkyl, lower alkoxy-lower alkyl, lower haloalkoxy-lower alkyl, cycloalkoxy-lower alkyl, cycloalkoxy-cycloalkyl, lower alkylthio-lower alkyl, lower haloalkylthio-lower alkyl, lower alkanesulfonyl-lower alkyl, lower haloalkanesulfonyl-lower alkyl, lower alkylthio-cycloalkyl, lower haloalkylthio-cycloalkyl, lower alkanesulfonyl-cycloalkyl, lower haloalkanesulfonyl-cycloalkyl, aryl, aryl-lower alkyl, aryl-lower hydroxyalkyl, arylcycloalkyl, aryloxy-lower alkyl, aryloxy cycloalkyl, arylthio-lower alkyl, arylsulfonyl-lower alkyl, arylthio-cycloalkyl, or arylsulfonyl-cycloalkyl wherein said aryl, aryloxy, arylthio and arylsulfonyl groups are optionally substituted with up to four groups independently selected from halo, cyano, nitro, optionally halogenated lower alkyl, optionally halogenated lower alkoxy, optionally halogenated lower alkylthio, optionally halogenated lower alkanesulfonyl, amino, lower alkylamino, di-lower allkylamino, and lower alkoxycarbonyl;

### or R⁸ is OR⁹ or NR⁹R¹⁰;

R⁹ is selected from 1) hydrogen, lower alkyl, lower haloalkyl, lower alkenyl, (C₈-C₁₅)alkyl, (C₈-C₁₅)haloalkyl, cycloalkyl, halocycloalkyl, lower alkyl-cycloalkyl, cycloalkyl-lower alkyl, halocycloalkyl-lower alkyl, lower alkoxy-loweralkyl, lower haloalkoxy-lower alkyl, cycloalkoxy-lower alkyl, cycloalkoxy-cycloalkyl, lower alkylthio-lower alkyl, lower haloalkylthio-lower alkyl, lower alkanesulfonyl-lower alkyl, lower haloalkanesulfonyl-lower alkyl, lower alkylthio-cycloalkyl, lower haloalkylthio-cycloalkyl, lower alkanesulfonyl-cycloalkyl, lower haloalkanesulfonyl-cycloalkyl, aminocarbonyl-lower alkyl, lower alkyl-amonocarbonyl-lower alkyl, or di(lower alkyl)-amonocarbonyl-lower alkyl, or 2) aryl, aryl-lower alkyl, aryloxy-lower alkyl, arylthio-lower alkyl, or arylsulfonyl-lower alkyl
wherein aryl is optionally substituted with up to four groups independently selected from halo, cyano, optionally halogenated lower alkyl, optionally halogenated lower alkoxy, optionally halogenated lower alkylthio, and optionally halogenated lower alkanesulfonyl;
   R¹⁰ is 1) hydrogen, lower alkyl, lower haloalkyl, (C₈-C₁₅)alkyl, (C₈-C₁₅)haloalkyl, cycloalkyl, halocycloalkyl, cycloalkyl-lower alkyl, halocycloalkyl-lower alkyl, lower alkoxy-lower alkyl, lower haloalkoxy-lower alkyl, alkylthio-lower alkyl, lower haloalkylthio-lower alkyl, lower alkanesulfonyl-lower alkyl, or lower haloalkanesulfonyl-lower alkyl, or 2) aryl or aryl-lower alkyl
wherein aryl is optionally substituted with up to four groups independently selected from halo, cyano, optionally halogenated lower alkyl, optionally halogenated lower alkoxy, optionally halogenated lower alkylthio, and optionally halogenated lower alkanesulfonyl;
   or R⁹ and R¹⁰ taken together with the nitrogen to which they are attached form a 4-, 5-, 6- or 7-membered heterocyclic ring composed of carbon atoms and 0 or 1 N, O, or S atoms in addition to the nitrogen atom to which R⁹ and R¹⁰ are attached, said ring atoms being substituted with the appropriate number of hydrogen atoms and optionally substituted with up to four groups independently selected from halogen, (C₁-C₆)alkyl, halo(C₁-C₆)alkyl, lower alkanoyl, lower alkoxycarbonyl, aryl, aryl-lower alkyl, and oxo, such that substitution of one oxo group on a carbon atom forms a carbonyl group and substitution of one or two oxo groups on sulfur forms sulfoxide or sulfone groups respectively; wherein the aryl and arylalkyl groups are substituted with up to four groups independently selected from halo, cyano, optionally halogenated lower alkyl, optionally halogenated lower alkoxy, optionally halogenated lower alkylthio, and optionally halogenated lower alkanesulfonyl;
   and the enantiomers, diastereomers, and salts thereof.

A preferred embodiment of the invention is a compound of the formula Ia

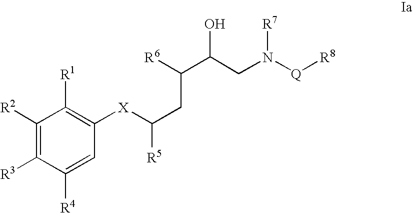

and the enantiomers, diastereomers, and salts thereof.

Another embodiment of the invention is a compound of formula Ia wherein

R¹ is hydrogen;
R² is (C₁-C₄)alkoxy-(C₁-C₄)alkoxy, (C₁-C₄)alkoxy-(C₁-C₄)alkyl, or cycloalkyl-lower alkoxy;
R³ is fluoro, chloro, bromo, cyano, (C₁-C₄)alkyl, (C₁-C₄)haloalkyl, aryl, (C₁-C₄)alkoxy, or (C₁-C₄)haloalkoxy;
R⁴ is hydrogen;
X is methylene;
R⁵ is (C₃-C₅)alkyl;
R⁶ is amino;
R⁷ is hydrogen or methyl;
Q is carbonyl or sulfonyl;
R⁸ is (C₃-C₁₁)alkyl, (C₃-C₁₁)haloalkyl, (C₃-C₇)cycloalkyl, (C₃-C₁₁)cycloalkylalkyl, (C₃-C₁₁)-alkoxyalkyl, aryl, aryl(C₁-C₃)alkyl, aryl(C₃-C₆)cycloalkyl, arylhydroxy(C₁-C₃)alkyl, aryloxy(C₁-C₅)alkyl, or aryloxy(C₃-C₆)cycloalkyl wherein aryl or aryloxy may be unsubstituted or substituted with one to three groups independently selected from halogen, cyano, (C₁-C₃)alkyl, halo(C₁-C₃)alkyl, (C₁-C₃)alkoxy, halo(C₁-C₃)alkoxy;

### or R⁸ is NR⁹R¹⁰;

R⁹ is 1) hydrogen, (C₁-C₁₀)alkyl, (C₃-C₇)alkenyl, (C₃-C₇)cycloalkyl, (C₃-C₆)cycloalkyl(C₁-C₅)alkyl, (C₁-C₆)alkoxy(C₁-C₆)alkyl, or aminocarbonyl(C₁-C₅)alkyl, or 2) aryl or aryl(C₁-C₄)alkyl
wherein aryl is optionally substituted with up to 4 groups independently selected from fluorine, chlorine, cyano, (C₁-C₃)alkyl, halo(C₁-C₃)alkyl, (C₁-C₃)alkoxy, halo(C₁-C₃)alkoxy, and (C₁-C₃)alkanesulfonyl;
   R¹⁰ is hydrogen, lower alkyl, or lower haloalkyl; or
   R⁸ and R⁹ taken together are with the nitrogen to which they are attached form an azetidine, pyrrolidine, piperidine, azepine, piperazine, morpholine, or thiomorpholine ring said ring being optionally substituted with up to two groups independently selected from halogen, (C₁-C₃)alkyl, halo(C₁-C₃)alkyl, and oxo, such that substitution of one oxo group on a carbon atom forms a carbonyl group and substitution of one or two oxo groups on sulfur forms sulfoxide or sulfone groups respectively;
   and the enantiomers, diastereomers, and salts thereof.

Another embodiment of the invention is compounds of formula Ia wherein:

R¹ is hydrogen;
R² is 3-methoxypropoxy, 3-ethoxypropoxy, 4-methoxybutyl, or 2-(cyclopropyl)ethoxy;
R³ is fluoro, chloro, bromo, cyano, methyl, ethyl, isopropyl or tert-butyl, trifluoromethyl, pentafluoroethyl, phenyl, methoxy, difluoromethoxy, or trifluoromethoxy;
R⁴ is hydrogen;
X is methylene;
R⁵ is branched (C₃-C₅)alkyl;
R⁶ is amino;
R⁷ is hydrogen;
Q is carbonyl or sulfonyl; and
R⁸ is propyl, 2,2-dimethylpropyl, butyl, tert-butyl, n-pentyl, 2-methyl-2-butyl, hexyl, 2-hexyl, 2-methyl-2-pentyl, 2,2-dimethylpentyl, 3-heptyl, 2-methyl-2-hexyl, 2,4,4-trimethylpentyl, 2,2,2-trifluoroethyl, 3,3,3-trifluoropropyl, 4,4,4-trifluorobutyl, 1,1,1,3,3,3-hexafluoro-2-methyl-2-propyl, cyclohexyl, 1-methylcyclohexyl, 4-methylcyclohexyl, cyclopropylmethyl, cyclopentylmethyl, 1-cyclopentyl-1-pentyl, cyclohexylmethyl, 2-cyclohexyl-2-propyl, 2-cyclopropyl-1,1-dimethylethyl, 3-cyclopropyl-2-methyl-2-butyl, 3-methoxypropyl, 2-propoxy-2-propyl, phenyl, benzyl, 3-methyl-benzyl, 2-fluorobenzyl, 3-fluorobenzyl, 4-fluorobenzyl, 2,4-difluorobenzyl, 2,3-difluorobenzyl, 3,4-difluorobenzyl, 4-cyanobenzyl, 2-(trifluoromethyl)benzyl, 3-(trifluoromethyl)benzyl, 4-(trifluoro-methyl)benzyl, 4-(trifluoromethoxy)benzyl, phenethyl, 3-phenylpropyl, 2-phenyl-2-propyl, 3-(4-fluorophenyl)-3-pentyl, 1-phenyl-1-cyclopropyl, 1-(4-methylphenyl)-1-cyclopropyl, 1-(4-fluoro-phenyl)-1-cyclopropyl, 1-(4-methoxyphenyl)-1-cyclopropyl, 1-(2,4-dichlorophenyl)-1-cyclopropyl, 1-phenyl-1-cyclopentyl, 1-phenyl-1-cyclohexyl, 1-(4-fluorophenyl)-1-cyclohexyl, 3-hydroxy-2-methyl-3-phenyl-2-propyl, 2-(4-cyanophenoxy)-2-propyl, or 2-(4-chlorophenoxy)-2-propyl;

### or R⁸ is NR⁹R¹⁰;

R⁹ is hydrogen, butyl, isobutyl, t-butyl, pentyl, hexyl, 2,2-dimethyl-1-pentyl, 2-methyl-2-hexyl, 2,4,4-trimethyl-2-pentyl, allyl, 2-(cyclopropyl)ethyl, cyclohexylmethyl, 2-(cyclohexyl)methyl, cyclohexyl, 2-methoxyethyl, benzyl, 2-phenylethyl, 3-phenylpropyl, 3-(4-fluorophenyl)-2-methyl-2-propyl, 3-fluorophenyl, 3-(trifluoromethyl)phenyl, or 2-(aminocarbonyl)-2-methyl-1-propyl,
R¹⁰ is hydrogen, methyl, or isobutyl;

### or R⁹-R¹⁰ is —(CH₂)₅— or —(CH₂)₂O(CH₂)₂—;

and the enantiomers, diastereomers, and salts thereof.

Especially effective are those compounds of formula Ia wherein at least one, two, or preferably all three of the asymmetric carbon atoms of the main chain have the stereochemical configuration shown in formula Ib

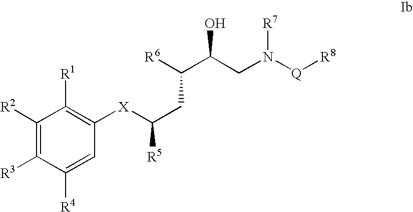

and the pharmaceutically acceptable salts thereof.

Preferred compounds of formulae I, Ia, and Ib are those wherein X is methylene and R⁵ is isopropyl.

Especially preferred are the pharmaceutically acceptable salts of compounds of formulae I, Ia, and Ib.

Another embodiment of the invention is each of the following compounds and their enantiomers, diastereomers and salts:

| Cpd. No. | Name |
|---|---|
| I-1 | N-((2S,3S,5S)-5-(3-(3-methoxypropoxy)-4-methoxybenzyl)-3-amino-2-hydroxy-6- |
| | methylheptyl)butyramide |
| I-2 | N-((2S,3S,5S)-5-(3-(3-methoxypropoxy)-4-methoxybenzyl)-3-amino-2-hydroxy-6- |
| | methylheptyl)-2-cyclopropylacetamide |
| I-3 | N-((2S,3S,5S)-5-(3-(3-methoxypropoxy)-4-methoxybenzyl)-3-amino-2-hydroxy-6- |
| | methylheptyl)pentanamide |
| I-4 | N-((2S,3S,5S)-5-(3-(3-methoxypropoxy)-4-methoxybenzyl)-3-amino-2-hydroxy-6- |
| | methylheptyl)pivalamide |
| I-5 | N-((2S,3S,5S)-5-(3-(2-cyclopropylethoxy)benzyl)-3-amino-2-hydroxy-6- |
| | methylheptyl)-2,2-dimethylhexanamide |
| I-6 | N-((2S,3S,5S)-5-(3-(3-methoxypropoxy)-4-methoxybenzyl)-3-amino-2-hydroxy-6- |
| | methylheptyl)hexanamide |
| I-7 | N-((2S,3S,5S)-5-(3-(3-methoxypropoxy)-4-methoxybenzyl)-3-amino-2-hydroxy-6- |
| | methylheptyl)-2,2-dimethylbutanamide |
| I-8 | N-((2S,3S,5S)-5-(3-(3-methoxypropoxy)-4-methoxybenzyl)-3-amino-2-hydroxy-6- |
| | methylheptyl)-3,3-dimethylbutanamide |
| I-9 | N-((2S,3S,5S)-5-(3-(3-methoxypropoxy)-4-methoxybenzyl)-3-amino-2-hydroxy-6- |
| | methylheptyl)-4-methoxybutanamide |
| I-10 | N-((2S,3S,5S)-5-(3-(3-methoxypropoxy)-4-methoxybenzyl)-3-amino-2-hydroxy-6- |
| | methylheptyl)benzamide |
| I-11 | N-((2S,3S,5S)-5-(3-(3-methoxypropoxy)-4-methoxybenzyl)-3-amino-2-hydroxy-6- |
| | methylheptyl)-3,3,3-trifluoropropanamide |
| I-12 | N-((2S,3S,5S)-5-(3-(3-methoxypropoxy)-4-methoxybenzyl)-3-amino-2-hydroxy-6- |
| | methylheptyl)-2-cyclopentylacetamide |
| I-13 | N-((2S,3S,5S)-5-(3-(3-methoxypropoxy)-4-methoxybenzyl)-3-amino-2-hydroxy-6- |
| | methylheptyl)cyclohexanecarboxamide |
| I-14 | N-((2S,3S,5S)-5-(3-(3-ethoxypropoxy)benzyl)-3-amino-2-hydroxy-6-methylheptyl)- |
| | 2,2-dimethylhexanamide |
| I-15 | N-((2S,3S,5S)-5-(3-(3-methoxypropoxy)-4-methoxybenzyl)-3-amino-2-hydroxy-6- |
| | methylheptyl)heptanamide |
| I-16 | N-((2S,3S,5S)-5-(3-(3-methoxypropoxy)-4-methoxybenzyl)-3-amino-2-hydroxy-6- |
| | methylheptyl)-2,2-dimethylpentanamide |
| I-17 | N-((2S,3S,5S)-5-(3-(3-methoxypropoxy)-4-methoxybenzyl)-3-amino-2-hydroxy-6- |
| | methylheptyl)-2-methylhexanamide |
| I-18 | N-((2S,3S,5S)-5-(3-(3-methoxypropoxy)-4-methoxybenzyl)-3-amino-2-hydroxy-6- |
| | methylheptyl)-2-phenylacetamide |
| I-19 | (2S,3S,5S)-5-(3-(3-methoxypropoxy)-4-methoxybenzyl)-3-amino-6-methyl-1- |
| | (butanesulfonylamino)heptan-2-ol |
| I-20 | N-((2S,3S,5S)-5-(3-(3-methoxypropoxy)-4-methoxybenzyl)-3-amino-2- |
| | hydroxyheptyl)-4,4,4-trifluorobutanamide |
| I-21 | N-((2S,3S,5S)-5-(3-(3-methoxypropoxy)-4-methoxybenzyl)-3-amino-2-hydroxy-6- |
| | methylheptyl)-2-cyclohexylacetamide |
| I-22 | 1-((2S,3S,5S)-5-(3-(3-methoxypropoxy)-4-methoxybenzyl)-3-amino-2-hydroxy-6- |
| | methylheptyl)-3-(1-(4-fluorophenyl)-2-methylpropan-2-yl)urea |
| I-23 | N-((2S,3S,5S)-5-(3-(3-methoxypropoxy)-4-methoxybenzyl)-3-amino-2-hydroxy-6- |
| | methylheptyl)-1-methylcyclohexanecarboxamide |
| I-24 | N-((2S,3S,5S)-5-(3-(3-methoxypropoxy)-4-methoxybenzyl)-3-amino-2-hydroxy-6- |
| | methylheptyl)-4-methylcyclohexanecarboxamide |
| I-25 | N-((2S,3S,5S)-5-(3-(3-methoxypropoxy)-4-ethylbenzyl)-3-amino-2-hydroxy-6- |
| | methylheptyl)-2,2-dimethylhexanamide |
| I-26 | N-((2R,3S,5S)-5-(3-(3-methoxypropoxy)-4-ethylbenzyl)-3-amino-2-hydroxy-6- |
| | methylheptyl)-2,2-dimethylhexanamide |
| I-27 | N-((2S,3S,5S)-5-(3-(3-methoxypropoxy)-4-methoxybenzyl)-3-amino-2-hydroxy-6- |
| | methylheptyl)-N-isopropylpentanamide |
| I-28 | N-((2S,3S,5S)-5-(3-(3-methoxypropoxy)-4-methoxybenzyl)-3-amino-2-hydroxy-6- |
| | methylheptyl)-2,2-dimethylhexanamide |
| I-29 | N-((2R,3R,5S)-5-(3-(3-methoxypropoxy)-4-methoxybenzyl)-3-amino-2-hydroxy-6- |
| | methylheptyl)-2,2-dimethylhexanamide |
| I-30 | N-((2S,3S,5S)-5-(3-(3-methoxypropoxy)-4-methoxybenzyl)-3-amino-2-hydroxy-6- |
| | methylheptyl)-3,3-dimethylhexanamide |
| I-31 | N-((2S,3S,5S)-5-(3-(3-methoxypropoxy)-4-methoxybenzyl)-3-amino-2-hydroxy-6- |
| | methylheptyl)-2-ethylhexanamide |
| I-32 | N-((2S,3S,5S)-5-(3-(3-methoxypropoxy)-4-methoxybenzyl)-3-amino-2-hydroxy-6- |
| | methylheptyl)-2-methyl-2-propoxypropanamide |
| I-33 | N-((2S,3S,5S)-5-(3-(3-methoxypropoxy)-4-methoxybenzyl)-3-amino-2-hydroxy-6- |
| | methylheptyl)-3-ethoxy-2,2-dimethylpropanamide |
| I-34 | N-((2S,3S,5S)-5-(3-(3-methoxypropoxy)-4-methoxybenzyl)-3-amino-2-hydroxy-6- |
| | methylheptyl)-3-phenylpropanamide |
| I-35 | N-((2S,3S,5S)-5-(3-(3-methoxypropoxy)-4-methoxybenzyl)-3-amino-2-hydroxy-6- |
| | methylheptyl)-2-m-tolylacetamide |
| I-36 | (2S,3S,5S)-5-(3-(3-methoxypropoxy)-4-methoxybenzyl)-3-amino-6-methyl-1- |
| | (pentanesulfonylamino)heptan-2-ol |
| I-37 | N-((2S,3S,5S)-5-(3-(3-methoxypropoxy)-4-methoxybenzyl)-3-amino-2-hydroxy-6- |
| | methylheptyl)-2-(2-fluorophenyl)acetamide |
| I-38 | N-((2S,3S,5S)-5-(3-(3-methoxypropoxy)-4-methoxybenzyl)-3-amino-2-hydroxy-6- |
| | methylheptyl)-2-(3-fluorophenyl)acetamide |
| I-39 | N-((2S,3S,5S)-5-(3-(3-methoxypropoxy)-4-methoxybenzyl)-3-amino-2-hydroxy-6- |
| | methylheptyl)-2-(4-fluorophenyl)acetamide |
| I-40 | N-((2S,3S,5S)-5-(3-(3-methoxypropoxy)-4-methoxybenzyl)-3-amino-2-hydroxy-6- |
| | methylheptyl)-2-(4-fluorophenyl)acetamide |
| I-41 | N-((2S,3S,5S)-5-(3-(3-methoxypropoxy)-4-methoxybenzyl)-3-amino-2-hydroxy-6- |
| | methylheptyl)-5,5,5-trifluoropentanamide |
| I-42 | N-((2S,3S,5S)-5-(3-(3-methoxypropoxy)-4-methoxybenzyl)-3-amino-2-hydroxy-6- |
| | methylheptyl)-4-cyclopropyl-2,2-dimethylbutanamide |
| I-43 | N-((2R,3S,5S)-5-(3-(3-methoxypropoxy)-4-methoxybenzyl)-3-amino-2-hydroxy-6- |
| | methylheptyl)-4-cyclopropyl-2,2-dimethylbutanamide |
| I-44 | (2S,3S,5S)-5-(3-(3-methoxypropoxy)-4-methoxybenzyl)-3-amino-6-methyl-1- |
| | (benzenesulfonylamino)heptan-2-ol |
| I-45 | N-((2S,3S,5S)-5-(3-(3-methoxypropoxy)-4-methoxybenzyl)-3-amino-2-hydroxy-6- |
| | methylheptyl)-3,5,5-trimethylhexanamide |
| I-46 | N-((2S,3S,5S)-5-(3-(3-methoxypropoxy)-4-methoxybenzyl)-3-amino-2-hydroxy-6- |
| | methylheptyl)-N,2,2-trimethylhexanamide |
| I-47 | N-((2S,3S,5S)-5-(3-(3-methoxypropoxy)-4-methoxybenzyl)-3-amino-2-hydroxy-6- |
| | methylheptyl)-2-(4-cyanophenyl)acetamide |
| I-48 | N-((2S,3S,5S)-5-(3-(3-methoxypropoxy)-4-methoxybenzyl)-3-amino-2-hydroxy-6- |
| | methylheptyl)-1-phenylcyclopropanecarboxamide |
| I-49 | N-((2S,3S,5S)-5-(3-(3-methoxypropoxy)-4-methoxybenzyl)-3-amino-2-hydroxy-6- |
| | methylheptyl)-4-phenylbutanamide |
| I-50 | N-((2S,3S,5S)-5-(3-(3-methoxypropoxy)-4-methoxybenzyl)-3-amino-2-hydroxy-6- |
| | methylheptyl)-2-methyl-2-phenylpropanamide |
| I-51 | N-((2R,3S,5S)-5-(3-(3-methoxypropoxy)-4-methoxybenzyl)-3-amino-2-hydroxy-6- |
| | methylheptyl)-2-methyl-2-phenylpropanamide |
| I-52 | N-((2S,3S,5S)-5-(3-(3-methoxypropoxy)-4-methoxybenzyl)-3-amino-2-hydroxy-6- |
| | methylheptyl)-2-cyclohexyl-2-methylpropanamide |
| I-53 | N-((2S,3S,5S)-5-(3-(3-methoxypropoxy)-4-methoxybenzyl)-3-amino-2-hydroxy-6- |
| | methylheptyl)-2-(3,4-difluorophenyl)acetamide |
| I-54 | N-((2S,3S,5S)-5-(3-(3-methoxypropoxy)-4-methoxybenzyl)-3-amino-2-hydroxy-6- |
| | methylheptyl)-2-(2,4-difluorophenyl)acetamide |
| I-55 | N-((2S,3S,5S)-5-(3-(3-methoxypropoxy)-4-methoxybenzyl)-3-amino-2-hydroxy-6- |
| | methylheptyl)-2-(2,3-difluorophenyl)acetamide |
| I-56 | (2S,3S,5S)-5-(3-(3-methoxypropoxy)-4-methoxybenzyl)-3-amino-6-methyl-1- |
| | (benzylsulfonylamino)heptan-2-ol |
| I-57 | N-((2S,3S,5S)-5-(3-(3-methoxypropoxy)-4-methoxybenzyl)-3-amino-2-hydroxy-6- |
| | methylheptyl)-1-p-tolylcyclopropanecarboxamide |
| I-58 | (2S,3S,5S)-5-(3-(3-methoxypropoxy)-4-methoxybenzyl)-3-amino-1-(N-isopropyl-N- |
| | butanesulfonylamino)-6-methylheptan-2-ol |
| I-59 | (2R,3S,5S)-5-(3-(3-methoxypropoxy)-4-methoxybenzyl)-3-amino-1-(N-isopropyl-N- |
| | butanesulfonylamino)-6-methylheptan-2-ol |
| I-60 | N-((2S,3S,5S)-5-(3-(3-methoxypropoxy)-4-methoxybenzyl)-3-amino-2-hydroxy-6- |
| | methylheptyl)-2-(4-fluorophenyl)-2-methylpropanamide |
| I-61 | N-((2R,3S,5S)-5-(3-(3-methoxypropoxy)-4-methoxybenzyl)-3-amino-2-hydroxy-6- |
| | methylheptyl)-2-(4-fluorophenyl)-2-methylpropanamide |
| I-62 | N-((2S,3S,5S)-5-(3-(3-methoxypropoxy)-4-methoxybenzyl)-3-amino-2-hydroxy-6- |
| | methylheptyl)-2-(4-fluorophenyl)-2-methylpropanamide |
| I-63 | N-((2S,3S,5S)-5-(3-(3-methoxypropoxy)-4-methoxybenzyl)-3-amino-2-hydroxy-6- |
| | methylheptyl)-2-cyclopentylhexanamide |
| I-64 | N-((2S,3S,5S)-5-(3-(3-methoxypropoxy)-4-methoxybenzyl)-3-amino-2-hydroxy-6- |
| | methylheptyl)-1-phenylcyclopentanecarboxamide |
| I-65 | N-((2S,3S,5S)-5-(3-(3-methoxypropoxy)-4-phenylbenzyl)-3-amino-2-hydroxy-6- |
| | methylheptyl)-2,2-dimethylhexanamide |
| I-66 | N-((2S,3S,5S)-5-(5-(3-methoxypropoxy)-2-phenylbenzyl)-3-amino-2-hydroxy-6- |
| | methylheptyl)-2,2-dimethylhexanamide |
| I-67 | N-((2S,3S,5S)-5-(3-(3-methoxypropoxy)-4-methoxybenzyl)-3-amino-2-hydroxy-6- |
| | methylheptyl)-1-(4-methoxyphenyl)cyclopropanecarboxamide |
| I-68 | N-((2S,3S,5S)-5-(3-(3-methoxypropoxy)-4-bromobenzyl)-3-amino-2-hydroxy-6- |
| | methylheptyl)-2,2-dimethylhexanamide |
| I-69 | N-((2S,3S,5S)-5-(3-(3-methoxypropoxy)-4-methoxybenzyl)-3-amino-2-hydroxy-6- |
| | methylheptyl)-3-hydroxy-2,2-dimethyl-3-phenylpropanamide |
| I-70 | N-((2S,3S,5S)-5-(3-(3-methoxypropoxy)-4-methoxybenzyl)-3-amino-2-hydroxy-6- |
| | methylheptyl)-1-(4-chlorophenyl)cyclopropanecarboxamide |
| I-71 | (2S,3S,5S)-5-(3-(3-methoxypropoxy)-4-methoxybenzyl)-3-amino-1-(N-isopropyl-N- |
| | benzenesulfonylamino)-6-methylheptan-2-ol |
| I-72 | N-((2S,3S,5S)-5-(3-(3-methoxypropoxy)-4-methoxybenzyl)-3-amino-2-hydroxy-6- |
| | methylheptyl)-2-(2-(trifluoromethyl)phenyl)acetamide |
| I-73 | N-((2S,3S,5S)-5-(3-(3-methoxypropoxy)-4-methoxybenzyl)-3-amino-2-hydroxy-6- |
| | methylheptyl)-2-(3-(trifluoromethyl)phenyl)acetamide |
| I-74 | N-((2S,3S,5S)-5-(3-(3-methoxypropoxy)-4-methoxybenzyl)-3-amino-2-hydroxy-6- |
| | methylheptyl)-2-(4-(trifluoromethyl)phenyl)acetamide |
| I-75 | N-((2S,3S,5S)-5-(3-(3-methoxypropoxy)-4-methoxybenzyl)-3-amino-2-hydroxy-6- |
| | methylheptyl)-1-phenylcyclohexanecarboxamide |
| I-76 | N-((2S,3S,5S)-5-(3-(3-methoxypropoxy)-4-methoxybenzyl)-3-amino-2-hydroxy-6- |
| | methylheptyl)-2-(4-cyanophenoxy)-2-methylpropanamide |
| I-77 | N-((2S,3S,5S)-5-(3-(3-methoxypropoxy)-4-methoxybenzyl)-3-amino-2-hydroxy-6- |
| | methylheptyl)-2,2-bis(trifluoromethyl)propanamide |
| I-78 | N-((2S,3S,5S)-5-(3-(3-methoxypropoxy)-4-methoxybenzyl)-3-amino-2-hydroxy-6- |
| | methylheptyl)-2-ethyl-2-(4-fluorophenyl)butanamide |
| I-79 | (2S,3S,5S)-5-(3-(3-methoxypropoxy)-4-methoxybenzyl)-3-amino-1-(N-isopropyl-N- |
| | benzylsulfonylamino)-6-methylheptan-2-ol |
| I-80 | N-((2S,3S,5S)-5-(3-(3-methoxypropoxy)-4-methoxybenzyl)-3-amino-2-hydroxy-6- |
| | methylheptyl)-2-(4-chlorophenoxy)-2-methylpropanamide |
| I-81 | N-((2S,3S,5S)-5-(3-(3-methoxypropoxy)-4-methoxybenzyl)-3-amino-2-hydroxy-6- |
| | methylheptyl)-2-(4-(trifluoromethoxy)phenyl)acetamide |
| I-82 | N-((2S,3S,5S)-5-(3-(3-methoxypropoxy)-4-methoxybenzyl)-3-amino-2-hydroxy-6- |
| | methylheptyl)-1-(4-fluorophenyl)cyclohexanecarboxamide |
| I-83 | N-((2S,3S,5S)-5-(3-(3-methoxypropoxy)-4-methoxybenzyl)-3-amino-2-hydroxy-6- |
| | methylheptyl)-1-(2,4-dichlorophenyl)cyclopropanecarboxamide |
| I-84 | 1-((2S,3S,5S)-5-(3-(3-methoxypropoxy)-4-methoxybenzyl)-3-amino-2-hydroxy-6- |
| | methylheptyl)urea |
| I-85 | 1-((2S,3S,5S)-5-(3-(3-methoxypropoxy)-4-methoxybenzyl)-3-amino-2-hydroxy-6- |
| | methylheptyl)-3-butylurea |
| I-86 | 1-((2S,3S,5S)-5-(3-(3-methoxypropoxy)-4-methoxybenzyl)-3-amino-2-hydroxy-6- |
| | methylheptyl)-3-tert-butylurea |
| I-87 | isobutyl (2S,3S,5S)-5-(3-(3-methoxypropoxy)-4-methoxybenzyl)-3-amino-2-hydroxy- |
| | 6-methylheptylcarbamate |
| I-88 | N-((2S,3S,5S)-5-(3-(3-methoxypropoxy)-4-methoxybenzyl)-3-amino-2-hydroxy-6- |
| | methylheptyl)piperidine-1-carboxamide |
| I-89 | 1-((2S,3S,5S)-5-(3-(3-methoxypropoxy)-4-methoxybenzyl)-3-amino-2-hydroxy-6- |
| | methylheptyl)-3-(2-cyclopropylethyl)urea |
| I-90 | N-((2S,3S,5S)-5-(3-(3-methoxypropoxy)-4-methoxybenzyl)-3-amino-2-hydroxy-6- |
| | methylheptyl)morpholine-4-carboxamide |
| I-91 | 1-((2S,3S,5S)-5-(3-(3-methoxypropoxy)-4-methoxybenzyl)-3-amino-2-hydroxy-6- |
| | methylheptyl)-3-pentylurea |
| I-92 | 1-((2S,3S,5S)-5-(3-(3-methoxypropoxy)-4-methoxybenzyl)-3-amino-2-hydroxy-6- |
| | methylheptyl)-3-pentylurea |
| I-93 | 1-((2R,3S,5S)-5-(3-(3-methoxypropoxy)-4-methoxybenzyl)-3-amino-2-hydroxy-6- |
| | methylheptyl)-3-pentylurea |
| I-94 | pentyl (2S,3S,5S)-5-(3-(3-methoxypropoxy)-4-methoxybenzyl)-3-amino-2-hydroxy-6- |
| | methylheptylcarbamate |
| I-95 | 1-((2S,3S,5S)-5-(3-(3-methoxypropoxy)-4-methoxybenzyl)-3-amino-2-hydroxy-6- |
| | methylheptyl)-3-(3-methoxypropyl)urea |
| I-96 | 1-((2S,3S,5S)-5-(3-(3-methoxypropoxy)-4-methoxybenzyl)-3-amino-2-hydroxy-6- |
| | methylheptyl)-3-(2-ethoxyethyl)urea |
| I-97 | 1-((2S,3S,5S)-5-(3-(3-methoxypropoxy)-4-methoxybenzyl)-3-amino-2-hydroxy-6- |
| | methylheptyl)-3-cyclohexylurea |
| I-98 | 1-((2S,3S,5S)-5-(3-(3-methoxypropoxy)-4-methoxybenzyl)-3-amino-2-hydroxy-6- |
| | methylheptyl)-3-hexylurea |
| I-99 | 3-((2S,3S,5S)-5-(3-(3-methoxypropoxy)-4-methoxybenzyl)-3-amino-2-hydroxy-6- |
| | methylheptyl)-1-methyl-1-pentylurea |
| I-100 | 1-((2S,3S,5S)-5-(3-(3-methoxypropoxy)-4-methoxybenzyl)-3-amino-2-hydroxy-6- |
| | methylheptyl)-1-methyl-1-pentylurea |
| I-101 | 1-((2S,3S,5S)-5-(3-(3-methoxypropoxy)-4-methoxybenzyl)-3-amino-2-hydroxy-6- |
| | methylheptyl)-3-pentylthiourea |
| I-102 | 1-((2S,3S,5S)-5-(3-(3-methoxypropoxy)-4-methoxybenzyl)-3-amino-2-hydroxy-6- |
| | methylheptyl)-3-benzylurea |
| I-103 | benzyl (2S,3S,5S)-5-(3-(3-methoxypropoxy)-4-methoxybenzyl)-3-amino-2-hydroxy- |
| | 6-methylheptylcarbamate |
| I-104 | (2S,3S,5S)-5-(3-(3-methoxypropoxy)-4-methoxybenzyl)-3-amino-1- |
| | (butylaminosulfonylamino)-6-methylheptan-2-ol |
| I-105 | 1-((2S,3S,5S)-5-(3-(3-methoxypropoxy)-4-methoxybenzyl)-3-amino-2-hydroxy-6- |
| | methylheptyl)-3-(3-fluorophenyl)urea |
| I-106 | 1-((2S,3S,5S)-5-(3-(3-methoxypropoxy)-4-methoxybenzyl)-3-amino-2-hydroxy-6- |
| | methylheptyl)-3-(cyclohexylmethyl)urea |
| I-107 | 3-((2S,3S,5S)-5-(3-(3-methoxypropoxy)-4-methoxybenzyl)-3-amino-2-hydroxy-6- |
| | methylheptyl)-1-cyclohexyl-1-methylurea |
| I-108 | (2S,3S,5S)-5-(3-(3-methoxypropoxy)-4-methoxybenzyl)-3-amino-1-(N- |
| | (butylaminosulfonyl)-N-isopropylamino)-6-methylheptan-2-ol |
| I-109 | 1-((2S,3S,5S)-5-(3-(3-methoxypropoxy)-4-methoxybenzyl)-3-amino-2-hydroxy-6- |
| | methylheptyl)-3-(2,2-dimethylpentyl)urea |
| I-110 | 1-((2S,3S,5S)-5-(3-(3-methoxypropoxy)-4-methoxybenzyl)-3-amino-2-hydroxy-6- |
| | methylheptyl)-3-(2-methylhexan-2-yl)urea |
| I-111 | 1-((2S,3S,5S)-5-(3-(3-methoxypropoxy)-4-methoxybenzyl)-3-amino-2-hydroxy-6- |
| | methylheptyl)-3-(2-carbamoyl-2-methylpropyl)urea |
| I-112 | 1-((2S,3S,5S)-5-(3-(3-methoxypropoxy)-4-methoxybenzyl)-3-amino-2-hydroxy-6- |
| | methylheptyl)-3-phenethylurea |
| I-113 | (2S,3S,5S)-5-(3-(3-methoxypropoxy)-4-methoxybenzyl)-3-amino-1- |
| | (pentylaminosulfonylamino)-6-methylheptan-2-ol |
| I-114 | 1-((2S,3S,5S)-5-(3-(3-methoxypropoxy)-4-methoxybenzyl)-3-amino-2-hydroxy-6- |
| | methylheptyl)-3-(2-cyclohexylethyl)urea |
| I-115 | 1-((2S,3S,5S)-5-(3-(3-methoxypropoxy)-4-methoxybenzyl)-3-amino-2-hydroxy-6- |
| | methylheptyl)-3-(2,4,4-trimethylpentan-2-yl)urea |
| I-116 | 3-((2S,3S,5S)-5-(3-(3-methoxypropoxy)-4-methoxybenzyl)-3-amino-2-hydroxy-6- |
| | methylheptyl)-1,1-diisobutylurea |
| I-117 | 1-((2S,3S,5S)-5-(3-(3-methoxypropoxy)-4-methoxybenzyl)-3-amino-2-hydroxy-6- |
| | methylheptyl)-3-(3-phenylpropyl)urea |
| I-118 | (2S,3S,5S)-5-(3-(3-methoxypropoxy)-4-methoxybenzyl)-3-amino-1-(N- |
| | (allylaminosulfonyl)-N-isopropylamino)-6-methylheptan-2-ol |
| I-119 | 1-((2S,3S,5S)-5-(3-(3-methoxypropoxy)-4-methoxybenzyl)-3-amino-2-hydroxy-6- |
| | methylheptyl)-3-(3-(trifluoromethyl)phenyl)urea and |
| I-120 | 1-((2S,3S,5S)-5-(3-(3-methoxypropoxy)-4-methoxybenzyl)-3-amino-2-hydroxy-6- |
| | methylheptyl)-3-(1-(4-fluorophenyl)-2-methylpropan-2-yl)urea. |

A preferred embodiment of the invention is each of the following compounds or their enantiomers, diastereomers, and pharmaceutically acceptable salts:

| Cpd. No. | Name |
|---|---|
| I-6 | N-((2S,3S,5S)-5-(3-(3-methoxypropoxy)-4-methoxybenzyl)-3-amino-2-hydroxy-6- |
| | methylheptyl)hexanamide |
| I-16 | N-((2S,3S,5S)-5-(3-(3-methoxypropoxy)-4-methoxybenzyl)-3-amino-2-hydroxy-6- |
| | methylheptyl)-2,2-dimethylpentanamide |
| I-17 | N-((2S,3S,5S)-5-(3-(3-methoxypropoxy)-4-methoxybenzyl)-3-amino-2-hydroxy-6- |
| | methylheptyl)-2-methylhexanamide |
| I-21 | N-((2S,3S,5S)-5-(3-(3-methoxypropoxy)-4-methoxybenzyl)-3-amino-2-hydroxy-6- |
| | methylheptyl)-2-cyclohexylacetamide |
| I-26 | N-((2R,3S,5S)-5-(3-(3-methoxypropoxy)-4-ethylbenzyl)-3-amino-2-hydroxy-6- |
| | methylheptyl)-2,2-dimethylhexanamide |
| I-28 | N-((2S,3S,5S)-5-(3-(3-methoxypropoxy)-4-methoxybenzyl)-3-amino-2-hydroxy-6- |
| | methylheptyl)-2,2-dimethylhexanamide |
| I-28 | N-((2S,3S,5S)-5-(3-(3-methoxypropoxy)-4-methoxybenzyl)-3-amino-2-hydroxy-6- |
| | methylheptyl)-2,2-dimethylhexanamide |
| I-31 | N-((2S,3S,5S)-5-(3-(3-methoxypropoxy)-4-methoxybenzyl)-3-amino-2-hydroxy-6- |
| | methylheptyl)-2-ethylhexanamide |
| I-33 | N-((2S,3S,5S)-5-(3-(3-methoxypropoxy)-4-methoxybenzyl)-3-amino-2-hydroxy-6- |
| | methylheptyl)-3-ethoxy-2,2-dimethylpropanamide |
| I-39 | N-((2S,3S,5S)-5-(3-(3-methoxypropoxy)-4-methoxybenzyl)-3-amino-2-hydroxy-6- |
| | methylheptyl)-2-(4-fluorophenyl)acetamide |
| I-40 | N-((2S,3S,5S)-5-(3-(3-methoxypropoxy)-4-methoxybenzyl)-3-amino-2-hydroxy-6- |
| | methylheptyl)-2-(4-fluorophenyl)acetamide |
| I-42 | N-((2S,3S,5S)-5-(3-(3-methoxypropoxy)-4-methoxybenzyl)-3-amino-2-hydroxy-6- |
| | methylheptyl)-4-cyclopropyl-2,2-dimethylbutanamide |
| I-50 | N-((2S,3S,5S)-5-(3-(3-methoxypropoxy)-4-methoxybenzyl)-3-amino-2-hydroxy-6- |
| | methylheptyl)-2-methyl-2-phenylpropanamide |
| I-52 | N-((2S,3S,5S)-5-(3-(3-methoxypropoxy)-4-methoxybenzyl)-3-amino-2-hydroxy-6- |
| | methylheptyl)-2-cyclohexyl-2-methylpropanamide |
| I-60 | N-((2S,3S,5S)-5-(3-(3-methoxypropoxy)-4-methoxybenzyl)-3-amino-2-hydroxy-6- |
| | methylheptyl)-2-(4-fluorophenyl)-2-methylpropanamide |
| I-62 | N-((2S,3S,5S)-5-(3-(3-methoxypropoxy)-4-methoxybenzyl)-3-amino-2-hydroxy-6- |
| | methylheptyl)-2-(4-fluorophenyl)-2-methylpropanamide |
| I-64 | N-((2S,3S,5S)-5-(3-(3-methoxypropoxy)-4-methoxybenzyl)-3-amino-2-hydroxy-6- |
| | methylheptyl)-1-phenylcyclopentanecarboxamide |
| I-74 | N-((2S,3S,5S)-5-(3-(3-methoxypropoxy)-4-methoxybenzyl)-3-amino-2-hydroxy-6- |
| | methylheptyl)-2-(4-(trifluoromethyl)phenyl)acetamide |
| I-75 | N-((2S,3S,5S)-5-(3-(3-methoxypropoxy)-4-methoxybenzyl)-3-amino-2-hydroxy-6- |
| | methylheptyl)-1-phenylcyclohexanecarboxamide |
| I-78 | N-((2S,3S,5S)-5-(3-(3-methoxypropoxy)-4-methoxybenzyl)-3-amino-2-hydroxy-6- |
| | methylheptyl)-2-ethyl-2-(4-fluorophenyl)butanamide |
| I-82 | N-((2S,3S,5S)-5-(3-(3-methoxypropoxy)-4-methoxybenzyl)-3-amino-2-hydroxy-6- |
| | methylheptyl)-1-(4-fluorophenyl)cyclohexanecarboxamide |
| I-85 | 1-((2S,3S,5S)-5-(3-(3-methoxypropoxy)-4-methoxybenzyl)-3-amino-2-hydroxy-6- |
| | methylheptyl)-3-butylurea |
| I-91 | 1-((2S,3S,5S)-5-(3-(3-methoxypropoxy)-4-methoxybenzyl)-3-amino-2-hydroxy-6- |
| | methylheptyl)-3-pentylurea |
| I-92 | 1-((2S,3S,5S)-5-(3-(3-methoxypropoxy)-4-methoxybenzyl)-3-amino-2-hydroxy-6- |
| | methylheptyl)-3-pentylurea |
| I-98 | 1-((2S,3S,5S)-5-(3-(3-methoxypropoxy)-4-methoxybenzyl)-3-amino-2-hydroxy-6- |
| | methylheptyl)-3-hexylurea |
| I-99 | 3-((2S,3S,5S)-5-(3-(3-methoxypropoxy)-4-methoxybenzyl)-3-amino-2-hydroxy-6- |
| | methylheptyl)-1-methyl-1-pentylurea |
| I-109 | 1-((2S,3S,5S)-5-(3-(3-methoxypropoxy)-4-methoxybenzyl)-3-amino-2-hydroxy-6- |
| | methylheptyl)-3-(2,2-dimethylpentyl)urea |
| I-115 | 1-((2S,3S,5S)-5-(3-(3-methoxypropoxy)-4-methoxybenzyl)-3-amino-2-hydroxy-6- |
| | methylheptyl)-3-(2,4,4-trimethylpentan-2-yl)urea and |
| I-116 | 3-((2S,3S,5S)-5-(3-(3-methoxypropoxy)-4-methoxybenzyl)-3-amino-2-hydroxy-6- |
| | methylheptyl)-1,1-diisobutylurea. |

A more preferred embodiment of the invention is each of the following compounds or their enantiomers, diastereomers, and pharmaceutically acceptable salts:

| Cpd. No. | Name |
|---|---|
| I-28 | N-((2S,3S,5S)-5-(3-(3-methoxypropoxy)-4-methoxybenzyl)-3-amino-2-hydroxy-6- |
| | methylheptyl)-2,2-dimethylhexanamide |
| I-42 | N-((2S,3S,5S)-5-(3-(3-methoxypropoxy)-4-methoxybenzyl)-3-amino-2-hydroxy-6- |
| | methylheptyl)-4-cyclopropyl-2,2-dimethylbutanamide |
| I-50 | N-((2S,3S,5S)-5-(3-(3-methoxypropoxy)-4-methoxybenzyl)-3-amino-2-hydroxy-6- |
| | methylheptyl)-2-methyl-2-phenylpropanamide |
| I-52 | N-((2S,3S,5S)-5-(3-(3-methoxypropoxy)-4-methoxybenzyl)-3-amino-2-hydroxy-6- |
| | methylheptyl)-2-cyclohexyl-2-methylpropanamide and |
| I-62 | N-((2S,3S,5S)-5-(3-(3-methoxypropoxy)-4-methoxybenzyl)-3-amino-2-hydroxy-6- |
| | methylheptyl)-2-(4-fluorophenyl)-2-methylpropanamide. |

Another embodiment of the invention is each of the following compounds and their enantiomers, diastereomers, and pharmaceutically acceptable salts:

| Cpd. No. | Name |
|---|---|
| I-6 | N-((2S,3S,5S)-5-(3-(3-methoxypropoxy)-4-methoxybenzyl)-3-amino-2-hydroxy-6- |
| | methylheptyl)hexanamide |
| I-17 | N-((2S,3S,5S)-5-(3-(3-methoxypropoxy)-4-methoxybenzyl)-3-amino-2-hydroxy-6- |
| | methylheptyl)-2-methylhexanamide |
| I-21 | N-((2S,3S,5S)-5-(3-(3-methoxypropoxy)-4-methoxybenzyl)-3-amino-2-hydroxy-6- |
| | methylheptyl)-2-cyclohexylacetamide |
| I-26 | N-((2R,3S,5S)-5-(3-(3-methoxypropoxy)-4-ethylbenzyl)-3-amino-2-hydroxy-6- |
| | methylheptyl)-2,2-dimethylhexanamide |
| I-31 | N-((2S,3S,5S)-5-(3-(3-methoxypropoxy)-4-methoxybenzyl)-3-amino-2-hydroxy-6- |
| | methylheptyl)-2-ethylhexanamide |
| I-33 | N-((2S,3S,5S)-5-(3-(3-methoxypropoxy)-4-methoxybenzyl)-3-amino-2-hydroxy-6- |
| | methylheptyl)-3-ethoxy-2,2-dimethylpropanamide |
| I-39 | N-((2S,3S,5S)-5-(3-(3-methoxypropoxy)-4-methoxybenzyl)-3-amino-2-hydroxy-6- |
| | methylheptyl)-2-(4-fluorophenyl)acetamide |
| I-40 | N-((2S,3S,5S)-5-(3-(3-methoxypropoxy)-4-methoxybenzyl)-3-amino-2-hydroxy-6- |
| | methylheptyl)-2-(4-fluorophenyl)acetamide |
| I-42 | N-((2S,3S,5S)-5-(3-(3-methoxypropoxy)-4-methoxybenzyl)-3-amino-2-hydroxy-6- |
| | methylheptyl)-4-cyclopropyl-2,2-dimethylbutanamide |
| I-74 | N-((2S,3S,5S)-5-(3-(3-methoxypropoxy)-4-methoxybenzyl)-3-amino-2-hydroxy-6- |
| | methylheptyl)-2-(4-(trifluoromethyl)phenyl)acetamide |
| I-75 | N-((2S,3S,5S)-5-(3-(3-methoxypropoxy)-4-methoxybenzyl)-3-amino-2-hydroxy-6- |
| | methylheptyl)-1-phenylcyclohexanecarboxamide |
| I-78 | N-((2S,3S,5S)-5-(3-(3-methoxypropoxy)-4-methoxybenzyl)-3-amino-2-hydroxy-6- |
| | methylheptyl)-2-ethyl-2-(4-fluorophenyl)butanamide |
| I-82 | N-((2S,3S,5S)-5-(3-(3-methoxypropoxy)-4-methoxybenzyl)-3-amino-2-hydroxy-6- |
| | methylheptyl)-1-(4-fluorophenyl)cyclohexanecarboxamide |
| I-85 | 1-((2S,3S,5S)-5-(3-(3-methoxypropoxy)-4-methoxybenzyl)-3-amino-2-hydroxy-6- |
| | methylheptyl)-3-butylurea |
| I-91 | 1-((2S,3S,5S)-5-(3-(3-methoxypropoxy)-4-methoxybenzyl)-3-amino-2-hydroxy-6- |
| | methylheptyl)-3-pentylurea |
| I-92 | 1-((2S,3S,5S)-5-(3-(3-methoxypropoxy)-4-methoxybenzyl)-3-amino-2-hydroxy-6- |
| | methylheptyl)-3-pentylurea |
| I-98 | 1-((2S,3S,5S)-5-(3-(3-methoxypropoxy)-4-methoxybenzyl)-3-amino-2-hydroxy-6- |
| | methylheptyl)-3-hexylurea |
| I-99 | 3-((2S,3S,5S)-5-(3-(3-methoxypropoxy)-4-methoxybenzyl)-3-amino-2-hydroxy-6- |
| | methylheptyl)-1-methyl-1-pentylurea |
| I-109 | 1-((2S,3S,5S)-5-(3-(3-methoxypropoxy)-4-methoxybenzyl)-3-amino-2-hydroxy-6- |
| | methylheptyl)-3-(2,2-dimethylpentyl)urea |
| I-115 | 1-((2S,3S,5S)-5-(3-(3-methoxypropoxy)-4-methoxybenzyl)-3-amino-2-hydroxy-6- |
| | methylheptyl)-3-(2,4,4-trimethylpentan-2-yl)urea and |
| I-116 | 3-((2S,3S,5S)-5-(3-(3-methoxypropoxy)-4-methoxybenzyl)-3-amino-2-hydroxy-6- |
| | methylheptyl)-1,1-diisobutylurea. |

Another more preferred embodiment of the invention is each of the following compounds or their enantiomers, diastereomers, and pharmaceutically acceptable salts:

| Cpd. | |
|---|---|
| No. | Name |
| I-42 | N-((2S,3S,5S)-5-(3-(3-methoxypropoxy)-4-methoxybenzyl)-3- |
| | amino-2-hydroxy-6-methylheptyl)-4-cyclopropyl- |
| | 2,2-dimethylbutanamide |

Other embodiments of the invention are the intermediates used for the preparation of the compounds of the invention, especially the intermediates resulting in the preferred compounds of formula I, to processes for their preparation, and to their use as intermediates. This primarily relates to compounds of formulae II and III:

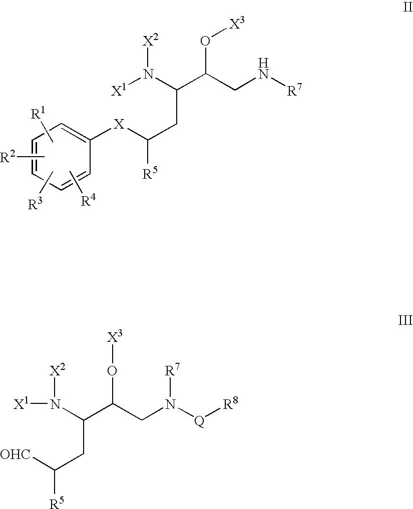

which are suitable as intermediates for the preparation of compounds of formula 1.

Thus, another embodiment of the invention is a compound of formula II, wherein

R¹ is hydrogen, halogen, hydroxy, lower alkoxy, cycloalkoxy, lower alkoxy-lower alkoxy, lower alkylthio-lower alkoxy, cyano-lower alkoxy, hydroxy-lower alkoxy, carboxy-lower alkoxy, lower alkoxycarbonyl-lower alkoxy, carbamoyl-lower alkoxy, N-mono- or N,N-di-lower alkylcarbamoyl-lower alkoxy, or aryl;
R² is hydrogen, halogen, cyano, carbamoyl, lower alkyl, lower haloalkyl, cycloalkyl, halocycloalkyl, cycloalkyl-lower alkyl, halocycloalkyl-loweralkyl, cyano-lower alkyl, hydroxy-lower alkyl, lower alkoxy-lower alkyl, lower haloalkoxy-lower alkyl, lower alkoxy-lower alkyl, lower haloalkoxy-lower alkyl, cycloalkoxy-lower alkyl, halocycloalkoxy-lower alkyl, hydroxy, lower alkanoyloxy-lower alkoxy, hydroxy-lower alkoxy, halo-(hydroxy)-lower alkoxy, lower alkanesulfonyl-(hydroxy)-lower alkoxy, amino-lower alkyl, lower alkylamino-lower alkyl, di-lower alkylamino-lower alkyl, lower alkanoylamino-lower alkyl, lower alkoxycarbonyl-amino-lower alkyl, aminocarbonylamino-lower alkyl, lower alkylaminocarbonylamino-lower alkyl, di(lower alkyl)aminocarbonylamino-lower alkyl, aminosulfonylamino-lower alkyl, lower alkylaminosulfonylamino-lower alkyl, di(lower alkyl)aminosulfonylamino-lower alkyl, amino-lower alkoxy, lower alkylamino-lower alkoxy, di-lower alkylamino-lower alkoxy, lower alkanoylamino-lower alkoxy, lower alkoxycarbonyl-amino-lower alkoxy, aminocarbonylamino-lower alkoxyl, lower alkylaminocarbonylamino-lower alkoxy, di(lower alkyl)aminocarbonylamino-lower alkoxy, aminosulfonylamino-lower alkoxy, lower alkylaminosulfonylamino-lower alkoxy, di(lower alkyl)aminosulfonylamino-lower alkoxy, oxo-lower alkoxy, lower alkoxy, lower haloalkoxy, cycloalkoxy, lower halocycloalkoxy, cycloalkyl-lower alkoxy, halocycloalkyl-lower alkoxy, lower alkenyloxy, cycloalkoxy-lower alkoxy, halocycloalkoxy-lower alkoxy, lower alkoxy-lower alkoxy, lower haloalkoxy-lower alkyl, lower alkoxy-lower alkenyl, lower alkenyloxy-lower alkoxy, lower alkoxy-lower alkenyloxy, lower alkenyloxy-lower alkyl, lower alkanoyl-lower alkoxy, lower alkylthio-lower alkoxy, lower alkanesulfonyl-lower alkoxy, lower alkylthio-(hydroxy)-lower alkoxy, aryl-lower alkoxy, optionally N-oxidized pyridyl-lower alkoxy, thiazolylthio-lower alkoxy or thiazolinylthio-lower alkoxy, imidazolylthio-lower alkoxy, optionally N-oxidized pyridylthio-lower alkoxy, pyrimidinylthio-lower alkoxy, cyano-lower alkoxy, carboxy-lower alkoxy, lower alkoxycarbonyl-lower alkoxy, carbamoyl-lower alkoxy, N-mono- or N,N-di-lower alkylcarbamoyl-lower alkoxy, carboxy-lower alkyl, lower alkoxycarbonyl-lower alkyl, carbamoyl-lower alkyl, or N-mono- or N,N-di-lower alkylcarbamoyl-lower alkyl;
R³ is hydrogen, halogen, cyano, carbamoyl, lower alkyl, lower haloalkyl, lower alkoxy-lower alkyl, cycloalkoxy-lower alkyl, hydroxy-lower alkyl, lower alkylthio-lower alkyl, lower alkanesulfonyl-lower alkyl, optionally partially hydrogenated or N-oxidized pyridyl-lower alkyl, thiazolyl-thio-lower alkyl or thiazolinylthio-lower alkyl, imidazolylthio-lower alkyl, optionally N-oxidized pyridylthio-lower alkyl, pyrimidinylthio-lower alkyl, amino-lower alkyl, lower alkylamino-lower alkyl, di-lower alkylamino-lower alkyl, lower alkanoyl-amino-lower alkyl, lower alkanesulfonylamino-lower alkyl, polyhalo-lower alkane-sulfonylamino-lower alkyl, pyrrolidino-lower alkyl, piperidino-lower alkyl, piperazino-lower alkyl, N′-lower alkylpiperazino-lower alkyl or N′-lower alkanoylpiperazino-lower alkyl, morpholino-lower alkyl, thiomorpholino-lower alkyl, S-oxothiomorpholino-lower alkyl or S,S-dioxothio-morpholino-lower alkyl, cyano-lower alkyl, carboxy-lower alkyl, lower alkoxy-carbonyl-lower alkyl, carbamoyl-lower alkyl, N-mono- or N,N-di-lower alkyl-carbamoyl-lower alkyl, cycloalkyl; phenyl or naphthyl that is unsubstituted or substituted with one to three groups independently selected from lower alkyl, lower alkoxy, hydroxy, lower alkylamino, di-lower alkylamino, halogen, trifluoromethyl, trifluoromethoxy, and cyano; hydroxy, lower alkoxy, cycloalkoxy, lower alkoxy-lower alkoxy, cycloalkoxy-lower alkoxy, hydroxy-lower alkoxy, aryl, lower haloalkoxy, lower alkylthio-lower alkoxy, lower haloalkylthio-lower alkoxy, lower alkanesulfonyl-lower alkoxy, lower haloalkanesulfonyl-lower alkoxy, optionally hydrogenated heteroaryl-lower alkoxy, heterocyclyl-lower alkoxy, optionally partially or fully hydrogenated heteroarylthio-lower alkoxy, such as thiazolylthio-lower alkoxy or thiazolinylthio-lower alkoxy, imidazolylthio-lower alkoxy, optionally N-oxidized pyridylthio-lower alkoxy, pyrimidinylthio-lower alkoxy, amino-lower alkoxy, lower alkylamino-lower alkoxy, di-lower alkylamino-lower alkoxy, lower alkanoylamino-lower alkoxy, lower alkanesulfonylamino-lower alkoxy, polyhalo-lower alkanesulfonylamino-lower alkoxy, pyrrolidino-lower alkoxy, piperidino-lower alkoxy, piperazino-lower alkoxy, N′-lower alkylpiperazino-lower alkoxy or N′-lower alkanoylpiperazino-lower alkoxy, morpholino-lower alkoxy, thiomorpholino-lower alkoxy, S-oxothiomorpholino-lower alkoxy or S,S-dioxothiomorpholino-lower alkoxy, cyano-lower alkoxy, carboxy-lower alkoxy, lower alkoxycarbonyl-lower alkoxy, carbamoyl-lower alkoxy, N-mono- or N,N-di-lower alkylcarbamoyl-lower alkoxy, carboxy-lower alkyl, lower alkoxycarbonyl-lower alkyl, carbamoyl-lower alkyl, or N-mono- or N,N-di-lower alkylcarbamoyl-lower alkyl; or
R² and R³ taken together with the atoms through which they are attached form a fused dioxolane, dioxane, benzene or cyclohexene ring, wherein said ring is substituted with up to 2 substituents independently selected from lower alkyl and lower alkoxy-lower alkyl;
R⁴ is hydrogen, lower alkyl, hydroxy, lower alkoxy, cycloalkoxy, lower alkoxy-lower alkoxy, or cycloalkyl-lower alkoxy; or
R³ and R⁴ taken together with the atoms through which they are attached form a fused dioxolane, dioxane, benzene or cyclohexene ring, wherein said ring is substituted with up to 2 substituents independently selected from lower alkyl and lower alkoxy-lower alkyl; provided that R³ does not form a ring with R²;
X is methylene or hydroxymethylene;
R⁵ is lower alkyl, lower haloalkyl, cycloalkyl, halocycloalkyl, lower alkyl-cycloalkyl, lower haloalkyl-cycloalkyl, cycloalkyl-lower alkyl, aryl, aryl-lower alkyl, heterocyclyl, heterocyclyl-lower alkyl;
R⁶ is amino, lower alkylamino, di-lower alkylamino, or lower alkanoylamino;
R⁷ is hydrogen, lower alkyl, lower haloalkyl, cycloalkyl, lower alkoxy-lower alkyl, or lower haloalkoxy-lower alkyl;
X¹ is an amino-protecting group;
X² is hydrogen or together with X³ is a bivalent protecting group;
X³ is hydrogen or a hydroxy-protecting group;
and the enantiomers, diastereomers, and salts thereof;
and their use as intermediates for the preparation of medicinal active ingredients, especially of compounds of formula I.

Another embodiment of the invention is a compound of formula IIa

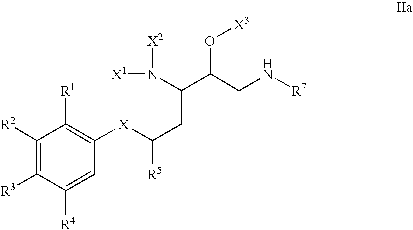

in which the substituents R¹-R⁵, R⁶, R⁷, X and X¹-X³ are as defined for formula II.

Another embodiment of the invention is a compound of formula IIa, wherein

R¹ is hydrogen or aryl;
R² is hydrogen, lower alkyl, cycloalkyl-lower alkyl, lower alkoxy-lower alkyl, lower haloalkoxy-lower alkyl, lower alkoxy-lower alkoxy, lower haloalkoxy-lower alkoxy, lower alkoxy-lower alkyl; cycloalkyl-lower alkoxy, phenyl-lower alkoxy that is unsubstituted or substituted by lower alkyl, lower alkoxy, hydroxy, halogen, nitro and/or by amino; optionally N-oxidized pyridyl-lower alkoxy, lower alkylthio-lower alkoxy, lower alkane-sulfonyl-lower alkoxy, lower alkanoyl-lower alkoxy, cyano-lower alkoxy, carboxy-lower alkoxy, lower alkoxycarbonyl-lower alkoxy, carbamoyl-lower alkoxy, lower alkylcarbamoyl-lower alkoxy, or di-lower alkylcarbamoyl-lower alkoxy;
R³ is hydrogen, halogen, cyano, lower alkyl, lower haloalkyl, aryl, hydroxy, lower alkoxy, or polyhalo-lower alkoxy; or
R² and R³ taken together with the atoms through which they are attached form a fused dioxolane ring, which is substituted with up to 2 substituents independently selected from lower alkyl and lower alkoxy-lower alkyl;
R⁴ is hydrogen, lower alkoxy-lower alkoxy, lower alkoxy-lower alkyl, or cycloalkyl-lower alkoxy; or
R³ and R⁴ taken together with the atoms through which they are attached form a fused dioxolane ring which is substituted with up to 2 substituents independently selected from lower alkyl and lower alkoxy-lower alkyl; provided that R³ does not form a ring with R²;
X is methylene or hydroxymethylene;
R⁵ is lower alkyl or cycloalkyl;
R⁷ is hydrogen or methyl;
X¹ is lower alkoxycarbonyl, 2-(trialkylsily)ethoxycarbonyl, or α-phenyl- or α,α-diphenyl-lower alkoxycarbonyl that is unsubstituted or substituted by lower alkyl, lower alkoxy, nitro and/or by halogen, or is 2-halo-lower alkoxycarbonyl;
X² is hydrogen or together with X³ is carbonyl or lower alkylidene;
X³ is hydrogen, tri-lower alkylsilyl;
and the enantomers, diastereomers, and salts thereof.

Another embodiment of the invention is a compound formula IIa wherein

R¹ is hydrogen;
R² is (C₁-C₄)alkoxy-(C₁-C₄)alkoxy, (C₁-C₄)alkoxy-(C₁-C₄)alkyl, or cycloalkyl-lower alkoxy;
R³ is fluoro, chloro, bromo, cyano, (C₁-C₄)alkyl, (C₁-C₄)haloalkyl, aryl, (C₁-C₄)alkoxy, or (C₁-C₄)haloalkoxy;
R⁴ is hydrogen;
X is methylene;
R⁵ is (C₃-C₅)alkyl;
R⁷ is hydrogen;
X¹ is lower alkoxycarbonyl, or α-phenyl-lower alkoxycarbonyl that is unsubstituted or substituted by lower alkyl, lower alkoxy, nitro, and/or by halogen;
X² and X³ are both hydrogen, or taken together are lower alkylidene;
and the enantiomers, diastereomers, and salts thereof.

Another embodiment of the invention is compounds of formula IIa wherein:

R¹ is hydrogen;
R² is 3-methoxypropoxy, 3-ethoxypropoxy, 4-methoxybutyl, or 2-(cyclopropyl)ethoxy;
R³ is fluoro, chloro, bromo, cyano, methyl, ethyl, isopropyl or tert-butyl, trifluoromethyl, pentafluoroethyl, phenyl, methoxy, difluoromethoxy, or trifluoromethoxy;
R⁴ is hydrogen;
X is methylene;
R⁵ is branched (C₃-C₅)alkyl;
R⁷ is hydrogen;
X¹ is lower alkoxycarbonyl, or α-phenyl-lower alkoxycarbonyl that is unsubstituted or substituted by lower alkyl, lower alkoxy, nitro, and/or by halogen;
X² and X³ are both hydrogen, or taken together are lower alkylidene;
and the enantiomers, diastereomers, and salts thereof.

Another embodiment of the invention is a compound of formula IIa wherein at least one, for example one, two or preferably all, of the asymmetric carbon atoms of the main chain have the stereochemical configuration shown in formula IIb

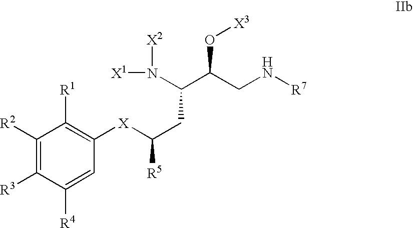

the variables each being as defined for formula IIa, and the salts thereof.

Another embodiment of the invention is a compound of formula lib wherein

R¹ and R⁴ is each hydrogen;
R² is 3-methoxypropoxy, 3-ethoxypropoxy, 4-methoxybutyl, or 2-(cyclopropyl)ethoxy;
R³ is fluoro, chloro, bromo, cyano, methyl, ethyl, isopropyl or tert-butyl, trifluoromethyl, pentafluoroethyl, phenyl, methoxy, difluoromethoxy, or trifluoromethoxy;
X is methylene;
R⁵ is isopropyl;
R⁷ is hydrogen;
X¹ is tert-butoxycarbonyl; and
X² and X³ are both hydrogen, or taken together are isopropylidene
and the salts thereof.

Preferred are compounds of formulae II, IIa, and IIb in the Examples and the salts thereof.

Another embodiment of the invention is compounds of formula III

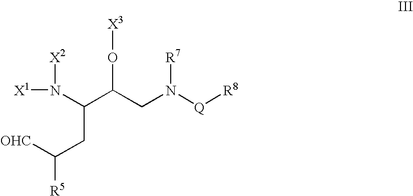

wherein
R⁵ is lower alkyl or cycloalkyl;
R⁷ is hydrogen, lower alkyl, lower haloalkyl, cycloalkyl, lower alkoxy-lower alkyl, or lower haloalkoxy-lower alkyl
Q is carbonyl, thiocarbonyl, or sulfonyl;
R⁸ is lower alkyl, lower haloalkyl, C₈-C₁₅alkyl, C₈-C₁₅haloalkyl, cycloalkyl, halocycloalkyl, lower alkyl-cycloalkyl, cycloalkyl-lower alkyl, halocycloalkyl-lower alkyl, lower alkoxy-loweralkyl, lower haloalkoxy-lower alkyl, cycloalkoxy-lower alkyl, cycloalkoxy-cycloalkyl, lower alkylthio-lower alkyl, lower haloalkylthio-lower alkyl, lower alkanesulfonyl-lower alkyl, lower haloalkanesulfonyl-lower alkyl, lower alkylthio-cycloalkyl, lower haloalkylthio-cycloalkyl, lower alkanesulfonyl-cycloalkyl, lower haloalkanesulfonyl-cycloalkyl, aryl, aryl-lower alkyl, aryl-lower hydroxyalkyl, arylcycloalkyl, aryloxy-lower alkyl, aryloxy cycloalkyl, arylthio-lower alkyl, arylsulfonyl-lower alkyl, arylthio-cycloalkyl, arylsulfonyl-cycloalkyl, lower alkanoyl-lower alkyl, hydroxy-lower alkyl, amino-lower alkyl, lower alkanoylamino-lower alkyl, N-mono-lower alkylamino-lower alkyl, N,N-di-lower alkylamino-lower alkyl, piperidino-lower alkyl, hydroxypiperidino-lower alkyl, lower alkoxypiperidino-lower alkyl, morpholino-lower alkyl, dimethylmorpholino-lower alkyl, thiomorpholino-lower alkyl, S,S-dioxothiomorpholino-lower alkyl, carboxy-lower alkyl, lower alkoxycarbonyl-lower alkyl, carbamoyl-lower alkyl, N-mono-lower alkylcarbamoyl-lower alkyl, N,N-di-lower alkylcarbamoyl-lower alkyl, carboxy-(hydroxy)-lower alkyl, lower alkoxycarbonyl-(hydroxy)-lower alkyl, carbamoyl-(hydroxy)-lower alkyl, N-mono-lower alkylcarbamoyl-(hydroxy)-lower alkyl, N,N-di-lower alkylcarbamoyl-(hydroxy)-lower alkyl, 5- or 6-membered carboxycycloalkyl-lower alkyl, 5- or 6-membered lower alkoxycarbonyl-cycloalkyl-lower alkyl, 5- or 6-membered carbamoylcycloalkyl-lower alkyl, 5- or 6-membered N-mono-alkylcarbamoylcycloalkyl-lower alkyl, N,N-di-lower alkylcarbamoylcycloalkyl-lower alkyl, cyano-lower alkyl, sulfamoyl-lower alkyl, lower alkylsulfamoyl-lower alkyl, or di-lower alkylsulfamoyl-lower alkyl, imidazolyl-lower alkyl, oxopyrrolidinyl-lower alkyl, benzimidazolyl-lower alkyl, oxadiazolyl-lower alkyl, pyridyl-lower alkyl, oxopiperidinyl-lower alkyl or quinolinyl-lower alkyl, piperidin-4-yl-lower alkyl, or lower alkanoylpiperidin-4-yl-lower alkyl, wherein said aryl, imidazolyl, benzimidazolyl, oxadiazolyl, pyridyl, quinolinyl, aryloxy, arylthio and arylsulfonyl groups are optionally substituted with up to four groups independently selected from halo, cyano, nitro, optionally halogenated lower alkyl, optionally halogenated lower alkoxy, optionally halogenated lower alkylthio, optionally halogenated lower alkanesulfonyl, and lower alkoxycarbonyl;

### or R⁸ is OR⁹ or NR⁹R¹⁰

R⁹ is 1) hydrogen, lower alkyl, lower haloalkyl, lower alkenyl, (C₈-C₁₅)alkyl, (C₈-C₁₅)haloalkyl, cycloalkyl, halocycloalkyl, lower alkyl-cycloalkyl, cycloalkyl-lower alkyl, halocycloalkyl-lower alkyl, lower alkoxy-loweralkyl, lower haloalkoxy-lower alkyl, cycloalkoxy-lower alkyl, cycloalkoxy-cycloalkyl, lower alkylthio-lower alkyl, lower haloalkylthio-lower alkyl, lower alkanesulfonyl-lower alkyl, lower haloalkanesulfonyl-lower alkyl, lower alkylthio-cycloalkyl, lower haloalkylthio-cycloalkyl, lower alkanesulfonyl-cycloalkyl, lower haloalkanesulfonyl-cycloalkyl, aminocarbonyl-lower alkyl, lower alkyl-amonocarbonyl-lower alkyl, or di(lower alkyl)-amonocarbonyl-lower alkyl, or 2) aryl, aryl-lower alkyl, aryloxy-lower alkyl, arylthio-lower alkyl, or arylsulfonyl-lower alkyl
wherein aryl is optionally substituted with up to four groups independently selected from halo, cyano, optionally halogenated lower alkyl, optionally halogenated lower alkoxy, optionally halogenated lower alkylthio, and optionally halogenated lower alkanesulfonyl;
   R¹⁰ is 1) hydrogen, lower alkyl, lower haloalkyl, (C₈-C₁₅)alkyl, (C₈-C₁₅)haloalkyl, cycloalkyl, halocycloalkyl, cycloalkyl-lower alkyl, halocycloalkyl-lower alkyl, lower alkoxy-lower alkyl, lower haloalkoxy-lower alkyl, alkylthio-lower alkyl, lower haloalkylthio-lower alkyl, lower alkanesulfonyl-lower alkyl, or lower haloalkanesulfonyl-lower alkyl, or 2) aryl or aryl-lower alkyl
wherein aryl is optionally substituted with up to four groups independently selected from halo, cyano, optionally halogenated lower alkyl, optionally halogenated lower alkoxy, optionally halogenated lower alkylthio, and optionally halogenated lower alkanesulfonyl;
   or R⁹ and R¹⁰ taken together with the nitrogen to which they are attached form a 4-, 5-, 6- or 7-membered heterocyclic ring composed of carbon atoms and 0 or 1 N, O, or S atoms in addition to the nitrogen atom to which R⁹ and R¹⁰ are attached, said ring atoms being substituted with the appropriate number of hydrogen atoms and optionally substituted with up to four groups independently selected from halogen, (C₁-C₆)alkyl, halo(C₁-C₆)alkyl, lower alkanoyl, lower alkoxycarbonyl, aryl, aryl-lower alkyl, and oxo, such that substitution of one oxo group on a carbon atom forms a carbonyl group and substitution of one or two oxo groups on sulfur forms sulfoxide or sulfone groups respectively; wherein the aryl and arylalkyl groups are substituted with up to four groups independently selected from halo, cyano, optionally halogenated lower alkyl, optionally halogenated lower alkoxy, optionally halogenated lower alkylthio, and optionally halogenated lower alkanesulfonyl;
   X¹ is an amino-protecting group;
   X² is hydrogen or together with X³ is a bivalent protecting group;
   X³ is hydrogen or a hydroxy-protecting group;
   and the enantiomers, diastereomers, and salts thereof;
   and their use as intermediates for the preparation of medicinal active ingredients, especially of formula I.

Another embodiment of the invention is a compound of formula III in which at least one, two, or preferably all three of the asymmetric carbon atoms of the main chain have the stereochemical configuration shown in formula IIIa

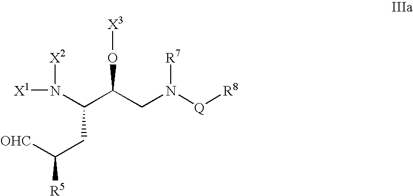

in which the substituents R⁵, R⁷, Q, R⁸, X¹, X², and X³ are as defined for formula III and the salts thereof.

Another embodiment of the invention is a compound of formula IIIa, wherein

R⁵ is lower alkyl or cycloalkyl;
R⁷ is hydrogen or methyl;
Q is carbonyl or sulfonyl;
R⁸ is lower alkyl, lower haloalkyl, C₈-C₁₅ alkyl, C₈-C₁₅ haloalkyl, cycloalkyl, halocycloalkyl, lower alkyl-cycloalkyl, cycloalkyl-lower alkyl, halocycloalkyl-lower alkyl, lower alkoxy-lower alkyl, lower haloalkoxy-lower alkyl, cycloalkoxy-lower alkyl, cycloalkoxy-cycloalkyl, lower alkylthio-lower alkyl, lower haloalkylthio-lower alkyl, lower alkanesulfonyl-lower alkyl, lower haloalkanesulfonyl-lower alkyl, lower alkylthio-cycloalkyl, lower haloalkylthio-cycloalkyl, lower alkanesulfonyl-cycloalkyl, lower haloalkanesulfonyl-cycloalkyl, aryl, aryl-lower alkyl, aryl-lower hydroxyalkyl, arylcycloalkyl, aryloxy-lower alkyl, aryloxy cycloalkyl, arylthio-lower alkyl, arylsulfonyl-lower alkyl, arylthio-cycloalkyl, or arylsulfonyl-cycloalkyl wherein said aryl, aryloxy, arylthio and arylsulfonyl groups are optionally substituted with up to four groups independently selected from halo, cyano, nitro, optionally halogenated lower alkyl, optionally halogenated lower alkoxy, optionally halogenated lower alkylthio, optionally halogenated lower alkanesulfonyl, and lower alkoxycarbonyl;

### or R⁸ is OR⁹ or NR⁹R¹⁰

R⁹ is 1) hydrogen, lower alkyl, lower haloalkyl, lower alkenyl, (C₈-C₁₅)alkyl, (C₈-C₁₅)haloalkyl, cycloalkyl, halocycloalkyl, lower alkyl-cycloalkyl, cycloalkyl-lower alkyl, halocycloalkyl-lower alkyl, lower alkoxy-loweralkyl, lower haloalkoxy-lower alkyl, cycloalkoxy-lower alkyl, cycloalkoxy-cycloalkyl, lower alkylthio-lower alkyl, lower haloalkylthio-lower alkyl, lower alkanesulfonyl-lower alkyl, lower haloalkanesulfonyl-lower alkyl, lower alkylthio-cycloalkyl, lower haloalkylthio-cycloalkyl, lower alkanesulfonyl-cycloalkyl, lower haloalkanesulfonyl-cycloalkyl, aminocarbonyl-lower alkyl, lower alkyl-amonocarbonyl-lower alkyl, or di(lower alkyl)-amonocarbonyl-lower alkyl, or 2) aryl, aryl-lower alkyl, aryloxy-lower alkyl, arylthio-lower alkyl, or arylsulfonyl-lower alkyl
wherein aryl is optionally substituted with up to four groups independently selected from halo, cyano, optionally halogenated lower alkyl, optionally halogenated lower alkoxy, optionally halogenated lower alkylthio, and optionally halogenated lower alkanesulfonyl;
   R¹⁰ is 1) hydrogen, lower alkyl, lower haloalkyl, (C₈-C₁₅)alkyl, (C₈-C₁₅)haloalkyl, cycloalkyl, halocycloalkyl, cycloalkyl-lower alkyl, halocycloalkyl-lower alkyl, lower alkoxy-lower alkyl, lower haloalkoxy-lower alkyl, alkylthio-lower alkyl, lower haloalkylthio-lower alkyl, lower alkanesulfonyl-lower alkyl, or lower haloalkanesulfonyl-lower alkyl, or 2) aryl or aryl-lower alkyl
wherein aryl is optionally substituted with up to four groups independently selected from halo, cyano, optionally halogenated lower alkyl, optionally halogenated lower alkoxy, optionally halogenated lower alkylthio, and optionally halogenated lower alkanesulfonyl;
   or R⁹ and R¹⁰ taken together with the nitrogen to which they are attached form a 4-, 5-, 6- or 7-membered heterocyclic ring composed of carbon atoms and 0 or 1 N, O, or S atoms in addition to the nitrogen atom to which R⁹ and R¹⁰ are attached, said ring atoms being substituted with the appropriate number of hydrogen atoms and optionally substituted with up to four groups independently selected from halogen, (C₁-C₆)alkyl, halo(C₁-C₆)alkyl, lower alkanoyl, lower alkoxycarbonyl, aryl, aryl-lower alkyl, and oxo, such that substitution of one oxo group on a carbon atom forms a carbonyl group and substitution of one or two oxo groups on sulfur forms sulfoxide or sulfone groups respectively; wherein the aryl and arylalkyl groups are substituted with up to four groups independently selected from halo, cyano, optionally halogenated lower alkyl, optionally halogenated lower alkoxy, optionally halogenated lower alkylthio, and optionally halogenated lower alkanesulfonyl;
   X¹ is lower alkoxycarbonyl, or α-phenyl- or α,α-diphenyl-lower alkoxycarbonyl that is unsubstituted or substituted by lower alkyl, lower alkoxy, nitro and/or by halogen, or is 2-halo-lower alkoxycarbonyl;
   X² is hydrogen or together with X³ is carbonyl or lower alkylidene;
   X³ is hydrogen, tri-lower alkylsilyl;
   and the salts thereof.

Another embodiment of the invention is a compound formula IIIa wherein

R⁵ is (C₃-C₅)alkyl;
R⁷ is hydrogen;
Q is carbonyl or sulfonyl;
R⁸ is (C₃-C₁₁)alkyl, (C₃-C₁₁)haloalkyl, (C₃-C₇)cycloalkyl, (C₃-C₁₁)cycloalkylalkyl, (C₃-C₁₁)alkoxy-alkyl, aryl, aryl(C₁-C₃)alkyl, aryl(C₃-C₆)cycloalkyl, arylhydroxy(C₁-C₃)alkyl, aryloxy(C₁-C₅)alkyl, or aryloxy(C₃-C₆)cycloalkyl wherein aryl or aryloxy may be unsubstituted or substituted with one to three groups independently selected from halogen, cyano, (C₁-C₃)alkyl, halo(C₁-C₃)alkyl, (C₁-C₃)alkoxy, and halo(C₁-C₃)alkoxy;

### or R⁸ is NR⁹R¹⁰;

R⁹ is 1) hydrogen, (C₁-C₁₀)alkyl, (C₃-C₇)alkenyl, (C₃-C₇)cycloalkyl, (C₃-C₆)cycloalkyl(C₁-C₅)alkyl, (C₁-C₆)alkoxy(C₁-C₆)alkyl, or aminocarbonyl(C₁-C₅)alkyl, or 2) aryl or aryl(C₁-C₄)alkyl
wherein aryl is optionally substituted with up to 4 groups independently selected from fluorine, chlorine, cyano, (C₁-C₃)alkyl, halo(C₁-C₃)alkyl, (C₁-C₃)alkoxy, halo(C₁-C₃)alkoxy, and (C₁-C₃)alkanesulfonyl;
   R¹⁰ is hydrogen, lower alkyl, or lower haloalkyl; or
   R⁸ and R⁹ taken together are with the nitrogen to which they are attached form an azetidine, pyrrolidine, piperidine, azepine, piperazine, morpholine, or thiomorpholine ring said ring being optionally substituted with up to two groups independently selected from halogen, (C₁-C₃)alkyl, halo(C₁-C₃)alkyl, and oxo, such that substitution of one oxo group on a carbon atom forms a carbonyl group and substitution of one or two oxo groups on sulfur forms sulfoxide or sulfone groups respectively;
   X¹ is tert-butoxycarbonyl;
   X² together with X³ is isopropylidene;
   and the salts thereof.

Another embodiment of the invention is a compound formula IIIa wherein

R⁵ is branched (C₃-C₅)alkyl;
R⁷ is hydrogen;
Q is carbonyl or sulfonyl;
R⁸ is propyl, 2,2-dimethylpropyl, butyl, tert-butyl, n-pentyl, 2-methyl-2-butyl, hexyl, 2-hexyl, 2-methyl-2-pentyl, 2,2-dimethylpentyl, 3-heptyl, 2-methyl-2-hexyl, 2,4,4-trimethylpentyl, 2,2,2-trifluoroethyl, 3,3,3-trifluoropropyl, 4,4,4-trifluorobutyl, 1,1,1,3,3,3-hexafluoro-2-methyl-2-propyl, cyclohexyl, 1-methylcyclohexyl, 4-methylcyclohexyl, cyclopropylmethyl, cyclopentylmethyl, 1-cyclopentyl-1-pentyl, cyclohexylmethyl, 2-cyclohexyl-2-propyl, 2-cyclopropyl-1,1-dimethylethyl, 3-cyclopropyl-2-methyl-2-butyl, 3-methoxypropyl, 2-propoxy-2-propyl, phenyl, benzyl, 3-methylbenzyl, 2-fluorobenzyl, 3-fluorobenzyl, 4-fluorobenzyl, 2,4-difluorobenzyl, 2,3-difluorobenzyl, 3,4-difluorobenzyl, 4-cyanobenzyl, 2-(trifluoromethyl)benzyl, 3-(trifluoromethyl)-benzyl, 4-(trifluoromethyl)benzyl, 4-(trifluoromethoxy)benzyl, phenethyl, 3-phenylpropyl, 2-phenyl-2-propyl, 3-(4-fluorophenyl)-3-pentyl, 1-phenyl-1-cyclopropyl, 1-(4-methylphenyl)-1-cyclopropyl, 1-(4-fluorophenyl)-1-cyclopropyl, 1-(4-methoxyphenyl)-1-cyclopropyl, 1-(2,4-dichlorophenyl)-1-cyclopropyl, 1-phenyl-1-cyclopentyl, 1-phenyl-1-cyclohexyl, 1-(4-fluorophenyl)-1-cyclohexyl, 3-hydroxy-2-methyl-3-phenyl-2-propyl, 2-(4-cyanophenoxy)-2-propyl or 2-(4-chlorophenoxy)-2-propyl;

### or R⁸ is NR⁹R¹⁰;

R⁹ is hydrogen, butyl, isobutyl, t-butyl, pentyl, hexyl, 2,2-dimethyl-1-pentyl, 2-methyl-2-hexyl, 2,4,4-trimethyl-2-pentyl, allyl, 2-(cyclopropyl)ethyl, cyclohexylmethyl, 2-(cyclohexyl)methyl, cyclohexyl, 2-methoxyethyl, benzyl, 2-phenylethyl, 3-phenylpropyl, 3-(4-fluorophenyl)-2-methyl-2-propyl, 3-fluorophenyl, 3-(trifluoromethyl)phenyl, or 2-(aminocarbonyl)-2-methyl-1-propyl,
R¹⁰ is hydrogen, methyl, or isobutyl;

### or R⁹-R¹⁰ is —(CH₂)₅— or —(CH₂)₂O(CH₂)₂—;

X¹ is tert-butoxycarbonyl;
X² together with X³ is isopropylidene;
and the salts thereof;

Preferred are compounds of formulae III and IIIa in the Examples and the salts thereof.

The following terms are used herein.

Aryl and aryl in aryloxy, arylthio, arylsulfonyl, aryl-lower alkoxy, aryl-lower alkyl and the like are, for example, phenyl or naphthyl that is unsubstituted or mono-, di- or tri-substituted by optionally halogenated lower alkyl, optionally halogenated lower alkoxy, hydroxy, amino, lower alkylamino, di-lower alkylamino, halogen, cyano, carbamoyl, lower alkoxycarbonyl, trifluoromethoxy, and/or by trifluoromethyl.

Cycloalkoxy and cycloalkoxy in cycloalkoxy-lower alkoxy is, for example, 3- to 8-membered, preferably 3-, 5- or 6-membered, cycloalkoxy, such as cyclopropyloxy, cyclopentyloxy, cyclohexyloxy, also cyclobutyloxy, cycloheptyloxy, or cyclooctyloxy.

Cycloalkyl is, for example, 3- to 8-membered, preferably 3-, 5- or 6-membered, cycloalkyl, such as cyclopropyl, cyclopentyl, cyclohexyl, also cyclobutyl, cycloheptyl, or cyclooctyl.

Heterocyclyl is, for example, a 3- to 8-membered, preferably a 5- or 6-membered, saturated heterocycle, for example tetrahydrofuryl, tetrahydrothienyl, pyrrolidinyl, tetrahydropyranyl, tetrahydrothiopyranyl and piperidinyl.

Free or esterified or amidated carboxy-lower alkoxy is, for example, carboxy-lower alkoxy, lower alkoxycarbonyl-lower alkoxy, carbamoyl-lower alkoxy, or N-mono- or N,N-di-lower alkylcarbamoyl-lower alkoxy.

Optionally lower alkanoylated, halogenated or sulfonylated hydroxy-lower alkoxy is, for example, lower alkanoyloxy-lower alkyl, hydroxy-lower alkoxy, halo-(hydroxy)-lower alkoxy, or lower alkanesulfonyl-(hydroxy)-lower alkoxy.

Optionally hydrogenated heteroaryl-lower alkoxy is, for example, optionally partially hydrogenated or N-oxidized pyridyl-lower alkoxy, thiazolyl-lower alkoxy, thiazolinyl-lower alkoxy or especially morpholino-lower alkoxy.

Optionally hydrogenated heteroarylthio-lower alkoxy is, for example, optionally partially or fully hydrogenated heteroarylthio-lower alkoxy, such as thiazolylthio-lower alkoxy, thiazolinylthio-lower alkoxy, imidazolylthio-lower alkoxy, imidazolinylthio-lower alkoxy optionally N-oxidized pyridlylthio-lower alkoxy, or pyrimidinylthio-lower alkoxy.

Free or esterified or amidated carboxy-lower alkyl is, for example, carboxy-lower alkyl, lower alkoxycarbonyl-lower alkyl, carbamoyl-lower alkyl, or N-mono- or N,N-di-lower alkylcarbamoyl-lower alkyl.

Optionally halogenated lower alkyl is, for example, lower alkyl, monohalo-lower alkyl or polyhalo-lower alkyl.

Optionally halogenated lower alkoxy is, for example, lower alkoxy, monohalo-lower alkoxy or polyhalo-lower alkoxy.

Optionally S-oxidized lower alkylthio-lower alkyl is, for example, lower alkylthio-lower alkyl, lower alkanesulfinyl-lower alkyl, or lower alkanesulfonyl-lower alkyl.

Optionally S-oxidized lower alkylthio-lower alkoxy is, for example, lower alkylthio-lower alkoxy, lower alkanesulfinyl-lower alkoxy or lower alkanesulfonyl-lower alkoxy.

Optionally hydrogenated heteroaryl-lower alkyl or optionally N-oxidized heteroaryl-lower alkyl is, for example, optionally partially hydrogenated or N-oxidized pyridyl-lower alkyl.

Optionally hydrogenated heteroarylthio-lower alkyl or optionally N-oxidized heteroarylthio-lower alkyl is, for example, thiazolylthio-lower alkyl or thiazolinylthio-lower alkyl, imidazolylthio-lower alkyl, optionally N-oxidized pyridylthio-lower alkyl, or pyrimidinylthio-lower alkyl.

Amino-lower alkyl that is unsubstituted or N-mono- or N,N-di-lower alkylated, N-lower alkanoylated or N-lower alkanesulfonylated or N,N-disubstituted by lower alkylene, by unsubstituted or N′-lower alkylated or N′-lower alkanoylated aza-lower alkylene, by oxa-lower alkylene or by optionally S-oxidized thia-lower alkylene is, for example, amino-lower alkyl, lower alkylamino-lower alkyl, di-lower alkylamino-lower alkyl, lower alkanoylamino-lower alkyl, lower alkanesulfonylamino-lower alkyl, polyhalo-lower alkanesulfonylamino-lower alkyl, pyrrolidino-lower alkyl, piperidino-lower alkyl, piperazino-, N′-lower alkylpiperazino- or N′-lower alkanoylpiperazino-lower alkyl, morpholino-lower alkyl, thiomorpholino-, S-oxothiomorpholino-, or S,S-dioxothiomorpholino-lower alkyl.

Amino-lower alkoxy that is unsubstituted or N-mono- or N,N-di-lower alkylated, N-lower alkanoylated or N-lower alkanesulfonylated or N,N-disubstituted by lower alkylene, by unsubstituted or N′-lower alkylated amino-lower alkylene or lower alkanoylated-amino-lower alkylene, by oxa-lower alkylene or by optionally S-oxidized thia-lower alkylene is, for example, amino-lower alkoxy, lower alkylamino-lower alkoxy, di-lower alkylamino-lower alkoxy, lower alkanoylamino-lower alkoxy, lower alkanesulfonylamino-lower alkoxy, polyhalo-lower alkanesulfonylamino-lower alkoxy, pyrrolidino-lower alkoxy, piperidino-lower alkoxy, piperazino-, N′-lower alkylpiperazino- or N′-lower alkanoylpiperazino-lower alkoxy, morpholino-lower alkoxy, thiomorpholino-, S-oxothiomorpholino-, or S,S-dioxothio-morpholino-lower alkoxy.

Unsubstituted or N-mono- or N,N-di-lower alkylated or N-lower alkanoylated amino is, for example, amino, lower alkylamino, di-lower alkylamino, or lower alkanoylamino.

Free or aliphatically esterified or etherified hydroxy-lower alkyl is, for example, hydroxy-lower alkyl, lower alkanoyloxy-lower alkyl, lower alkoxy-lower alkyl, or lower alkenyloxy-lower alkyl.

Amino-lower alkyl that is unsubstituted or N-lower alkanoylated, N-mono- or N,N-di-lower alkylated or N,N-disubstituted by lower alkylene, by hydroxy-, lower alkoxy- or lower alkanoyloxy-lower alkylene, by unsubstituted or lower alkanoylated-amino-lower alkylene, by oxa-lower alkylene or by optionally S-oxidized thia-lower alkylene is, for example, amino-lower alkyl, lower alkanoylamino-lower alkyl, N-mono- or N,N-di-lower alkylamino-lower alkyl, optionally hydroxylated or lower alkoxylated piperidino-lower alkyl, such as piperidino-lower alkyl, hydroxypiperidino-lower alkyl or lower alkoxy-piperidino-lower alkyl, piperazino-, ω-lower alkylpiperazino- or N′-lower alkanoyl-piperazino-lower alkyl, unsubstituted or lower alkylated morpholino-lower alkyl, such as morpholino-lower alkyl or dimethylmorpholino-lower alkyl, or optionally S-oxidized thio-morpholino-lower alkyl, such as thiomorpholino-lower alkyl or S,S-dioxothiomorpholino-lower alkyl.

Free or esterified or amidated carboxy-(hydroxy)-lower alkyl is, for example, carboxy-(hydroxy)-lower alkyl, lower alkoxycarbonyl-(hydroxy)-lower alkyl or carbamoyl-(hydroxy)-lower alkyl.

Free or esterified or amidated carboxycycloalkyl-lower alkyl is, for example, 5- or 6-membered carboxycycloalkyl-lower alkyl, lower alkoxycarbonylcycloalkyl-lower alkyl, carbamoylcycloalkyl-lower alkyl, or N-mono- or N,N-di-lower alkylcarbamoylcyclo-alkyl-lower alkyl.

Unsubstituted or N-mono- or N,N-di-lower alkylated sulfamoyl-lower alkyl is, for example, sulfamoyl-lower alkyl, lower alkylsulfamoyl-lower alkyl, or di-lower alkyl-sulfamoyl-lower alkyl.

Lower radicals and compounds are, for example, those having up to and including 7, preferably up to and including 4, carbon atoms.

5- or 6-Membered carboxycycloalkyl-lower alkyl, lower alkoxycarbonylcycloalkyl-lower alkyl, carbamoylcycloalkyl-lower alkyl, N-mono- or N,N-di-lower alkylcarbamoylcyclo-alkyl-lower alkyl is, for example, ω-(1-carboxycycloalkyl)-C₁-C₄ alkyl, ω-(1-lower alkoxycarbonylcycloalkyl)-C₁-C₄ alkyl, ω-(1-carbamoylcycloalkyl)-C₁-C₄ alkyl, ω-(1-lower alkylcarbamoylcycloalkyl)-C₁-C₄ alkyl, or ω-(1-di-lower alkylcarbamoylcycloalkyl)-C₁-C₄ alkyl, wherein cycloalkyl is, for example, cyclopentyl or cyclohexyl; lower alkoxycarbonyl is, for example, C₁-C₄ alkoxycarbonyl, such as methoxy- or ethoxycarbonyl; lower alkylcarbamoyl is, for example, C₁-C₄ alkylcarbamoyl, such as methylcarbamoyl; di-lower alkylcarbamoyl is, for example, di-C₁-C₄ alkylcarbamoyl, such as dimethylcarbamoyl; and lower alkyl is, for example, C₁-C₄ alkyl, such as methyl, ethyl, propyl, or butyl, especially (1-carboxycyclopentyl)methyl.

5- or 6-Membered cycloalkoxy-lower alkoxy is, for example, cyclopentyloxy-(C₁-C₄)alkoxy or cyclohexyloxy-(C₁-C₄)alkoxy, such as cyclopentyloxy-methoxy, cyclohexyloxy-methoxy, 2-cyclopentyloxy-ethoxy, 2-cyclohexyloxy-ethoxy, 2- or 3-cyclopentyloxy-propyloxy, 2- or 3-cyclohexyloxy-propyloxy, 4-cyclopentyloxy-butyloxy or 4-cyclohexyloxy-butyloxy, especially cyclopentyloxy-methoxy or cyclohexyloxy-methoxy.

5- or 6-Membered cycloalkoxy-lower alkyl is, for example, cyclopentyloxy-(C₁-C₄)alkyl or cyclohexyloxy-(C₁-C₄)alkyl, such as cyclopentyloxy-methyl, cyclohexyloxy-methyl, 2-cyclopentyloxy-ethyl, 2-cyclohexyloxy-ethyl, 2- or 3-cyclopentyloxy-propyl, 2- or 3-cyclohexyloxy-propyl, 2-cyclopentyloxy-2-methyl-propyl, 2-cyclohexyloxy-2-methyl-propyl, 2-cyclopentyloxy-2-ethyl-butyl, 2-cyclohexyloxy-2-ethyl-butyl, 4-cyclopentyloxy-butyl or 4-cyclohexyloxy-butyl, especially cyclopentyloxy-methyl or cyclohexyloxy-methyl.

Amino-lower alkoxy is, for example, amino-C₁-C₄ alkoxy, such as 2-aminoethoxy or 5-aminopentyloxy, also 3-aminopropyloxy or 4-aminobutyloxy.

Amino-lower alkyl is, for example, amino-C₁-C₄alkyl, such as 2-aminoethyl, 3-aminopropyl or 4-aminobutyl.

Carbamoyl-(hydroxy)-lower alkyl is, for example, carbamoyl-C₁-C₇ (hydroxy)alkyl, such as 1-carbamoyl-2-hydroxyethyl.

Carbamoyl-lower alkoxy is, for example, carbamoyl-C₁-C₄ alkoxy, such as carbamoylmethoxy, 2-carbamoylethoxy, 3-carbamoylpropyloxy, or 4-carbamoylbutyloxy, especially carbamoylmethoxy.

Carbamoyl-lower alkyl is, for example, carbamoyl-C₁-C₇ alkyl, such as carbamoylmethyl, 2-carbamoylethyl, 3-carbamoylpropyl, 2-(3-carbamoyl)propyl, 2-carbamoylpropyl, 3-(1-carbamoyl)propyl, 2-(2-carbamoyl)propyl, 2-(carbamoyl-2-methyl)propyl, 4-carbamoylbutyl, 1-carbamoylbutyl, 1-(1-carbamoyl-2-methyl)butyl, or 3-(4-carbamoyl-2-methyl)butyl.

Carboxy-(hydroxy)-lower alkyl is, for example, carboxy-C₁-C₇ (hydroxy)alkyl, such as 1-carboxy-2-hydroxy-ethyl.

Carboxy-lower alkoxy is, for example, carboxy-C₁-C₄ alkoxy, such as carboxymethoxy, 2-carboxyethoxy, 2- or 3-carboxypropyloxy, or 4-carboxybutyloxy, especially carboxy-methoxy.

Carboxy-lower alkyl is, for example, carboxy-C₁-C₄ alkyl, such as carboxymethyl, 2-carboxyethyl, 2- or 3-carboxypropyl, 2-carboxy-2-methyl-propyl, 2-carboxy-2-ethyl-butyl, or 4-carboxybutyl, especially carboxymethyl.

Cyano-lower alkoxy is, for example, cyano-C₁-C₄ alkoxy, such as cyanomethoxy, 2-cyano-ethoxy, 2- or 3-cyanopropyloxy, or 4-cyanobutyloxy, especially cyanomethoxy.

Cyano-lower alkyl is, for example, cyano-C₁-C₄ alkyl, such as cyanomethyl, 2-cyanoethyl, 2- or 3-cyanopropyl, 2-cyano-2-methyl-propyl, 2-cyano-2-ethyl-butyl, or 4-cyanobutyl, especially cyanomethyl.

Di-(N-mono- or N,N-di-lower alkylcarbamoyl)-lower alkyl is, for example, di-(N-mono- or N,N-di-C₁-C₄ alkylcarbamoyl)-C₁-C₄ alkyl, such as 1,2-di-(N-mono- or N,N-di-C₁-C₄ alkylcarbamoyl)ethyl, or 1,3-di-(N-mono- or N,N-di-C₁-C₄ alkylcarbamoyl)propyl.

Dicarbamoyl-lower alkyl is, for example, dicarbamoyl-C₁-C₄ alkyl, such as 1,2-dicarbamoylethyl or 1,3-dicarbamoylpropyl.

Dimethylmorpholino-lower alkoxy can be N-oxidized and is, for example, 2,6-dimethylmorpholino- or 3,5-dimethylmorpholino-C₁-C₄ alkoxy, such as 2,6-dimethylmorpholino- or 3,5-dimethylmorpholino-methoxy, 2-(2,6-dimethylmorpholino- or 3,5-dimethylmorpholino)-ethoxy, 3-(2,6-dimethylmorpholino- or 3,5-dimethylmorpholino)-propyloxy, 2-(2,6-dimethylmorpholino- or 3,5-dimethylmorpholino-3-methyl)propyloxy, or 1- or 2-[4-(2,6-dimethylmorpholino- or 3,5-dimethylmorpholino)]-butyloxy.

Dimethylmorpholino-lower alkyl can be N-oxidized and is, for example, 2,6-dimethylmorpholino- or 3,5-dimethylmorpholino-C₁-C₄ alkyl, such as 2,6-dimethylmorpholino- or 3,5-dimethylmorpholino-methoxy, 2-(2,6-dimethylmorpholino- or 3,5-dimethylmorpholino)-ethoxy, 3-(2,6-dimethylmorpholino- or 3,5-dimethylmorpholino)-propyl, 2-(2,6-dimethylmorpholino- or 3,5-dimethylmorpholino-3-methyl)-propyl, or 1- or 2-[4-(2,6-dimethylmorpholino- or 3,5-dimethylmorpholino)]-butyl.

Di-lower alkylamino is, for example, di-C₁-C₄ alkylamino, such as dimethylamino, N-methyl-N-ethylamino, diethylamino, N-methyl-N-propylamino, or N-butyl-N-methylamino.

Di-lower alkylamino-lower alkoxy is, for example, N,N-di-C₁-C₄ alkylamino-C₁-C₄ alkoxy, such as 2-dimethylaminoethoxy, 3-dimethylaminopropyloxy, 4-dimethylaminobutyloxy, 2-diethylaminoethoxy, 2-(N-methyl-N-ethyl-amino)ethoxy, or 2-(N-butyl-N-methyl-amino)ethoxy.

Di-lower alkylamino-lower alkyl is, for example, N,N-di-C₁-C₄ alkylamino-C₁-C₄ alkyl, such as 2-dimethylaminoethyl, 3-dimethylaminopropyl, 4-dimethylaminobutyl, 2-diethylaminoethyl, 2-(N-methyl-N-ethyl-amino)ethyl, or 2-(N-butyl-N-methyl-amino)ethyl.

Di-lower alkylcarbamoyl-lower alkoxy is, for example, N,N-di-C₁-C₄ alkylcarbamoyl-C₁-C₄ alkoxy, such as methyl- or dimethyl-carbamoyl-C₁-C₄ alkoxy, such as N-methyl-, N-butyl- or N,N-dimethyl-carbamoylmethoxy, 2-(N-methylcarbamoyl)ethoxy, 2-(N-butylcarbamoyl)ethoxy, 2-(N,N-dimethylcarbamoyl)ethoxy, 3-(N-methylcarbamoyl)propyloxy, 3-(N-butylcarbamoyl)propyloxy, 3-(N,N-dimethylcarbamoyl)propyloxy or 4-(N-methylcarbamoyl)butyloxy, 4-(N-butylcarbamoyl)-butyloxy, or 4-(N,N-dimethylcarbamoyl)butyloxy, especially N-methyl-, N-butyl- or N,N-dimethyl-carbamoylmethoxy.

Di-lower alkylcarbamoyl-lower alkyl is, for example, N,N-di-C₁-C₄ alkylcarbamoyl-C₁-C₄ alkyl, such as 2-dimethylcarbamoylethyl, 3-dimethylcarbamoylpropyl, 2-dimethylcarbamoylpropyl, 2-(dimethylcarbamoyl-2-methyl)propyl, or 2-(1-dimethylcarbamoyl-3-methyl)butyl.

Di-lower alkylsulfamoyl-lower alkyl is, for example, N,N-di-C₁-C₄ alkylsulfamoyl-C₁-C₄ alkyl, N,N-dimethylsulfamoyl-C₁-C₄ alkyl, such as N,N-dimethylsulfamoylmethyl, 2-(N,N-dimethylcarbamoyl)ethyl, 3-(N,N-dimethylcarbamoyl)propyl, or 4-(N,N-dimethylcarbamoyl)butyl, especially N,N-dimethylcarbamoylmethyl.

Unsubstituted or N-lower alkanoylated piperidyl-lower alkyl is, for example, 1-C₁-C₇-lower alkanoylpiperidin-4-yl-C₁-C₄ alkyl, such as 1-acetylpiperidinylmethyl or 2-(1-acetyl-piperidinyl)ethyl.

Optionally partially hydrogenated pyridyl-lower alkoxy or N-oxidized pyridyl-lower alkoxy is, for example, optionally partially hydrogenated pyridyl-C₁-C₄ alkoxy or N-oxopyridyl-C₁-C₄ alkoxy, such as pyridyl-methoxy, dihydropyridyl-methoxy or N-oxopyridyl-methoxy, 2-(pyridyl)ethoxy, 2-(pyridyl)propyloxy, 3-(pyridyl)propyloxy, or 4-(pyridyl)butyloxy, especially (3-pyridyl)methoxy or (4-pyridyl)methoxy.

Optionally partially hydrogenated pyridyl-lower alkyl or N-oxidized pyridyl-lower alkyl is, for example, optionally partially hydrogenated pyridyl-C₁-C₄ alkyl or N-oxopyridyl-C₁-C₄ alkyl, such as pyridyl-methyl, dihydropyridyl-methyl, N-oxopyridyl-methyl, 2-(pyridyl)ethyl, 2-(pyridyl)propyl, 3-(pyridyl)propyl, or 4-(pyridyl)butyl, especially (3-pyridyl)methyl or (4-pyridyl)methyl.

Halo-(hydroxy)-lower alkoxy is, for example, halo-C₁-C₇ (hydroxy)alkoxy, especially halo-C₂-C₄ (hydroxy)alkoxy, such as 3-halo-, such as 3-chloro-2-hydroxy-propyloxy.

Hydroxy-lower alkoxy is, for example, hydroxy-C₂-C₇ alkoxy, especially hydroxy-C₂-C₄ alkoxy, such as 2-hydroxybutyloxy, 3-hydroxypropyloxy or 4-hydroxybutyloxy.

Hydroxy-lower alkyl is, for example, hydroxy-C₂-C₇ alkyl, especially hydroxy-C₂-C₄ alkyl, such as 2-hydroxyethyl, 3-hydroxypropyl or 4-hydroxybutyl.

Hydroxypiperidino-lower alkyl is, for example, 3- or 4-hydroxypiperidino-C₁-C₄ alkyl, such as 3-hydroxypiperidinomethyl, 4-hydroxypiperidinomethyl, 2-(3-hydroxypiperidino)ethyl, 2-(4-hydroxypiperidino)ethyl, 3-(3-hydroxypiperidino)propyl, 3-(4-hydroxypiperidino)propyl, 4-(3-hydroxypiperidino)butyl or 4-(4-hydroxypiperidino)butyl.

Imidazolyl-lower alkyl is, for example, imidazolyl-C₁-C₄ alkyl, such as imidazol-4-yl-methyl, 2-(imidazol-4-yl)ethyl, 3-(imidazol-4-yl)propyl, or 4-(imidazol-4-yl)butyl.

Imidazolyl-lower alkoxy is, for example, imidazolyl-C₁-C₄ alkoxy, such as imidazol-4-yl-methoxy, 2-(imidazol-4-yl)ethoxy, 3-(imidazol-4-yl)propyloxy, or 4-(imidazol-4-yl)butyloxy.

Morpholinocarbonyl-lower alkyl is, for example, morpholinocarbonyl-C₁-C₄ alkyl, such as 1-morpholinocarbonylethyl, 3-morpholinocarbonylpropyl, or 1-(morpholinocarbonyl-2-methyl)propyl.

Morpholino-lower alkyl can be N-oxidized and is, for example, N-oxomorpholino-C₁-C₄ alkyl, such as N-oxomorpholinomethyl, 2-(N-oxomorpholino)ethyl, 3-(N-oxomorpholino)propyl, or 4-(N-oxomorpholino)butyl.

Morpholino-lower alkoxy is, for example, morpholino-C₁-C₄ alkoxy, such as 1-morpholinoethoxy, 3-morpholinopropyloxy, or 1-(morpholino-2-methyl)propyloxy.

Morpholino-lower alkoxy can be N-oxidized and is, for example, N-oxomorpholino-C₁-C₄ alkoxy, such as N-oxomorpholinomethoxy, 2-(N-oxomorpholino)ethoxy, 3-(N-oxomorpholino)propyloxy, or 4-(N-oxomorpholino)butyloxy.

Lower alkanoyl is, for example, C₁-C₇ alkanoyl, especially C₂-C₆ alkanoyl, such as acetyl, propionyl, butyryl, isobutyryl or pivaloyl.

Lower alkanoylamino is, for example, N—C₁-C₇ alkanoylamino, such as acetylamino or pivaloylamino.

Lower alkanoylamino-lower alkyl is, for example, N—C₁-C₄ alkanoylamino-C₁-C₄ alkyl, such as 2-acetylaminoethyl.

Lower alkanoyl-lower alkoxy (oxo-lower alkoxy) carries the lower alkanoyl group in a position higher than the α-position and is, for example, C₁-C₇ alkanoyl-C₁-C₄ alkoxy, such as 4-acetoxy-butoxy.

Lower alkanoyloxy-lower alkyl carries the lower alkanoyloxy group in a position higher than the α-position and is, for example, C₁-C₇ alkanoyloxy-C₁-C₄ alkyl, such as 4-acetoxy-butyl.

Lower alkanesulfonyl-(hydroxy)-lower alkoxy is, for example, C₁-C₇ alkanesulfonyl-C₁-C₄ (hydroxy)alkoxy, such as 3-methanesulfonyl-2-hydroxy-propyloxy.

Lower alkanesulfonyl-lower alkoxy is, for example, C₁-C₇ alkanesulfonyl-C₁-C₄ alkoxy, such as methanesulfonylmethoxy or 3-methanesulfonyl-propyloxy.

Lower alkanesulfonylamino-lower alkoxy is, for example, C₁-C₇ alkanesulfonylamino-C₁-C₄ alkoxy, such as ethanesulfonylaminomethoxy, 2-ethanesulfonylaminoethoxy, 3-ethane-sulfonylaminopropyloxy, or 3-(1,1-dimethylethanesulfonylamino)propyloxy.

Lower alkanesulfonylamino-lower alkyl is, for example, C₁-C₇ alkanesulfonylamino-C₁-C₄ alkyl, such as ethanesulfonylaminomethyl, 2-ethanesulfonylaminoethyl, 3-ethanesulfonyl-aminopropyl, or 3-(1,1-dimethylethanesulfonylamino)propyl.

Lower alkanesulfonyl-lower alkyl is, for example, C₁-C₇ alkanesulfonyl-C₁-C₄ alkyl, such as ethanesulfonylmethyl, 2-ethanesulfonylethyl, 3-ethanesulfonylpropyl, or 3-(1,1-dimethyl-ethanesulfonyl)propyl.

Lower alkenyl is, for example, C₂-C₇ alkenyl, such as vinyl or allyl.

Lower alkenyloxy is, for example, C₂-C₇ alkenyloxy, such as allyloxy.

Lower alkenyloxy-lower alkoxy is, for example, C₃-C₇ alkenyloxy-C₁-C₄ alkoxy, such as allyloxymethoxy.

Lower alkenyloxy-lower alkyl is, for example, C₃-C₇ alkenyloxy-C₁-C₄ alkyl, such as allyloxymethyl.

Lower alkoxy is, for example, C₁-C₇ alkoxy, preferably C₁-C₅ alkoxy, such as methoxy, ethoxy, propyloxy, isopropyloxy, butyloxy, isobutyloxy, secondary butyloxy, tertiary butyloxy, pentyloxy, or a hexyloxy or heptyloxy group.

Lower alkoxycarbonyl is, for example, C₁-C₇ alkoxycarbonyl, preferably C₁-C₅ alkoxycarbonyl, such as methoxycarbonyl, ethoxycarbonyl, propyloxycarbonyl, isopropyloxycarbonyl, butyloxycarbonyl, isobutyloxycarbonyl, secondary butyloxycarbonyl, tertiary butyloxy, pentyloxycarbonyl, or a hexyloxycarbonyl or heptyloxycarbonyl group.

Lower alkoxycarbonyl-(hydroxy)-lower alkyl is, for example, C₁-C₄ alkoxycarbonyl-C₁-C₇ (hydroxy)alkyl, such as 1-methoxycarbonyl- or 1-ethoxycarbonyl-2-hydroxy-ethyl.

Lower alkoxycarbonylamino-lower alkoxy is, for example, C₁-C₇ alkoxycarbonylamino-C₂-C₇ alkoxy, preferably C₂-C₅ alkoxycarbonylamino-C₂-C₇ alkoxy, such as methoxycarbonylamino-C₂-C₇ alkoxy, ethoxycarbonylamino-C₂-C₇ alkoxy, propyloxycarbonylamino-C₂-C₇ alkoxy, isobutyloxycarbonylamino-C₂-C₇ alkoxy, butyloxycarbonylamino-C₂-C₇ alkoxy, isobutyloxycarbonylamino-C₂-C₇ alkoxy, secondary butyloxycarbonylamino-C₂-C₇ alkoxy or tertiary butyloxyamino-C₂-C₇ alkoxy, wherein C₂-C₇ alkoxy is, for example, methoxy, ethoxy, propyloxy, butyloxy, pentyloxy, or hexyloxy.

Lower alkoxycarbonylamino-lower alkyl is, for example, C₁-C₇ alkoxycarbonylamino-C₂-C₇ alkyl, preferably C₂-C₅ alkoxycarbonylamino-C₂-C₇ alkyl, such as methoxycarbonyl-C₂-C₇ alkyl, ethoxycarbonylamino-C₂-C₇-alkyl, propyloxycarbonylamino-C₂-C₇ alkyl isopropyloxy-carbonylamino-C₂-C₇ alkyl, butyloxycarbonylamino-C₂-C₇ alkyl, isobutyloxycarbonylamino-C₂-C₇ alkyl, secondary butyloxycarbonylamino-C₂-C₇ alkyl, or tertiary butyloxyamino-C₂-C₇ alkyl, wherein C₂-C₇ alkyl is, for example, ethyl, propyl, butyl, pentyl, or hexyl.

Lower alkoxycarbonyl-lower alkoxy is, for example, C₁-C₄ alkoxycarbonyl-C₁-C₄ alkoxy, such as methoxycarbonyl- or ethoxycarbonyl-methoxy, 2-methoxycarbonyl- or 2-ethoxycarbonyl-ethoxy, 2- or 3-methoxycarbonyl- or 2- or 3-ethoxycarbonyl-propyloxy or 4-methoxycarbonyl- or 4-ethoxycarbonyl-butyloxy, especially methoxycarbonyl- or ethoxycarbonyl-methoxy or 3-methoxycarbonyl- or 3-ethoxycarbonyl-propyloxy.

Lower alkoxycarbonyl-lower alkyl is, for example, C₁-C₄ alkoxycarbonyl-C₁-C₄ alkyl, such as methoxycarbonyl-methyl, ethoxycarbonyl-methyl, 2-methoxycarbonyl-ethyl, 2-ethoxycarbonyl-ethyl, 3-methoxycarbonyl-propyl, 3-ethoxycarbonyl-propyl or 4-ethoxycarbonyl-butyl.

Lower alkoxy-lower alkenyl is, for example, C₁-C₄ alkoxy-C₂-C₄ alkenyl, such as 4-methoxybut-2-enyl.

Lower alkoxy-lower alkoxy is, for example, C₁-C₄ alkoxy-C₂-C₄ alkoxy, such as 2-methoxy-, 2-ethoxy- or 2-propyloxy-ethoxy, 3-methoxy- or 3-ethoxy-propyloxy, or 4-methoxybutyloxy, especially 3-methoxypropyloxy or 4-methoxybutyloxy.

Lower alkoxy-lower alkyl is, for example, C₁-C₄ alkoxy-C₁-C₄ alkoxy-C₁-C₄ alkyl, such as 2-methoxy-, 2-ethoxy- or 2-propyloxy-ethoxymethyl, 2-(2-methoxy-, 2-ethoxy- or 2-propyloxy-ethoxy)ethyl, 3-(3-methoxy- or 3-ethoxy-propyloxy)propyl, or 4-(2-methoxybutyloxy)-butyl, especially 2-(3-methoxypropyloxy)ethyl or 2-(4-methoxybutyloxy)ethyl.

Lower alkoxy-lower alkyl is, for example, C₁-C₄ alkoxy-C₁-C₄ alkyl, such as ethoxymethyl, propyloxymethyl, butyloxymethyl, 2-methoxy-, 2-ethoxy- or 2-propyloxy-ethyl, 3-methoxy- or 3-ethoxy-propyl or 4-methoxybutyl, especially 3-methoxypropyl, or 4-methoxybutyl.

Piperidino-lower alkyl is, for example, piperidino-C₁-C₄ alkyl or hydroxypiperidino-C₁-C₄ alkyl, such as piperidinomethyl or 4-hydroxypiperidinomethyl.

Lower alkoxypiperidino-lower alkyl is, for example, C₁-C₄ alkoxypiperidino-C₁-C₄ alkyl, such as 4-(C₁-C₄ alkoxy)-piperidinomethyl, especially 4-methoxypiperidinomethyl.

Lower alkyl may be straight-chained or branched and/or bridged and is, for example, corresponding C₁-C₇ alkyl, such as methyl, ethyl, propyl, isopropyl, butyl, isobutyl, secondary butyl or tertiary butyl, or a pentyl, hexyl or heptyl group. Lower alkyl R² or R³ is especially C₂-C₇ alkyl; lower alkyl R⁵ or R⁷ is especially branched C₃-C₇ alkyl; and lower alkyl R⁸ or R³ is, for example, straight-chained, branched or bridged C₃-C₇ alkyl.

Lower alkylamino is, for example, C₁-C₄ alkylamino, such as methylamino, ethylamino, propylamino, butylamino, isobutylamino, secondary butylamino, or tertiary butylamino.

Lower alkylamino-lower alkoxy is, for example, C₁-C₄ alkylamino-C₁-C₄ alkoxy, such as propylaminomethoxy, 2-methylamino-, 2-ethylamino-, 2-propylamino- or 2-butylamino-ethoxy, 3-ethylamino- or 3-propylamino-propyloxy or 4-methylaminobutoxy.

Lower alkylamino-lower alkyl is, for example, C₁-C₄ alkylamino-C₁-C₄ alkyl, such as propylaminomethyl, 2-methylamino-, 2-ethylamino-, 2-propylamino- or 2-butylamino-ethyl, 3-ethylamino- or 3-propylamino-propyl or 4-methylaminobutyl.

Lower alkylcarbamoyl-lower alkoxy is, for example, N—C₁-C₇ alkylcarbamoyl-C₁-C₄ alkoxy, such as methyl- or dimethyl-carbamoyl-C₁-C₄ alkoxy, e.g., methylcarbamoylmethoxy, 2-methylcarbamoylethoxy, or 3-methylcarbamoylpropyloxy.

Lower alkylenedioxy is, for example, methylenedioxy or ethylenedioxy, but can also be 1,3- or 1,2-propylenedioxy.

Lower alkylsulfamoyl-lower alkyl is, for example, N—C₁-C₇ alkylsulfamoyl-C₁-C₄ alkyl, such as N-methyl-, N-ethyl-, N-propyl- or N-butyl-sulfamoyl-C₁-C₄ alkyl, such as N-methyl-, N-ethyl-, N-propyl- or N-butyl-sulfamoylmethyl, 2-(N-methylsulfamoyl)ethyl, 2-(N-butylsulfamoyl)ethyl, 3-(N-methylsulfamoyl)propyl, 3-(N-butylsulfamoyl)propyl, or 4-(N-methylsulfamoyl)butyl, 4-(N-butylsulfamoyl)butyl or 4-(N,N-dimethylsulfamoyl)butyl, especially N-methyl-, N-butyl-, or N,N-dimethyl-sulfamoylmethyl.

Lower alkylthio-(hydroxy)-lower alkoxy is, for example, C₁-C₄ alkylthio-C₁-C₄ (hydroxy)alkoxy, such as 2-hydroxy-3-methylthiopropyloxy.

Lower alkylthio-lower alkoxy is, for example, C₁-C₄ alkylthio-C₁-C₄ alkoxy, such as methylthio-C₁-C₄ alkoxy, e.g. methylthiomethoxy, 2-methylthioethoxy, or 3-methylthiopropyloxy.

Lower alkylthio-lower alkyl is, for example, C₁-C₄ alkylthio-C₁-C₄ alkyl, such as methylthio-C₁-C₄ alkyl, e.g. methylthiomethyl, 2-methylthioethyl, or 3-methylthiopropyl.

N′-Lower alkanoylpiperazino-lower alkoxy is, for example, N′-lower alkanoylpiperazino-C₁-C₄ alkoxy, such as 4-acetylpiperazinomethoxy.

N′-Lower alkanoylpiperazino-lower alkyl is, for example, N′-C₂-C₇-lower alkanoyl-piperazino-C₁-C₄ alkyl, such as 4-acetylpiperazinomethyl.

N′-Lower alkylpiperazino-lower alkyl is, for example, N′-C₁-C₄ alkylpiperazino-C₁-C₄ alkyl, such as 4-methylpiperazinomethyl.

Oxo-lower alkoxy is, for example, oxo-C₁-C₄ alkoxy, such as 3,3-dimethyl-2-oxo-butyloxy.

Piperazino-lower alkyl is, for example, piperazino-C₁-C₄ alkyl, such as piperazinomethyl, 2-piperazinoethyl, or 3-piperazinopropyl.

Piperidino-lower alkoxy is, for example, piperidino-C₁-C₄ alkoxy, such as piperidinomethoxy, 2-piperidinoethoxy, or 3-piperidinopropyloxy.

Piperidino-lower alkyl is, for example, piperidino-C₁-C₄ alkyl, such as piperidinomethyl, 2-piperidinoethyl, or 3-piperidinopropyl.

Polyhalo-lower alkanesulfonylamino-lower alkoxy is, for example, trifluoro-C₁-C₇ alkanesulfonyl-C₁-C₄ alkoxy, such as trifluoromethanesulfonylaminobutyloxy.

Polyhalo-lower alkanesulfonylamino-lower alkyl is, for example, trifluoro-C₁-C₇ alkanesulfonyl-C₁-C₄ alkyl, such as trifluoromethanesulfonylaminobutyl.

Pyrimidinylthio-lower alkoxy is, for example, pyrimidinylthio-C₁-C₄ alkoxy, such as pyrimidinylthiomethoxy, 2-(pyrimidinylthio)ethoxy, or 3-(pyrimidinylthio)propyloxy.

Pyrrolidino-lower alkoxy is, for example, pyrrolidino-C₂-C₄ alkoxy, such as 2-pyrrolidinoethoxy, or 3-pyrrolidinopropyloxy.

Pyrrolidino-lower alkyl is, for example, pyrrolidino-C₁-C₄ alkyl, such as pyrrolidinomethyl, 2-pyrrolidinoethyl, or 3-pyrrolidinopropyl.

S,S-Dioxothiomorpholino-lower alkyl is, for example, S,S-dioxothiomorpholino-C₁-C₄ alkyl, such as S,S-dioxothiomorpholinomethyl or 2-(S,S-dioxo)thiomorpholinoethyl.

S-Oxothiomorpholino-lower alkyl is, for example, S-oxothiomorpholino-C₁-C₄ alkyl, such as S-oxothiomorpholinomethyl or 2-(S-oxo)thiomorpholinoethyl.

Sulfamoyl-lower alkyl is, for example, sulfamoyl-C₁-C₄ alkyl, such as sulfamoyl-C₁-C₄ alkyl, such as sulfamoylmethyl, 2-sulfamoylethyl, 3-sulfamoylpropyl, or 4-sulfamoylbutyl.

Thiazolinyl-lower alkoxy is, for example, thiazolinyl-C₁-C₄ alkoxy, such as thiazolinylmethoxy, 2-(thiazolinyl)ethoxy or 3-(thiazolinyl)propyloxy.

Thiazolinyl-lower alkyl is, for example, thiazolinyl-C₁-C₄ alkyl, such as thiazolinylmethyl, 2-(thiazolinyl)ethyl, or 3-(thiazolinyl)propyl.

Thiazolyl-lower alkoxy is, for example, thiazolyl-C₁-C₄ alkoxy, such as thiazolylmethoxy, 2-(thiazolyl)ethoxy, or 3-(thiazolyl)propyloxy.

Thiazolyl-lower alkyl is, for example, thiazolyl-C₁-C₄ alkyl, such as thiazolylmethyl, 2-(thiazolyl)ethyl, or 3-(thiazolyl)propyl.

Thiomorpholino-lower alkyl or S,S-dioxothiomorpholino-lower alkyl is, for example, thiomorpholino-C₁-C₄ alkyl, such as -methyl or -ethyl, or S,S-dioxothiomorpholino-C₁-C₄ alkyl, such as -methyl or -ethyl.

Depending on whether asymmetric carbon atoms are present, the compounds of the invention can be present as mixtures of isomers, especially as racemates, or in the form of pure isomers, especially optical antipodes.

Salts of compounds having salt-forming groups are especially acid addition salts, salts with bases or, where several salt-forming groups are present, can also be mixed salts or internal salts.

Salts are especially the pharmaceutically acceptable or non-toxic salts of compounds of formula 1.

Such salts are formed, for example, by compounds of formula I having an acid group, for example a carboxy group or a sulfo group, and are, for example, salts thereof with suitable bases, such as non-toxic metal salts derived from metals of groups Ia, Ib, IIa and IIb of the Periodic Table of the Elements, for example alkali metal salts, especially lithium, sodium or potassium salts, or alkaline earth metal salts, for example magnesium or calcium salts, also zinc salts or ammonium salts, as well as salts formed with organic amines, such as unsubstituted or hydroxy-substituted mono-, di- or tri-alkylamines, especially mono-, di- or tri-lower alkylamines, or with quaternary ammonium bases, for example with methyl-, ethyl-, diethyl- or triethyl-amine, mono-, his- or tris-(2-hydroxy-lower alkyl)-amines, such as ethanol-, diethanol- or triethanol-amine, tris-(hydroxymethyl)-methylamine or 2-hydroxy-tert-butylamines, N,N-di-lower alkyl-N-(hydroxy-lower alkyl)-amines, such as N,N-dimethyl-N-(2-hydroxyethyl)-amine, or N-methyl-D-glucamine, or quaternary ammonium hydroxides, such as tetrabutylammonium hydroxide. The compounds of formula I having a basic group, for example an amino group, can form acid addition salts, for example with suitable inorganic acids, for example hydrohalic acids, such as hydrochloric acid or hydrobromic acid, or sulfuric acid with replacement of one or both protons, phosphoric acid with replacement of one or more protons, e.g., orthophosphoric acid or metaphosphoric acid, or pyrophosphoric acid with replacement of one or more protons, or with organic carboxylic, sulfonic, sulfo or phosphonic acids or N-substituted sulfamic acids, for example, acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, as well as with amino acids, such as the .alpha.-amino acids mentioned hereinbefore, and with methanesulfonic acid, ethanesulfonic acid, 2-hydroxyethanesulfonic acid, ethane-1,2-disulfonic acid, benzenesulfonic acid, 4-toluenesulfonic acid, naphthalene-2-sulfonic acid, 2- or 3-phosphoglycerate, glucose-6-phosphate, or N-cyclohexylsulfamic acid (forming cyclamates) or with other acidic organic compounds, such as ascorbic acid. Compounds of formula I having acid and basic groups can also form internal salts.

For isolation and purification purposes it is also possible to use pharmaceutically unacceptable salts.

Another embodiment of the invention is a pharmaceutical composition comprising an effective amount of compounds of formula I, Ia, or Ib and a pharmaceutically acceptable carrier therefor.

The compounds of the invention may be used, for example, in the preparation of pharmaceutical compositions that comprise an effective amount of the active ingredient together or in admixture with a significant amount of inorganic or organic, solid or liquid, pharmaceutically acceptable carriers.

The pharmaceutical compositions of the invention are compositions for enteral, such as nasal, rectal or oral, or parenteral, such as intramuscular or intravenous, administration to warm-blooded animals (mammals, especially human beings) that comprise an effective dose of the pharmacologically active ingredient alone or together with a pharmaceutically acceptable carrier. The dose of the active ingredient depends on the species of warm-blooded animal, body weight, age and individual condition, individual pharmacokinetic data, the disease to be treated, and the mode of administration.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragees, tablets, or capsules.

The pharmaceutical compositions of the invention are prepared in a manner known per se, for example by means of conventional dissolving, lyophilising, mixing, granulating, or confectioning processes.

Solutions of the active ingredient, and also suspensions, and especially isotonic aqueous solutions or suspensions, are preferably used, it being possible, for example in the case of lyophilised compositions that comprise the active ingredient alone or together with a carrier, for such solutions or suspensions to be made up prior to use. The pharmaceutical compositions may be sterilised and/or may comprise excipients, for example preservatives, stabilisers, wetting agents and/or emulsifiers, solubilisers, salts for regulating the osmotic pressure and/or buffers, and are prepared in a manner known per se, for example by means of conventional dissolving or lyophilising processes. The said solutions or suspensions may comprise conventional viscosity-increasing substances, such as sodium carboxymethylcellulose, carboxymethylcellulose, dextran, polyvinylpyrrolidone, and gelatin.

Suspensions in oil comprise as the oil component the vegetable, synthetic or semi-synthetic oils customary for injection purposes, for example, liquid fatty acid esters that contain as the acid component a long-chained fatty acid having from 8 to 22, especially from 12 to 22, carbon atoms. Examples include lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brassidic acid or linoleic acid, if desired with the addition of antioxidants, for example vitamin E, α-carotene, or 3,5-di-tert-butyl-4-hydroxytoluene. The alcohol component of those fatty acid esters has a maximum of 6 carbon atoms and is a mono- or poly-hydric, for example a mono-, di- or tri-hydric, alcohol, for example methanol, ethanol, propanol, butanol or pentanol, or the isomers thereof, but especially glycol and glycerol. Examples of fatty acid esters include ethyl oleate, isopropyl myristate, isopropyl palmitate, polyoxyethylene glycerol trioleate, triglyceride of saturated fatty acids with a chain length of C₈-C₁₂, but especially vegetable oils, such as cottonseed oil, almond oil, olive oil, castor oil, sesame oil, soybean oil, and groundnut oil.

The injectable compositions are prepared in the customary manner under sterile conditions. The same applies to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. They can also be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tri-calcium phosphate or calcium hydrogen phosphate, and also binders, such as starch pastes using, for example, corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, also carboxy-methyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragee cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and also soft, sealed capsules made of gelatin and a plasticiser, such as glycerol or sorbitol. The dry-filled capsules may comprise the active ingredient in the form of granules, for example with fillers, such as lactose, binders, such as starches, and/or glidants, such as talc or magnesium stearate, and if desired with stabilisers. In soft capsules the active ingredient is preferably dissolved or suspended in suitable oily excipients, such as fatty oils, paraffin oil or liquid polyethylene glycols, it likewise being possible for stabilisers and/or antibacterial agents to be added. Dyes or pigments may be added to the tablets or dragee coatings or to the capsule casings, for example for identification purposes or to indicate different doses of active ingredient.

The compositions of the invention are renin inhibitors. Said compositions contain compounds having a mean inhibition constant (IC₅₀) against renin of between about 50,000 nM to about 0.001 nM; preferably between about 100 nM to about 0.001 nM; and more preferably between about 10 nM to about 0.001 nM. The compositions of the invention may have additionally utility as inhibitors of other aspartic proteases including, but not limited to, HIV protease, plasmepsin and β-secretase.

The compositions of the invention reduce blood pressure. Said compositions include compounds having an IC₅₀ for renin of between about 50,000 nM to about 0.001 nM; preferably between about 100 nM to about 0.001 nM; and more preferably between about 10 nM to about 0.001 nM.

The invention includes a therapeutic method for treating or ameliorating a renin mediated disorder in a subject in need thereof comprising administering to a subject in need thereof an effective amount of a compound of formula I, or the enantiomers, diastereomers, or salts thereof or composition thereof. Renin mediated disorders include hypertension, congestive heart failure, cardiac hypertrophy, cardiac fibrosis, cardiomyopathy post-infarction, complications resulting from diabetes, such as nephropathy, vasculopathy and neuropathy, diseases of the coronary vessels, post-surgical hypertension, restenosis following angioplasty, raised intra-ocular pressure, glaucoma, abnormal vascular growth, hyperaldosteronism, anxiety states, and cognitive disorders (Fisher N.D.; Hollenberg N. K. Expert Opin. Investig. Drugs. 2001, 10, 417-26).

Administration methods include administering an effective amount (i.e., a therapeutically effective amount) of a compound or composition of the invention at different times during the course of therapy or concurrently in a combination form. The methods of the invention include all known therapeutic treatment regimens.

“Prodrug” means a pharmaceutically acceptable form of an effective derivative of a compound (or a salt thereof) of the invention, wherein the prodrug may be: 1) a relatively active precursor which converts in vivo to a compound of the invention; 2) a relatively inactive precursor which converts in vivo to a compound of the invention; or 3) a relatively less active component of the compound that contributes to therapeutic activity after becoming available in vivo (i.e., as a metabolite). See “Design of Prodrugs”, ed. H. Bundgaard, Elsevier, 1985.

“Metabolite” means a pharmaceutically acceptable form of a metabolic derivative of a compound (or a salt thereof) of the invention, wherein the derivative is an active compound that contributes to therapeutic activity after becoming available in vivo.

“Effective amount” means that amount of active compound agent that elicits the desired biological response in a subject. Such response includes alleviation of the symptoms of the disease or disorder being treated. The effective amount of a compound of the invention in such a therapeutic method to be administered to warm-blooded animals, for example human beings, of, for example, approximately 70 kg body weight, especially the doses effective in the inhibition of the enzyme renin, in lowering blood pressure and/or in improving the symptoms of glaucoma, are from approximately 3 mg to approximately 3 g, preferably from approximately 10 mg to approximately 1 g, for example approximately from 20 mg to 200 mg, per person per day, divided preferably into 1 to 4 single doses which may, for example, be of the same size. Usually, children receive about half of the adult dose. The dose necessary for each individual can be monitored, for example by measuring the serum concentration of the active ingredient, and adjusted to an optimum level.

The invention includes the use of a compound of the invention for the preparation of a composition for treating or ameliorating a renin mediated chronic disorder or disease or infection in a subject in need thereof, wherein the composition comprises a mixture one or more compounds of the invention and an optional pharmaceutically acceptable carrier.

“Pharmaceutically acceptable carrier” means compounds and compositions that are of sufficient purity and quality for use in the formulation of a composition of the invention and that, when appropriately administered to an animal or human, do not produce an adverse reaction.

“Renin mediated disorder or disease” includes disorders or diseases associated with the elevated expression or overexpression of renin and conditions that accompany such diseases.

An embodiment of the invention includes administering a renin inhibiting compound of formula I or composition thereof in a combination therapy (see U.S. Pat. No. 5,821,232, U.S. Pat. No. 6,716,875, U.S. Pat. No. 5,663,188, Fossa, A. A.; DePasquale, M. J.; Ringer, L. J.; Winslow, R. L. “Synergistic effect on reduction in blood pressure with coadministration of a renin inhibitor or an angiotensin-converting enzyme inhibitor with an angiotensin II receptor antagonist” Drug Development Research 1994, 33(4), 422-8) with one or more additional agents for the treatment of hypertension including α-blockers, β-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitor, aldosterone-receptor antagonists, or endothelin receptor antagonist.

α-Blockers include doxazosin, prazosin, tamsulosin, and terazosin.

β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts.

Calcium channel blockers include dihydropyridines (DHPs) and non-DHPs. The preferred DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts. Non-DHPs are selected from flunarizine, prenylamine, diltiazem, fendiline, gallopamil, mibefradil, anipamil, tiapamil, and verampimil and their pharmaceutically acceptable salts.

A diuretic is, for example, a thiazide derivative selected from amiloride, chlorothiazide, hydrochlorothiazide, methylchlorothiazide, and chlorothalidon.

ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril.

Dual ACE/NEP inhibitors are, for example, omapatrilat, fasidotril, and fasidotrilat.

Preferred ARBs include candesartan, eprosartan, irbesartan, losartan, olmesartan, tasosartan, telmisartan, and valsartan.

Preferred aldosterone synthase inhibitors are anastrozole, fadrozole, and exemestane.

Preferred aldosterone-receptor antagonists are spironolactone and eplerenone.

A preferred endothelin antagonist is, for example, bosentan, enrasentan, atrasentan, darusentan, sitaxentan, and tezosentan and their pharmaceutically acceptable salts.

Combination therapy includes co-administration of the compound of the invention and said other agent, sequential administration of the compound and the other agent, administration of a composition containing the compound and the other agent, or simultaneous administration of separate compositions containing of the compound and the other agent.

The compounds of the invention have enzyme-inhibiting properties. In particular, they inhibit the action of the natural enzyme renin. The latter passes from the kidneys into the blood where it effects the cleavage of angiotensinogen, releasing the decapeptide angiotensin I which is then cleaved in the blood, lungs, the kidneys and other organs by angiotensin converting enzyme to form the octapeptide angiotensin II. The octapeptide increases blood pressure both directly by binding to its receptor, causing arterial vasoconstriction, and indirectly by liberating from the adrenal glands the sodium-ion-retaining hormone aldosterone, accompanied by an increase in extracellular fluid volume. That increase can be attributed to the action of angiotensin II. Inhibitors of the enzymatic activity of renin bring about a reduction in the formation of angiotensin I. As a result a smaller amount of angiotensin II is produced. The reduced concentration of that active peptide hormone is the direct cause of the hypotensive effect of renin inhibitors.

The action of renin inhibitors in vitro is demonstrated experimentally by means of a test which measures the increase in fluorescence of an internally quenched peptide substrate. The sequence of this peptide corresponds to the sequence of human angiotensinogen. The following test protocol is used: All reactions are carried out in a flat bottom white opaque microtiter plate. A 4 μL aliquot of 400 μM renin substrate (DABCYL-γ-Abu-Ile-His-Pro-Phe-His-Leu-Val-Ile-His-Thr-EDANS) in 192 μL assay buffer (50 mM BES, 150 mM NaCl, 0.25 mg/mL bovine serum albumin, pH7.0) is added to 4 μL of test compound in DMSO at various concentrations ranging from 10 μM to 1 nM final concentrations. Next, 100 μL of trypsin-activated recombinant human renin (final enzyme concentration of 0.2-2 nM) in assay buffer is added, and the solution is mixed by pipetting. The increase in fluorescence at 495 nm (excitation at 340 nm) is measured for 60-360 minutes at room temperature using a Perkin-Elmer Fusion microplate reader. The slope of a linear portion of the plot of fluorescence increase as a function of time is then determined, and the rate is used for calculating percent inhibition in relation to uninhibited control. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor. In the in vitro systems the compounds of the invention exhibit inhibiting activities at minimum concentrations of from approximately 5×10⁻⁵ M to approximately 10⁻¹² M. Preferred compounds of the invention exhibit inhibiting activities at minimum concentrations of from approximately 5×10⁻⁸ M to approximately 10⁻¹² M. More preferred compounds of the invention exhibit inhibiting activities at minimum concentrations of from approximately 10⁻⁸ M to approximately 10⁻¹² M. (Wang G. T. et al. Anal. Biochem. 1993, 210, 351; Nakamura, N. et al. J. Biochem. (Tokyo) 1991, 109, 741; Murakami, K. et al. Anal Biochem. 1981, 110, 232).

The action of renin inhibitors in vitro in human plasma is demonstrated experimentally by the decrease in plasma renin activity (PRA) levels observed in the presence of the compounds. Incubations mixtures contained in the final volume of 250 μL 95.5 mM N,N-bis(2-hydroxyethyl)-2-aminoethanesulfonic acid, pH 7.0, 8 mM EDTA, 0.1 mM neomycin sulfate, 1 mg/mL sodium azide, 1 mM phenylmethanesulfonyl fluoride, 2% DMSO and 87.3% of pooled mixed-gender human plasma stabilized with EDTA. For plasma batches with low PRA (less than 1 ng/ml/hr) ˜2 pM of recombinant human renin was added to achieve PRA of 3-4 ng/mL/hr. The cleavage of endogenous angiotensinogen in plasma was carried out at 37° C. for 90 min and the product angiotensin I was measured by competitive radioimmunoassay using DiaSorin PRA kit. Uninhibited incubations containing 2% DMSO and fully inhibited controls with 2 μM of isovaleryl-Phe-Nle-Sta-Ala-Sta-OH were used for deriving percent of inhibition for each concentration of inhibitors and fitting dose-response data into a four parametric model from which IC₅₀ values, defined as concentrations of inhibitors at which 50% inhibition occurs, were determined.

The cardiac and systemic hemodynamic efficacy of selective renin inhibitors were evaluated in vivo in sodium-depleted, normotensive cynomolgus monkeys, in sodium-depleted, normotensive beagle dogs following a single oral and intravenous administration of the test compound. Arterial blood pressure was monitored by telemetry in freely moving, conscious animals.

Cynomolgus Monkey: Six male naive cynomolgus monkeys weighing between 2.5 and 3.5 kg were used in the studies. At least 4 weeks, before the experiment, the monkeys were anesthetized with ketamine hydrochloride (15 mg/kg, i.m.) and xylazine hydrochloride (0.7 mg/kg, i.m.), and were implanted into the abdominal cavity with a transmitter (Model #TL11M2-D70-PCT, Data Sciences, St. Paul, Minn.). The pressure catheter was inserted into the lower abdominal aorta via the femoral artery. The bipotential leads were placed in Lead II configuration. The animals were housed under constant temperature (19-25° C.), humidity (>40%) and lighting conditions (12 h light and dark cycle), were fed once daily, and were allowed free access to water. The animals were sodium depleted by placing them on a low sodium diet (0.026%, Expanded Primate Diet 829552 MP-VENaCI (P), Special Diet Services, Ltd., UK) 7 days before the experiment and furosemide (3 mg/kg, intramuscularly i.m., Aventis Pharmaceuticals) was administered at −40 h and −16 h prior to administration of test compound.

For oral dosing, the renin inhibitors were formulated in 0.5% methylcellulose at dose levels of 10 and 30 mg/kg (5 mL/kg) by infant feeding tubes. For intravenous delivery, a silastic catheter was implanted into posterior vena cava via a femoral vein. The catheter was attached to the delivery pump via a tether system and a swivel joint. Test compound (dose levels of 0.1 to 10 mg/kg, formulated at 5% dextrose) was administered by continuous infusion (1.67 mL/kg/h) or by bolus injection (3.33 mL/kg in 2 min).

Arterial blood pressures (systolic, diastolic and mean) and body temperature were recorded continuously at 500 Hz and 50 Hz, respectively, using the Dataquest™ A.R.T. (Advanced Research Technology) software. Heart rate was derived from the phasic blood pressure tracing. During the recording period, the monkeys were kept in a separate room without human presence to avoid pressure changes secondary to stress. All data were expressed as mean ±SEM. Effects of the renin inhibitors on blood pressure were assessed by ANOVA, taking into account the factors dose and time compared with the vehicle group.

Beagle Dogs: Non-naive Beagle dogs (2 per sex) weighing between 9 and 11 kg were used in the studies. Each animal was implanted subcutaneously with a telemetry transmitter (Data Sciences) and the blood pressure catheter was inserted into the left femoral artery. The electrocardiogram leads were also tunneled subcutaneously to the appropriate anatomical regions. The animals were housed under constant temperature and lighting conditions, were fed once daily, and were allowed free access to water. A sodium depleted state was produced by placing them on a low-sodium diet (<4 meq/day, a combination of canned Prescription Diet canine h/d, from Hill's Pet Products and dry pellets from Bio-Serv Inc., Frenchtown, N.J.) beginning 10 days before the experiment, and furosemide (3 mg/kg i.m.; Aventis Pharmaceuticals) was administered at −40 and -16 hr prior to administration of test compound.

A renin inhibitor was orally administered by orogastric gavage to all overnight fasted animals at a dose level of 30 mg/kg (4 mL/kg formulated in 0.5% methylcellulose). Food was given 4 h postdose. In some experiments, the renin inhibitor was administered by bolus i.v. at increasing dose levels of 1, 3 and 6 mg/kg (2, 6 and 20 mg/mL formulated in sterile saline). Cardiovascular parameters were collected continuously at least 80 min predose and 3 h postdose, followed by every 10 min for 5 h and every 30 min for 16 h postdose. The Dataquest™ ART (version 2.2) software package from DSI (Data Sciences International) was used to collect telemetered cardiovascular data.

The efficacy of the renin inhibitors was also evaluated in vivo in double transgenic rats engineered to express human renin and human angiotensinogen (Bohlender J, Fukamizu A, Lippoidt A, Nomura T, Dietz R, Menard J, Murakami K, Luft F C, Ganten D. High human renin hypertension in transgenic rats. Hypertension 1997, 29, 428-434).

Experiments were conducted in 6-week-old double transgenic rats (dTGRs). The model has been described in detail earlier. Briefly, the human renin construct used to generate transgenic animals made up the entire genomic human renin gene (10 exons and 9 introns), with 3.0 kB of the 5′-promoter region and 1.2 kB of 3′ additional sequences. The human angiotensinogen construct made up the entire human angiotensinogen gene (5 exons and 4 introns), with 1.3 kB of 5′-flanking and 2.4 kB of 3′-flanking sequences. The rats were purchased from RCC Ltd (Füllinsdorf, Switzerland). Radio telemetry transmitters were surgically implanted at 4 weeks of age. The telemetry system provided 24-h recordings of systolic, mean, diastolic arterial pressure (SAP, MAP, DAP, respectively) and heart rate (HR). Beginning on day 42, animals were transferred to telemetry cages. A 24 h telemetry reading was obtained. Rats were then dosed orally on the following 4 consecutive days (days 43-46). The rats were monitored continuously and allowed free access to standard 0.3%-sodium rat chow and drinking water.

The compounds of the invention are useful for ameliorating or treating disorders or diseases in which decreasing the levels of renin products is effective in treating a disease state. In hypertension elevated levels of angiotensin 1, the product of renin catalyzed cleavage of angioteninogen are present. Thus, the compounds of the invention can be used in the treatment of hypertension, congestive heart failure, cardiac hypertrophy, cardiac fibrosis, cardiomyopathy post-infarction, complications resulting from diabetes, such as nephropathy, vasculopathy and neuropathy, diseases of the coronary vessels, proteinuria, albumenuria, post-surgical hypertension, metabolic syndrome, obesity, restenosis following angioplasty, raised intra-ocular pressure, glaucoma, abnormal vascular growth, hyperaldosteronism, anxiety states, and cognitive disorders (Fisher N. D.; Hollenberg N. K. Expert Opin. Investig. Drugs. 2001, 10, 417-26).

The first process of the invention for the preparation of compounds of formula I wherein Q=C═O comprises

1) reacting a compound of formula II with a compound of formula IV

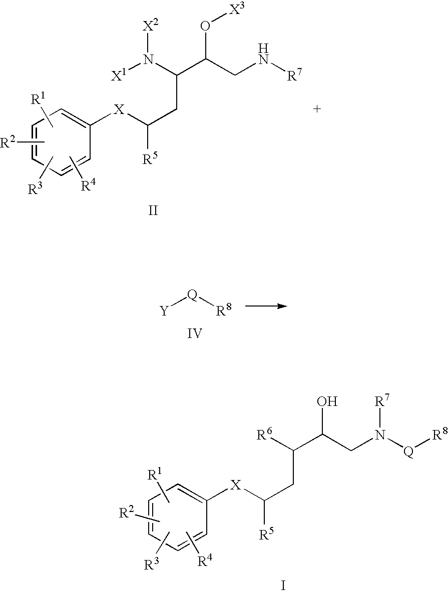

wherein
X¹ is lower alkyl, lower alkanoyl, or an amino-protecting group;
X² is H or together with X³ is a bivalent protecting group;
X³ is H or a hydroxy-protecting group; and
R¹, R², R³, R⁴, X, R⁵, and R⁷ in II are as defined for formula I,
R⁸ in IV has one of the meanings given for formula I, Q is C═O and Y is either OH, Cl, F, Br, or OR¹¹ such that OR¹¹ is an activated ester, mixed or symmetrical anhydride linkage, or acyl carbonate to form an amide bond, and
2) removing any protecting groups present,
or, and if desired, converting the compound of formula I produced having at least one salt-forming group obtainable into its salt,
or converting an obtainable salt into the free compound or into a different salt and/or separating mixtures of isomers that may be obtainable.

Functional groups in starting materials which are prone to participate in undesired side reactions, especially amino, carboxy, hydroxy, and mercapto groups, can be protected by suitable conventional protecting groups which are customarily used in the synthesis of peptide compounds, and also in the synthesis of cephalosporins and penicillins as well as nucleic acid derivatives and sugars. Those protecting groups may already be present in the precursors and are intended to protect the functional groups in question against undesired secondary reactions, such as acylation, etherification, esterification, oxidation, solvolysis, etc. In certain cases the protecting groups can additionally cause the reactions to proceed selectively, for example stereoselectively. It is characteristic of protecting groups that they can be removed easily, i.e. without undesired secondary reactions taking place, for example by acid treatment, fluoride treatment, solvolysis, reduction, photolysis, and also enzymatically, for example under physiological conditions. Protecting groups may also be present in the end products. Compounds of formula I having protected functional groups may have greater metabolic stability or pharmacodynamic properties that are better in some other way than the corresponding compounds having free functional groups.

The protection of functional groups by such protecting groups, the protecting groups themselves, and the reactions for their removal are described, for example, in standard works such as T. W. Greene and P. G. M. Wuts “Protective Groups in Organic Synthesis” John Wiley & Sons, Inc., New York 1999.

In compounds of formula II, amino-protecting groups X¹ are, for example, acyl groups other than lower alkanoyl, also arylmethyl, lower alkylthio, 2-acyl-lower alk-1-enyl or silyl. The group X¹—N(X²)— can also be in the form of an azido group.

Acyl groups other than lower alkanoyl are, for example, halo-lower alkanoyl, for example 2-haloacetyl, such as 2-chloro-, 2-bromo-, 2-iodo-, 2,2,2-trifluoro- or 2,2,2-trichloro-acetyl, unsubstituted or substituted, for example halo-, lower alkoxy- or nitro-substituted, benzoyl, for example benzoyl, 4-chlorobenzoyl, 4-methoxybenzoyl or 4-nitrobenzoyl, or lower alkoxycarbonyl that is branched in the 1-position of the lower alkyl radical or suitably substituted in the 1- or 2-position, for example tertiary lower alkoxycarbonyl, such as tert-butoxycarbonyl, arylmethoxy-carbonyl having one or two aryl radicals which are phenyl that is unsubstituted or mono- or poly-substituted, for example, by lower alkyl, for example tertiary lower alkyl, such as tertiary butyl, lower alkoxy, such as methoxy, hydroxy, halogen, such as chlorine, and/or by nitro, for example benzyloxycarbonyl, unsubstituted or substituted benzyloxycarbonyl, such as 4-nitrobenzyl-oxycarbonyl, diphenylmethoxycarbonyl, fluorenylmethoxycarbonyl or substituted diphenylmethoxycarbonyl, such as di(4-methoxyphenyl)methoxycarbonyl, aroylmethoxycarbonyl wherein the aroyl group is preferably benzoyl that is unsubstituted or substituted, for example, by halogen, such as bromine, for example phenacyloxycarbonyl, 2-halo-lower alkoxycarbonyl, for example 2,2,2-trichloroethoxycarbonyl, 2-bromoethoxycarbonyl or 2-iodo-ethoxycarbonyl, 2-(tri-substituted silyl)-lower alkoxycarbonyl, for example 2-tri-lower alkylsilyl-lower alkoxycarbonyl, for example 2-trimethylsilylethoxycarbonyl or 2-(di-n-butyl-methyl-silyl)-ethoxycarbonyl, or triarylsilyl-lower alkoxycarbonyl, for example 2-triphenylsilylethoxycarbonyl.

In a 2-acyl-lower alk-1-enyl radical that can be used as an amino-protecting group, acyl is, for example, the corresponding radical of a lower alkanecarboxylic acid, of a benzoic acid that is unsubstituted or substituted, for example, by lower alkyl, such as methyl or tertiary butyl, lower alkoxy, such as methoxy, halogen, such as chlorine, and/or by nitro, or especially of a carbonic acid semiester, such as a carbonic acid lower alkyl semiester. Corresponding protecting groups are especially 1-lower alkanoyl-prop-1-en-2-yl, for example 1-acetyl-prop-1-en-2-yl, or lower alkoxycarbonyl-prop-1-en-2-yl, for example 1-ethoxy-carbonyl-prop-1-en-2-yl.

Silylamino groups are, for example, tri-lower alkylsilylamino groups, for example trimethylsilylamino, triisopropylamino and t-butyldimethylsilylamino.

An amino group can also be protected by conversion into the protonated form; suitable corresponding anions are especially those of strong inorganic acids, such as sulfuric acid, phosphoric acid or hydrohalic acids, for example the chlorine or bromine anion, or of organic sulfonic acids, such as p-toluenesulfonic acid.

Preferred amino-protecting groups X¹ are acyl radicals of carbonic acid semiesters, such as lower alkoxycarbonyl, especially tert-butyloxycarbonyl or fluorenylmethoxycarbonyl, unsubstituted or lower alkyl-, lower alkoxy-, nitro- and/or halo-substituted \acute{α}-phenyl- or .\acute{α},\acute{α}-diphenyl-lower alkoxycarbonyl, such as benzyloxycarbonyl, p-nitrobenzyloxy-carbonyl or diphenylmethoxycarbonyl, or 2-halo-lower alkoxycarbonyl, e.g., 2,2,2-trichloroethoxycarbonyl, or 2-(trialkylsyl)ethoxycarbonyl e.g. 2-(trimethylsilyl)ethoxycarbonyl, also trityl or formyl.

Hydroxy-protecting groups X³ are, for example, acyl groups, for example lower alkanoyl that is substituted by halogen, such as chlorine, for example 2,2-dichloroacetyl, or especially acyl radicals of a carbonic acid semiester mentioned for protected amino groups. A preferred hydroxy-protecting group is, for example, 2,2,2-trichloroethoxycarbonyl, 4-nitrobenzyloxy-carbonyl, diphenylmethoxycarbonyl or trityl. A further suitable hydroxy-protecting group X₃ is tri-lower alkylsilyl, for example trimethylsilyl, triisopropylsilyl or dimethyl-tert-butylsilyl, a readily removable etherifying group, for example an alkyl group, such as tertiary lower alkyl, for example tertiary butyl, an oxa- or a thia-aliphatic or -cycloaliphatic, especially 2-oxa- or 2-thia-aliphatic or -cycloaliphatic, hydrocarbon radical, for example 1-lower alkoxy-lower alkyl or 1-lower alkylthio-lower alkyl, for example methoxymethyl, 1-methoxyethyl, 1-ethoxyethyl, methylthiomethyl, 1-methylthioethyl or 1-ethylthioethyl, or 2-oxa- or 2-thia-cycloalkyl having from 5 to 7 ring atoms, for example 2-tetrahydrofuryl or 2-tetrahydropyranyl, or a corresponding thia analogue, and also 1-phenyl-lower alkyl, for example benzyl, diphenylmethyl or trityl, wherein the phenyl radicals can be substituted, for example, by halogen, for example chlorine, lower alkoxy, for example methoxy, and/or by nitro.

Bivalent protecting groups formed by X² and X³ together are, for example, methylene groups substituted by one or two alkyl radicals and are accordingly unsubstituted or substituted alkylidene, such as lower alkylidene, for example isopropylidene, cycloalkylidene, such as cyclohexylidene, also carbonyl or benzylidene; or dialkylsilyl groups, such dimethylsilyl.

In compounds of formula IV, Y is a reactively etherified or esterified hydroxy, and is, for example, in the form of an activated ester or anhydride. The reactive acid derivatives can also be formed in situ.

Such activated esters of compounds of formula IV are especially esters unsaturated at the linking carbon atom of the esterifying radical, for example of the vinyl ester type, such as vinyl esters (obtainable, for example, by transesterification of a corresponding ester with vinyl acetate; activated vinyl ester method), carbamoyl esters (obtainable, for example, by treatment of the corresponding acid with an isoxazolium reagent; 1,2-oxazolium or Woodward method), or 1-lower alkoxyvinyl esters (obtainable, for example, by treatment of the corresponding acid with a lower alkoxyacetylene; ethoxyacetylene method), or esters of the amidino type, such as N,N′-disubstituted amidino esters (obtainable, for example, by treatment of the corresponding acid with a suitable N,N′-disubstituted carbodiimide, for example N,N′-dicyclohexylcarbodiimide; carbodiimide method), or N,N-disubstituted amidino esters (obtainable, for example, by treatment of the corresponding acid with an N,N-disubstituted cyanamide; cyanamide method), suitable aryl esters, especially phenyl esters suitably substituted by electron-attracting substituents (obtainable, for example, by treatment of the corresponding acid with a suitably substituted phenol, for example 4-nitrophenol, 4-methylsulfonylphenol, 2,4,5-trichlorophenol, 2,3,4,5,6-pentachlorophenol or 4-phenyldiazophenol, in the presence of a condensation agent, such as N,N′-dicyclohexylcarbodiimide; activated aryl esters method), cyanomethyl esters (obtainable, for example, by treatment of the corresponding acid with chloroacetonitrile in the presence of a base; cyanomethyl esters method), thioesters, especially unsubstituted or substituted, for example nitro-substituted, phenylthio esters (obtainable, for example, by treatment of the corresponding acid with unsubstituted or substituted, for example nitro-substituted, thiophenols, inter alia by the anhydride or carbodiimide method; activated thiol esters method), or especially amino or amido esters (obtainable, for example, by treatment of the corresponding acid with an N-hydroxyamino or N-hydroxyamido compound, for example N-hydroxysuccinimide, N-hydroxypiperidine, N-hydroxyphthalimide, N-hydroxy-5-norbornene-2,3-dicarboxylic acid imide, 1-hydroxybenzotriazole or 3-hydroxy-3,4-dihydro-1,2,3-benzotriazin-4-one, for example by the anhydride or carbodiimide method; activated N-hydroxy esters method). Internal esters, for example .gamma.-lactones, can also be used.

Anhydrides of acids of formula IV may be symmetric or preferably mixed anhydrides of those acids, for example anhydrides with inorganic acids, such as acid halides, especially acid chlorides (obtainable, for example, by treatment of the corresponding acid with thionyl chloride, phosphorus pentachloride or oxalyl chloride; acid chloride method), azides (obtainable, for example, from a corresponding acid ester via the corresponding hydrazide and treatment thereof with nitrous acid; azide method), anhydrides with carbonic acid semiesters, for example carbonic acid lower alkyl semiesters (obtainable, for example, by treatment of the corresponding acid with chloroformic acid lower alkyl esters or with a 1-lower alkoxycarbonyl-2-lower alkoxy-1,2-dihydroquinoline; mixed O-alkyl-carbonic acid anhydrides method), or anhydrides with dihalogenated, especially dichlorinated, phosphoric acid (obtainable, for example, by treatment of the corresponding acid with phosphorus oxychloride; phosphorus oxychloride method), anhydrides with other phosphoric acid derivatives (for example those obtainable with phenyl-N-phenylphosphoramidochloridate) or with phosphorous acid derivatives, or anhydrides with organic acids, such as mixed anhydrides with organic carboxylic acids (obtainable, for example, by treatment of the corresponding acid with an unsubstituted or substituted lower alkane- or phenyl-lower alkane-carboxylic acid halide, for example phenylacetic acid chloride, pivalic acid chloride or trifluoroacetic acid chloride; mixed carboxylic acid anhydrides method) or with organic sulfonic acids (obtainable, for example, by treatment of a salt, such as an alkali metal salt, of the corresponding acid with a suitable organic sulfonic acid halide, such as a lower alkane- or aryl-, for example methane- or p-toluene-sulfonic acid chloride; mixed sulfonic acid anhydrides method) and symmetric anhydrides (obtainable, for example, by condensation of the corresponding acid in the presence of a carbodiimide).

Amine compounds of formula II can be prepared, for example, by reacting an epoxide compound of formula V with an amine of formula VI:

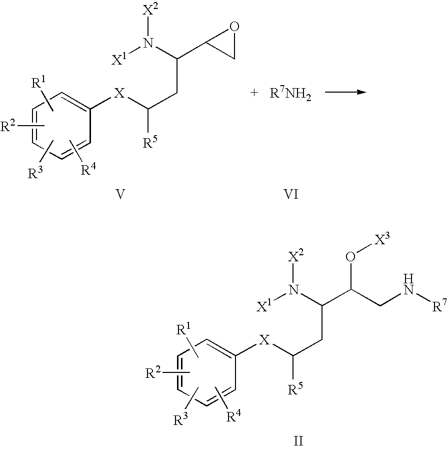

where R⁷ is defined as in formula I; followed by appropriate protecting group manipulation.

Amine compounds of formula II wherein R⁷═H can also be prepared by reduction of azide compounds of formula VII using hydrogen gas in the presence of a transition metal catalyst, for example Raney nickel or platinum or palladium catalysts, for example platinum or palladium on active carbon, or with triphenylphosphine in an aqueous-organic solvent mixture (Staudinger reduction). Azide compounds VII can be prepared by reacting by reacting an epoxide compound of formula V with nucleophilic azide source such as sodium azide in an organic solvent such as DMF or acetonitrile:

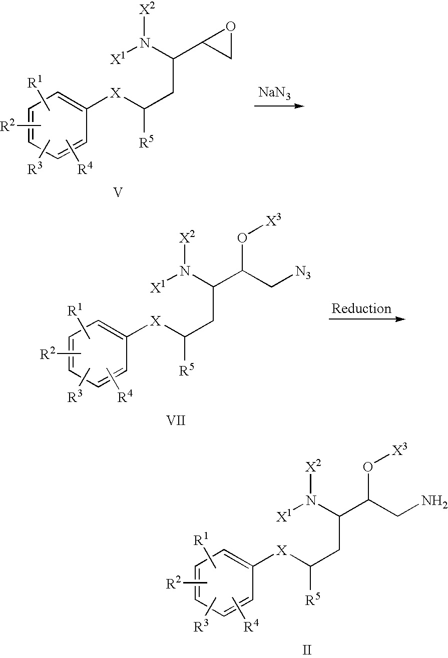

In a second process of the invention, compounds of formula I wherein Q is SO₂ are prepared, for example, by 1) treatment of epoxide compounds of formula V with compounds of formula VIII wherein Q=SO₂, followed by 2) protecting group removal:

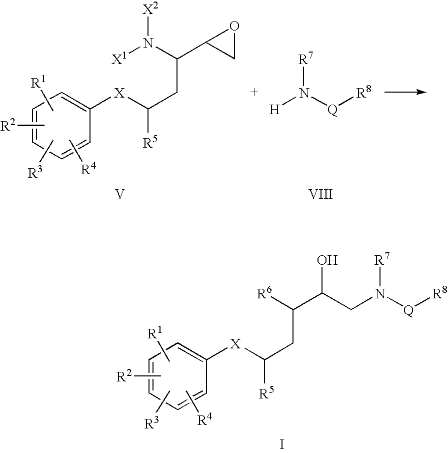

In a third process of the invention, compounds of formula I wherein Q=S are prepared, for example, by treatment of optionally protected compounds of formula I in which Q=O with P₂S₅ or Lawesson's reagent, followed by protecting group removal.

Epoxide compounds of formula V can, in turn, be prepared in a number of ways including, for example, by reacting with aldehyde compounds of formula IX with trimethylsulfoxonium Iodide or trimethylsulfonium iodide (J. Aube “Epoxidation and Related Processes” Chapter 3.2 in Volume 1 of “Comprehensive Organic Synthesis” Edited by B. M. Trost, I. Fleming and Stuart L. Schreiber, Pergamon Press New York, 1992).

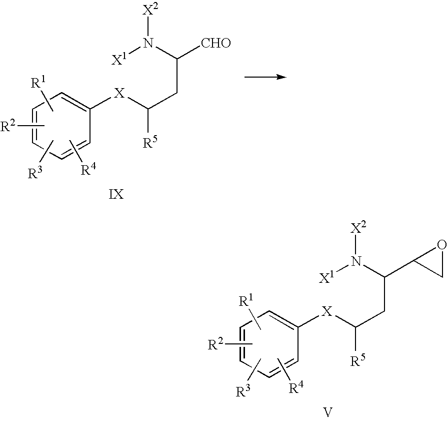

Compounds of formula IX can be prepared from compounds of formula X, wherein R¹⁰ is lower alkyl or aryl-lower alkyl, in a number of ways. For example, compounds of formula X can be converted to compounds of formula IX:

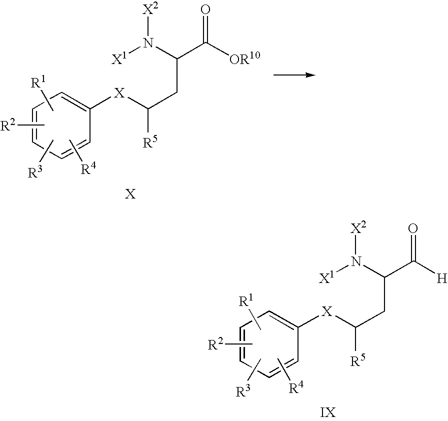

by direct reduction from ester to aldehyde using specialized reagents and conditions known to minimize over-reduction (I. T. Harrison and S. Harrison “Compendium of Organic Synthetic Methods” Section 53, pp 152-153, John Wiley and Sons, New York 1971). One method of carrying out this transformation is by treatment with diisobutyl aluminum hydride in an organic solvent at lowered temperatures. The synthesis of compounds of Formula IX is described in U.S. Pat. No. 5,559,111 at columns 25-26.

Alternately, compounds of formula IX can be prepared from alcohol compounds of formula XI:

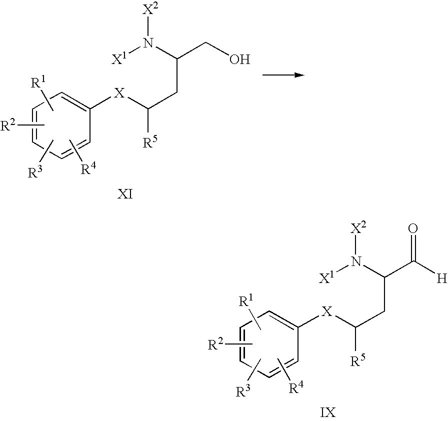

using one of several oxidation protocols which are designed to minimize overoxidation (I. T. Harrison and S. Harrison “Compendium of Organic Synthetic Methods” Section 48, pp 137-143, John Wiley and Sons, New York 1971). Such oxidation protocols include oxalyl chloride/dimethyl sulfoxide (Swern oxidation), (1,1,1-triacetoxy)-1,1-dihydro-1,2-dihydro-1,2-benziodoxol-3(1H)-one (Dess-Martin periodinane), sulfur trioxide/pyridine or tetrapropylammonium perruthenate (TPAP).

Alcohol compounds of formula XI are prepared from ester compounds of formula X by a variety of reducing agents (I. T. Harrison and S. Harrison “Compendium of Organic Synthetic Methods” Section 38, pp 87-91, John Wiley and Sons, New York 1971) including, for example, lithium aluminum hydride.

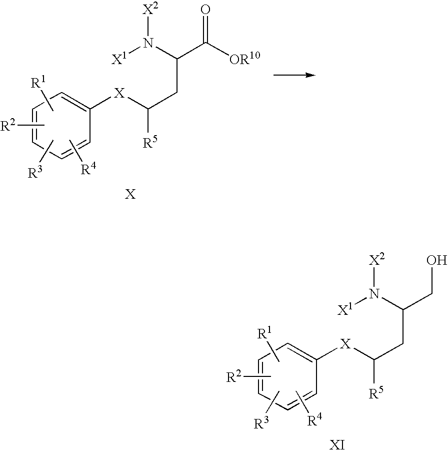

As another example, compounds of formula X can be hydrolyzed to carboxylic acid compounds of formula XII (I. T. Harrison and S. Harrison “Compendium of Organic Synthetic Methods” Section 23, pp 42-46, John Wiley and Sons, New York 1971). Compounds of formula XII can be converted to alcohol compounds of formula XI using a wide variety of reducing agents and conditions (I. T. Harrison and S. Harrison “Compendium of Organic Synthetic Methods” Section 32, pp 76-78, John Wiley and Sons, New York 1971).

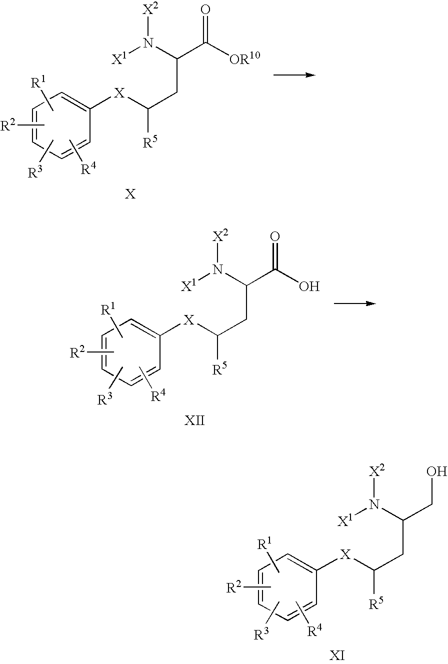

Alternately, epoxide compounds of formula V can be prepared from alkene compounds of formula XIII by epoxidation of the alkene with for example mCPBA, monoperphthalic acid, peracetic acid, dimethyldioxirane, H₂O₂/benzonitrile.

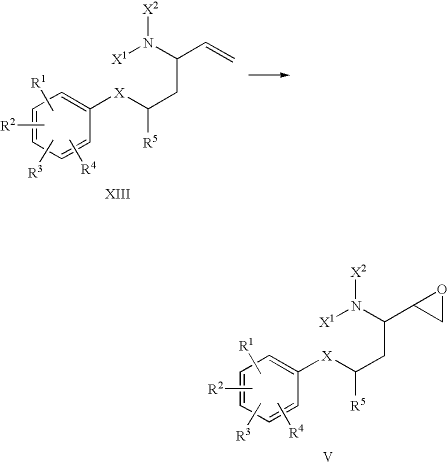

Alkene compounds of formula XIII are prepared from aldehyde compounds of formula IX utilizing the Wittig reaction or the Tebbe reagent.

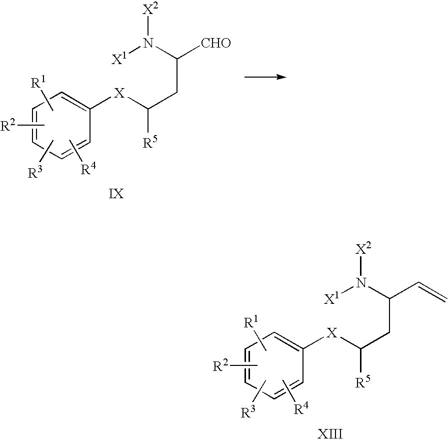

Compounds of formula II in which R⁷ is a lower alkyl, certain lower haloalkyl groups, lower cycloalkyl, certain lower alkoxyalkyl groups or certain lower haloalkoxy-lower alkyl groups are prepared by reductive alkylation of primary amines of formula II wherein R⁷═H with aldehydes of formula XIV wherein R^{7a} is the lower homolog of R⁷ (E. W. Baxter and A. B. Reitz “Reductive animations of carbonyl compounds with borohydride and borane reducing agents” in Organic Reactions Volume 59 pp 1-714, Edited by L. E. Overman, John Wiley and Sons, New York, 2002).

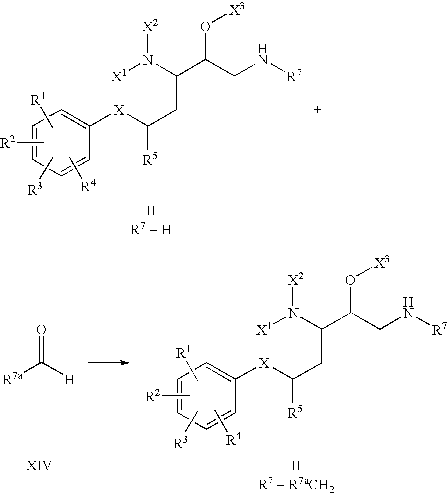

In a fourth process of the invention, compounds of formula I wherein X═CH₂ are prepared by 1) hydrogenolysis or deoxygenation of optionally protected compounds of formula I wherein X═CH(OH), or an ester thereof such as an acetate, using for example hydrogen gas and a transition metal catalyst, for example Raney nickel or platinum or palladium catalysts, for example platinum or palladium on active carbon; followed by 2) protecting group removal:

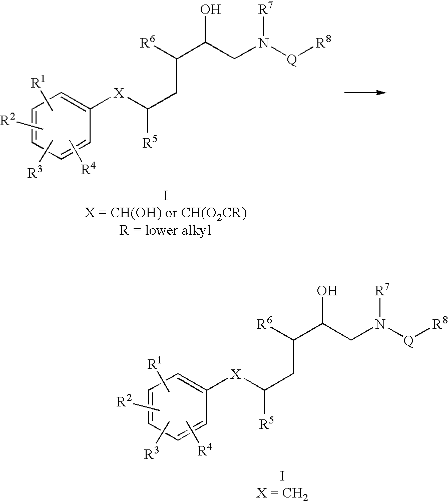

In a fifth process of the invention, compounds of formula I wherein X═CH(OH) are prepared by addition of organometallic compounds of formula XV wherein M is for example Li, MgCl, MgBr or MgI to aldehyde compounds of formula III:

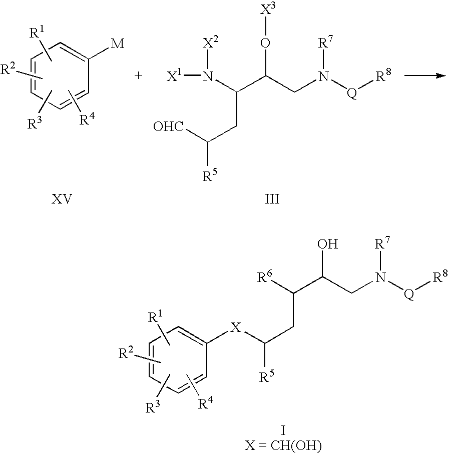

Aldehyde compounds of formula III are prepared by oxidation of alcohols of formula XVI using, for example, oxalyl chloride/dimethyl sulfoxide (Swern oxidation), (1,1,1-triacetoxy)-1,1-dihydro-1,2-dihydro-1,2-benziodoxol-3(1H)-one (Dess-Martin periodinane), sulfur trioxide/pyridine or tetrapropylammonium perruthenate (TPAP).

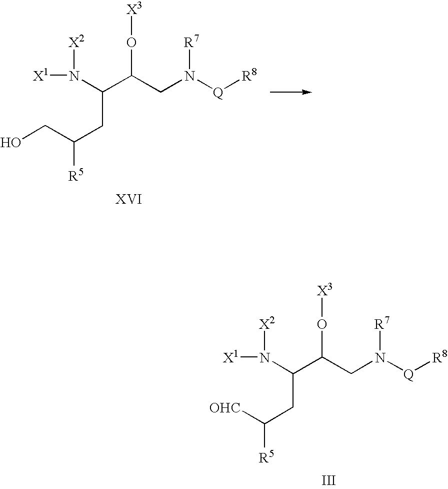

Alcohols of formula XVI are obtained from protected alcohols of formula XVII wherein X⁴ is an alcohol protecting group that can be removed selectively in the presence of the protecting groups X¹, X² and X³, for example a benzyl group or a trialkylsilyl ether.

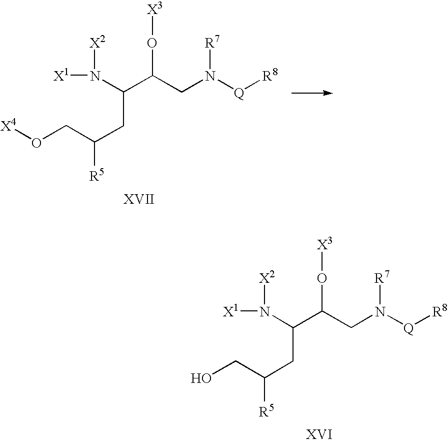

Compounds of formula XVII wherein Q is C═O are prepared from amines of formula XVIII by coupling with a carboxylic acid derivative of formula IV wherein Q is C═O:

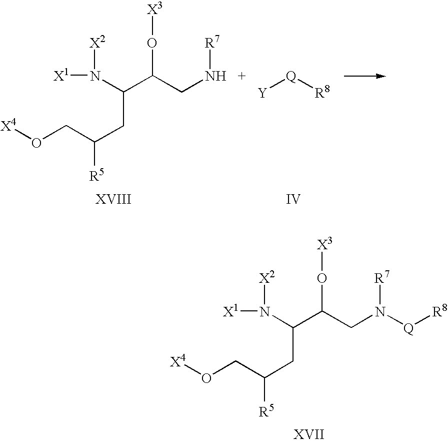

Compounds of formula XVIII are prepared by opening epoxides of formula XIX with amines of formula VI:

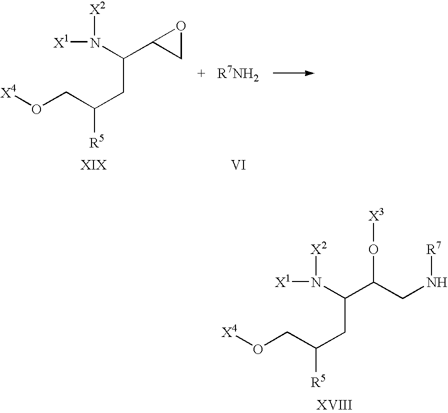

Amine compounds of formula XVIII wherein R⁷═H can also be prepared by reduction of azide compounds of formula XX using hydrogen gas in the presence of a transition metal catalyst, for example Raney nickel or platinum or palladium catalysts, for example platinum or palladium on active carbon, or with triphenylphosphine in the a mixed aqueous-organic solvent (Staudinger reduction). Azide compounds XX can be prepared by reacting by reacting an epoxide compound of formula XIX with nucleophillic azide source such as sodium azide in an organic solvent such as DMF or acetonitrile:

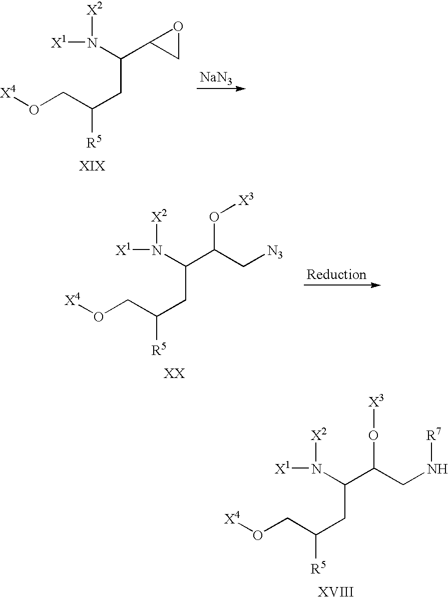

Compounds of formula XVII wherein Q=SO₂ are prepared, for example, by treatment of epoxide compounds of formula XIX with compounds of formula VIII wherein Q=SO₂, followed by protecting group removal:

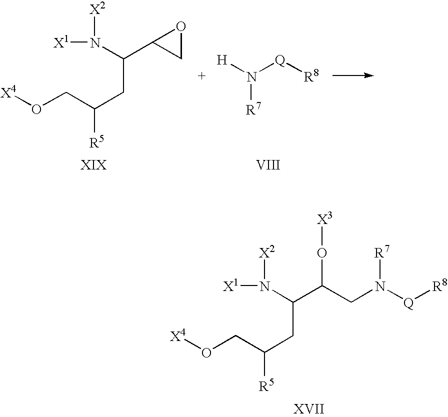

Epoxides of formula XIX are prepared by appropriate adaptations of the various procedures described above for the preparation of epoxides of formula V.

A sixth process of the invention for the preparation of compounds of formula I wherein Q is C═O or C═S and R¹⁰ is hydrogen comprises 1) reacting a amine compound of formula II with an isocyanate or isothiocyanate of formula XXI:

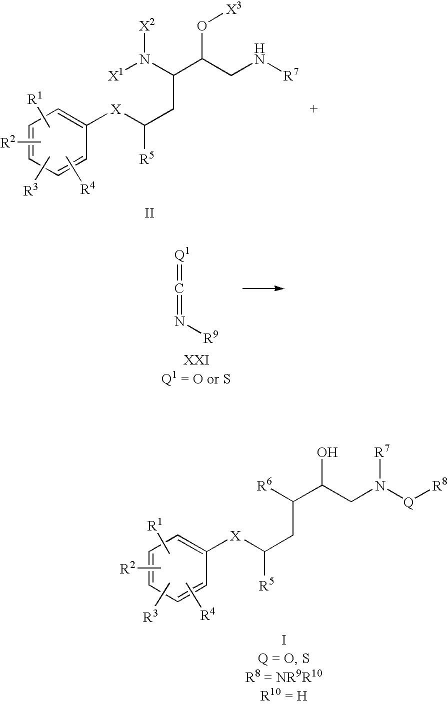

and 2) removing any protecting groups present.

In each of the processes mentioned above, the starting compounds may also be used in the form of salts, provided that the reaction conditions allow it.

A free amino group present in a compound of formula I obtainable in accordance with the process can be acylated or alkylated, for example to introduce a radical R⁶ other than hydrogen. The acylation, sulfonylation and the alkylation can be carried out in accordance with one of the methods mentioned for protecting groups or according to known processes.

Furthermore, a free hydroxy group present in a compound of formula I obtainable in accordance with the process, for example as a constituent of the radical R⁸, can be acylated. The acylation can be carried out with acylating reagents in accordance with one of the methods mentioned for protecting groups or according to known processes.

In compounds of formula I in which R¹, R², R³, and/or R⁴ are hydroxy it is also possible to replace hydroxy by one of the etherified hydroxy groups mentioned under formula I by reacting the corresponding compound of formula I wherein R¹, R², R³, and/or R⁴ is hydroxy in customary manner, for example in the presence of a basic condensation agent, with a compound of the formula (e) R′¹-Y, R′²-Y, R′³—Y, and/or R′⁴-Y, wherein R′¹ is lower alkyl or free or esterified or amidated carboxy-lower alkyl, R′² is lower alkyl, lower alkoxy-lower alkyl, lower alkoxy-lower alkyl, cycloalkoxy-lower alkyl, optionally lower alkanoylated, halogenated or sulfonylated hydroxy-lower alkyl, oxo-lower alkyl, lower alkyl, lower alkenyl, cycloalkoxy-lower alkyl, lower alkoxy-lower alkyl, lower alkoxy-lower alkenyl, lower alkenyloxy-lower alkyl, lower alkenyloxy-lower alkyl, lower alkenyloxy-lower alkyl, lower alkanoyl-lower alkyl, optionally S-oxidized lower alkyl-thio-lower alkyl, lower alkylthio-(hydroxy)-lower alkyl, aryl-lower alkyl, optionally hydrogenated heteroaryl-lower alkyl, optionally hydrogenated heteroarylthio-lower alkyl, cyano-lower alkyl or free or esterified or amidated carboxy-lower alkyl, R¹³ is lower alkyl, lower alkoxy-lower alkyl, hydroxy-lower alkyl, aryl-lower alkyl, halogenated lower alkyl, cyano-lower alkyl or free or esterified or amidated carboxy-lower alkyl, and R¹⁴ is lower alkyl, and Y is reactive esterified hydroxy, especially hydroxy esterified by a mineral acid, by sulfuric acid or by an organic sulfonic acid, such as halogen, preferably chlorine, bromine or iodine, lower alkanesulfonyloxy or unsubstituted or substituted benzenesulfonyloxy, especially methane-, ethane-, benzene-, p-toluene- or p-bromobenzene-sulfonyl. The reaction is preferably carried out in the presence of a basic condensation agent, such as an alkali metal carbonate, for example potassium carbonate, in an inert solvent, such as a lower alkanol, such as methanol, ethanol, butanol, tert-butanol or especially amyl alcohol, advantageously at elevated temperature, for example in a temperature range of approximately from 40-140° C., if necessary with removal of the resulting water of reaction by distillation, for example by azeotropic distillation.

It is also possible for salts of compounds of formula I obtainable in accordance with the process to be converted in a manner known per se into the free compounds, for example by treatment with a base, such as an alkali metal hydroxide, a metal carbonate or metal hydrogen carbonate, or ammonia, or another of the salt-forming bases mentioned at the beginning, or with an acid, such as a mineral acid, for example with hydrochloric acid, or another of the salt-forming acids mentioned at the beginning.

Resulting salts can be converted into different salts in a manner known per se: acid addition salts, for example, by treatment with a suitable metal salt, such as a sodium, barium or silver salt, of a different acid in a suitable solvent in which an inorganic salt being formed is insoluble and is therefore eliminated from the reaction equilibrium, and basic salts by freeing of the free acid and conversion into a salt again.

The compounds of formula I, including their salts, may also be obtained in the form of hydrates or may include the solvent used for crystallization.

As a result of the close relationship between the novel compounds in free form and in the form of their salts, any reference herein to the free compounds and their salts is to be understood as including also the corresponding salts and free compounds, respectively, as appropriate and expedient.

Stereoisomeric mixtures, i.e., mixtures of diastereoisomers and/or enantiomers, such as racemic mixtures, can be separated into the corresponding isomers in a manner known per se by suitable separating processes. For example, mixtures of diastereoisomers can be separated into the individual diastereoisomers by fractional crystallization, chromatography, solvent partition, etc. Racemates can be separated from one another, after conversion of the optical antipodes into diastereoisomers, for example by reaction with optically active compounds, for example optically active acids or bases, by chromatography on column materials charged with optically active compounds or by enzymatic methods, for example by selective reaction of only one of the two enantiomers. This separation can be carried out either at the stage of one of the starting materials or with the compounds of formula I themselves.

In a compound of formula I the configuration at individual chirality centers can be selectively reversed. For example, the configuration of asymmetric carbon atoms that carry nucleophilic substituents, such as amino or hydroxy, can be reversed by second order nucleophilic substitution, optionally after conversion of the bonded nucleophilic substituent into a suitable nucleofugal leaving group and reaction with a reagent introducing the original substituent, or the configuration at carbon atoms having hydroxy groups can be reversed by oxidation and reduction, analogously to patent application EP 236,734.

Another embodiment of the invention is those forms of the process in which a compound obtainable as an intermediate at any stage is used as a starting material and the remaining steps are carried out or the process is interrupted at any stage, or a starting material is formed under the reaction conditions or is used in the form of a reactive derivative or salt, or a compound obtained in accordance with the process of the invention is formed under the process conditions and further processed in situ. It is preferable to use those starting materials which result in the compounds described above.

Representative compounds of the invention can be synthesized in accordance with the general synthetic schemes described above and are illustrated in the examples that follow. The methods for preparing the various starting materials used in the schemes and examples are well within the knowledge of persons skilled in the art

The following abbreviations have the indicated meanings:
aq aqueous
Boc tert-butoxy carbonyl or t-butoxy carbonyl
(Boc)₂O di-tert-butyl dicarbonate
brine saturated aqueous sodium chloride
CH₂Cl₂ methylene chloride
CH₃CN or MeCN acetonitrile
Cpd compound
d day
DBU 1,8-diazabicyclo[5.4.0]undec-7-ene
DMAP 4-(dimethylamino)pyridine
DMF N,N-dimethyl formamide
DMSO Dimethyl sulfoxide
DMPU 1,3-dimethyl-3,4,5,6-tetrahydro-2(1H)-pyrimidinone
EDC.HCl 1-[3-(dimethylamino)propyl]-3-ethylcarbodiimide hydrochloride
eq, equiv equivalents
Et ethyl
EtOAc ethyl acetate
Fmoc 1-[[(9H-fluoren-9-ylmethoxy)carbonyl]oxy]-
Fmoc-OSu 1-[[(9H-fluoren-9-ylmethoxy)carbonyl]oxy]-2,5-pyrrolidinedione
h, hr hour
HBTU O-benzotriazol-1-yl-N,N,N′,N′-tetramethyluronium hexafluorophosphate
HOBt 1-hydroxybenzotriazole
KHMDS potassium hexamethyldisilazane
LAH or LiALH₄ lithium aluminum hydride
LHMDS lithium hexamethyldisilazane
Me methyl
MeOH methanol
MsCl methanesulfonyl chloride
min minute
MS mass spectrum
NaH sodium hydride
NaHCO₃ sodium bicarbonate
NaN₃ sodium azide
NaOH sodium hydroxide
Na₂SO₄ sodium sulfate
Pd₂(dba)₃ tris(dibenzylideneacetone)dipalladium(0)
Ph or PH phenyl
RT/rt/r.t. room temperature
satd saturated
SOCl₂ thionyl chloride
TEA triethylamine or Et₃N
Teoc 1-[2-(trimethylsilyl)ethoxycarbonyloxy]-
Teoc-OSu 1-[2-(trimethylsilyl)ethoxycarbonyloxy]pyrrolidin-2,5-dione
TFA trifluoroacetic acid
THF tetrahydrofuran
tlc thin layer chromatography
TMSCl chlorotrimethylsilane or trimethylsilyl chloride
t_{R} retention time

### Analytical Methods

LC-MS (3 min)
Column: Chromolith SpeedRod, RP-18e, 50×4.6 mm; Mobil phase: A:
0.01% TFA/water, B: 0.01% TFA/CH₃CN; Flow rate: 1 mL/min; Gradient:

| Time (min) | A % | B % |
|---|---|---|
| 0.0 | 90 | 10 |
| 2.0 | 10 | 90 |
| 2.4 | 10 | 90 |
| 2.5 | 90 | 10 |
| 3.0 | 90 | 10 |

### EXAMPLE 1

### tert-Butyl (3S)-3-(3-(3-methoxypropoxy)-4-methoxybenzyl)-4-methyl-1-(oxiran-2-yl)pentylcarbamate

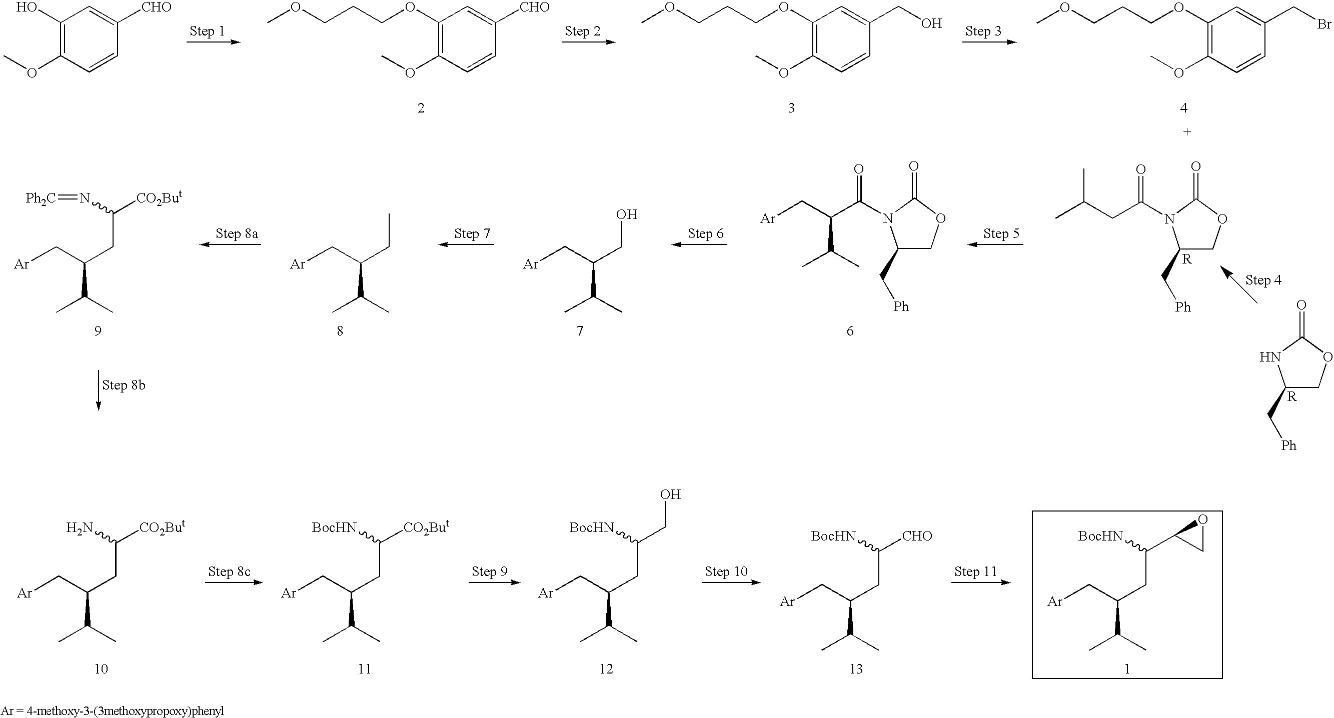

### Step 1

To a mixture of 3-hydroxy-4-methoxy-benzaldehyde (26.60 g, 0.175 mol, 1.0 equiv), triphenylphosphine (60.80 g, 1.3 equiv), and 3-methoxy-1-propanol (16.00 g, 1.0 equiv) in THF (100 mL) and toluene (300 mL) was added a solution of DIAD (47.0 g, 1.3 equiv) in toluene (100 mL) dropwise. The resulting mixture was evacuated and then stirred for 24 h at room temperature. The reaction mixture was concentrated in vacuo. The crude product was carried on to the next step without further purification. An analytical sample of 4-methoxy-3-(3-methoxy-propoxy)-benzaldehyde (2) was obtained by chromatography (33% to 50% ethyl acetate in hexanes). ¹H NMR (400 MHz, CDCl₃) δ (ppm): 9.84 (s, 1H), 7.46-7.42 (m, 2H), 6.97 (d, J=8.4 Hz, 1H), 4.18 (t, J=6.4 Hz, 2H), 3.95 (s, 3H), 3.57 (t, J=6.2 Hz, 2H), 3.35 (s, 3H), 2.13 (p, J=6.3 Hz, 2H).

### Step 2

A mixture of crude 4-methoxy-3-(3-methoxy-propoxy)-benzaldehyde (2) and ethanol (300 mL) was treated with a suspension of NaBH₄ (15.0 g) and ethanol (150 mL). The resulting mixture was stirred overnight at room temperature. The reaction mixture was concentrated in vacuo. The residue was treated with 10% Na₂CO₃ and extracted three times with CH₂Cl₂. The organic phase was dried over Na₂SO₄, filtered and concentrated in vacuo. The residue was filtered through silica gel column (33% to 75% ethyl acetate in hexanes) to give the crude 4-methoxy-3-(3-methoxy-propoxy)-benzyl alcohol (3). An analytical sample was obtained by further chromatography. ¹H NMR (400 MHz, CDCl₃) δ (ppm): 6.95-6.83 (m, 3H), 4.60 (s, 2H), 4.12 (t, J=6.4 Hz, 2H), 3.85 (s, 3H), 3.57 (t, J=6.2 Hz, 2H), 3.34 (s, 3H), 2.10 (p, J=6.3 Hz, 2H), 1.75 (br s, 1H).

### Step 3

To a 2-L round bottom flask of crude 4-methoxy-3-(3-methoxy-propoxy)-benzyl alcohol (3) was added Et₂O (400 mL) and pyridine (0.26 mL). The flask was evacuated and refilled with N₂. PBr₃ (20.93 g) was then added slowly to the stirred solution at room temperature. After 3 h, the reaction mixture was quenched with satd aq NaHCO₃ and extracted three times with ethyl acetate. The organic phase was dried over Na₂SO₄, filtered and concentrated in vacuo. A mixture of the crude product in Et₂O (100 mL) and hexane (400 mL) was vigorously stirred for 0.5 h. The mixture was filtered and the solid collected was washed with hexane. The filtrate was concentrated in vacuo to leave a residue which was purified on silica gel chromatography (25% to 33% ethyl acetate in hexanes) to afford 4-methoxy-3-(3-methoxy-propoxy)-benzyl bromide (4). ¹H NMR (400 MHz, CDCl₃) δ (ppm): 6.96-6.93 (m, 2H), 6.81 (d, J=8.8 Hz, 1H), 4.49 (s, 2H), 4.12 (t, J=6.4 Hz, 2H), 3.86 (s, 3H), 3.57 (t, J=6.2 Hz, 2H), 3.36 (s, 3H), 2.11 (p, J=6.3 Hz, 2H); ¹³C NMR (100 MHz, CDCl₃) δ (ppm): 149.6, 148.5, 130.2, 121.6, 113.8, 111.4, 69.2, 66.0, 58.7, 56.0, 34.4, 29.5.

### Step 4

A 250-mL round bottom flask was charged with (R)-(+)-4-benzyl-2-oxazolidinone (7.520 g, 42.4 mmol, 1.0 equiv) and THF (100 mL). The flask was evacuated and refilled with N₂. The mixture was cooled with a dry ice-acetone bath and 1.6 M n-BuLi in hexanes (30 mL, 48 mmol, 1.13 equiv) was added slowly. After 0.5 h, isovaleroyl chloride (5.5 mL, 45.1 mmol, 1.06 equiv) was added. After 10 min, the dry ice-acetone bath was removed and replaced with an ice bath. After an additional 2.5 h, the reaction mixture was quenched with 10% aq Na₂CO₃ (65 mL) and vigorously stirred for 3 h. The mixture was extracted three times with ethyl acetate. The organic phase was dried over Na₂SO₄, filtered and concentrated in vacuo. The residue was purified by chromatography on silica gel (25% to 33% ethyl acetate in hexanes) to afford (4R)-benzyl-3-(3-methyl-butyryl)-2-oxazolidinone (5) (10.5308 g, 95%). ¹H NMR (400 MHz, CDCl₃) δ (ppm): 7.36-7.21 (m, 5H), 4.71-4.65 (m, 1H), 4.22-4.11 (m, 2H), 3.31 (dd, J=13.3, 3.4 Hz, 1H), 2.89 (dd, J=16.1, 6.7 Hz, 1H), 2.78 (dd, J=16.3, 7.2 Hz, 1H), 2.75 (dd, J=13.2, 9.7 Hz, 1H), 2.27-2.17 (m, 1H), 1.02 (d, J=6.7 Hz, 3H), 1.00 (d, J=6.7 Hz, 3H).

### Step 5

To a 250-mL round bottom flask of compound (4R)-benzyl-3-(3-methyl-butyryl)-2-oxazolidinone (5) (5.500 g, 21.0 mmol) was added THF (60 mL). The flask was evacuated and refilled with N₂. The mixture was cooled with a dry ice-acetone bath and 11.0M LiHMDS in THF (23.5 mL, 23.5 mmol) was added dropwise. After 0.5 h, a solution of 4-methoxy-3-(3-methoxy-propoxy)-benzyl bromide (4) (5.8043 g, 20.1 mmol) in THF (30 mL) was added slowly via cannula. The resulting mixture was allowed to slowly warm to room temperature while stirring overnight. The mixture was quenched with satd aq NH₄Cl and extracted three times with ethyl acetate. The organic phase was dried over Na₂SO₄, filtered and concentrated in vacuo. The residue was purified by chromatography on silica gel (25% to 33% ethyl acetate in hexanes) to afford (R)-3-((R)-2-(3-(3-methoxypropoxy)-4-methoxybenzyl)-3-methylbutanoyl)-4-benzyloxazolidin-2-one (6) (8.349 g, 84%). LC-MS (3 min) t_{R}=2.05 min m/z 492 (M+Na⁺), 470 (M+H⁺), 293, 261; ¹H NMR (400 MHz, CDCl₃) δ (ppm): 7.24-7.20 (m, 3H), 6.93-6.91 (m, 2H), 6.85 (d, J=1.8 Hz, 1H), 6.77 (dd, J=8.2, 1.8 Hz, 1H), 6.73 (d, J=8.2 Hz, 1H), 4.63-4.57 (m, 1H), 4.28-4.23 (m, 1H), 4.09-4.03 (m, 3H), 3.96 (dd, J=8.9, 2.5 Hz, 1H), 3.78 (s, 3H), 3.55-3.49 (m, 2H), 3.31 (s, 3H), 2.97-2.80 (m, 3H), 2.19 (dd, J=13.5, 9.4 Hz, 1H), 2.11-1.97 (m, 3H), 1.06 (d, J=7.0 Hz, 3H), 1.03 (d, J=6.7 Hz, 3H); ¹³C NMR (100 MHz, CDCl₃) δ (ppm): 175.9, 153.0, 148.2, 147.8, 135.2, 131.9, 129.3, 128.8, 127.1, 121.4, 114.1, 111.4, 69.4, 65.9, 65.3, 58.6, 56.0, 55.0, 50.1, 37.3, 35.4, 31.4, 29.5, 20.7, 19.5.

### Step 6

To a 100-mL round bottom flask of (R)-3-((R)-2-(3-(3-methoxypropoxy)-4-methoxybenzyl)-3-methylbutanoyl)-4-benzyloxazolidin-2-one (6) (2.1475 g, 4.57 mmol) was added Et₂O (50 mL) and H₂O (0.18 mL). The flask was evacuated and refilled with N₂. The mixture was cooled with an ice bath and 2.0 M LiBH₄ in THF (5.5 mL, 11.0 mmol) was added dropwise. After 10 min, the cooling bath was removed and the mixture was stirred for an additional 0.5 h. The mixture was then cooled with an ice bath, quenched with 1 N aq NaOH (20 mL) and extracted three times with CH₂Cl₂. The organic phase was dried over Na₂SO₄, filtered and concentrated in vacuo. The residue was purified by chromatography on silica gel (33% to 50% ethyl acetate in hexanes) to afford (R)-2-(3-(3-methoxypropoxy)-4-methoxybenzyl)-3-methylbutan-1-ol (7) (0.7894 g, 58%). LC-MS (3 min) t_{R}=1.60 min m/z 319 (MNa⁺), 297 (MH⁺), 209; ¹H NMR (400 MHz, CDCl₃) δ (ppm): 6.80-6.71 (m, 3H), 4.10 (t, J=6.6 Hz, 2H), 3.84 (s, 3H), 3.59-3.55 (m, 4H), 3.36 (s, 3H), 2.65 (dd, J=13.8, 5.6 Hz, 1H), 2.45 (dd, J=13.8, 9.4 Hz, 1H), 2.10 (p, J=6.3 Hz, 2H), 1.88-1.80 (m, 1H), 1.66-1.59 (m, 1H), 1.41 (br s, 1H), 0.97 (d, J=7.0 Hz, 3H), 0.96 (d, J=7.0 Hz, 3H); ¹³C NMR (100 MHz, CDCl₃) δ (ppm): 148.2, 147.5, 133.9, 121.1, 114.0, 111.6, 69.3, 65.9, 63.0, 58.7, 56.0, 48.8, 34.1, 29.5, 27.9, 19.7, 19.5.

### Step 7

A 100 mL round bottom flask was charged with triphenylphosphine (1.3055 g, 4.98 mmol, 1.2 equiv) and CH₂Cl₂ (20 mL). Imidazole (0.5590 g, 8.21 mmol, 2.0 equiv) and iodine (1.4547 g, 5.73 mmol, 1.4 equiv) were added. The flask was evacuated and refilled with N₂. A solution of (R)-2-(3-(3-methoxypropoxy)-4-methoxybenzyl)-3-methylbutan-1-ol (7) (1.1992 g, 4.04 mmol, 1.0 equiv) in CH₂Cl₂ (20 mL) was added to the resulting suspension via cannula. After 3 h, the solvents were removed in vacuo. The residue was purified by chromatography on silica gel (25% to 33% ethyl acetate in hexanes) to give 2-(3-methoxypropoxy)-4-((R)-2-(iodomethyl)-3-methylbutyl)-1-methoxybenzene (8) (1.4742 g, 90%). LC-MS (3 min) t_{R}=2.33 min, m/z 407 (MH⁺), 375, 177; ¹H NMR (400 MHz, CDCl₃) δ (ppm): 6.80-6.73 (m, 3H), 4.11 (t, J=6.4 Hz, 2H), 3.84 (s, 3H), 3.58 (t, J=6.2 Hz, 2H), 3.36 (s, 3H), 3.21 (dd, J=10.0, 4.7 Hz, 1H), 3.09 (dd, J=10.0, 4.4 Hz, 1H), 2.77 (dd, J=13.9, 4.8 Hz, 1H), 2.34 (dd, J=13.8, 9.7 Hz, 1H), 2.11 (p, J=6.3 Hz, 2H), 1.75-1.65 (m, 1H), 1.16-1.10 (m, 1H), 1.01 (d, J=6.8 Hz, 3H), 0.95 (d, J=6.7 Hz, 3H); ¹³C NMR (100 MHz, CDCl₃) δ (ppm): 148.2, 147.7, 132.9, 121.1, 114.0, 111.7, 69.3, 65.9, 58.7, 56.0, 47.6, 36.6, 30.5, 29.5, 19.8, 19.5, 14.5.

### Step 8

A flame dried 100-mL round bottom flask was charged with N-(diphenylmethylene)glycine tert-butyl ester (0.6625 g, 2.24 mmol, 1.25 equiv), THF (10 mL) and HMPA (1 mL). The flask was evacuated and refilled with N₂. The mixture was cooled with a dry ice-acetone bath and 1.0 M LiHMDS in THF (2.5 mL, 2.5 mmol) was added dropwise. After 15 min, a solution of 2-(3-methoxypropoxy)-4-((R)-2-(iodomethyl)-3-methylbutyl)-1-methoxybenzene (8) (0.7301 g, 1.80 mmol, 1.0 equiv) in THF (10 mL) was added slowly via cannula. The resulting mixture was allowed to slowly warm to room temperature while stirring overnight. The mixture was quenched with saturated brine and extracted three times with ethyl acetate. The organic phase was dried over Na₂SO₄, filtered and concentrated in vacuo to afford crude (4S)-tert-butyl 4-(3-(3-methoxypropoxy)-4-methoxybenzyl)-2-(diphenylmethyleneamino)-5-methylhexanoate (9) which was used without further purification.

A mixture of crude alkylation product 9, THF (30 mL) and 1 M aq citric acid (35 mL) was vigorously stirred overnight. The solvent was removed in vacuo. The aqueous phase was carefully treated with Na₂CO₃ (6.5 g) and extracted three times with CH₂Cl₂. The organic phase was dried over Na₂SO₄, filtered and concentrated in vacuo. The crude (4S)-tert-butyl 4-(3-(3-methoxypropoxy)-4-methoxybenzyl)-2-amino-5-methylhexanoate (10) was stirred overnight with Boc₂O (1.5 g, mmol) in CH₂Cl₂. The solvent was removed in vacuo and the residue was purified on silica gel chromatography (20% to 33% ethyl acetate in hexanes) to give 0.6581 g (72%) of tert-butyl (3S)-1-(tert-butoxycarbonyl)-3-(3-(3-methoxypropoxy)-4-ethylbenzyl)-4-methylpentyl-carbamate (11). LC-MS (3 min) t_{R}=2.36 m/z 532 (M+Na⁺), 410, 354; ¹H NMR (400 MHz, CDCl₃) δ (ppm): 6.79-6.65 (m, 3H), 4.90 (d, J=8.5 Hz, 1H), 4.22 (q, J=7.9 Hz, 1H), 4.09 (t, J=6.3 Hz, 2H), 3.82 (s, 3H), 3.57 (t, J=6.3 Hz, 2H), 3.35 (s, 3H), 2.58 (dd, J=13.6, 6.6 Hz, 1H), 2.45 (dd, J=13.3, 8.1 Hz, 1H), 2.13-2.06 (m, 2H), 1.78-1.73 (m, 1H), 1.65 (br s, 1H), 1.52-1.47 (m, 2H), 1.44 (s, 9H), 1.43 (s, 9H), 0.86 (d, J=6.8 Hz, 3H), 0.83 (d, J=6.8 Hz, 3H); ¹³C NMR (100 MHz, CDCl₃) δ (ppm): 172.3, 155.3, 148.0, 147.3, 133.7, 121.1, 114.1, 111.4, 81.3, 79.2, 69.2, 65.7, 58.4, 55.8, 52.2, 41.9, 36.1, 33.4, 29.4, 28.1, 27.8, 27.3, 19.3, 18.3, 18.2, 17.3.

### Step 9

To a −78° C. solution of tert-butyl (3S)-1-(tert-butoxycarbonyl)-3-(3-(3-methoxypropoxy)-4-ethylbenzyl)-4-methylpentylcarbamate (11) (0.7012 g, 1.38 mmol) in THF (15 mL) was added 1.0 M diisobutylaluminum hydride in hexanes (8 mL, 8.0 mmol) dropwise. The mixture was allowed to slowly warm to room temperature while stirring overnight. The reaction mixture was carefully quenched with MeOH (9 mL). After 1 h, the mixture was diluted with saturated Rochelle's salt and extracted three times with ethyl acetate. The organic phase was dried over Na₂SO₄, filtered and concentrated in vacuo. The residue was purified by chromatography on silica gel (50% ethyl acetate in hexanes) to give tert-butyl (4S)-4-(3-(3-methoxypropoxy)-4-methoxybenzyl)-1-hydroxy-5-methylhexan-2-ylcarbamate (12) (0.5049 g, 83%).

### Step 10

To a 100-mL round bottom flask of tert-butyl (4S)-4-(3-(3-methoxypropoxy)-4-methoxybenzyl)-1-hydroxy-5-methylhexan-2-ylcarbamate 12 (0.5049 g, 1.15 mmol, 1.0 equiv) were added DMSO (5 mL) and triethylamine (2 mL). The flask was cooled with an ice bath. A mixture of pyridine-sulphur trioxide complex (1.85 g, 10 equiv) in dry DMSO (5 mL) was added. After 0.5 h, the ice bath was removed. The reaction mixture was allowed to stir at room temperature for an additional 0.5 h. The mixture was poured into ice water and extracted three times with ethyl acetate. The combined organic phase was washed with 10% aq citric acid, sat'd aq NaHCO₃ and brine, dried over Na₂SO₄, filtered and concentrated in vacuo. The residue was purified by chromatography on silica gel (20% to 50% ethyl acetate in hexanes) to afford tert-butyl (3S)-3-(3-(3-methoxypropoxy)-4-methoxybenzyl)-1-formyl-4-methylpentylcarbamate (13) (0.4909 g, 98%).

### Step 11

A flame-dried 100-mL round bottom flask was charged with 60% sodium hydride in oil (0.247 g, 6.17 mmol) and trimethyloxosulfonium iodide (1.356 g, 6.16 mmol). The flask was evacuated and refilled with N₂. Dry DMSO (8 mL) was added. The mixture was stirred at room temperature for 1 h. When H₂ evolution had ceased, the resulting solution was clear.

A second 100-mL round bottom flask was charged with tert-butyl (3S)-3-(3-(3-methoxypropoxy)-4-methoxybenzyl)-1-formyl-4-methylpentylcarbamate (13) (0.4602 g, 1.05 mmol) and 6 mL of THF (6 mL). The flask was evacuated and refilled with N₂ and an aliquot of the ylid solution prepared above (2 mL, 1.5 mmol, 1.5 equiv) was added by syringe. The resulting mixture was stirred for 1 h at room temperature. The reaction mixture was quenched with brine and extracted three times with ethyl acetate. The organic phase was dried over Na₂SO₄, filtered and concentrated in vacuo. The residue was purified by chromatography on silica gel (33% ethyl acetate in hexanes) to afford tert-butyl (3S)-3-(3-(3-methoxypropoxy)-4-methoxybenzyl)-4-methyl-1-(oxiran-2-yl)pentylcarbamate (1) (0.250 g, 53%) as a mixture of four isomers, of which tert-butyl (1S,3S)-3-(3-(3-methoxypropoxy)-4-methoxybenzyl)-4-methyl-1-((R)-oxiran-2-yl)pentylcarbamate was the major isomer.

### EXAMPLE 2

### Halides

The following halides were prepared following the procedures of Example 1 Steps 5, 6, and 7:

1-(((S)-2-(bromomethyl)-3-methylbutoxy)methyl)benzene (chloromethyl benzyl ether was used in Step 5 in place of 4-methoxy-3-(3-methoxy-propoxy)-benzyl bromide) 1-((3-((R)-2-(bromomethyl)-3-methylbutyl)phenoxy)methyl)benzene (3-benzyloxybenzyl bromide was used in Step 5 in place of 4-methoxy-3-(3-methoxy-propoxy)-benzyl bromide).

### EXAMPLE 3

### Tert-butyl (1S,3S)-3-(3-(3-methoxypropoxy)-4-methoxybenzyl)-4-methyl-1-((R)-oxiran-2-yl)pentylcarbamate

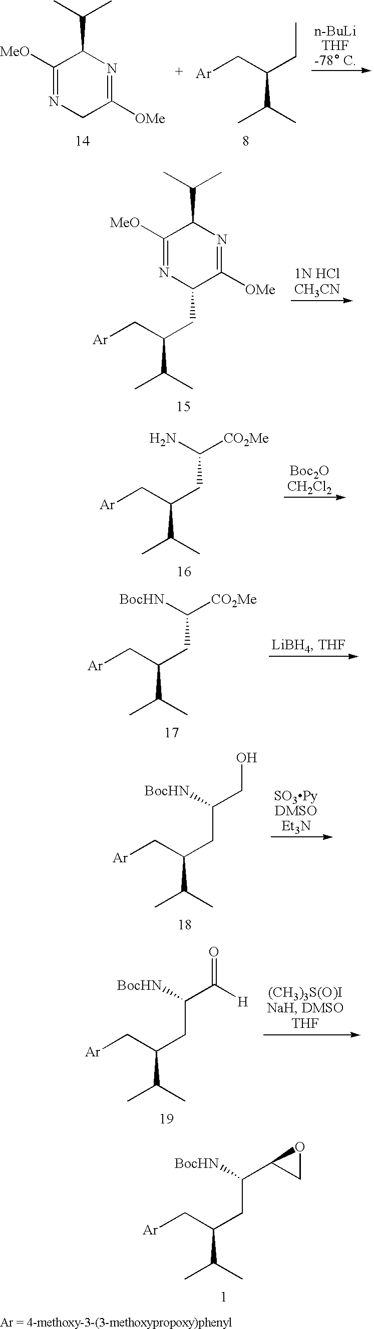

### Step 1

A flame-dried 100-mL round bottom flask was charged with (R)-2,5-dihydro-3,6-dimethoxy-2-isopropylpyrazine (14) (2.4080 g, 13.07 mmol) and THF (20 mL), and evacuated and refilled with N₂. The mixture was cooled with a dry ice-acetone bath and 2.5 M n-BuLi in hexanes (5.2 mL, 13.00 mmol) was added dropwise over 15 min. After an additional 0.5 h, a solution of 2-(3-methoxypropoxy)-4-((R)-2-(iodomethyl)-3-methylbutyl)-1-methoxybenzene (8) (3.3023 g, 8.13 mmol, 0.62 equiv) from Example 1 Step 7 in THF (20 mL) was added dropwise via cannula over 10 min. The reaction mixture was allowed to stir at −78° C. for 16 h and quenched with brine (20 mL) at −78° C. After warming to room temperature, the mixture was extracted three times with ethyl acetate. The organic phase was dried over Na₂SO₄, filtered and concentrated in vacuo. The crude (2S,5R)-2-((S)-2-(3-(3-methoxypropoxy)-4-methoxybenzyl)-3-methylbutyl)-2,5-dihydro-5-isopropyl-3,6-dimethoxypyrazine (15) (4.85 g, 80%) was carried on to the next step without further purification. LC-MS (3 min) t_{R}=2.41 min m/z 463 (M+H⁺).

### Step 2

A mixture of crude (2S,5R)-2-((S)-2-(3-(3-methoxypropoxy)-4-methoxybenzyl)-3-methylbutyl)-2,5-dihydro-5-isopropyl-3,6-dimethoxypyrazine (15) (4.85 g, 10.49 mmol) in acetonitrile (100 mL) and 1 Naq HCl (100 mL, 100 mmol) was vigorously stirred at room temperature for 3 h. The solvent was removed in vacuo. The aqueous phase was cooled with an ice bath, carefully treated with Na₂CO₃ (7.06 g, 66.6 mmol) and extracted three times with CH₂Cl₂. The organic phase was dried over Na₂SO₄, filtered and concentrated in vacuo to afford (2S,4S)-methyl 4-(3-(3-methoxypropoxy)-4-methoxybenzyl)-2-amino-5-methylhexanoate (16) (4.58 g) which was carried on to the next step without further purification.

### Step 3

A mixture of (2S,4S)-methyl 4-(3-(3-methoxypropoxy)-4-methoxybenzyl)-2-amino-5-methylhexanoate (16) (4.58 g, 12.46 mmol) and Boc₂O (7.33 g, 33.58 mmol, 2.57 equiv) in CH₂Cl₂ (100 mL) was stirred at room temperature for 14 h. The solvent was removed in vacuo and the residue was purified by chromatography on silica gel (20% to 33% ethyl acetate in hexanes) to give tert-butyl (1S,3S)-1-(methoxycarbonyl)-3-(3-(3-methoxypropoxy)-4-methoxybenzyl)-4-methylpentylcarbamate (17) (3.3224 g, 87% from 2-(3-methoxypropoxy)-4-((R)-2-(iodomethyl)-3-methylbutyl)-1-methoxybenzene). R_{f}=0.29 (30% ethyl acetate in hexanes); LC-MS (3 min) t_{R}=2.07 min in 3 min chromatography, m/z 490 (MNa⁺), 368; ¹H NMR (400 MHz, CDCl₃) δ (ppm): 6.77-6.67 (m, 3H), 4.89 (d, J=8.8 Hz, 1H), 4.36 (q, J=7.7 Hz, 1H), 4.10 (t, J=6.4 Hz, 2H), 3.83 (s, 3H), 3.71 (s, 3H), 3.57 (t, J=6.2 Hz, 2H), 3.35 (s, 3H), 2.64 (dd, J=13.8, 5.3 Hz, 1H), 2.43 (dd, J=13.6, 8.6 Hz, 1H), 2.09 (p, J=6.3 Hz, 2H), 1.74-1.53 (m, 4H), 1.44 (s, 9H), 0.83 (d, J=6.5 Hz, 3H), 0.82 (d, J=6.7 Hz, 3H); ¹³C NMR (100 MHz, CDCl₃) δ (ppm): 173.9, 155.5, 148.2, 147.5, 133.6, 121.3, 114.2, 111.5, 79.8, 69.4, 65.9, 58.6, 56.0, 52.2, 51.8, 41.9, 36.5, 33.2, 31.6, 29.6, 28.3, 27.7, 22.6, 20.0, 17.0, 14.1.

### Step 4

To a solution of tert-butyl (1S,3S)-1-(methoxycarbonyl)-3-(3-(3-methoxypropoxy)-4-methoxybenzyl)-4-methylpentylcarbamate (17) (3.2926 g, 7.04 mmol) in THF (50 mL) was slowly added 2.0 M LiBH4 in THF (11 mL, 22 mmol, 3 equiv). The mixture was allowed to stir at room temperature for 15 h. The reaction mixture was diluted with ethyl acetate (60 mL) and carefully quenched with 1 N aq HCl (60 mL). After the emulsion disappeared, the organic layer was separated. The aqueous layer was extracted three times with ethyl acetate. The combined organic phase was dried over Na₂SO₄, filtered and concentrated in vacuo. The residue was purified by chromatography on silica gel (50% to 66% ethyl acetate in hexanes) to afford tert-butyl (2S,4S)-4-(3-(3-methoxypropoxy)-4-methoxybenzyl)-1-hydroxy-5-methylhexan-2-ylcarbamate (18) (3.1192 g, 100%). LC-MS (3 min) t_{R}=1.82 min m/z 462 (M+Na⁺), 340; ¹H NMR (400 MHz, CDCl₃) δ (ppm): 6.78-6.67 (m, 3H), 4.56 (br s, 1H), 4.10 (t, J=6.6 Hz, 2H), 3.83 (s, 3H), 3.64 (br s, 1H), 3.57 (t, J=6.3 Hz, 2H), 3.45-3.41 (m, 1H), 3.35 (s, 3H), 2.48 (d, J=7.3 Hz, 2H), 2.09 (p, J=6.4 Hz, 2H), 1.99 (br s, 2H), 1.77-1.69 (m, 1H), 1.58-1.52 (m, 1H), 1.47-1.40 (m, 1H), 1.44 (s, 9H), 1.27-1.21 (m, 1H), 0.88 (d, J=6.5 Hz, 3H), 0.86 (d, J=6.5 Hz, 3H); ¹³C NMR (100 MHz, CDCl₃) δ (ppm): 156.4, 148.2, 147.5, 134.0, 121.2, 114.3, 111.5, 79.4, 69.4, 66.0, 60.4, 58.6, 56.0, 50.9, 42.3, 36.9, 31.4, 29.5, 28.3, 21.0, 19.7, 17.7, 14.2.

### Step 5

To a 250-mL round bottom flask of tert-butyl (2S,4S)-4-(3-(3-methoxypropoxy)-4-methoxybenzyl)-1-hydroxy-5-methylhexan-2-ylcarbamate (18) (3.0542 g, 6.95 mmol, 1.0 equiv) was added DMSO (25 mL) and triethylamine (10 mL). The flask was cooled with an ice bath. A mixture of pyridine-sulphur trioxide complex (11.6 g, 72.9 mmol, 10.5 equiv) and dry DMSO (25 mL) was added. After 0.5 h, the ice bath was removed. The reaction mixture was allowed to stir at room temperature for an additional 0.5 h. The mixture was poured into ice water and extracted three times with ethyl acetate. The combined organic phase was washed with 10% aq citric acid, satd aq NaHCO₃, brine, dried over Na₂SO₄, filtered and concentrated in vacuo. The crude tert-butyl (1S,3S)-3-(3-(3-methoxypropoxy)-4-methoxybenzyl)-1-formyl-4-methylpentyl-carbamate (19) (3.2205 g, 100%) was carried on to the next step without further purification. R_{f}=0.27 (30% ethyl acetate in hexanes); ¹H NMR (400 MHz, CDCl₃) δ (ppm): 9.51 (s, 1H), 6.78-6.68 (m, 3H), 4.91 (d, J=7.6 Hz, 1H), 4.14-4.08 (m, 3H), 3.83 (s, 3H), 3.57 (t, J=6.2 Hz, 2H), 3.35 (s, 3H), 2.62-2.47 (m, 2H), 2.14-2.05 (m, 2H), 1.78-1.58 (m, 4H), 1.44 (s, 9H), 0.87 (d, J=6.8 Hz, 3H), 0.84 (d, J=6.8 Hz, 3H).

### Step 5

A flame-dried 250-mL round bottom flask was charged with 60% sodium hydride in oil (1.4483 g, 36.2 mmol) and trimethyloxosulfonium iodide (8.0500 g, 36.5 mmol). The flask was evacuated, refilled with N₂ and dry DMSO (50 mL) was added. The mixture was stirred at room temperature for 1 h. When H₂ evolution had ceased, the resulting ylid solution was clear.

A second 250-mL round bottom flask was charged with crude tert-butyl (1S,3S)-3-(3-(3-methoxypropoxy)-4-methoxybenzyl)-1-formyl-4-methylpentylcarbamate (19) (3.2205 g, 6.97 mmol) and THF (30 mL). The flask was evacuated and refilled with N₂. An aliquot of the ylid solution prepared above (14.5 mL, 10.5 mmol, 1.5 equiv) was added through a syringe. The resulting mixture was stirred for 1 h at room temperature. The reaction mixture was quenched with brine and extracted three times with ethyl acetate. The organic phase was dried over Na₂SO₄, filtered and concentrated in vacuo. The residue was purified on silica gel chromatography (33% ethyl acetate in hexanes) to afford tert-butyl (1S,3S)-3-(3-(3-methoxypropoxy)-4-methoxybenzyl)-4-methyl-1-((R)-oxiran-2-yl)pentylcarbamate (1) (1.4458 g, 46%). R_{f}=0.30 (30% ethyl acetate in hexanes); LC-MS (3 min) t_{R}=2.06 min m/z 474 (M+Na⁺), 396; ¹H NMR (400 MHz, CDCl₃) δ (ppm): 6.79-6.66 (m, 3H), 4.31 (d, J=9.7 Hz, 1H), 4.14-4.07 (m, 2H), 3.97 (br s, 1H), 3.83 (s, 3H), 3.59-3.55 (m, 2H), 3.35 (s, 3H), 2.93 (br s, 1H), 2.72-2.66 (m, 2H), 2.57 (dd, J=4.8, 2.8 Hz, 1H), 2.41 (dd, J=13.5, 9.1 Hz, 1H), 2.13-2.06 (m, 2H), 1.74-1.49 (m, 3H), 1.43 (s, 9H), 1.37-1.30 (m, 1H), 0.88-0.82 (m, 6H); ¹³C NMR (100 MHz, CDCl₃) δ (ppm): 155.7, 148.1, 147.4, 133.8, 121.2, 114.2, 111.4, 79.2, 69.3, 65.8, 58.6, 55.9, 54.1, 53.8, 47.2, 44.3, 42.0, 36.9, 33.3, 29.5, 28.2, 20.2, 19.3, 17.9, 16.8.

### EXAMPLE 4

### Epoxides

The following epoxides were prepared by following the procedures of Example 2:

tert-butyl (1S,3S)-3-((benzyloxy)methyl)-4-methyl-1-((R)-oxiran-2-yl)pentylcarbamate, by using 1-(((S)-2-(bromomethyl)-3-methylbutoxy)methyl)benzene in place of 2-(3-methoxypropoxy)-4-((R)-2-(iodomethyl)-3-methylbutyl)-1-methoxybenzene in Step 1. tert-butyl (1S,3S)-3-(3-(benzyloxy)benzyl)-4-methyl-1-((R)-oxiran-2-yl)pentylcarbamate, by using 1-((3-((R)-2-(bromomethyl)-3-methylbutyl)phenoxy)methyl)benzene in place of 2-(3-methoxypropoxy)-4-((R)-2-(iodomethyl)-3-methylbutyl)-1-methoxybenzene in Step 1.

### EXAMPLE 5

### N-((2S,3S,5S)-5-(3-(3-methoxypropoxy)-4-methoxybenzyl)-3-amino-2-hydroxy-6-methylheptyl)cyclohexanecarboxamide (I-13)

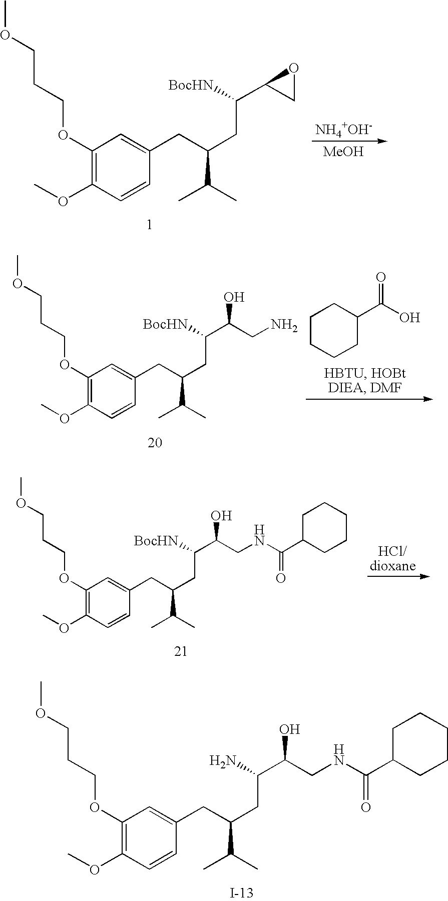

### Step 1

To a solution of tert-butyl (1S,3S)-3-(3-(3-methoxypropoxy)-4-methoxybenzyl)-4-methyl-1-((R)-oxiran-2-yl)pentylcarbamate (1) (0.50 g, 1.11 mmol) in methanol (10 mL) was added ammonium hydroxide solution (10 mL, excess). The resulting clear solution was stirred overnight at room temperature. The solvent was removed to dryness to give crude tert-butyl (2S,3S,5S)-5-(3-(3-methoxypropoxy)-4-methoxybenzyl)-1-amino-2-hydroxy-6-methylheptan-3-ylcarbamate (20) (0.52 g, quant.), which was used for next step without purification. MS m/z 469 (M+1).

### Step 2

To a solution of tert-butyl (2S,3S,5S)-5-(3-(3-methoxypropoxy)-4-methoxybenzyl)-1-amino-2-hydroxy-6-methylheptan-3-ylcarbamate (20) (20.1 mg, 0.043 mmol) in DMF (0.4 mL) was added diisopropylethylamine (0.1 mL), followed by cyclohexanecarboxylic acid (6.1 mg, 0.047 mmol), O-benzotriazol-1-yl-N,N,N′,N′-tetramethyluronium hexafluorophosphate (17.9 mg, 0.047 mmol), and HOBt (6.3 mg, 0.047 mmol). The resulting mixture was stirred at room temperature until the reaction was complete (2-3 h) and purified by preparative HPLC to give tert-butyl (2S,3S,5S)-5-(3-(3-methoxypropoxy)-4-methoxybenzyl)-1-(cyclohexanecarbonyl)amino-2-hydroxy-6-methylheptan-3-ylcarbamate (21) (12.2 mg, 49%), MS m/z 579 (M+1).

### Step 3

tert-Butyl (2S,3S,5S)-5-(3-(3-methoxypropoxy)-4-methoxybenzyl)-1-(cyclohexane-carbonyl)amino-2-hydroxy-6-methylheptan-3-ylcarbamate (21) (12.2 mg, 0.021 mmol) was treated with 4 M HCl in dioxane (2 mL, 8 mmol) at room temperature for 1 hr. The solvent was removed in vacuo to give N-((2S,3S,5S)-5-(3-(3-methoxypropoxy)-4-methoxybenzyl)-3-amino-2-hydroxy-6-methylheptyl)cyclohexanecarboxamide (I-13) as the HCl salt in quantitative yield. ¹H NMR (CD₃OD) δ (ppm): 6.88-6.73 (m, 3H), 4.05 (t, J=6.4 Hz, 2H), 3.80 (s, 3H), 3.75-3.66 (m, 1H), 3.58 (t, J=6.4 Hz, 2H), 3.35 (s, 3H), 3.26-3.22 (m, 1H), 3.10-3.05 (m, 1H), 2.87-2.82 (m, 1H), 2.62 (dd, J=13.6, 6.4 Hz, 1H), 2.39 (d, J=13.6, 8.0 Hz, 1H), 2.22-2.16 (m, 1H), 2.02 (m, 2H), 1.76-1.68 (m, 7H), 1.62-1.60 (m, 1H), 1.43-1.26 (m, 6H), 0.96-0.89 (m, 6H); MS m/z 479 (M+1).

### EXAMPLE 6

The following compounds of formula I were prepared using the procedures of Example 5 Steps 2 and 3, replacing the cyclohexanecarboxylic acid used in Step 2 with other carboxylic acids and in some cases using alternative amide forming coupling reagents well known in the art such as EDC·HCl in place HBTU:

| Cpd. No. | Name |
|---|---|
| I-1 | N-((2S,3S,5S)-5-(3-(3-methoxypropoxy)-4-methoxybenzyl)-3-amino-2-hydroxy-6- |
| | methylheptyl)butyramide |
| I-2 | N-((2S,3S,5S)-5-(3-(3-methoxypropoxy)-4-methoxybenzyl)-3-amino-2-hydroxy-6- |
| | methylheptyl)-2-cyclopropylacetamide |
| I-3 | N-((2S,3S,5S)-5-(3-(3-methoxypropoxy)-4-methoxybenzyl)-3-amino-2-hydroxy-6- |
| | methylheptyl)pentanamide |
| I-4 | N-((2S,3S,5S)-5-(3-(3-methoxypropoxy)-4-methoxybenzyl)-3-amino-2-hydroxy-6- |
| | methylheptyl)pivalamide |
| I-6 | N-((2S,3S,5S)-5-(3-(3-methoxypropoxy)-4-methoxybenzyl)-3-amino-2-hydroxy-6- |
| | methylheptyl)hexanamide |
| I-7 | N-((2S,3S,5S)-5-(3-(3-methoxypropoxy)-4-methoxybenzyl)-3-amino-2-hydroxy-6- |
| | methylheptyl)-2,2-dimethylbutanamide |
| I-8 | N-((2S,3S,5S)-5-(3-(3-methoxypropoxy)-4-methoxybenzyl)-3-amino-2-hydroxy-6- |
| | methylheptyl)-3,3-dimethylbutanamide |
| I-9 | N-((2S,3S,5S)-5-(3-(3-methoxypropoxy)-4-methoxybenzyl)-3-amino-2-hydroxy-6- |
| | methylheptyl)-4-methoxybutanamide |
| I-10 | N-((2S,3S,5S)-5-(3-(3-methoxypropoxy)-4-methoxybenzyl)-3-amino-2-hydroxy-6- |
| | methylheptyl)benzamide |
| I-11 | N-((2S,3S,5S)-5-(3-(3-methoxypropoxy)-4-methoxybenzyl)-3-amino-2-hydroxy-6- |
| | methylheptyl)-3,3,3-trifluoropropanamide |
| I-12 | N-((2S,3S,5S)-5-(3-(3-methoxypropoxy)-4-methoxybenzyl)-3-amino-2-hydroxy-6- |
| | methylheptyl)-2-cyclopentylacetamide |
| I-15 | N-((2S,3S,5S)-5-(3-(3-methoxypropoxy)-4-methoxybenzyl)-3-amino-2-hydroxy-6- |
| | methylheptyl)heptanamide |
| I-16 | N-((2S,3S,5S)-5-(3-(3-methoxypropoxy)-4-methoxybenzyl)-3-amino-2-hydroxy-6- |
| | methylheptyl)-2,2-dimethylpentanamide |
| I-17 | N-((2S,3S,5S)-5-(3-(3-methoxypropoxy)-4-methoxybenzyl)-3-amino-2-hydroxy-6- |
| | methylheptyl)-2-methylhexanamide |
| I-18 | N-((2S,3S,5S)-5-(3-(3-methoxypropoxy)-4-methoxybenzyl)-3-amino-2-hydroxy-6- |
| | methylheptyl)-2-phenylacetamide |
| I-20 | N-((2S,3S,5S)-5-(3-(3-methoxypropoxy)-4-methoxybenzyl)-3-amino-2-hydroxyheptyl)-4,4,4- |
| | trifluorobutanamide |
| I-21 | N-((2S,3S,5S)-5-(3-(3-methoxypropoxy)-4-methoxybenzyl)-3-amino-2-hydroxy-6- |
| | methylheptyl)-2-cyclohexylacetamide |
| I-22 | 1-((2S,3S,5S)-5-(3-(3-methoxypropoxy)-4-methoxybenzyl)-3-amino-2-hydroxy-6- |
| | methylheptyl)-3-(1-(4-fluorophenyl)-2-methylpropan-2-yl)urea |
| I-23 | N-((2S,3S,5S)-5-(3-(3-methoxypropoxy)-4-methoxybenzyl)-3-amino-2-hydroxy-6- |
| | methylheptyl)-1-methylcyclohexanecarboxamide |
| I-24 | N-((2S,3S,5S)-5-(3-(3-methoxypropoxy)-4-methoxybenzyl)-3-amino-2-hydroxy-6- |
| | methylheptyl)-4-methylcyclohexanecarboxamide |
| I-28 | N-((2S,3S,5S)-5-(3-(3-methoxypropoxy)-4-methoxybenzyl)-3-amino-2-hydroxy-6- |
| | methylheptyl)-2,2-dimethylhexanamide |
| I-29 | N-((2R,3R,5S)-5-(3-(3-methoxypropoxy)-4-methoxybenzyl)-3-amino-2-hydroxy-6- |
| | methylheptyl)-2,2-dimethylhexanamide |
| I-30 | N-((2S,3S,5S)-5-(3-(3-methoxypropoxy)-4-methoxybenzyl)-3-amino-2-hydroxy-6- |
| | methylheptyl)-3,3-dimethylhexanamide |
| I-31 | N-((2S,3S,5S)-5-(3-(3-methoxypropoxy)-4-methoxybenzyl)-3-amino-2-hydroxy-6- |
| | methylheptyl)-2-ethylhexanamide |
| I-32 | N-((2S,3S,5S)-5-(3-(3-methoxypropoxy)-4-methoxybenzyl)-3-amino-2-hydroxy-6- |
| | methylheptyl)-2-methyl-2-propoxypropanamide |
| I-33 | N-((2S,3S,5S)-5-(3-(3-methoxypropoxy)-4-methoxybenzyl)-3-amino-2-hydroxy-6- |
| | methylheptyl)-3-ethoxy-2,2-dimethylpropanamide |
| I-34 | N-((2S,3S,5S)-5-(3-(3-methoxypropoxy)-4-methoxybenzyl)-3-amino-2-hydroxy-6- |
| | methylheptyl)-3-phenylpropanamide |
| I-35 | N-((2S,3S,5S)-5-(3-(3-methoxypropoxy)-4-methoxybenzyl)-3-amino-2-hydroxy-6- |
| | methylheptyl)-2-m-tolylacetamide |
| I-37 | N-((2S,3S,5S)-5-(3-(3-methoxypropoxy)-4-methoxybenzyl)-3-amino-2-hydroxy-6- |
| | methylheptyl)-2-(2-fluorophenyl)acetamide |
| I-38 | N-((2S,3S,5S)-5-(3-(3-methoxypropoxy)-4-methoxybenzyl)-3-amino-2-hydroxy-6- |
| | methylheptyl)-2-(3-fluorophenyl)acetamide |
| I-39 | N-((2S,3S,5S)-5-(3-(3-methoxypropoxy)-4-methoxybenzyl)-3-amino-2-hydroxy-6- |
| | methylheptyl)-2-(4-fluorophenyl)acetamide |
| I-40 | N-((2S,3S,5S)-5-(3-(3-methoxypropoxy)-4-methoxybenzyl)-3-amino-2-hydroxy-6- |
| | methylheptyl)-2-(4-fluorophenyl)acetamide |
| I-41 | N-((2S,3S,5S)-5-(3-(3-methoxypropoxy)-4-methoxybenzyl)-3-amino-2-hydroxy-6- |
| | methylheptyl)-5,5,5-trifluoropentanamide |
| I-42 | N-((2S,3S,5S)-5-(3-(3-methoxypropoxy)-4-methoxybenzyl)-3-amino-2-hydroxy-6- |
| | methylheptyl)-4-cyclopropyl-2,2-dimethylbutanamide |
| I-43 | N-((2R,3S,5S)-5-(3-(3-methoxypropoxy)-4-methoxybenzyl)-3-amino-2-hydroxy-6- |
| | methylheptyl)-4-cyclopropyl-2,2-dimethylbutanamide |
| I-45 | N-((2S,3S,5S)-5-(3-(3-methoxypropoxy)-4-methoxybenzyl)-3-amino-2-hydroxy-6- |
| | methylheptyl)-3,5,5-trimethylhexanamide |
| I-47 | N-((2S,3S,5S)-5-(3-(3-methoxypropoxy)-4-methoxybenzyl)-3-amino-2-hydroxy-6- |
| | methylheptyl)-2-(4-cyanophenyl)acetamide |
| I-48 | N-((2S,3S,5S)-5-(3-(3-methoxypropoxy)-4-methoxybenzyl)-3-amino-2-hydroxy-6- |
| | methylheptyl)-1-phenylcyclopropanecarboxamide |
| I-49 | N-((2S,3S,5S)-5-(3-(3-methoxypropoxy)-4-methoxybenzyl)-3-amino-2-hydroxy-6- |
| | methylheptyl)-4-phenylbutanamide |
| I-50 | N-((2S,3S,5S)-5-(3-(3-methoxypropoxy)-4-methoxybenzyl)-3-amino-2-hydroxy-6- |
| | methylheptyl)-2-methyl-2-phenylpropanamide |
| I-51 | N-((2R,3S,5S)-5-(3-(3-methoxypropoxy)-4-methoxybenzyl)-3-amino-2-hydroxy-6- |
| | methylheptyl)-2-methyl-2-phenylpropanamide |
| I-52 | N-((2S,3S,5S)-5-(3-(3-methoxypropoxy)-4-methoxybenzyl)-3-amino-2-hydroxy-6- |
| | methylheptyl)-2-cyclohexyl-2-methylpropanamide |
| I-53 | N-((2S,3S,5S)-5-(3-(3-methoxypropoxy)-4-methoxybenzyl)-3-amino-2-hydroxy-6- |
| | methylheptyl)-2-(3,4-difluorophenyl)acetamide |
| I-54 | N-((2S,3S,5S)-5-(3-(3-methoxypropoxy)-4-methoxybenzyl)-3-amino-2-hydroxy-6- |
| | methylheptyl)-2-(2,4-difluorophenyl)acetamide |
| I-55 | N-((2S,3S,5S)-5-(3-(3-methoxypropoxy)-4-methoxybenzyl)-3-amino-2-hydroxy-6- |
| | methylheptyl)-2-(2,3-difluorophenyl)acetamide |
| I-57 | N-((2S,3S,5S)-5-(3-(3-methoxypropoxy)-4-methoxybenzyl)-3-amino-2-hydroxy-6- |
| | methylheptyl)-1-p-tolylcyclopropanecarboxamide |
| I-60 | N-((2S,3S,5S)-5-(3-(3-methoxypropoxy)-4-methoxybenzyl)-3-amino-2-hydroxy-6- |
| | methylheptyl)-2-(4-fluorophenyl)-2-methylpropanamide |
| I-61 | N-((2R,3S,5S)-5-(3-(3-methoxypropoxy)-4-methoxybenzyl)-3-amino-2-hydroxy-6- |
| | methylheptyl)-2-(4-fluorophenyl)-2-methylpropanamide |
| I-62 | N-((2S,3S,5S)-5-(3-(3-methoxypropoxy)-4-methoxybenzyl)-3-amino-2-hydroxy-6- |
| | methylheptyl)-2-(4-fluorophenyl)-2-methylpropanamide |
| I-63 | N-((2S,3S,5S)-5-(3-(3-methoxypropoxy)-4-methoxybenzyl)-3-amino-2-hydroxy-6- |
| | methylheptyl)-2-cyclopentylhexanamide |
| I-64 | N-((2S,3S,5S)-5-(3-(3-methoxypropoxy)-4-methoxybenzyl)-3-amino-2-hydroxy-6- |
| | methylheptyl)-1-phenylcyclopentanecarboxamide |
| I-67 | N-((2S,3S,5S)-5-(3-(3-methoxypropoxy)-4-methoxybenzyl)-3-amino-2-hydroxy-6- |
| | methylheptyl)-1-(4-methoxyphenyl)cyclopropanecarboxamide |
| I-69 | N-((2S,3S,5S)-5-(3-(3-methoxypropoxy)-4-methoxybenzyl)-3-amino-2-hydroxy-6- |
| | methylheptyl)-3-hydroxy-2,2-dimethyl-3-phenylpropanamide |
| I-70 | N-((2S,3S,5S)-5-(3-(3-methoxypropoxy)-4-methoxybenzyl)-3-amino-2-hydroxy-6- |
| | methylheptyl)-1-(4-chlorophenyl)cyclopropanecarboxamide |
| I-72 | N-((2S,3S,5S)-5-(3-(3-methoxypropoxy)-4-methoxybenzyl)-3-amino-2-hydroxy-6- |
| | methylheptyl)-2-(2-(trifluoromethyl)phenyl)acetamide |
| I-73 | N-((2S,3S,5S)-5-(3-(3-methoxypropoxy)-4-methoxybenzyl)-3-amino-2-hydroxy-6- |
| | methylheptyl)-2-(3-(trifluoromethyl)phenyl)acetamide |
| I-74 | N-((2S,3S,5S)-5-(3-(3-methoxypropoxy)-4-methoxybenzyl)-3-amino-2-hydroxy-6- |
| | methylheptyl)-2-(4-(trifluoromethyl)phenyl)acetamide |
| I-75 | N-((2S,3S,5S)-5-(3-(3-methoxypropoxy)-4-methoxybenzyl)-3-amino-2-hydroxy-6- |
| | methylheptyl)-1-phenylcyclohexanecarboxamide |
| I-76 | N-((2S,3S,5S)-5-(3-(3-methoxypropoxy)-4-methoxybenzyl)-3-amino-2-hydroxy-6- |
| | methylheptyl)-2-(4-cyanophenoxy)-2-methylpropanamide |
| I-77 | N-((2S,3S,5S)-5-(3-(3-methoxypropoxy)-4-methoxybenzyl)-3-amino-2-hydroxy-6- |
| | methylheptyl)-2,2-bis(trifluoromethyl)propanamide |
| I-78 | N-((2S,3S,5S)-5-(3-(3-methoxypropoxy)-4-methoxybenzyl)-3-amino-2-hydroxy-6- |
| | methylheptyl)-2-ethyl-2-(4-fluorophenyl)butanamide |
| I-80 | N-((2S,3S,5S)-5-(3-(3-methoxypropoxy)-4-methoxybenzyl)-3-amino-2-hydroxy-6- |
| | methylheptyl)-2-(4-chlorophenoxy)-2-methylpropanamide |
| I-81 | N-((2S,3S,5S)-5-(3-(3-methoxypropoxy)-4-methoxybenzyl)-3-amino-2-hydroxy-6- |
| | methylheptyl)-2-(4-(trifluoromethoxy)phenyl)acetamide |
| I-82 | N-((2S,3S,5S)-5-(3-(3-methoxypropoxy)-4-methoxybenzyl)-3-amino-2-hydroxy-6- |
| | methylheptyl)-1-(4-fluorophenyl)cyclohexanecarboxamide |
| I-83 | N-((2S,3S,5S)-5-(3-(3-methoxypropoxy)-4-methoxybenzyl)-3-amino-2-hydroxy-6- |
| | methylheptyl)-1-(2,4-dichlorophenyl)cyclopropanecarboxamide. |

### EXAMPLE 7

The following compounds of formula I were prepared by following the procedures of Example 5, replacing the ammonium hydroxide in Step 1 with methylamine or isopropylamine, and substituting the cyclohexanecarboxylic acid used in Step 2 with 2,2-dimethylhexanoic acid or pentanoic acid:

| Cpd. | |
|---|---|
| No. | Name |
| I-46 | N-((2S,3S,5S)-5-(3-(3-methoxypropoxy)-4-methoxybenzyl)- |
| | 3-amino-2-hydroxy-6-methylheptyl)-N,2,2- |
| | trimethylhexanamide |
| I-27 | N-((2S-3S-5S)-5-(3-(3-methoxypropoxy)-4-methoxybenzyl)- |
| | 3-amino-2-hydroxy-6-methylheptyl)-N-isopropylpentanamide. |

### EXAMPLE 8

### N-((2S,3S,5S)-5-(3-(3-methoxypropoxy)-4-ethylbenzyl)-3-amino-2-hydroxy-6-methylheptyl)-2,2-dimethylhexanamide (I-25)

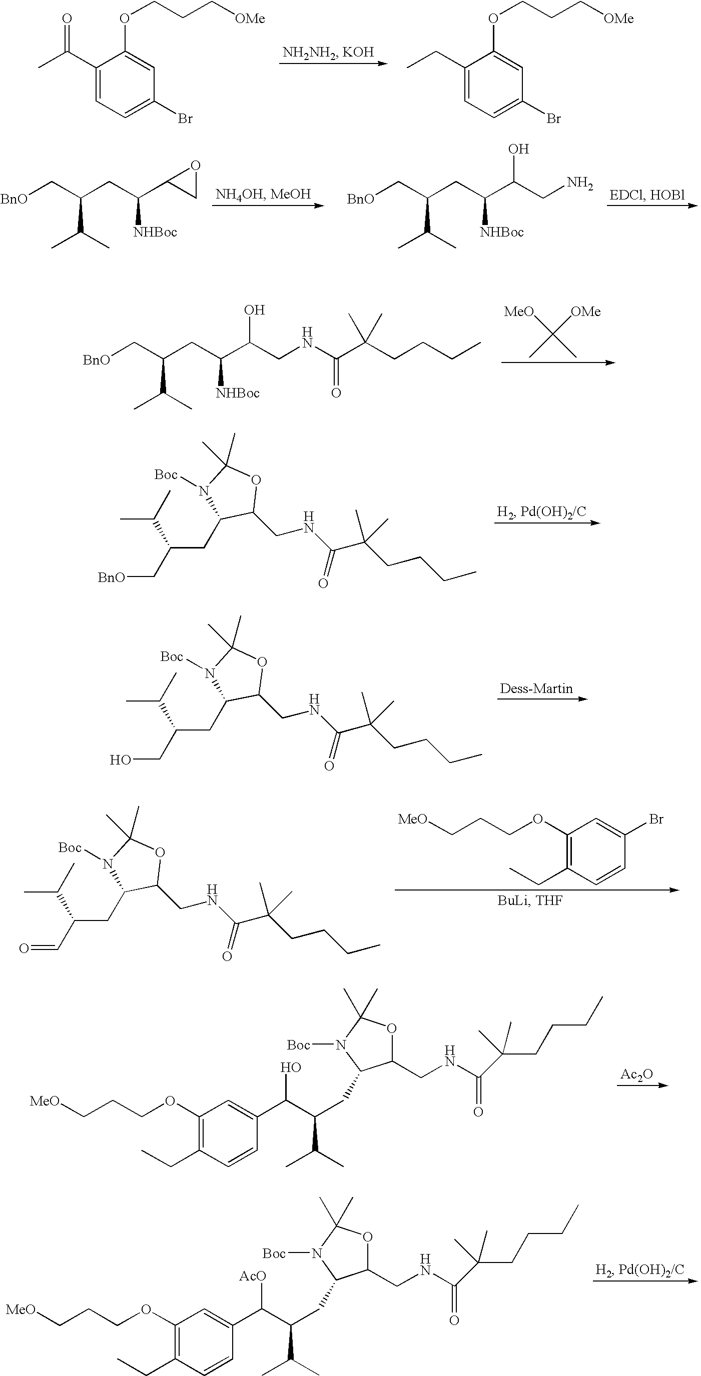

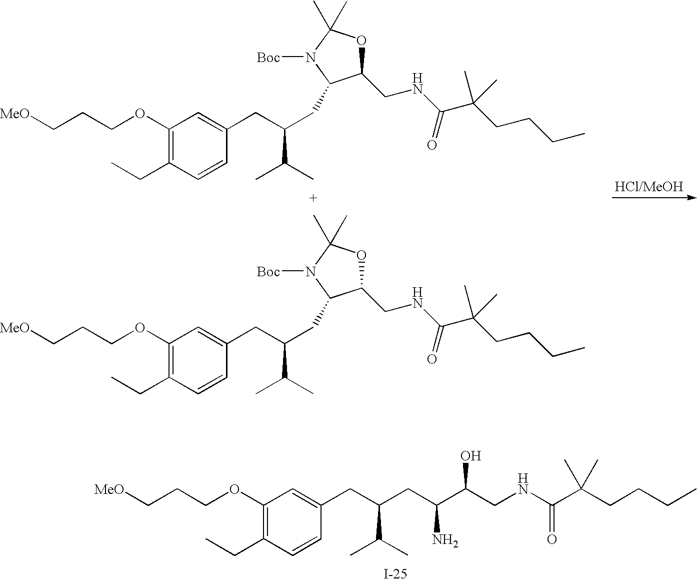

### Step 1

A mixture of 1-[4-bromo-2-(3-methoxy-propoxy)-phenyl]-ethanone (6.0 g, 21.0 mmol), KOH (4.7 g, 84 mmol) and NH₂NH₂ (2.7 g, 86 mmol) in 2-(2-hydroxy-ethoxy)-ethanol (50 mL) was stirred at 195° C. overnight. The resulting mixture was cooled to 0° C. and 5% aq HCl was added until the mixture had pH=1-2. The aqueous layer was extracted with ethylacetate (2×). The combined organic phase was washed with brine, dried over Na₂SO₄, filtered and concentrated in vacuo. The residue was purified by column chromatography to afford 4-bromo-1-ethyl-2-(3-(methoxy)propoxy)benzene (5.0 g, 87%). MS m/z 273 (M+H⁺)

### Step 2

To a room temperature solution of tert-butyl (1S,3S)-3-((benzyloxy)methyl)-4-methyl-1-(oxiran-2-yl)pentylcarbamate (182 mg, 0.50 mmol) in MeOH (1.5 mL) was added 28% aq NH₄OH(3 mL). The resulting clear solution was stirred at room temperature overnight. The solvent and excess ammonia was removed to give tert-butyl (3S,5S)-1-amino-5-((benzyloxy)-methyl)-2-hydroxy-6-methylheptan-3-ylcarbamate (180 mg, 0.47 mmol, 94% yield). ¹H NMR (400 MHz, CD₃OD) δ 7.40-7.20 (m, 5H), 4.45 (s, 1H), 3.60 (m, 2H), 3.45 (d, J=7.2 Hz, 2H), 2.65-2.35 (m, 2H), 1.80 (m, 1H), 1.70-1.20 (m, 3H), 1.45 (s, 9H), 1.00-0.70 (m, 6H); MS m/z 381 (M+H⁺).

### Step 3

To a solution of tert-butyl (3S,5S)-1-amino-5-((benzyloxy)methyl)-2-hydroxy-6-methylheptan-3-ylcarbamate (380 mg, 1.0 mmol) in dry CH₂Cl₂ (15 mL) were successively added 2,2-dimethyl-hexanoic acid (158 mg, 1.1 mmol), diisopropylethylamine (645 mg, 5.0 mmol), HOBt (270 mg, 2.0 mmol), and EDC.HCl (384 mg, 2.0 mmol) at 0° C. After stirred at 0° C. for 15 min, the reaction solution was washed with water and brine, dried over Na₂SO₄, filtered and concentrated in vacuo.

The residue was purified by column chromatography to afford N-((3S,5S)-3-(tert-butoxycarbonyl)amino-5-((benzyloxy)methyl)-2-hydroxy-6-methylheptyl)-2,2-dimethylhexanamide (240 mg, 47%) of acceptable purity based on LC-MS. MS m/z 507 (M+H⁺).

### Step 4

A solution of N-((3S,5S)-3-(tert-butoxycarbonyl)amino-5-((benzyloxy)methyl)-2-hydroxy-6-methylheptyl)-2,2-dimethylhexanamide (506 mg, 1.0 mmol) in acetone (8 mL) was cooled to 0° C. 2,2-dimethoxy-propane (832 mg, 8.0 mmol) was added followed by BF₃.Et₂O (0.1 mL). The solution was stirred at 0° C. for 30 min and at room temperature for 2 h. Triethylamine (0.5 mL) was added, the mixture was diluted with water and acetone was removed in vacuo. The aqueous layer was extracted with ethyl acetate and the organic phase was washed with brine, dried over Na₂SO₄, filtered and concentrated in vacuo. The residue was purified by column chromatography to afford (4S)-tert-butyl 5-((2,2-dimethylhexanamido)methyl)-4-((S)-2-((benzyloxy)methyl)-3-methylbutyl)-2,2-dimethyloxazolidine-3-carboxylate (379 mg, 0.69 mmol, 69.0% yield) of acceptable purity based on LC-MS. MS m/z 547 (M+H⁺).

### Step 5

A mixture of (4S)-tert-butyl 5-((2,2-dimethylhexanamido)methyl)-4-((S)-2-((benzyloxy)methyl)-3-methylbutyl)-2,2-dimethyloxazolidine-3-carboxylate (546 mg, 1.0 mmol) and 20% Pd(OH)₂/C (55 mg) in MeOH (10 mL) was hydrogenated at room temperature for 2 h. The mixture was filtered and the filtrate was concentrated in vacuo to give (4S)-tert-butyl 5-((2,2-dimethylhexanamido)methyl)-4-((S)-2-(hydroxymethyl)-3-methylbutyl)-2,2-dimethyloxazolidine-3-carboxylate (380 mg, 83%). ¹H NMR (400 MHz, CDCl₃) δ 6.04 (brs, 1H), 4.10-3.80 (m, 1H), 3.70-3.35 (m, 2H), 3.20 (m, 1H), 2.87 (m, 2H), 1.50 (m, 13H), 1.40-1.15 (m, 3H), 1.15 (s, 6H), 1.00-0.75 (m, 9H); MS m/z 457 (M+H⁺).

### Step 6

To a solution of (4S)-tert-butyl 5-((2,2-dimethylhexanamido)methyl)-4-((S)-2-(hydroxymethyl)-3-methylbutyl)-2,2-dimethyloxazolidine-3-carboxylate (456 mg, 1.0 mmol) in dry CH₂Cl₂ (20 mL), was added Dess-Martin periodinane (636 mg, 1.5 mmol). The mixture was stirred at room temperature for 5 h and filtered. The filtrate was concentrated in vacuo to give (4S)-tert-butyl 5-((2,2-dimethylhexanamido)methyl)-4-((S)-2-formyl-3-methylbutyl)-2,2-dimethyloxazolidine-3-carboxylate (410 mg, 0.90 mmol, 90% yield) of acceptable purity based on LC-MS. MS (E/Z): 455 (M+H⁺).

### Step 7

A solution of 4-bromo-1-ethyl-2-(3-methoxy-propoxy)-benzene (1.09 g, 4.0 mmol) in anhydrous THF (15 mL) was added dropwise a stirred solution of 2.5 M n-BuLi in hexanes (1.60 mL, 4.0 mmol) at −78° C. The mixture was stirred for 1 h at −78° C. A solution of (4S)-tert-butyl 5-((2,2-dimethylhexanamido)methyl)-4-((S)-2-formyl-3-methylbutyl)-2,2-dimethyloxazolidine-3-carboxylate (227 mg, 0.50 mmol) in anhydrous THF (2.0 mL) was added dropwise at −78° C. The mixture was stirred for 2 h at −78° C., and the temperature was raised from at −78° C. to room temperature during 2 h. After stirring for 18 h at room temperature, the reaction was quenched by addition of 10% aq NH₄Cl solution (10 mL). The product was extracted with ethyl acetate. The combined organic layers were washed with brine, dried over MgSO₄, and evaporated. The residue was purified by column chromatography to provide (4S)-tert-butyl 5-((2,2-dimethylhexanamido)methyl)-4-((S)-2-((3-(3-methoxypropoxy)-4-ethylphenyl)(hydroxy)methyl)-3-methylbutyl)-2,2-dimethyloxazolidine-3-carboxylate (97 mg, 30%) with acceptable purity based on LC-MS. MS m/z 649 (M+H⁺).

### Step 8

To a solution of (4S)-tert-butyl 5-((2,2-dimethylhexanamido)methyl)-4-((S)-2-((3-(3-methoxypropoxy)-4-ethylphenyl)(hydroxy)methyl)-3-methylbutyl)-2,2-dimethyloxazolidine-3-carboxylate (95 mg, 0.15 mmol) in dry CH₂Cl₂ (5 mL) was added acetic anhydride (0.5 mL) and pyridine (0.15 mL). The resulting solution was stirred at room temperature overnight. The solvent was removed in vacuo and the residue was purified by preparative tlc to afford (4S)-tert-butyl 5-((2,2-dimethylhexanamido)methyl)-4-((S)-2-((3-(3-methoxypropoxy)-4-ethylphenyl)-(acetoxy)methyl)-3-methylbutyl)-2,2-dimethyloxazolidine-3-carboxylate (68 mg, 67%) with acceptable purity based on LC-MS. MS m/z 691 (M+H⁺).

### Step 9

The mixture of (4S)-tert-butyl 5-((2,2-dimethylhexanamido)methyl)-4-((S)-2-((3-(3-methoxypropoxy)-4-ethylphenyl)(acetoxy)methyl)-3-methylbutyl)-2,2-dimethyloxazolidine-3-carboxylate (67 mg, 0.1 mmol) and 20% Pd(OH)₂/C (20 mg) in MeOH (4 mL) was hydrogenated at room temperature and 1 atm for 2 h. The mixture was filtered and the filtrate was concentrated in vacuo to leave a residue which was purified by preparative tlc to afford (4S,5S)-tert-butyl 5-((2,2-dimethylhexanamido)methyl)-4-((S)-2-(3-(3-methoxypropoxy)-4-ethylbenzyl)-3-methylbutyl)-2,2-dimethyloxazolidine-3-carboxylate (19 mg, 0.03 mmol) and (4S,5R)-tert-butyl 5-((2,2-dimethylhexanamido)methyl)-4-((S)-2-(3-(3-methoxypropoxy)-4-ethylbenzyl)-3-methylbutyl)-2,2-dimethyloxazolidine-3-carboxylate (24 mg, 0.04 mmol). MS m/z 633 (M+H⁺).

### Step 10

A solution of (4S,5S)-tert-butyl 5-((2,2-dimethylhexanamido)methyl)-4-((S)-2-(3-(3-methoxypropoxy)-4-ethylbenzyl)-3-methylbutyl)-2,2-dimethyloxazolidine-3-carboxylate (19 mg, 0.03 mmol) in 2N HCl-MeOH (3.0 mL) was stirred at 40° C. for 2 h. The solvent was removed in vacuo to afford N-((2S,3S,5S)-5-(3-(3-methoxypropoxy)-4-ethylbenzyl)-3-amino-2-hydroxy-6-methylheptyl)-2,2-dimethylhexanamide (I-25) (8.4 mg, 0.017 mmol, 56.7% yield) as its HCl salt. ¹H NMR (400 MHz, CD₃OD) δ 7.03 (d, J=8.0 Hz, 1H), 6.70 (m, 2H), 4.05 (t, J=6.0 Hz, 2H), 3.75 (brs, 1H), 3.60 (t, J=6.0 Hz, 2H), 3.37 (m, 1H), 3.35 (S, 3H), 3.28 (m, 1H), 3.00 (m, 1H), 2.60 (m, 3H), 2.43 (m, 1H), 2.05 (m, 2H), 1.90-1.65 (m, 4H), 1.55-1.20 (m, 9H), 1.15 (s, 6H), 0.96 (d, J=6.8 Hz, 3H), 0.95-0.70 (m, 6H); MS m/z 493 (M+H⁺).

### EXAMPLE 9

Treatment of (4S,5R)-tert-butyl 5-((2,2-dimethylhexanamido)methyl)-4-((S)-2-(3-(3-methoxypropoxy)-4-ethylbenzyl)-3-methylbutyl)-2,2-dimethyloxazolidine-3-carboxylate according to the procedure of Example 8 Step 10 afforded N-((2R,3S,5S)-5-(3-(3-methoxypropoxy)-4-ethylbenzyl)-3-amino-2-hydroxy-6-methylheptyl)-2,2-dimethylhexanamide (I-26) as its HCl salt. ¹H NMR (400 MHz, CD₃OD) δ 7.03 (m, 1H), 6.70 (m, 2H), 4.05 (t, J=6.0 Hz, 2H), 3.70 (m, 1H), 3.60 (t, J=6.0 Hz, 2H), 3.38 (m, 1H), 3.35 (S, 3H), 3.20 (m, 1H), 3.00 (m, 1H), 2.60 (m, 3H), 2.43 (m, 1H), 2.05 (m, 2H), 1.90-1.65 (m, 3H), 1.63 (m, 1H), 1.55-1.20 (m, 10H), 1.15 (s, 6H), 0.96 (d, J=6.8 Hz, 3H), 0.95-0.80 (m, 6H) (m, 9H); MS m/z 493 (M+H⁺)

### EXAMPLE 10

### N-((2S,3S,5S)-5-(3-(3-methoxypropoxy)-4-phenylbenzyl)-3-amino-2-hydroxy-6-methylheptyl)-2,2-dimethylhexanamide (I-65)

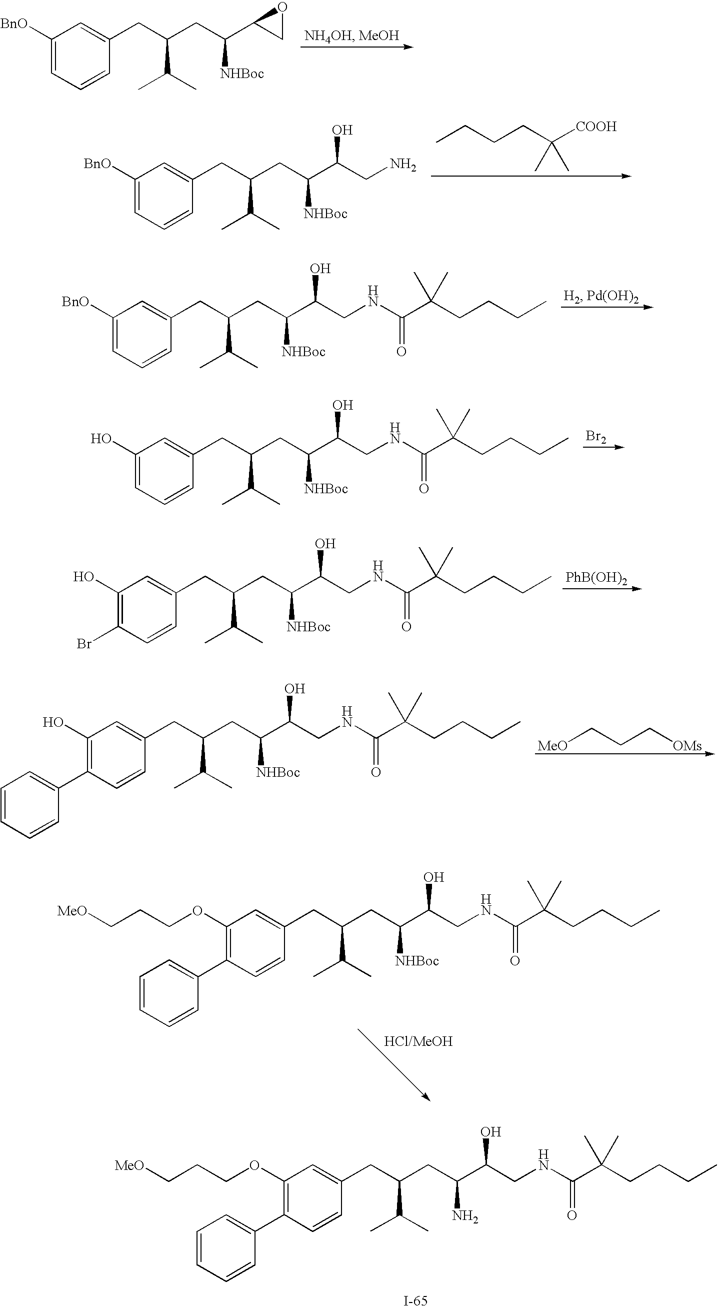

### Step 1

To a solution of tert-butyl (1S,3S)-3-(3-(benzyloxy)benzyl)-4-methyl-1-((R)-oxiran-2-yl)pentylcarbamate (1.1 g, 2.5 mmol) in MeOH (10 mL) at room temperature was added 28% aq NH₄OH (15 mL). The resulting clear solution was stirred at room temperature overnight. Solvent and excess ammonia were removed in vacuo to provide tert-butyl (2S,3S,5S)-5-(3-(benzyloxy)-benzyl)-1-amino-2-hydroxy-6-methylheptan-3-ylcarbamate (1.15 g, 100%). ¹H NMR (400 MHz, CD₃OD) δ 7.45-7.25 (m, 5H), 7.10 (m, 1H), 6.85-6.70 (m, 3H), 5.05 (s, 2H), 3.75-3.45 (m, 2H), 2.80-2.60 (m, 3H), 2.35 (m, 1H), 1.80-1.50 (m, 3H), 1.45 (s, 9H), 1.00-0.70 (m, 6H); MS m/z 457 (M+H⁺).

### Step 2

To a solution tert-butyl (2S,3S,5S)-5-(3-(benzyloxy)benzyl)-1-amino-2-hydroxy-6-methylheptan-3-ylcarbamate (1.14 g, 2.5 mmol) in dry CH₂Cl₂ (20 mL) were successively added 2,2-dimethyl-hexanoic acid (395 mg, 2.75 mmol), diisopropylethylamine (1.61 g, 12.5 mmol), HOBt (675 mg, 5.0 mmol), and EDC.HCl (960 mg, 5.0 mmol) at 0° C. After addition, the reaction mixture was stirred at 0° C. for 15 min and allowed to warm to room temperature for 1 h. The reaction solution was washed with water and brine, dried over Na₂SO₄, filtered and concentrated in vacuo. The residue was purified by chromatography on silica gel to afford N-((2S,3S,5S)-5-(3-benzyloxybenzyl)-3-(tert-butoxycarbonyl)amino-2-hydroxy-6-methylheptyl)-2,2-dimethyl-hexanamide (700 mg, 48%). ¹H NMR (400 MHz, CDCl₃) δ 7.45-7.25 (m, 5H), 7.10 (m, 1H), 6.80-6.70 (m, 3H), 5.05 (s, 2H), 4.75 (m, 1H), 3.80-3.50 (m, 3H), 3.30-3.00 (m, 2H), 2.80 (m, 1H), 2.60-2.50 (m, 1H), 1.80-1.50 (m, 3H), 1.45 (s, 9H), 1.50 0.72 (m, 21H); MS m/z 583 (M+H⁺).

### Step 3

A solution of N-((2S,3S,5)-5-(3-benzyloxybenzyl)-3-(tert-butoxycarbonyl)amino-2-hydroxy-6-methylheptyl)-2,2-dimethylhexanamide (582 mg, 1.0 mmol) and 20% Pd(OH)₂/C (60 mg) in MeOH (10 mL) was hydrogenated at room temperature for 2 h. The mixture was filtered and the filtrate was concentrated in vacuo to give N-((2S,3S,5S)-5-(3-hydroxybenzyl)-3-(tert-butoxycarbonyl)amino-2-hydroxy-6-methylheptyl)-2,2-dimethylhexanamide (410 mg, 83%). ¹H NMR (400 MHz, CDCl₃) δ 7.10 (m, 1H), 7.00 (s, 1H), 6.68 (m, 2H), 4.80 (d, J=7.2 Hz, 1H), 3.55 (m, 3H), 2.90 (m, 1H), 2.60-2.35 (m, 2H), 1.69 (m, 1H), 1.75-1.50 (m, 3H), 1.45 (S, 9H), 1.60-1.20 (m, 9H), 1.15 (s, 6H), 0.86 (m, 9H); MS m/z 493 (M+H⁺).

### Step 4

To a solution of N-((2S,3S,5S)-5-(3-hydroxybenzyl)-3-(tert-butoxycarbonyl)amino-2-hydroxy-6-methylheptyl)-2,2-dimethylhexanamide (492 mg, 1.0 mmol) in anhydrous THF (15 mL) was added NaHCO₃ (170 mg). The solution was cooled to −78° C. and a solution of Br₂ (192 mg, 1.2 mmol) in anhydrous THF (2 mL) was added dropwise. The mixture was stirred for 2 h at −78° C., and the temperature was raised from −78° C. to room temperature over 2 h. After stirring for 18 h at room temperature, the reaction was quenched by addition of aqueous NaHSO₃ and the mixture was extracted with ethyl acetate. The combined organic layers were washed with brine, dried over MgSO₄, and evaporated. The residue was purified by column chromatography to give N-((2S,3S,5S)-5-(3-hydroxy-4-bromobenzyl)-3-(tert-butoxycarbonyl)amino-2-hydroxy-6-methylheptyl)-2,2-dimethylhexanamide (277 mg, 49%). ¹H NMR (400 MHz, CDCl₃) δ 7.32 (d, J=8.4 Hz, 1H), 7.10 (d, J=2.4 Hz, 1H), 6.77 (brt, 1H), 6.65 (dd, J=8.4 & 2.4 Hz, 1H), 4.35 (d, J=9.2 Hz, 1H), 3.75-3.55 (m, 4H), 2.85 (m, 1H), 2.75 (m, 1H), 1.80-1.65 (m, 2H), 1.55-1.45 (m, 2H), 1.45 (s, 9H), 1.40-1.20 (m, 4H), 1.15 (s, 6H), 1.00-0.70 (m, 6H); MS m/z 571 (M+H⁺).

### Step 5

To a mixture of N-((2S,3S,5S)-5-(3-hydroxy-4-bromobenzyl)-3-(tert-butoxycarbonyl)-amino-2-hydroxy-6-methylheptyl)-2,2-dimethylhexanamide (285 mg, 0.50 mmol) in toluene (1.0 mL) and 2 M aqueous sodium carbonate (0.9 mL) under nitrogen were successively added PhB(OH)₂ (67 mg, 0.55 mmol) and tetrakis-(triphenylphosphine) palladium (17.5 mg, 0.015 mmol). The mixture was heated under reflux for 4 h and cooled. The mixture was partitioned between water and ether; the aqueous layer was extracted with ethyl acetate. The combined organic layers were washed with brine, dried over MgSO₄, and evaporated. The residue was purified by preparative HPLC to give N-((2S,3S,5S)-5-(3-hydroxy-4-phenylbenzyl)-3-(tert-butoxycarbonyl)amino-2-hydroxy-6-methylheptyl)-2,2-dimethylhexanamide (116 mg, 40%). MS m/z 569 (M+H⁺).

### Step 6

To a solution of N-((2S,3S,5S)-5-(3-hydroxy-4-phenylbenzyl)-3-(tert-butoxycarbonyl)-amino-2-hydroxy-6-methylheptyl)-2,2-dimethylhexanamide (284 mg, 0.50 mmol) in acetonitrile (5 mL) were added 3-methoxypropyl methanesulfonate (168 mg, 1.0 mmol) and K₂CO₃ (345 mg, 2.5 mmol). The mixture was refluxed for 15 h and the solvent was removed. The residue was diluted with water and extracted with ethyl acetate. The combined organic phase was washed by brine, and dried over Na₂SO₄, filtered and concentrated in vacuo. The residue was purified by preparative HPLC to give N-((2S,3S,5S)-5-(3-(3-methoxypropoxy)-4-phenylbenzyl)-3-(tert-butoxycarbonyl)amino-2-hydroxy-6-methylheptyl)-2,2-dimethylhexanamide (180 mg, 56%). MS m/z 641 (M+H⁺).

### Step 7

A solution of N-((2S,3S,5S)-5-(3-(3-methoxypropoxy)-4-phenylbenzyl)-3-(tert-butoxy-carbonyl)amino-2-hydroxy-6-methylheptyl)-2,2-dimethylhexanamide (32 mg, 0.05 mmol) in 2 N HCl-MeOH (2.0 mL, 4 mmol) was stirred at 40° C. for 2 h. The solvent was removed in vacuo and the residue was purified by preparative HPLC to afford N-((2S,3S,5S)-5-(3-(3-methoxypropoxy)-4-phenylbenzyl)-3-amino-2-hydroxy-6-methylheptyl)-2,2-dimethylhexanamide (I-65) as its trifluoroacetic acid salt (16 mg, 60%). ¹H NMR (400 MHz, CDCl₃) δ 7.45 (m, 1H), 7.38 (m, 1H), 7.30 (m, 3H), 7.10 (d, J=8.4 Hz, 1H), 6.80 (m, 1H), 6.50 (brs, 1H), 4.10 (t, J=6.4 Hz, 2H), 3.61 (t, J=6.0 Hz, 2H), 3.48 (m, 1H), 3.40 (s, 3H), 3.38 (m, 1H), 3.25 (m, 1H), 2.62 (m, 2H), 2.10 (m, 2H), 1.48 (m, 4H), 1.28 (m, 2H), 1.17 (s, 6H), 1.17 (m, 2H), 0.87 (m, 4H), 0.71 (d, J=7.2 Hz, 3H), 0.60 (d, J=7.2 Hz, 3H); MS m/z 541 (M+H⁺). Isomeric N-((2S,3S,5S)-5-(5-(3-methoxypropoxy)-2-phenylbenzyl)-3-amino-2-hydroxy-6-methylheptyl)-2,2-dimethylhexanamide (I-66) was isolated as a minor product.

### EXAMPLE 11

The following compounds of formula I were prepared by following the procedures of Example 10

| Cpd. | |
|---|---|
| No. | Name |
| I-5 | N-((2S,3S,5S)-5-(3-(2-cyclopropylethoxy)benzyl)-3-amino-2-hydroxy-6-methylheptyl)-2,2- |
| | dimethylhexanamide - by treatment of the product of Step 3 with 2-(cyclopropyl)ethyl |
| | methanesulfonate according to the conditions of Step 6 and deprotection by the method of |
| | Step 7. |
| I-14 | N-((2S,3S,5S)-5-(3-(3-ethoxypropoxy)benzyl)-3-amino-2-hydroxy-6-methylheptyl)-2,2- |
| | dimethylhexanamide - by treatment of the product of Step 3 with 3-(ethoxy)propyl |
| | methanesulfonate according to the conditions of Step 6 and deprotection by the method of |
| | Step 7. |
| I-68 | N-((2S,3S,5S)-5-(3-(3-methoxypropoxy)-4-bromobenzyl)-3-amino-2-hydroxy-6-methylheptyl)- |
| | 2,2-dimethylhexanamide - by treatment of the product of Step 4 with 3-methoxypropyl |
| | methanesulfonate according to the conditions of Step 6 and deprotection by the method of |
| | Step 7. |

### EXAMPLE 12

### (2S,3S,5S)-5-(3-(3-methoxypropoxy)-4-methoxybenzyl)-1-(phenylmethylsulfonylamino)-3-amino-6-methylheptan-2-ol (I-56)

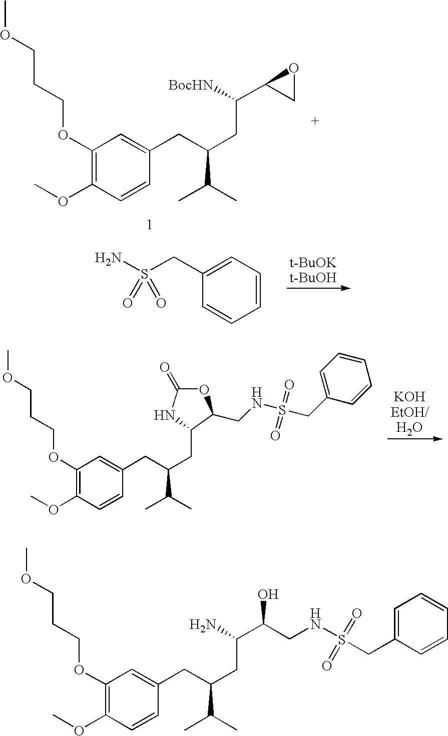

### Step 1

The mixture of tert-butyl (1S,3S)-3-(3-(3-methoxypropoxy)-4-methoxybenzyl)-4-methyl-1-((R)-oxiran-2-yl)pentylcarbamate (1) (22.0 mg, 0.047 mmol) and phenylmethanesulfonamide (80.5 mg, 0.47 mmol) was dissolved in 1 M KOt-Bu in t-BuOH (2 mL, 2 mmol). The resulting solution was heated in a CEM microwave synthesizer at 70° C. for 15 min, and the completion of reaction was confirmed by LC-MS. The mixture neutralized by addition of 6 N HCl and preparative HPLC gave (4S,5S)-4-((S)-2-(3-(3-methoxypropoxy)-4-methoxybenzyl)-3-methylbutyl)-5-((phenylmethanesulfonyl)aminomethyl)oxazolidin-2-one (3.3 mg, 13%) as a clear oil. ¹H NMR (CD₃OD) δ (ppm): 7.39 (s, 5H), 6.78 (dd, J=8.0, 6.4 Hz, 1H), 6.71 (s, 1H), 6.65 (d, J=8.0 Hz, 1H), 5.98, 5.83 (two s, 1H), 5.48, 5.32 (t, J=8.0 Hz, 1H), 4.28 (d, J=3.2 Hz, 2H), 4.09 (t, J=6.4 Hz, 2H), 3.98, 3.84 (two m, 1H), 3.83 (s, 3H), 3.58 (t, J=6.4 Hz, 2H), 3.61-3.57 (m, 1H), 3.52-3.49 (m, 2H), 3.35, 3.34 (two 8, 3H), 3.00-2.85 (m, 2H), 2.65-2.54 (m, 1H), 2.35-2.29 (m, 1H), 2.08 (m, 2H), 1.73-1.68 (m, 1H), 1.54-1.44 (m, 2H), 1.38-1.30 (m, 1H), 0.94-0.84 (m, 6H); MS: [M+H]⁺=549.

### Step 2

To a solution of (4S,5S)-4-((S)-2-(3-(3-methoxypropoxy)-4-methoxybenzyl)-3-methylbutyl)-5-((phenylmethanesulfonyl)aminomethyl)oxazolidin-2-one (3.3 mg, 0.006 mmol) in ethanol (0.7 mL) and water (0.33 mL) was added solid KOH (1 pellet, excess). The resulting solution was heated at 100° C. for 10 min in a CEM microwave apparatus. Reaction completion was confirmed by LC-MS, and the solution was acidified with 6 N HCl to neutral, and HPLC give (2S,3S,5S)-5-(3-(3-methoxypropoxy)-4-methoxybenzyl)-1-(phenylmethylsulfonylamino)-3-amino-6-methylheptan-2-ol (I-56) as its trifluoroacetic acid salt. ¹H NMR (CD₃OD) δ (ppm): 7.43-7.37 (m, 5H), 6.86-6.73 (m, 3H), 4.36 (d, J=4.4 Hz, 2H), 4.05 (t, J=6.0 Hz, 2H), 3.79 (s, 3H), 3.66, 3.50 (two m, 1H), 3.57 (t, J=6.4 Hz, 2H), 3.33 (s, 3H), 3.25, 3.14 (two m, 1H), 3.01-2.88 (m, 2H), 2.64-2.38 (m, 2H), 2.04-1.96 (m, 2H), 1.83-1.69 (m, 2H), 1.60, 1.30 (two m, 1H), 1.53 (t, J=6.8 Hz, 1H), 0.95-0.86 (m, 6H); MS m/z 523 (M+1).

### EXAMPLE 13

The following compounds of formula I were prepared by following the procedures of Example 12:

| Cpd. | |
|---|---|
| No. | Name |
| I-19 | (2S,3S,5S)-5-(3-(3-methoxypropoxy)-4-methoxybenzyl)-3-amino-6-methyl-1- |
| | (butanesulfonylamino)heptan-2-ol |
| I-36 | (2S,3S,5S)-5-(3-(3-methoxypropoxy)-4-methoxybenzyl)-3-amino-6-methyl-1- |
| | (pentanesulfonylamino)heptan-2-ol |
| I-44 | (2S,3S,5S)-5-(3-(3-methoxypropoxy)-4-methoxybenzyl)-3-amino-6-methyl-1- |
| | (benzenesulfonylamino)heptan-2-ol |
| I-58 | (2S,3S,5S)-5-(3-(3-methoxypropoxy)-4-methoxybenzyl)-3-amino-1-(N-isopropyl-N- |
| | butanesulfonylamino)-6-methylheptan-2-ol |
| I-59 | (2R,3S,5S)-5-(3-(3-methoxypropoxy)-4-methoxybenzyl)-3-amino-1-(N-isopropyl-N- |
| | butanesulfonylamino)-6-methylheptan-2-ol |
| I-71 | (2S,3S,5S)-5-(3-(3-methoxypropoxy)-4-methoxybenzyl)-3-amino-1-(N-isopropyl-N- |
| | benzenesulfonylamino)-6-methylheptan-2-ol |
| I-79 | (2S,3S,5S)-5-(3-(3-methoxypropoxy)-4-methoxybenzyl)-3-amino-1-(N-isopropyl-N- |
| | benzylsulfonylamino)-6-methylheptan-2-ol |

### EXAMPLE 14

### 1-((2S,3S,5S)-5-(3-(3-methoxypropoxy)-4-methoxybenzyl)-3-amino-2-hydroxy-6-methylheptyl)-3-benzylurea (I-102)

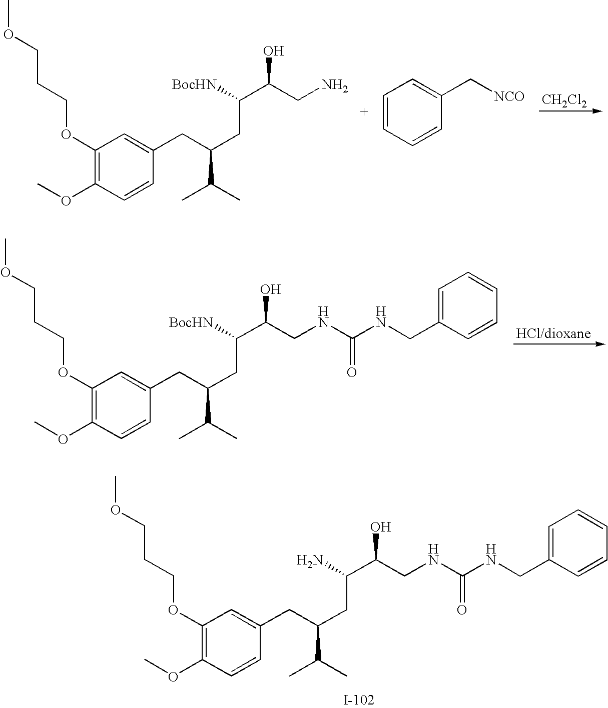

### Step 1

To a solution of tert-butyl (2S,3S,5S)-5-(3-(3-methoxypropoxy)-4-methoxybenzyl)-1-amino-2-hydroxy-6-methylheptan-3-ylcarbamate 2 (20.1 mg, 0.043 mmol) in acetonitrile (1 mL) was added benzyl isocyanate (5.8 mg, 0.43 mmol). The resulting solution was stirred at room temperature overnight and purified directly by preparative HPLC to give 1-((2S,3S,5S)-5-(3-(3-methoxypropoxy)-4-methoxybenzyl)-3-(tert-butoxycarbonylamino)-2-hydroxy-6-methylheptyl)-3-benzylurea (16.3 mg, 63%). MS m/z 602 [M+H]⁺.

### Step 2

1-((2S,3S,5S)-5-(3-(3-methoxypropoxy)-4-methoxybenzyl)-3-(tert-butoxycarbonylamino)-2-hydroxy-6-methylheptyl)-3-benzylurea (16.3 mg, 0.027 mmol) was treated with 4 M HCl in dioxane (2 mL, 8 mmol) at room temperature for 1 h. The solvent was removed in vacuo to give 1-((2S,3S,5S)-5-(3-(3-methoxypropoxy)-4-methoxybenzyl)-3-amino-2-hydroxy-6-methylheptyl)-3-benzylurea (I-102) as its HCl salt in quantitative yield. ¹H NMR (CD₃OD) δ 7.27-7.25 (m, 4H), 7.20 (m, 1H), 6.86-6.72 (m, 3H), 4.30 (m, 2H), 4.04 (t, J=6.4 Hz, 2H), 3.80 (s, 3H), 3.57 (t, J=6.4 Hz, 3H), 3.31 (s, 3H), 3.28 (m, 1H), 3.12 (m, 1H), 2.99-2.92 (m, 1H), 2.61 (dd, J=13.6, 6.4 Hz, 1H), 2.42 (d, J=13.6, 8.0 Hz, 1H), 2.05-1.98 (m, 2H), 1.87-1.70 (m, 3H), 1.60 (m, 1H), 0.97-0.88 (m, 6H); MS m/z 502 [M+H]⁺.

### EXAMPLE 15

The following compounds of formula I were prepared by the procedures of Example 14, substituting the appropriate isocyanate in Step 1:

| | |
|---|---|
| I-85 | 1-((2S,3S,5S)-5-(3-(3-methoxypropoxy)-4-methoxybenzyl)-3-amino-2-hydroxy-6-methylheptyl)-3- |
| | butylurea |
| I-86 | 1-((2S,3S,5S)-5-(3-(3-methoxypropoxy)-4-methoxybenzyl)-3-amino-2-hydroxy-6-methylheptyl)-3- |
| | tert-butylurea |
| I-89 | 1-((2S,3S,5S)-5-(3-(3-methoxypropoxy)-4-methoxybenzyl)-3-amino-2-hydroxy-6-methylheptyl)-3- |
| | (2-cyclopropylethyl)urea |
| I-91 | 1-((2S,3S,5S)-5-(3-(3-methoxypropoxy)-4-methoxybenzyl)-3-amino-2-hydroxy-6-methylheptyl)-3- |
| | pentylurea |
| I-92 | 1-((2S,3S,5S)-5-(3-(3-methoxypropoxy)-4-methoxybenzyl)-3-amino-2-hydroxy-6-methylheptyl)-3- |
| | pentylurea |
| I-93 | 1-((2R,3S,5S)-5-(3-(3-methoxypropoxy)-4-methoxybenzyl)-3-amino-2-hydroxy-6-methylheptyl)-3- |
| | pentylurea |
| I-95 | 1-((2S,3S,5S)-5-(3-(3-methoxypropoxy)-4-methoxybenzyl)-3-amino-2-hydroxy-6-methylheptyl)-3- |
| | (3-methoxypropyl)urea |
| I-96 | 1-((2S,3S,5S)-5-(3-(3-methoxypropoxy)-4-methoxybenzyl)-3-amino-2-hydroxy-6-methylheptyl)-3- |
| | (2-ethoxyethyl)urea |
| I-97 | 1-((2S,3S,5S)-5-(3-(3-methoxypropoxy)-4-methoxybenzyl)-3-amino-2-hydroxy-6-methylheptyl)-3- |
| | cyclohexylurea |
| I-98 | 1-((2S,3S,5S)-5-(3-(3-methoxypropoxy)-4-methoxybenzyl)-3-amino-2-hydroxy-6-methylheptyl)-3- |
| | hexylurea |
| I-100 | 1-((2S,3S,5S)-5-(3-(3-methoxypropoxy)-4-methoxybenzyl)-3-amino-2-hydroxy-6-methylheptyl)-1- |
| | methyl-3-pentylurea |
| I-105 | 1-((2S,3S,5S)-5-(3-(3-methoxypropoxy)-4-methoxybenzyl)-3-amino-2-hydroxy-6-methylheptyl)-3- |
| | (3-fluorophenyl)urea |
| I-106 | 1-((2S,3S,5S)-5-(3-(3-methoxypropoxy)-4-methoxybenzyl)-3-amino-2-hydroxy-6-methylheptyl)-3- |
| | (cyclohexylmethyl)urea |
| I-108 | (2S,3S,5S)-5-(3-(3-methoxypropoxy)-4-methoxybenzyl)-3-amino-1-(N-(butylaminosulfonyl)-N- |
| | isopropylamino)-6-methylheptan-2-ol |
| I-109 | 1-((2S,3S,5S)-5-(3-(3-methoxypropoxy)-4-methoxybenzyl)-3-amino-2-hydroxy-6-methylheptyl)-3- |
| | (2,2-dimethylpentyl)urea |
| I-110 | 1-((2S,3S,5S)-5-(3-(3-methoxypropoxy)-4-methoxybenzyl)-3-amino-2-hydroxy-6-methylheptyl)-3- |
| | (2-methylhexan-2-yl)urea |
| I-112 | 1-((2S,3S,5S)-5-(3-(3-methoxypropoxy)-4-methoxybenzyl)-3-amino-2-hydroxy-6-methylheptyl)-3- |
| | phenethylurea |
| I-114 | 1-((2S,3S,5S)-5-(3-(3-methoxypropoxy)-4-methoxybenzyl)-3-amino-2-hydroxy-6-methylheptyl)-3- |
| | (2-cyclohexylethyl)urea |
| I-115 | 1-((2S,3S,5S)-5-(3-(3-methoxypropoxy)-4-methoxybenzyl)-3-amino-2-hydroxy-6-methylheptyl)-3- |
| | (2,4,4-trimethylpentan-2-yl)urea |
| I-117 | 1-((2S,3S,5S)-5-(3-(3-methoxypropoxy)-4-methoxybenzyl)-3-amino-2-hydroxy-6-methylheptyl)-3- |
| | (3-phenylpropyl)urea |
| I-119 | 1-((2S,3S,5S)-5-(3-(3-methoxypropoxy)-4-methoxybenzyl)-3-amino-2-hydroxy-6-methylheptyl)-3- |
| | (3-(trifluoromethyl)phenyl)urea |
| I-120 | 1-((2S,3S,5S)-5-(3-(3-methoxypropoxy)-4-methoxybenzyl)-3-amino-2-hydroxy-6-methylheptyl)-3- |
| | (1-(4-fluorophenyl)-2-methylpropan-2-yl)urea. |

### EXAMPLE 16

### N-((2S,3S,5S)-5-(3-(3-methoxypropoxy)-4-methoxybenzyl)-3-amino-2-hydroxy-6-methylheptyl)piperidine-1-carboxamide (I-88)

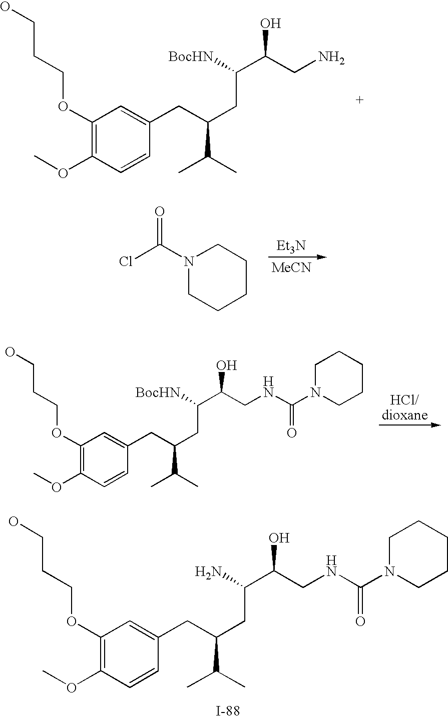

### Step 1

To a solution of tert-butyl (2S,3S,5S)-5-(3-(3-methoxypropoxy)-4-methoxybenzyl)-1-amino-2-hydroxy-6-methylheptan-3-ylcarbamate (20 mg, 0.043 mmol) in MeCN (0.4 mL) and Et₃N (0.1 mL), piperidine-1-carbonyl chloride (6.5 μL, 0.051 mmol) was added in one portion at room temperature. The resulting solution was stirred at room temperature until no starting remained (˜30 min), and purified by preparative HPLC to afford N-((2S,3S,5S)-5-(3-(3-methoxypropoxy)-4-methoxybenzyl)-3-(tert-butoxycarbonylamino)-2-hydroxy-6-methylheptyl)piperidine-1-carboxamide (18.5 mg, 76%). MS m/z 580 [M+H]⁺.

### Step 2

N-((2S,3S,5S)-5-(3-(3-methoxypropoxy)-4-methoxybenzyl)-3-(tert-butoxycarbonylamino)-2-hydroxy-6-methylheptyl)piperidine-1-carboxamide (18.5 mg, 0.032 mmol) was dissolved in 4 M HCl in dioxane (2 mL, 8 mmol) and stirred at room temperature for 1 h. The solvent was removed in vacuo to give N-((2S,3S,5S)-5-(3-(3-methoxypropoxy)-4-methoxybenzyl)-3-amino-2-hydroxy-6-methylheptyl)piperidine-1-carboxamide (I-88) as its HCl salt in quantitative yield. ¹H NMR (CD₃OD) δ 0.9 (m), 1.5-1.8 (m), 2.02 (m), 2.40 (m), 2.60 (m), 2.92 (m), 3.36 (s)m 3.38 (m), 3.60 (t), 3.80 (s), 4.06 (t), 6.75-6.9 (m). MS m/z 480 [M+H]⁺.

### EXAMPLE 17

The following compounds of Formula I were prepared by the procedure of Example 16 substituting the appropriate carbamoyl chloride in Step 1:
I-90 N-((2S,3S,5S)-5-(3-(3-methoxypropoxy)-4-methoxybenzyl)-3-amino-2-hydroxy-6-methylheptyl)morpholine-4-carboxamide
I-99 3-((2S,3S,5S)-5-(3-(3-methoxypropoxy)-4-methoxybenzyl)-3-amino-2-hydroxy-6-methylheptyl)-1-methyl-1-pentylurea
I-107 3-((2S,3S,5S)-5-(3-(3-methoxypropoxy)-4-methoxybenzyl)-3-amino-2-hydroxy-6-methylheptyl)-1-cyclohexyl-1-methylurea
I-116 3-((2S,3S,5S)-5-(3-(3-methoxypropoxy)-4-methoxybenzyl)-3-amino-2-hydroxy-6-methylheptyl)-1,1-diisobutylurea.

### EXAMPLE 18

### 1-((2S,3S,5S)-5-(3-(3-methoxypropoxy)-4-methoxybenzyl)-3-amino-2-hydroxy-6-methylheptyl)-3-(2-carbamoyl-2-methylpropyl)urea (I-111)

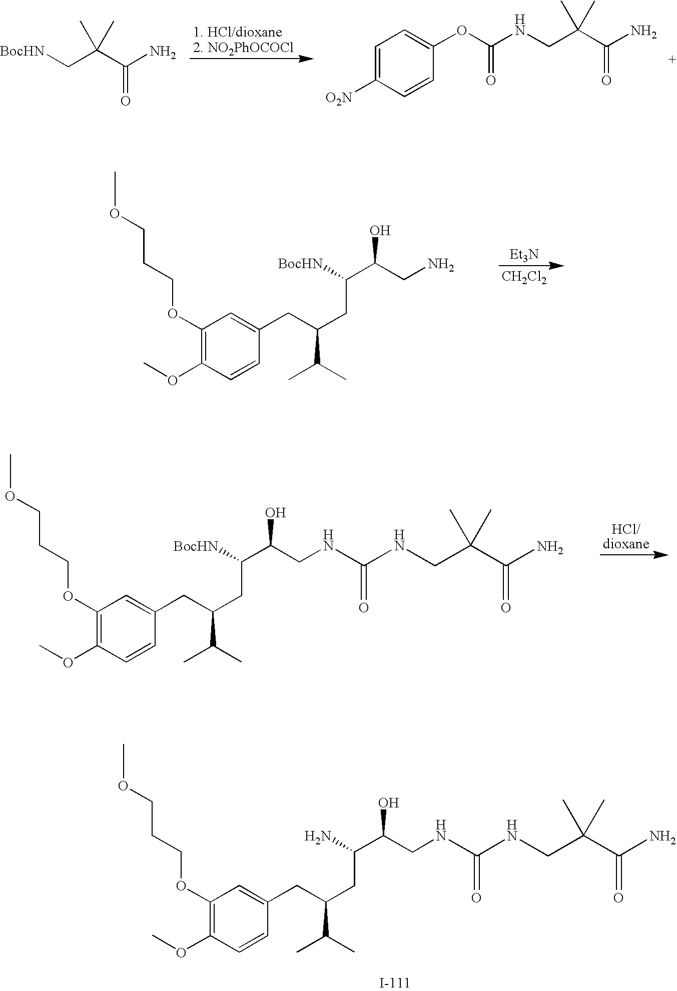

### Step 1

tert-Butyl 2-carbamoyl-2-methylpropylcarbamate (1) (1.01 g, 4.67 mmol) was dissolved in 4 M HCl in dioxane (10 mL, 40 mmol) at room temperature and stirred for 3 h. Removal of solvent afforded 2-(aminomethyl)-2-methylpropanamide as its HCl salt. This material was stirred with CH₂Cl₂ (10 mL) and pyridine (1.11 g, 14.0 mmol) was added, followed by 4-nitrophenyl chloroformate (1.07 g, 5.14 mmol). The resulting solution was stirred at room temperature for 30 min, diluted with CH₂Cl₂ (10 mL), washed with 1 N aq HCl (10 mL) and satd aq NaHCO₃ (10 mL), dried over Na₂SO₄, and concentrated to give 4-nitrophenyl 2-carbamoyl-2-methylpropyl-carbamate as a solid (1.54 g, quantitative) which was used for next in the next step without further purification. MS m/z 282 [M+H]⁺.

### Step 2

To a solution of tert-butyl (2S,3S,5S)-5-(3-(3-methoxypropoxy)-4-methoxybenzyl)-1-amino-2-hydroxy-6-methylheptan-3-ylcarbamate (21.6 mg, 0.046 mmol) in CH₂Cl₂ (0.5 mL) and Et₃N (0.1 mL), 4-nitrophenyl 2-carbamoyl-2-methylpropyl carbamate (13.0 mg, 0.046 mmol) was added. The resulting solution was stirred at room temperature until no starting material remained (˜30 min), and HPLC purification gave 1-((2S,3S,5S)-5-(3-(3-methoxypropoxy)-4-methoxybenzyl)-3-amino-2-hydroxy-6-methylheptyl)-3-(2-carbamoyl-2-methylpropyl)urea (3.3 mg, 9%) MS m/z 611 [M+H]⁺.

### Step 3

1-((2S,3S,5S)-5-(3-(3-methoxypropoxy)-4-methoxybenzyl)-3-amino-2-hydroxy-6-methylheptyl)-3-(2-carbamoyl-2-methylpropyl)urea (3.3 mg, 0.005 mmol) was treated with HCl/dioxane (4 M, 1 mL) at room temperature for 1 hr. Solvent was removed in vacuo to give 1-((2S,3S,5S)-5-(3-(3-methoxypropoxy)-4-methoxybenzyl)-3-amino-2-hydroxy-6-methylheptyl)-3-(2-carbamoyl-2-methylpropyl)urea (I-111) as its HCl salt in quantitative yield. ¹H NMR (CD₃OD) δ 0.9 (m), 1.16 (s), 1.60 (m), 0.7 (m), 2.02 (m), 2.40 (m), 2.60 (m), 3.24 (m), 3.36 (s), 3.60 (t), 3.80 (s), 4.06 (t), 6.7-6.9 (m). MS m/z 511 [M+H]⁺.

### EXAMPLE 19

### 1-((2S,3S,5S)-5-(3-(3-methoxypropoxy)-4-methoxybenzyl)-3-amino-202-hydroxy-6-methylheptyl)urea (I-84)

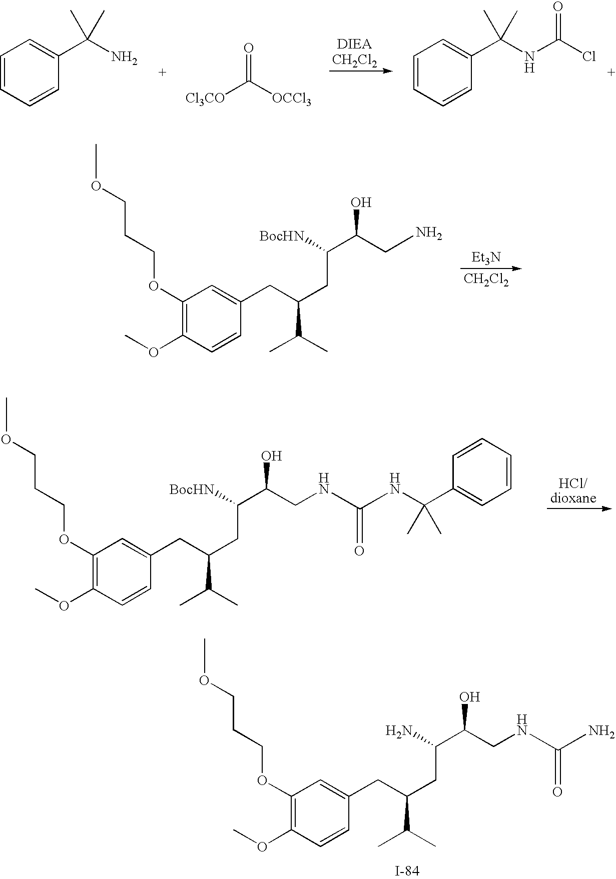

### Step 1

To the solution of 2-phenylpropan-2-amine (62.0 mg, 0.44 mmol) in CH₂Cl₂ (1.0 mL), diisopropylethylamine (0.38 mL, 2.2 mmol) was added followed by bis(trichloromethyl)carbonate (66.6 mg, 0.22 mmol). The resulting solution was stirred at room temperature for 30 min, diluted with CH₂Cl₂ (5 mL), washed with 1 N aq HCl (2 mL), satd aq NaHCO₃ (2 mL), brine (2 mL), dried over Na₂SO₄, and evaporated under reduced pressure to give (2-phenylpropan-2-yl)carbamic chloride, which was used in the next step without purification, MS m/z 198 [M+H]⁺.

### Step 2

To a solution of tert-butyl (2S,3S,5S)-5-(3-(3-methoxypropoxy)-4-methoxybenzyl)-1-amino-2-hydroxy-6-methylheptan-3-ylcarbamate (20.6 mg, 0.044 mmol) in CH₂Cl₂ (0.5 mL) and Et₃N (0.1 mL), was added (2-phenylpropan-2-yl)carbamic chloride (8.7 mg, 0.044 mmol). The resulting solution was stirred at room temperature until no starting material remained (˜30 min), and purified by preparative HPLC to give 1-((2S,3S,5S)-5-(3-(3-methoxypropoxy)-4-methoxybenzyl)-3-(tert-butoxycarbonylamino)-2-hydroxy-6-methylheptyl)urea (21.4 mg, 77%). MS m/z 630 [M+H]⁺.

### Step 3

1-((2S,3S,5S)-5-(3-(3-methoxypropoxy)-4-methoxybenzyl)-3-(tert-butoxycarbonylamino)-2-hydroxy-6-methylheptyl)urea (21.4 mg, 0.034 mmol) was dissolved in 4 M HCl in dioxane (2 mL, 8 mmol) and stirred at room temperature for 1 h. The crude product was submitted to preparative HPLC to afford 1-((2S,3S,5S)-5-(3-(3-methoxypropoxy)-4-methoxybenzyl)-3-amino-2-hydroxy-6-methylheptyl)urea (1-84) as its trifluoroacetic acid salt. ¹H NMR (CDCl₃) δ 0.9 (m), 1.4-1.8 (m), 2.04 (m), 2.24 (m), 2.68 (m), 3.02 (m), 3.16 (m), 3.36 (s), 3.60 (t), 3.80 (s), 4.06 (t), 6.74 (m), 6.78 (m), 7.5 (br). MS m/z 412 [M+H]⁺.

### EXAMPLE 20

### 1-((2S,3S,5S)-5-(3-(3-methoxypropoxy)-4-methoxybenzyl)-3-amino-2-hydroxy-6-methylheptyl)-3-pentylthiourea (I-101)

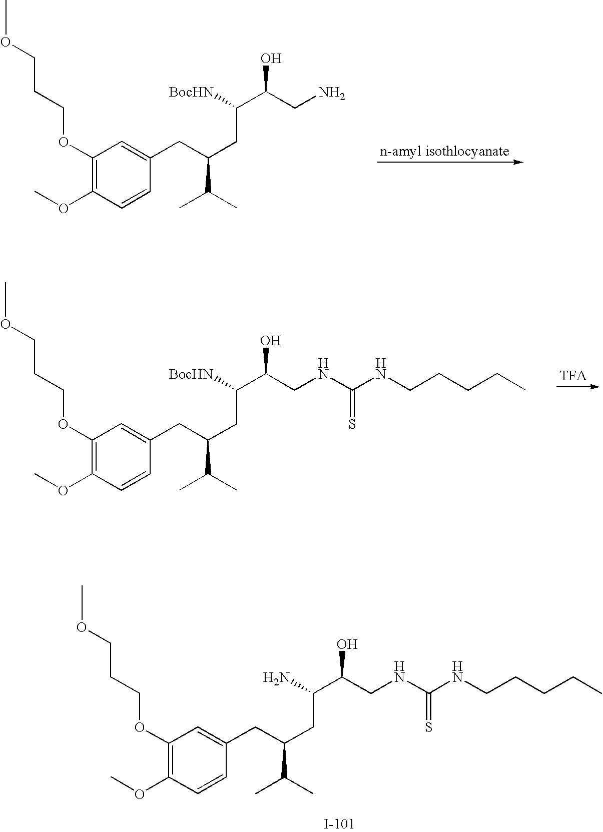

### Step 1

A mixture of tert-butyl (2S,3S,5S)-5-(3-(3-methoxypropoxy)-4-methoxybenzyl)-1-amino-2-hydroxy-6-methylheptan-3-ylcarbamate (68.4 mg, 0.146 mmol, 1.0 equiv) and n-amyl isothiocyanate (90.4 mg, 0.70 mmol, 4.8 equiv) in CH₂Cl₂ (3 mL) was stirred at room temperature for 23 h. The solvent was removed in vacuo and the residue was purified by reversed-phase preparative HPLC (Phenomenex® Luna 5μ C18(2) 100A, 150×10.00 mm, 5 micron, 10% →65% CH₃CN/H₂O, 0.1% CF₃COOH over 3 min and then 65% →90% CH₃CN/H₂O, 0.1% CF₃COOH over 22 min, flow rate 8 mL/min) to afford 1-((2S,3S,5S)-5-(3-(3-methoxypropoxy)-4-methoxybenzyl)-3-(tert-butoxycarbonylamino)-2-hydroxy-6-methylheptyl)-3-pentylthiourea (48.2 mg, 55%). LC-MS (3 min) t_{R}=2.14 min m/z 598 [M+H]⁺.

### Step 2

A solution of 1-((2S,3S,5S)-5-(3-(3-methoxypropoxy)-4-methoxybenzyl)-3-(tert-butoxycarbonylamino)-2-hydroxy-6-methylheptyl)-3-pentylthiourea (48.2 mg) in trifluoroacetic acid (2 mL) and CH₂Cl₂ (3 mL) was stirred at room temperature for 3 h. The solvents were removed in vacuo, the residue was purified by reversed-phase HPLC (XTerra® Prep MS C₁₈ OBD™ Column, 5 μm, 19×50 mm, 10% p90% CH₃CN/H₂O, 0.1% CF₃COOH over 8 min, flow rate 20 mL/min) to give N-[(2S,3S,5S)-5-[3-(3-methoxypropoxy)-4-methoxybenzyl]-3-amino-2-hydroxy-6-methylheptyl]-N′-pentylthiourea (I-101) as its trifluoroacetate salt. LC-MS (3 min) t_{R}=1.43 min m/z 498 [M+H]⁺; ¹H NMR (400 MHz, CD₃OD) δ 6.72-6.58 (m, 3H), 3.92 (t, J=6.3 Hz, 2H), 3.70 (br s, 1H), 3.65 (s, 3H), 3.60 (br s, 1H), 3.44 (t, J=6.2 Hz, 2H), 3.30-3.22 (m, 2H), 3.20 (s, 3H), 2.97-2.85 (m, 2H), 2.49 (dd, J=13.6, 6.3 Hz, 1H), 2.25 (dd, J=13.5, 7.6 Hz, 1H), 1.88 (p, J=6.3

### EXAMPLE 21

### Pentyl(2S,3S,5S)-5-(3-(3-methoxypropoxy)-4-methoxybenzyl)-3-amino-2-hydroxy-6-methylheptylcarbamate (I-94)

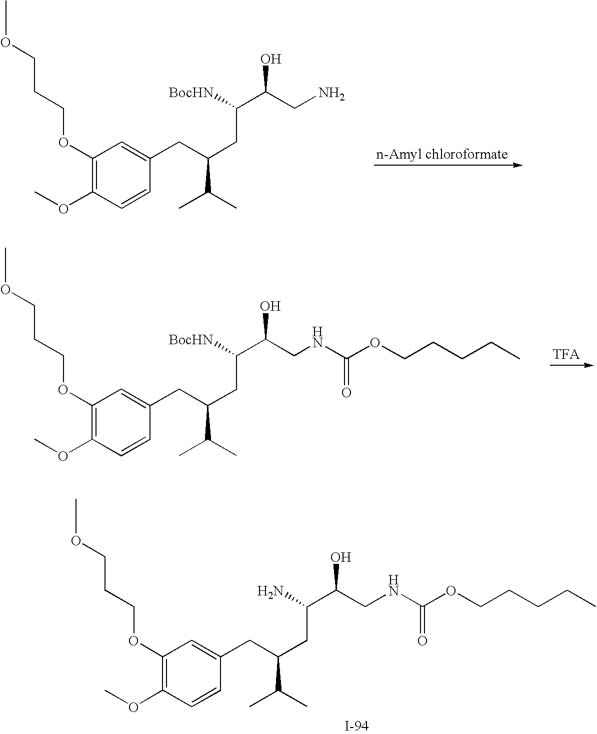

### Step 1

To a solution of tert-butyl (2S,3S,5S)-5-(3-(3-methoxypropoxy)-4-methoxybenzyl)-1-amino-2-hydroxy-6-methylheptan-3-ylcarbamate (67.3 mg, 0.144 mmol, 1.0 equiv) and triethylamine (0.15 mL, 1.08 mmol, 7.5 equiv) in CH₂Cl₂ (3 mL) was added n-amyl chloroformate (30 mg, 0.20 mmol, 1.4 equiv). The resulting mixture was stirred at room temperature for 6 h. After the solvents were removed in vacuo, the residue was purified by reversed-phase HPLC (Phenomenex® Luna 5μ C18(2) 100A, 150×10.00 mm, 5 micron, 10%→65% CH₃CN/H₂O, 0.1% CF₃COOH over 3 min and then 65%→90% CH₃CN/H₂O, 0.1% CF₃COOH over 22 min, flow rate 8 mL/min) to afford 0.0206 g (25%) of pentyl (2S,3S,5S)-5-(3-(3-methoxypropoxy)-4-methoxybenzyl)-3-(tert-butoxycarbonylamino)-2-hydroxy-6-methylheptylcarbamate. LC-MS (3 min) t_{R}=2.18 min m/z 605 [M+Na]⁺, 483 [M−Boc]⁺; ¹H NMR (400 MHz, CDCl₃) δ 6.77-6.67 (m, 3H), 5.42 (br s, 1H), 4.71 (br d, J=9.4 Hz, 1H), 4.10 (t, J=6.5 Hz, 2H), 4.03 (t, J=6.6 Hz, 2H), 3.88 (br s, 1H), 3.83 (s, 3H), 3.62-3.55 (m, 4H), 3.35 (s, 3H), 3.30 (br s, 1H), 3.06-3.02 (m, 1H), 2.53 (dd, J=13.6, 6.0 Hz, 1H), 2.42 (dd, J=13.5, 8.5 Hz, 1H), 2.08 (p, J=6.4 Hz, 2H), 1.44 (s, 9H), 1.69-1.19 (m, 10H), 0.90-0.83 (m, 9H).

### Step 2

A mixture of pentyl (2S,3S,5S)-5-(3-(3-methoxypropoxy)-4-methoxybenzyl)-3-(tert-butoxycarbonylamino)-2-hydroxy-6-methylheptylcarbamate (20.6 mg, 0.0353 mmol), trifluoroacetic acid (2 mL) and CH₂Cl₂ (2 mL) was stirred at room temperature for 4 h. After the solvents were removed in vacuo, the residue was purified by reversed-phase HPLC (XTerra® Prep MS C₁₈ OBD™ Column, 5 μm, 19×5 mm, 10%→90% CH₃CN/H₂O, 0.1% CF₃COOH over 8 min, flow rate 20 mL/min) to give 0.0175 g (83%) of pentyl (2S,3S,5S)-5-(3-(3-methoxypropoxy)-4-methoxybenzyl)-3-amino-2-hydroxy-6-methylheptylcarbamate (I-94) as its trifluoroacetate salt. LC-MS (3 min) t_{R}=1.45 min m/z 483 [M+H]⁺; ¹H NMR (400 MHz, CD₃OD) δ 6.74 (d, J=8.2 Hz, 1H), 6.68 (d, J=1.8 Hz, 1H), 6.62 (dd, J=8.2, 2.0 Hz, 1H), 3.95-3.89 (m, 4H), 3.67 (s, 3H), 3.48-3.40 (m, 3H), 3.22 (s, 3H), 3.08 (dd, J=14.4, 6.5 Hz, 1H), 2.94 (dd, J=14.4, 6.2 Hz, 1H), 2.82-2.78 (m, 1H), 2.50 (dd, J=13.6, 6.3 Hz, 1H), 2.28 (dd, J=13.6, 7.8 Hz, 1H), 1.90 (p, J=6.2 Hz, 2H), 1.62-1.58 (m, 2H), 1.50-1.45 (m, 4H) 1.22-1.19 (m, 4H), 0.84-0.77 (m, 9H).

### EXAMPLE 22

The following compounds of Formula I were prepared by the procedure of Example 21 substituting the appropriate chloroformate in Step 1

| | |
|---|---|
| I-87 | isobutyl (2S,3S,5S)-5-(3-(3-methoxypropoxy)-4-methoxybenzyl)- |
| | 3-amino-2-hydroxy-6-methylheptylcarbamate |
| I-103 | benzyl (2S,3S,5S)-5-(3-(3-methoxypropoxy)-4-methoxybenzyl)- |
| | 3-amino-2-hydroxy-6-methylheptylcarbamate. |

### EXAMPLE 23

### (2S,3S,5S)-5-(3-(3-methoxypropoxy)-4-methoxybenzyl)-3-amino-6-methyl-1-(pentylaminosulfonylamino)-heptan-2-ol (I-113)

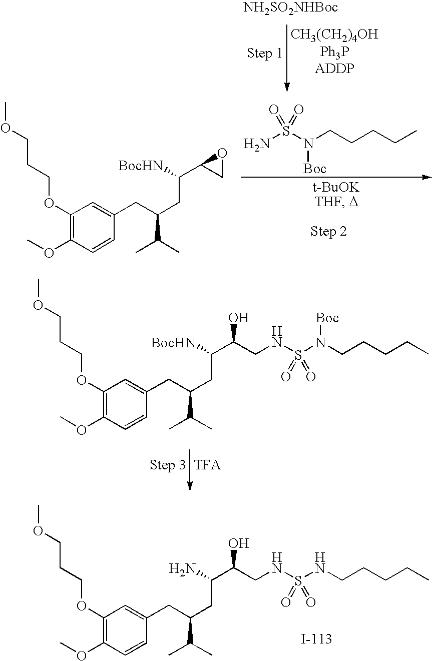

### Step 1

To a stirred mixture of 1-pentanol (0.43 g, 4.88 mmol, 1.0 equiv), N-tert-butoxycarbonyl-sulfamide {prepared from chlorosulfonyl isocyanate according to Y. Nishino et al., Organic Process Research & Development 2003, 7, 649-654} (1.02 g, 5.18 mmol, 1.06 equiv), triphenylphosphine (1.76 g, 6.71 mmol, 1.37 equiv) and ethyl acetate (5 mL) was added 1,1′-(azodicarbonyl)dipiperidine (ADDP) (1.55, 6.14 mmol, 1.26 equiv). The reaction mixture was stirred at room temperature for 14 h. After the solvents were removed in vacuo, the residue was purified by chromatography on silica gel (10% to 20% ethyl acetate in hexanes) to afford N-aminosulfonyl-tert-butyl pentylcarbamate (0.849 g, 65%). LC-MS (3 min) t_{R}=1.74 min m/z 251 [M−CH₃]⁺, 210 [M−C₄H₈]⁺; ¹H NMR (400 MHz, CDCl₃) δ 5.29 (br s, 2H), 3.68-3.64 (m, 2H), 1.69-1.61 (m, 2H), 1.53 (s, 9H), 1.37-1.24 (m, 4H), 0.90 (t, J=7.0 Hz, 3H); ¹³C NMR (100 MHz, CDCl₃) δ 152.5, 84.1, 47.6, 29.0, 28.4, 27.9, 22.1, 14.0.

### Step 2

A mixture of N-aminosulfonyl-tert-butyl pentylcarbamate (0.207 g, 0.77 mmol) and 1 M KOt-Bu in THF (0.75 mL, 0.75 mmol) in THF (4 mL) was heated at 65° C. for 2.5 h. A solution of tert-butyl (1S,3S)-3-(3-(3-methoxypropoxy)-4-methoxybenzyl)-4-methyl-1-((R)-oxiran-2-yl)pentylcarbamate (0.0349 g, 0.077 mmol) in THF (3 mL) was added and then the solvents were removed in vacuo. The neat residue was heated at 65° C. for 9 h and purified by chromatography on silica gel (20% to 50% ethyl acetate in hexanes) to afford (2S,3S,5S)-5-(3-(3-methoxypropoxy)-4-methoxybenzyl)-3-(tert-butoxycarbonylamino)-6-methyl-1-(N-pentyl-N-(tert-butoxycarbonyl)aminosulfonylamino)-heptan-2-ol (0.0289 g, 52%). LC-MS (3 min) t_{R}=2.42 min m/z 740 [M+Na]⁺, 618 [M−Boc]⁺, 518 [M−2Boc]⁺.

### Step 3

A mixture of (2S,3S,5S)-5-(3-(3-methoxypropoxy)-4-methoxybenzyl)-3-(tert-butoxycarbonylamino)-6-methyl-1-(N-pentyl-N-(tert-butoxycarbonyl)aminosulfonylamino)-heptan-2-ol (27.5 mg), trifluoroacetic acid (3 mL) and CH₂Cl₂ (3 mL) was stirred at room temperature for 2 h. After the solvents were removed in vacuo, the residue was purified by reversed-phase HPLC (Phenomenex® Luna 5μ C18(2) 100A, 150×10.00 mm, 5 micron, 10%→90% CH₃CN/H₂O, 0.1% CF₃COOH over 14 min, flow rate 8 mL/min) to give (2S,3S,5S)-5-(3-(3-methoxypropoxy)-4-methoxybenzyl)-3-amino-6-methyl-1-(pentylaminosulfonylamino)-heptan-2-ol (I-113) as its trifluoroacetate salt. LC-MS (3 min) t_{R}=1.37 m/z 518 [M+H]⁺; ¹H NMR (400 MHz, CD₃OD) δ 6.76-6.62 (m, 3H), 3.94 (t, J=6.2 Hz, 2H), 3.68 (s, 3H), 3.55-3.50 (m, 1H), 3.47 (t, J=6.2 Hz, 2H), 3.23 (s, 3H), 3.08-3.03 (m, 1H), 2.91-2.81 (m, 4H), 2.55-2.31 (m, 2H), 1.94-1.87 (m, 2H), 1.74-1.40 (m, 6H), 1.25-1.21 (m, 4H), 0.85-0.77 (m, 9H).

### EXAMPLE 24

The following compounds of Formula I were prepared by the procedure of Example 23

| | |
|---|---|
| I-104 | (2S,3S,5S)-5-(3-(3-methoxypropoxy)-4-methoxybenzyl)- |
| | 3-amino-1-(butylaminosulfonylamino)-6-methylheptan-2-ol |
| I-118 | (2S,3S,5S)-5-(3-(3-methoxypropoxy)-4-methoxybenzyl)- |
| | 3-amino-1-(N-(allylaminosulfonyl)-N-isopropylamino)- |
| | 6-methylheptan-2-ol. |

The following are compounds of the invention:

Table of Compounds

| | LC-MS | | | |
|---|---|---|---|---|
| | (3 min) | | | |
| Cpd. | t_{R} | Mass | ¹H NMR | |
| No. | (min) | Observed | solvent | Selected ¹H NMR resonances |
| I-1 | 1.24 | 439 | CD₃OD | 0.9 (m), 1.2-1.9 (m), 2.02 (m), 2.18 (m), 2.40 (m), 2.64 (m), |
| | | | | 2.82 (m), 3.08 (m), 3.36 (s), 3.60 (t), 3.62 (m), 3.80 (m), 4.06 (t), |
| | | | | 6.7-6.9 (m) |
| I-2 | 1.24 | 451 | CD₃OD | 0.2 (m), 0.52 (m), 0.9 (m), 1.3-1.9 (m), 2.02 (m), 1.10 (m), 2.40 (m), |
| | | | | 2.62 (m), 2.86 (m), 3.12 (m), 3.36 (s), 3.60 (t), 3.80 (t), 4.06 (t), |
| | | | | 6.7-6.9 (m) |
| I-3 | 1.25 | 453 | CDCl₃ | 6.79-6.66 (m, 3H), 6.13 (br s, 1H), 4.10 (t, J = 6.4 Hz, 2H), 3.83 (s, |
| | | | | 3H), 3.58 (t, J = 6.0 Hz, 2H), 3.54-1.48 (m, 1H), 3.36 (s, 3H), |
| | | | | 3.31-3.23 (m, 1H), 3.13-3.02 (m, 2H), 2.71-2.37 (m, 5H), 2.20-2.06 (m, |
| | | | | 5H), 1.78-1.55 (m, 4H), 1.38-1.28 (m, 4H), 0.92-0.85 (m, 9H) |
| I-4 | 1.29 | 453 | CD₃OD | 0.92 (m), 1.18 (s), 1.6 (m), 2.02 (m), 2.40 (m), 2.62 (m), 3.34 (s), |
| | | | | 3.58 (t), 3.80 (s), 4.04 (t), 6.76 (m), 6.80 (d), 6.84 (m) |
| I-5 | | 461 | CD₃OD | 0.12 (m, 2H), 0.48 (m, 2H), 1.15 (s, 6H), 4.02 (t, 2H), 6.75 (m, 3H), 7.17 (t, 1H) |
| I-6 | 1.37 | 467 | CD₃OD | 0.9 (m), 1.32 (m), 1.6-1.8 (m), 2.02 (m), 2.20 (t), 2.40 (m), 2.62 (m), |
| | | | | 2.84 (m), 3.10 (m), 3.36 (s), 3.38 (m), 3.60 (t), 3.80 (s), 4.06 (t), |
| | | | | 6.7-6.9 (m) |
| I-7 | 1.35 | 467 | CD₃OD | 0.8-1.0 (m), 1.14 (s), 1.4-1.9 (m), 2.02 (m), 2.40 (m), 2.60 (m), |
| | | | | 2.90 (m), 3.10 (m), 3.36 (s), 3.40 (m), 3.60 (t), 3.80 (s), 4.06 (t), |
| | | | | 6.7-6.9 (m) |
| I-8 | 1.35 | 467 | CD₃OD | 0.9 (m), 1.00 (s), 1.6-1.8 (m), 2.02 (m), 2.04 (m), 2.40 (m), 2.62 (m), |
| | | | | 2.84 (m), 3.06 (m), 3.36 (s), 3.38 (m), 3.58 (m), 3.60 (t), 3.80 (s), |
| | | | | 4.04 (t), 6.7-6.9 (m) |
| I-9 | 1.16 | 469 | CD₃OD | 0.9 (m), 1.4-1.9 (m), 2.02 (m), 2.24 (m), 2.40 (m), 2.62 (m), |
| | | | | 2.80 (m), 3.08 (m), 3.26 (m), 3.36 (s), 3.40 (t), 3.60 (t), 3.80 (s), 4.04 (t), |
| | | | | 6.7-6.9 (m) |
| I-10 | 1.30 | 473 | CD₃OD | 0.9 (m), 1.30 (m), 1.66 (m), 2.02 (m), 2.40 (m), 2.60 (m), 2.98 (m), |
| | | | | 3.26 (m), 3.36 (s), 3.58 (t), 3.60 (m), 3.68 (m), 3.76 (s), 4.04 (t), |
| | | | | 6.7-6.9 (m), 7.48 (dd), 7.58 (dd), 7.84 (d) |
| I-11 | 1.26 | 479 | CD₃OD | 0.9 (m), 1.4-1.9 (m), 2.02 (m), 2.40 (m), 2.60 (m), 2.82 (m), |
| | | | | 3.0-3.3 (m), 3.36 (s), 3.60 (t), 3.80 (s), 4.06 (t), 6.7-6.9 (m) |
| I-12 | 1.38 | 479 | CD₃OD | 0.9 (m), 1.1-1.9 (m), 2.02 (m), 2.20 (m), 2.40 (m), 2.62 (m), |
| | | | | 2.84 (m), 3.08 (m), 3.36 (s), 3.60 (t), 3.7 (m), 3.80 (s), 4.06 (t), |
| | | | | 6.7-6.9 (m) |
| I-13 | 1.36 | 479 | CD₃OD | 0.9 (m), 1.2-1.5 (m), 1.6-1.85 (m), 2.02 (m), 2.20 (m), 2.40 (m), |
| | | | | 2.60 (m), 2.84 (m), 3.06 (m), 3.36 (s), 3.38 (m), 3.60 (t), 3.70 (m), |
| | | | | 3.80 (s), 4.06 (t), 6.7-6.9 (m) |
| I-14 | | 479 | CD₃OD | 1.12 (s, 6H), 3.60 (m, 2H), 4.02 (m, 2H), 6.73 (m, 3H), 7.17 (t, 1H) |
| I-15 | 1.45 | 481 | CD₃OD | 0.9 (m), 1.30 (m), 1.6-1.9 (m), 2.02 (m), 2.20 (t), 2.40 (m), 2.62 (m), |
| | | | | 2.84 (m), 3.10 (m), 3.36 (s), 3.38 (m), 3.60 (t), 3.80 (s), 4.06 (t), |
| | | | | 6.7-6.9 (m) |
| I-16 | 1.43 | 481 | CD₃OD | 0.9 (m), 1.16 (s), 1.20 (m), 1.4-1.9 (m), 2.02 (m), 2.40 (m), 2.60 (m), |
| | | | | 2.92 (m), 3.14 (m), 3.36 (s), 3.40 (m), 3.60 (t), 3.80 (s), 4.06 (t), |
| | | | | 6.7-6.9 (m) |
| I-17 | 1.48 | 481 | CD₃OD | 0.80-1.00 (m), 1.10 (m), 1.30 (m), 1.60 (m), 1.70 (m), 2.00 (m), |
| | | | | 2.30 (m), 2.40 (m), 2.60 (m), 3.00 (m), 3.10 (m), 3.30 (s), 3.40 (m), |
| | | | | 3.60 (m), 3.80 (s), 4.05 (t), 6.70-7.00 (m) |
| I-18 | 1.33 | 487 | CD₃OD | 0.9 (m), 1.3-1.9 (m), 2.02 (m), 2.30 (m), 2.56 (m), 2.78 (m), |
| | | | | 3.14 (m), 3.36 (s), 3.38 (m), 3.50 (s), 3.60 (t), 3.68 (m), 3.80 (s), 4.06 (t), |
| | | | | 6.7-6.9 (m), 7.2 (m), 7.3 (m) |
| I-19 | 1.3 | 489 | | ND |
| I-20 | 1.32 | 493 | CD₃OD | 0.9 (m), 2.03 (q), 3.36 (s), 3.60 (t), 3.80 (s), 4.08 (t), 6.7-6.9 (m) |
| I-21 | 1.45 | 493 | CD₃OD | 0.9 (m), 1.1-1.8 (m), 2.04 (m), 2.40 (m), 2.60 (m), 2.84 (m), |
| | | | | 3.06 (m), 3.38 (s), 3.60 (t), 3.80 (s), 4.06 (t), 6.7-6.9 (m) |
| I-22 | | 493 | CD₃OD | 0.02 (m, 2H), 0.48 (m, 2H), 0.60 (m, 1H), 1.20 (s, 6H), 4.06 (t, 2H) |
| I-23 | 1.44 | 493 | CD₃OD | 0.9 (m), 1.12 (s), 1.2-1.8 (m), 1.98 (m), 2.02 (m), 2.40 (m), 2.60 (m), |
| | | | | 2.98 (m), 3.14 (m), 3.36 (s), 3.42 (m), 3.60 (t), 3.64 (m), 3.80 (s), |
| | | | | 4.04 (t), 6.7-6.9 (m) |
| I-24 | 1.48 | 493 | CD₃OD | 0.9 (m), 1.3-1.8 (m), 2.02 (m), 2.12 (m), 2.40 (m), 2.60 (m), |
| | | | | 2.84 (m), 3.08 (m), 3.36 (s), 3.38 (m), 3.58 (m), 3.60 (t), 3.80 (s), 4.04 (t), |
| | | | | 6.7-6.9 (m) |
| I-25 | | 493 | CD₃OD | 1.16 (s, 6H), 3.60 (t, 2H), 4.05 (t, 2H), 6.68 (s, 1H), 7.02 (d, 2H) |
| I-26 | | 493 | CD₃OD | 1.16 (s, 6H), 3.60 (t, 2H), 4.05 (t, 2H), 6.68 (s, 1H), 6.72 (d, 1H), 7.02 (d, 1H) |
| I-27 | 1.37 | 495 | CDCl₃ | 6.80-6.66 (m, 3H), 4.14-3.95 (m, 3H), 3.83 (s, 3H), 3.61-3.50 (m, |
| | | | | 3H), 3.35 (s, 3H), 2.80-1.22 (m, 18H), 1.08-0.81 (m, 15H) |
| I-28 | | 495 | CD₃OD | 1.15 (s, 6H), 1.20 (m, 2H), 1.28 (m, 2H), 2.02 (m, 2H), 3.58 (m, 2H), |
| | | | | 4.05 (t, 2H) |
| I-29 | 1.51 | 495 | CD₃OD | |
| I-30 | | 495 | CD₃OD | 0.92 (t, 3H), 0.97 (s, 6H), 1.30 (m, 4H), 2.05 (m, 4H), 3.56 (t, 2H), |
| | | | | 4.06 (m, 2H) |
| I-31 | 1.53 | 495 | CD₃OD | 0.80-1.00 (m), 1.20 (m), 1.50 (m), 1.60 (m), 1.70 (m), 2.00 (m), |
| | | | | 2.10 (m), 2.40 (m), 2.60 (m), 3.00 (m), 3.10 (m), 3.30 (s), 3.40 (m), |
| | | | | 3.60 (m), 3.80 (s), 4.05 (t), 6.70-7.00 (m) |
| I-32 | 1.40 | 497 | CD₃OD | 0.9 (m), 1.38 (s), 1.5-1.8 (m), 2.02 (m), 2.40 (m), 2.60 (m), 2.94 (m), |
| | | | | 3.20 (m), 3.36 (s), 3.38 (t), 3.60 (t), 3.64 (m), 3.80 (s), 4.06 (t), |
| | | | | 6.7-6.9 (m) |
| I-33 | 1.36 | 497 | CD₃OD | 0.9 (m), 1.16 (st), 1.4-1.8 (m), 2.02 (m), 2.40 (m), 2.60 (m), |
| | | | | 2.94 (m), 3.18 (m), 3.36 (s), 3.40 (m), 3.50 (q), 3.60 (t), 3.64 (m), |
| | | | | 3.80 (s), 4.06 (t), 6.7-6.9 (m) |
| I-34 | 1.40 | 501 | CD₃OD | 0.9 (m), 1.3-1.9 (m), 2.02 (m), 2.40 (m), 2.50 (m), 2.60 (m), |
| | | | | 2.78 (m), 2.90 (m), 3.06 (m), 3.38 (s), 3.56 (m), 3.60 (t), 3.80 (s), 4.06 (t), |
| | | | | 6.7-6.9 (m), 7.2 (m) |
| I-35 | 1.40 | 501 | DMSO- | 1.58 (m), 2.08 (m), 2.3-2.5 (m), 2.74 (m), 3.02 (s), 3.16 (m), |
| | | | d₆ | 3.72 (m), 3.92 (m), 4.02 (s), 4.20 (s), 4.26 (t), 4.32 (m), 4.46 (m), 4.50 (s), |
| | | | | 4.76 (t), 7.4-7.9 (m), 8.6 (br) |
| I-36 | 1.40 | 503 | CD₃OD | 0.9 (m), 1.40 (m), 1.5-1.9 (m), 2.02 (m), 2.44 (m), 2.60 (m), |
| | | | | 3.08 (m), 3.24 (m) 3.36 (s), 3.58 (t), 3.80 (s), 4.06 (t), 6.7-6.9 (m) |
| I-37 | 1.34 | 505 | DMSO- | 1.58 (m), 2.0-2.5 (m), 2.72 (m), 3.18 (m), 3.72 (m), 3.92 (m), |
| | | | d₆ | 4.00 (s), 4.26 (t), 4.30 (s), 4.44 (m), 4.50 (s), 4.78 (t), 4.80 (br), |
| | | | | 7.4-7.6 (m), 7.88 (dd), 8.1 (m), 8.6 (br) |
| I-38 | 1.36 | 505 | CD₃OD | 0.9 (m), 1.38 (m), 1.5-1.8 (m), 2.02 (m), 2.30 (m), 2.54 (m), |
| | | | | 2.78 (m), 3.14 (m), 3.36 (s), 3.38 (m), 3.54 (s), 3.60 (t), 3.70 (m), 3.80 (s), |
| | | | | 4.06 (t), 6.7-7.0 (m), 7.08 (m), 7.26 (m) |
| I-39 | 1.35 | 505 | CD₃OD | 0.9 (m), 1.3-1.8 (m), 2.02 (m), 2.36 (m), 2.44 (m), 2.84 (m), |
| | | | | 3.16 (m), 3.36 (s), 3.38 (m), 3.48 (s), 3.56 (t), 3.58 (m), 3.78 (s), 4.04 (t), |
| | | | | 6.7-6.9 (m), 7.04 (m), 7.34 (m) |
| I-40 | 1.35 | 505 | CD₃OD | 0.9 (m), 1.3-1.8 (m), 2.02 (m), 2.36 (m), 2.44 (m), 2.84 (m), |
| | | | | 3.16 (m), 3.36 (s), 3.38 (m), 3.48 (s), 3.56 (t), 3.58 (m), 3.78 (s), 4.04 (t), |
| | | | | 6.7-6.9 (m), 7.04 (m), 7.34 (m) |
| I-41 | 1.33 | 507 | CD₃OD | 0.9 (m), 1.4-1.9 (m), 2.02 (m), 2.20 (m), 2.26 (m), 2.40 (m), |
| | | | | 2.64 (m), 2.78 (m), 3.08 (m), 3.36 (s), 3.38 (m), 3.58 (m), 3.60 (t), |
| | | | | 3.80 (s), 4.06 (t), 6.7-6.9 (m) |
| I-42 | 1.53 | 507 | CD₃OD | 0.02 (m), 0.4 (m), 0.64 (m), 0.9 (m), 1.16 (m), 1.18 (s), 1.7 (m), |
| | | | | 2.04 (m), 2.44 (m), 2.64 (m), 3.96 (m), 316 (m), 3.36 (m), 3.38 (s), |
| | | | | 3.62 (t), 3.76 (m), 3.84 (s), 4.10 (t), 6.78-6.90 (m) |
| I-43 | | 507 | CD₃OD | 0.00 (m, 2H), 0.39 (m, 2H), 0.60 (m, 1H), 0.89 (d, 3H), 0.96 (d, 3H), |
| | | | | 3.35 (s, 3H), 3.82 (s, 3H) |
| I-44 | 1.33 | 509 | CD₃OD | 0.9 (m), 1.34 (m), 1.6-1.9 (m), 2.02 (m), 2.42 (m), 2.60 (m), |
| | | | | 2.88 (m), 3.20 (m), 3.36 (s), 3.60 (mt), 3.80 (s), 4.08 (t), 6.7-6.9 (m), |
| | | | | 7.26 (m), 7.88 (d) |
| I-45 | 1.55 | 509 | CD₃OD | 0.9 (m), 1.1-1.9 (m), 2.02 (m), 2.20 (m), 2.40 (m), 2.62 (m), |
| | | | | 2.84 (m), 3.08 (m), 3.36 (s), 3.58 (t), 3.7 (m), 3.80 (s), 4.06 (t), |
| | | | | 6.7-6.9 (m) |
| I-46 | 1.32 | 509 | CD₃OD | 0.9 (m), 1.06 (s), 1.2-1.9 (m), 2.02 (m), 2.28 (m), 2.66 (m), 2.76 (s), |
| | | | | 3.24 (m), 3.36 (s), 3.60 (t), 3.70 (m), 3.80 (s), 4.06 (t), 6.7-6.9 (m) |
| I-47 | 1.26 | 512 | CD₃OD | 0.9 (m), 1.3-1.8 (m), 2.02 (m), 2.26 (m), 2.52 (m), 2.78 (m), |
| | | | | 3.16 (m), 3.36 (s), 3.38 (m), 3.60 (mt), 3.80 (s), 4.04 (t), 6.74 (m), |
| | | | | 6.86 (m), 7.44 (m), 7.60 (m) |
| I-48 | 1.43 | 513 | CD₃OD | 0.9 (m), 1.08 (m), 1.4-1.8 (m), 2.02 (m), 2.40 (m), 2.60 (m), |
| | | | | 2.82 (m), 3.06 (m), 3.26 (m), 3.36 (s), 3.52 (m), 3.60 (t), 3.80 (s), 4.04 (t), |
| | | | | 6.7-6.9 (m), 7.38 (m) |
| I-49 | 1.42 | 515 | CD₃OD | 0.9 (m), 1.6-2.0 (m), 2.02 (m), 2.24 (t), 2.40 (m), 2.62 (m), 2.82 (m), |
| | | | | 3.08 (m), 3.36 (s), 3.38 (m), 3.60 (t), 3.80 (s), 4.06 (t), 6.7-6.9 (m), |
| | | | | 7.18 (m), 7.24 (m) |
| I-50 | 1.42 | 515 | CD₃OD | 0.9 (m), 1.4-1.8 (m), 2.02 (m), 2.36 (m), 2.56 (m), 2.84 (m), |
| | | | | 3.08 (m), 3.34 (s), 3.36 (m), 3.58 (m), 3.60 (t), 3.80 (s), 4.04 (t), |
| | | | | 6.7-6.9 (m), 7.20 (m), 7.36 (m) |
| I-51 | | 515 | CDCl₃ | 0.83 (d, 3H), 0.90 (d, 3H), 1.50 (s, 6H), 3.31 (S, 3H), 3.80 (s, 3H) |
| I-52 | 1.62 | 522 | CD₃OD | 0.80-1.00 (m), 1.1 (s), 1.50-1.80 (m), 1.85 (m) 2.00 (m), 2.40 (m), |
| | | | | 2.60 (m), 2.90 (m), 3.10 (m), 3.30 (s), 3.60 (m), 3.80 (s), 4.05 (t), |
| | | | | 6.60-6.80 (m) |
| I-53 | 1.40 | 523 | CD₃OD | 0.9 (m), 1.4-1.8 (m), 2.02 (m), 2.32 (m), 2.58 (m), 2.80 (m), |
| | | | | 3.18 (m), 3.36 (s), 3.38 (m), 3.52 (s), 3.60 (t), 3.70 (m), 3.80 (s), 4.04 (t), |
| | | | | 6.7-6.9 (m), 7.02-7.26 (m) |
| I-54 | 1.40 | 523 | CD₃OD | 0.9 (m), 1.3-1.9 (m), 2.02 (m), 2.38 (m), 2.58 (m), 2.84 (m), |
| | | | | 3.18 (m), 3.36 (s), 3.38 (m), 3.60 (ts), 3.70 (m), 3.80 (s), 4.06 (t), |
| | | | | 6.7-6.9 (m), 7.38 (m) |
| I-55 | 1.41 | 523 | CD₃OD | 0.9 (m), 1.4-1.8 (m), 2.02 (m), 2.38 (m), 2.58 (m), 2.84 (m), |
| | | | | 3.18 (m), 3.36 (s), 3.38 (m), 3.60 (ts), 3.70 (m), 3.80 (s), 4.04 (t), |
| | | | | 6.7-6.9 (m), 7.10 (m) |
| I-56 | 1.36 | 523 | CD₃OD | 0.9 (m), 1.34 (m), 1.5-1.9 (m), 2.00 (m), 2.42 (m), 2.60 (m), |
| | | | | 2.96 (m), 3.14 (m), 3.36 (s), 3.50, 3.64 (m), 3.58 (t), 3.80 (s), 4.06 (t), |
| | | | | 6.7-6.9 (m), 7.40 (m) |
| I-57 | 1.49 | 527 | CD₃OD | 0.9 (m), 1.04 (m), 1.3-1.8 (m), 2.02 (m), 2.32 (s), 2.40 (m), 2.60 (m), |
| | | | | 2.84 (m), 3.08 (m), 3.26 (m), 3.36 (s), 3.60 (t), 3.64 (m), 3.80 (s), |
| | | | | 4.04 (t), 6.7-6.9 (m), 7.1-7.3 (m) |
| I-58 | 1.47 | 531 | CDCl₃ | 6.78-6.66 (m, 3H), 4.09 (t, J = 6.4 Hz, 2H), 4.03-3.96 (m, 1H), |
| | | | | 3.83 (s, 3H), 3.57 (t, J = 6.0 Hz, 2H), 3.43-3.39 (m, 1H), 3.35 (s, 3H), |
| | | | | 3.13 (d, J = 6.4 Hz, 2H), 2.97-2.92 (m, 2H), 2.73-1.17 (m, 20H), |
| | | | | 0.96-0.86 (m, 9H) |
| I-59 | 1.45 | 531 | CDCl₃ | 6.78-6.68 (m, 3H), 4.11-3.95 (m, 3H), 3.83 (s, 3H), 3.59-3.55 (m, |
| | | | | 3H), 3.36 (s, 3H), 3.19-1.32 (m, 18H), 1.21-1.16 (m, 6H), |
| | | | | 0.97-0.87 (m, 9H) |
| I-60 | 1.45 | 533 | CD₃OD | 0.9 (m), 1.3-1.8 (m), 1.74 (s), 2.02 (m), 2.36 (m), 2.58 (m), 2.82 (m), |
| | | | | 3.36 (s), 3.38 (m), 3.58 (t), 3.64 (m), 3.80 (s), 4.04 (t), 6.74 (dd), |
| | | | | 6.78 (s), 6.84 (d), 7.02 (m), 7.38 (m) |
| I-61 | | 533 | CDCl₃ | 0.84 (d, 3H), 0.92 (d, 3H), 1.50 (s, 6H), 3.35 (s, 3H), 3.84 (s, 3H) |
| I-62 | | 533 | CDCl₃ | 0.85 (d, 3H), 0.90 (d, 3H), 1.51 (s, 6H), 3.32 (s, 3H), 3.80 (s, 3H) |
| I-63 | 1.67 | 544 | CD₃OD | 0.80-1.00 (m), 1.10-1.40 (m), 1.40-1.80 (m), 1.85 (m) 2.00 (m), |
| | | | | 2.40 (m), 2.60 (m), 3.00 (m), 3.10 (m), 3.30 (s), 3.40 (m), 3.60 (m), |
| | | | | 3.80 (s), 4.05 (t), 6.70-7.00 (m) |
| I-64 | 1.56 | 541 | CD₃OD | 0.86 (m), 1.5-1.8 (m), 1.98 (m), 2.02 (m), 2.24 (m), 2.48 (m), |
| | | | | 2.70 (m), 3.04 (m), 3.36 (m), 3.38 (m), 3.60 (t), 3.80 (s), 4.06 (t), 6.7 (m), |
| | | | | 6.84 (d), 7.18 (m), 7.24 (m), 7.38 (d) |
| I-65 | | 541 | CD₃OD | 1.18 (s, 6H), 3.60 (m, 2H), 4.10 (m, 2H), 6.79 (m, 2H), 7.10 (d, 1H), 7.22 (m, 2H), |
| | | | | 7.38 (m, 2H), 7.50 (d, 1H) |
| I-66 | | 541 | CD₃OD | 1.18 (s, 6H), 3.60 (m, 2H), 4.07 (m, 2H), 6.84 (m, 2H), 7.10 (d, 1H), 7.30 (m, 1H), |
| | | | | 7.38 (m, 2H), 7.45 (m, 2H) |
| I-67 | 1.44 | 543 | CD₃OD | 0.9 (m), 1.08 (m), 1.4-1.8 (m), 2.02 (m), 2.40 (m), 2.60 (m), |
| | | | | 2.82 (m), 3.06 (m), 3.26 (m), 3.36 (s), 3.52 (m), 3.60 (t), 3.78 (s), 3.80 (s), |
| | | | | 4.06 (t), 6.78 (m), 6.90 (m), 7.28 (m) |
| I-68 | | 543 | CD₃OD | 1.16 (s, 6H), 3.56 (t, 2H), 4.03 (t, 2H), 6.74 (d, 1H), 6.85 (s, 1H), 7.42 (d, 1H) |
| I-69 | 1.38 | 545 | CD₃OD | 0.9 (m), 1.08 (s), 1.3-1.9 (m), 2.02 (m), 2.40 (m), 2.60 (m), 3.36 (s), |
| | | | | 3.58 (t), 3.80 (s), 4.06 (t), 6.7-6.9 (m), 7.3 (m) |
| I-70 | 1.53 | 547 | CD₃OD | 0.9 (m), 1.08 (m), 1.4-1.8 (m), 2.02 (m), 2.40 (m), 2.60 (m), |
| | | | | 2.82 (m), 3.06 (m), 3.26 (m), 3.36 (s), 3.52 (m), 3.60 (t), 3.80 (s), 4.04 (t), |
| | | | | 6.7-6.9 (m), 7.38 (m) |
| I-71 | 1.45 | 551 | CDCl₃ | 7.84-7.79 (m, 2H), 7.60-7.48 (m, 3H), 6.79-6.68 (m, 3H), |
| | | | | 4.11-4.00 (m, 3H), 3.82 (s, 3H), 3.63-3.52 (m, 3H), 3.33 (s, 3H), 3.23-1.22 (m, |
| | | | | 12H), 1.05-0.86 (m, 12H) |
| I-72 | 1.45 | 555 | CD₃OD | 0.9 (m), 1.4-1.9 (m), 2.02 (m), 2.40 (m), 2.58 (m), 2.86 (m), |
| | | | | 3.16 (m), 3.36 (s), 3.38 (m), 3.60 (t), 3.70 (m), 3.78 (s), 3.80 (s), 4.06 (t), |
| | | | | 6.7-6.9 (m), 7.44 (m), 7.58 (dd), 7.66 (d) |
| I-73 | 1.50 | 555 | CD₃OD | 0.9 (m), 1.4-1.8 (m), 2.02 (m), 2.32 (m), 2.58 (m), 2.80 (m), |
| | | | | 3.18 (m), 3.36 (s), 3.38 (m), 3.60 (ts), 3.70 (m), 3.80 (s), 4.04 (t), |
| | | | | 6.7-6.9 (m), 7.44-7.64 (m) |
| I-74 | 1.50 | 555 | CD₃OD | 0.9 (m), 1.4-1.8 (m), 2.02 (m), 2.34 (m), 2.56 (m), 2.80 (m), |
| | | | | 3.16 (m), 3.36 (s), 3.38 (m), 3.60 (t), 3.62 (s), 3.70 (m), 3.80 (s), 4.06 (t), |
| | | | | 6.7-6.9 (m), 7.50 (d), 7.58 (d) |
| I-75 | 1.60 | 555 | CD₃OD | 0.82 (d), 0.88 (d), 1.3-1.7 (m), 1.82 (m), 2.02 (m), 2.24 (m), 2.40 (m), |
| | | | | 2.76 (m), 3.08 (m), 3.36 (s), 3.38 (m), 3.60 (t), 3.70 (m), 3.80 (s), |
| | | | | 4.06 (t), 6.70 (dd), 6.74 (d), 6.84 (d), 7.18 (dd), 7.26 (dd), 7.40 (d) |
| I-76 | 1.38 | 556 | CD₃OD | 0.9 (m), 1.58 (s), 1.7 (m), 2.02 (m), 2.38 (m), 2.58 (m), 2.88 (m), |
| | | | | 3.16 (m), 3.36 (s), 3.38 (m), 3.60 (t), 3.80 (s), 4.04 (t), 6.7-6.9 (m), |
| | | | | 7.02 (d), 7.64 (d) |
| I-77 | 1.49 | 561 | CD₃OD | 0.9 (m), 1.4-1.9 (m), 1.72 (s), 2.02 (m), 2.40 (m), 2.60 (m), 2.94 (m), |
| | | | | 3.20 (m), 3.36 (s), 3.56 (m), 3.60 (t), 3.62 (m), 3.80 (s), 4.06 (t), |
| | | | | .6.7-6.9 (m) |
| I-78 | 1.56 | 561 | CD₃OD | 0.76 (m), 0.9 (m), 1.4-1.8 (m), 2.0 (m), 2.36 (m), 2.56 (m), 2.84 (m), |
| | | | | 3.06 (m), 3.36 (s), 3.38 (m), 3.56 (m), 3.58 (t), 3.80 (s), 4.04 (t), |
| | | | | 6.74 (dd), 6.76 (d), 6.84 (d), 7.02 (m), 7.28 (m) |
| I-79 | 1.44 | 565 | CDCl₃ | 7.44-7.33 (m, 5H), 6.79-6.66 (m, 3H), 4.35-4.22 (m, 2H), |
| | | | | 4.11-4.07 (m, 2H), 3.83 and 3.82 (s, 3H), 3.74-3.65 (m, 1H), 3.57 (t, J = 6.0 Hz, |
| | | | | 2H), 3.35 (s, 3H), 3.37-3.31 (m, 1H), 3.08-2.97 (m, 2H), |
| | | | | 2.74-1.21 (m), 1.08 (d, J = 7.2 Hz, 3H), 1.06 (d, J = 7.2 Hz, 3H), 0.87 (d, |
| | | | | J = 6.8 Hz, 6H) |
| I-80 | 1.60 | 565 | CD₃OD | 0.9 (m), 1.48 (s), 1.4-1.8 (m), 2.02 (m), 2.40 (m), 2.60 (m), 2.96 (m), |
| | | | | 3.22 (m), 3.36 (s), 3.40 (m), 3.58 (t), 3.70 (m), 3.80 (s), 4.06 (m), |
| | | | | 6.76 (d), 6.80 (s), 6.86 (d), 6.96 (d), 7.26 (d) |
| I-81 | 1.55 | 571 | CD₃OD | 0.9 (m), 1.3-1.9 (m), 2.02 (m), 2.36 (m), 2.58 (m), 2.80 (m), |
| | | | | 3.16 (m), 3.36 (s), 3.38 (m), 3.60 (ts), 3.70 (m), 3.80 (s), 4.06 (t), |
| | | | | 6.7-6.9 (m), 7.18 (m), 7.40 (m) |
| I-82 | 1.63 | 573 | CD₃OD | 0.82 (d), 0.88 (d), 1.3-1.7 (m), 1.80 (m), 2.02 (m), 2.24 (m), 2.40 (m), |
| | | | | 2.52 (m), 2.76 (m), 3.10 (m), 3.36 (s), 3.38 (m), 3.60 (t), 3.66 (m), |
| | | | | 3.80 (s), 4.06 (t), 6.70 (dd), 6.74 (d), 6.84 (d), 7.02 (m), 7.42 (m) |
| I-83 | 1.57 | 582 | CD₃OD | 0.9 (m), 1.12 (m), 1.4-1.8 (m), 2.02 (m), 2.40 (m), 2.60 (m), |
| | | | | 2.98 (m), 3.04 (m), 3.36 (s), 3.38 (m), 3.56 (m), 3.60 (t), 3.80 (s), 4.04 (t), |
| | | | | 6.76 (m), 6.86 (m), 7.38-7.50 (m) |
| I-84 | 1.09 | 412 | CDCl₃ | 0.9 (m), 1.4-1.8 (m), 2.04 (m), 2.24 (m), 2.68 (m), 3.02 (m), |
| | | | | 3.16 (m), 3.36 (s), 3.60 (t), 3.80 (s), 4.06 (t), 6.74 (m), 6.78 (m), 7.5 (br) |
| I-85 | 1.25 | 468 | CDCl₃ | 6.80-6.68 (m, 3H), 4.97 (br s, 2H), 4.11-4.06 (m, 2H), 3.82 (s, 3H), |
| | | | | 3.60-3.55 (m, 2H), 3.35 (s, 3H), 3.40-1.24 (m, 20H), 0.92-0.84 (m, |
| | | | | 9H) |
| I-86 | 1.28 | 468 | CD₃OD | 6.77-6.62 (m, 3H), 3.96-3.92 (m, 2H), 3.69 (s, 3H), 3.54-3.50 (m, |
| | | | | 1H), 3.47 (t, J = 6.2 Hz, 2H), 3.43-3.39 (m, 1H), 3.23 (s, 3H), |
| | | | | 3.15-2.82 (m, 3H), 2.55-2.24 (m, 2H), 1.91 (p, J = 6.2 Hz, 2H), |
| | | | | 1.72-1.56 (m, 2H), 1.48 (tm, J = 7.0 Hz, 1H), 1.18 and 1.17 (s, 9H), |
| | | | | 0.87-0.78 (m, 6H) |
| I-87 | 1.36 | 469 | CD₃OD | 6.73 (d, J = 7.9 Hz, 1H), 6.67 (d, J = 2.0 Hz, 1H), 6.61 (dd, J = 8.0, |
| | | | | 1.9 Hz, 1H), 3.93 (t, J = 6.3 Hz, 2H), 3.70 (d, J = 6.5 Hz, 2H), |
| | | | | 3.67 (s, 3H), 3.47-3.39 (m, 3H), 3.22 (s, 3H), 3.09 (dd, J = 14.4, 6.5 Hz, |
| | | | | 1H), 2.93 (dd, J = 14.4, 6.5 Hz, 1H), 2.82-2.78 (m, 1H), 2.50 (dd, J = 13.5, |
| | | | | 6.2 Hz, 1H), 2.27 (dd, J = 13.6, 8.1 Hz, 1H), 1.90 (p, J = 6.3 Hz, |
| | | | | 2H), 1.81-1.71 (m, 1H), 1.62-1.57 (m, 2H), 1.48-1.44 (m, 2H), |
| | | | | 0.82 (d, J = 6.7 Hz, 6H), 0.79 (d, J = 7.0 Hz, 6H) |
| I-88 | 1.29 | 480 | CD₃OD | 0.9 (m), 1.5-1.8 (m), 2.02 (m), 2.40 (m), 2.60 (m), 2.92 (m), |
| | | | | 3.36 (s)m 3.38 (m), 3.60 (t), 3.80 (s), 4.06 (t), 6.7-6.9 (m) |
| I-89 | 1.30 | 480 | CD₃OD | 0.02 (m), 0.42 (m), 0.84 (m), 0.9 (m), 1.38 (m), 1.60 (m), 1.7 (m), |
| | | | | 2.02 (m), 2.40 (m), 2.60 (m), 3.0 (m), 3.36 (s), 3.60 (t), 3.80 (s), |
| | | | | 4.06 (t), 6.7-6.9 (m) |
| I-90 | 1.14 | 482 | CD₃OD | 0.9 (m), 1.60 (m), 1.7 (m), 2.02 (m), 2.40 (m), 2.60 (m), 2.9 (m), |
| | | | | 3.18 (m), 3.36 (s), 3.36 (m), 3.60 (t), 3.62 (m), 3.80 (s), 4.06 (t), |
| | | | | 6.7-6.9 (m) |
| I-91 | 1.38 | 482 | CD₃OD | 0.9 (m), 1.30 (m), 1.44 (m), 1.60 (m), 1.76 (m), 2.02 (m), 2.42 (m), |
| | | | | 2.60 (m), 2.98 (m), 3.12 (t), 3.26 (m), 3.36 (s), 3.60 (t), 3.80 (s) |
| | | | | 4.06 (t), 6.7-6.9 (m) |
| I-92 | 1.37 | 482 | CD₃OD | 0.9 (m), 1.30 (m), 1.46 (m), 1.60 (m), 1.74 (m), 2.02 (m), 2.42 (m), |
| | | | | 2.62 (m), 2.94 (m), 3.08 (t, m), 3.26 (m), 3.36 (s), 3.56 (m), 3.60 (t), |
| | | | | 3.80 (s), 4.06 (t), 6.7-6.9 (m) |
| I-93 | 1.38 | 482 | CD₃OD | 0.9 (m), 1.3-1.5 (m), 1.6-1.9 (m), 2.02 (m), 2.40 (m), 2.60 (m), |
| | | | | 3.0 (m), 3.08 (t), 3.22 (m), 3.36 (s), 3.60 (t), 3.66 (m), 3.80 (s), 4.06 (t), |
| | | | | 6.7-6.9 (m) |
| I-94 | 1.45 | 483 | CD₃OD | 6.74 (d, J = 8.2 Hz, 1H), 6.68 (d, J = 1.8 Hz, 1H), 6.62 (dd, J = 8.2, |
| | | | | 2.0 Hz, 1H), 3.95-3.89 (m, 4H), 3.67 (s, 3H), 3.48-3.40 (m, 3H), |
| | | | | 3.22 (s, 3H), 3.08 (dd, J = 14.4, 6.5 Hz, 1H), 2.94 (dd, J = 14.4, 6.2 Hz, |
| | | | | 1H), 2.82-2.78 (m, 1H), 2.50 (dd, J = 13.6, 6.3 Hz, 1H), 2.28 (dd, J = 13.6, |
| | | | | 7.8 Hz, 1H), 1.90 (p, J = 6.2 Hz, 2H), 1.62-1.58 (m, 2H), |
| | | | | 1.50-1.45 (m, 4H) 1.22-1.19 (m, 4H), 0.84-0.77 (m, 9H) |
| I-95 | 1.17 | 484 | CD₃OD | 0.9 (m), 1.4-1.8 (m), 2.02 (m), 2.40 (m), 2.60 (m), 2.94 (m), |
| | | | | 3.06 (m), 3.18 (t), 3.24 (m), 3.30 (s), 3.36 (s), 3.40 (t), 3.56 (m), 3.60 (t), |
| | | | | 3.80 (s), 4.06 (t), 6.7-6.9 (m) |
| I-96 | 1.20 | 484 | CD₃OD | 0.9 (m), 1.18 (t), 1.4-1.8 (m), 2.02 (m), 2.40 (m), 2.60 (m), 2.92 (m), |
| | | | | 3.06 (m), 3.26 (m), 3.36 (s), 3.46 (m), 3.60 (t), 3.80 (s), 4.04 (t), |
| | | | | 6.7-6.9 (m) |
| I-97 | 1.41 | 494 | CD₃OD | 0.9 (m), 1.1-1.4 (m), 1.6-1.9 (m), 2.02 (m), 2.42 (m), 2.60 (m), |
| | | | | 3.02 (m), 3.10 (m), 3.24 (m), 3.36 (s), 3.42 (m), 3.60 (t), 3.70 (m), |
| | | | | 3.80 (s), 4.06 (t), 6.7-6.9 (m) |
| I-98 | 1.47 | 496 | CD₃OD | 0.9 (m), 1.30 (m), 1.44 (m), 1.60 (m), 1.76 (m), 2.02 (m), 2.42 (m), |
| | | | | 2.60 (m), 2.98 (m), 3.12 (t), 3.24 (m), 3.36 (s), 3.60 (t), 3.80 (s) |
| | | | | 4.04 (t), 6.7-6.9 (m) |
| I-99 | | 496 | CD₃OD | 1.31 (m, 4H), 1.49 (m, 2H), 2.39 (m, 1H), 2.60 (m, 1H), 2.86 (s, 3H), |
| | | | | 3.59 (t, 2H), 4.05 (t, 2H) |
| I-100 | 1.44 | 496 | CD₃OD | 0.9 (m), 1.2-1.8 (m), 2.02 (m), 2.40 (m), 2.64 (m), 2.84 (s), 2.98 (m), |
| | | | | 3.12 (t), 3.36 (s), 3.54 (m), 3.60 (t), 3.63 (m), 3.80 (s), 4.06 (t), |
| | | | | 6.7-6.9 (m) |
| I-101 | 1.43 | 498 | CD₃OD | 6.72-6.58 (m, 3H), 3.92 (t, J = 6.4 Hz, 2H), 3.70 (br s, 1H), 3.65 (s, |
| | | | | 3H), 3.60 (br s, 1H), 3.44 (t, J = 6.2 Hz, 2H), 3.30-3.22 (m, 2H), |
| | | | | 3.20 (s, 3H), 2.89-2.85 (m, 1H), 2.49 (dd, J = 13.6, 6.4 Hz, 1H), 2.25 (dd, |
| | | | | J = 13.6, 7.6 Hz, 1H), 1.88 (p, J = 6.3 Hz, 2H), 1.62-1.37 (m, 5H), |
| | | | | 1.21-1.13 (m, 4H), 0.82-0.74 (m, 9H) |
| I-102 | 1.29 | 502 | CD₃OD | 0.9 (m), 1.40 (m), 1.60 (m), 1.7 (m), 2.02 (m), 2.40 (m), 2.60 (m), |
| | | | | 2.96 (m), 3.12 (m), 3.28 (m), 3.36 (s), 3.60 (t), 3.80 (s), 4.06 (t), |
| | | | | 4.30 (m), 6.7-6.9 (m), 7.20 (m), 7.26 (m) |
| I-103 | 1.42 | 503 | CD₃OD | 7.25-7.16 (m, 5H), 6.75-6.60 (m, 3H), 4.99 (s, 2H), 3.94 (t, J = 6.3 Hz, |
| | | | | 2H), 3.67 (s, 3H), 3.48-3.40 (m, 3H), 3.22 (s, 3H), 3.15-2.76 (m, |
| | | | | 4H), 2.53-2.45 (m, 1H), 2.30-2.24 (m, 1H), 1.94-1.86 (m, 2H), |
| | | | | 1.62-1.58 (m, 2H), 1.48 (t, J = 6.6 Hz, 1H), 0.84-0.78 (m, 6H) |
| I-104 | 1.32 | 504 | CD₃OD | 6.77-6.64 (m, 3H), 3.96 (t, J = 6.4 Hz, 2H), 3.69 (s, 3H), 3.48 (t, J = 6.2 Hz, |
| | | | | 2H), 3.24 (s, 3H), 3.16-3.14 (m, 1H), 2.92-2.82 (m, 4H), |
| | | | | 2.56-2.32 (m, 2H), 1.95-1.89 (m, 2H), 1.75-1.63 (m, 3H), |
| | | | | 1.51-1.23 (m, 6H), 0.86-0.79 (m, 9H) |
| I-105 | 1.47 | 506 | CD₃OD | 0.9 (m), 1.4-1.9 (m), 2.00 (m), 2.42 (m), 2.62 (m), 3.0 (m), 3.18 (m), |
| | | | | 3.26 (m), 3.36 (s), 3.60 (t), 3.62 (m), 3.78 (s), 4.04 (t), 6.66-6.82 (m), |
| | | | | 7.00 (d), 7.22 (dd), 7.40 (dd) |
| I-106 | 1.44 | 508 | CD₃OD | 0.9 (m), 1.1-1.5 (m), 1.6-1.8 (m), 2.02 (m), 2.40 (m), 2.60 (m), |
| | | | | 2.96 (d), 2.98 (m), 3.12 (m), 3.22 (m), 3.36 (s), 3.60 (t), 3.64 (m), |
| | | | | 3.80 (s), 4.06 (m), 6.7-6.9 (m) |
| I-107 | 1.44 | 508 | CD₃OD | 0.9 (m), 1.1-1.8 (m), 2.02 (m), 2.40 (m), 2.60 (m), 2.72 (s), 2.88, |
| | | | | 2.98 (m), 3.14 (m), 3.36 (sm), 3.60 (t), 3.80 (s), 3.92 (m), 4.04 (t), |
| | | | | 6.7-6.9 (m), 7.38 (m) |
| I-108 | 1.44 | 510 | CDCl₃ | 6.78-6.65 (m, 3H), 6.20 (br s, 1H), 4.26-4.19 (m, 1H), 4.08 (t, J = 6.6 Hz, |
| | | | | 2H), 3.82 (s, 3H), 3.56 (t, J = 6.2 Hz, 2H), 3.35 (s, 3H), |
| | | | | 3.31-3.27 (m, 1H), 3.22-3.11 (m, 3H), 2.67-2.34 (m, 3H), 2.09 (p, J = 6.3 Hz, |
| | | | | 2H), 1.77-1.27 (m, 9H), 1.10-1.07 (m, 6H), 0.92-0.86 (m, 9H) |
| I-109 | | 510 | CDCl₃ | 0.84 (s, 6H), 1.18 (m, 2H), 1.27 (m, 2H), 2.00 (m, 2H), 2.95 (dd, |
| | | | | 2H), |
| I-110 | | 510 | CD₃OD | 0.91 (m, 8H), 1.25 (s, 6H), 2.02 (m, 2H) |
| I-111 | 1.09 | 511 | CD₃OD | 0.9 (m), 1.16 (s), 1.60 (m), 0.7 (m), 2.02 (m), 2.40 (m), 2.60 (m), |
| | | | | 3.24 (m), 3.36 (s), 3.60 (t), 3.80 (s), 4.06 (t), 6.7-6.9 (m) |
| I-112 | 1.37 | 516 | CD₃OD | 0.9 (m), 1.6-1.8 (m), 2.02 (m), 2.42 (m), 2.62 (m), 2.78 (t), 2.94 (m), |
| | | | | 3.12 (m), 3.26 (m), 3.36 (s), 3.58 (t), 3.80 (s), 4.04 (t), 6.7-6.9 (m), |
| | | | | 7.2-7.3 (m) |
| I-113 | 1.37 | 518 | CD₃OD | 6.76-6.62 (m, 3H), 3.94 (t, J = 6.2 Hz, 2H), 3.68 (s, 3H), |
| | | | | 3.55-3.50 (m, 1H), 3.47 (t, J = 6.2 Hz, 2H), 3.23 (s, 3H), 3.08-3.03 (m, 1H), |
| | | | | 2.91-2.81 (m, 4H), 2.55-2.31 (m, 2H), 1.94-1.87 (m, 2H), |
| | | | | 1.74-1.40 (6H), 1.25-1.21 (m, 4H), 0.85-0.77 (m, 9H) |
| I-114 | 1.58 | 522 | CD₃OD | 0.92 (m), 1.2 (m), 1.6 (m), 2.02 (m), 2.40 (m), 2.60 (m), 3.36 (s), |
| | | | | 3.60 (t), 3.80 (s), 4.06 (t), 6.7-6.9 (m) |
| I-115 | 1.55 | 524 | CD₃OD | 0.9 (m), 1.00 (s), 1.34 (s), 1.6-1.8 (m), 2.02 (m), 2.40 (m), 2.60 (m), |
| | | | | 3.06 (m), 3.22 (m), 3.36 (s), 3.56 (m), 3.60 (t), 3.80 (s), 4.04 (t), |
| | | | | 6.7-6.9 (m) |
| I-116 | 1.62 | 524 | CD₃OD | 0.9 (m), 1.4-1.9 (m), 1.98 (m), 2.02 (m), 2.40 (m), 2.60 (m), |
| | | | | 3.10 (m), 3.36 (s), 2.60 (t), 3.80 (s), 4.06 (t), 6.7-6.9 (m) |
| I-117 | 1.45 | 530 | CD₃OD | 0.9 (m), 1.6-1.8 (m), 2.02 (m), 2.42 (m), 2.62 (mt), 2.96 (m), 3.14 (t), |
| | | | | 3.24 (m), 3.36 (s), 3.58 (m), 3.60 (t), 3.80 (s), 4.04 (t), 6.7-6.9 (m), |
| | | | | 7.1-7.3 (m) |
| I-118 | 1.33 | 530 | CDCl₃ | 6.79-6.68 (m, 3H), 5.92-5.81 (m, 1H), 5.28-5.14 (m, 2H), 4.09 (t, J = 6.6 Hz, |
| | | | | 2H), 4.03 (p, J = 6.7 Hz, 1H), 3.83 (s, 3H), 3.65-3.61 (m, |
| | | | | 2H), 3.57 (t, J = 6.2 Hz, 2H), 3.45-3.40 (m, 1H), 3.35 (s, 3H), |
| | | | | 3.25-3.06 (m, 2H), 2.53 (dd, J = 14.0, 7.2 Hz, 1H), 2.43 (dd, J = 13.8, 7.4 Hz, |
| | | | | 1H), 2.09 (p, J = 6.3 Hz, 2H), 1.79-1.31 (m, 3H), 1.22-1.17 (m, |
| | | | | 6H), 0.89-0.86 (m, 6H) |
| I-119 | 1.55 | 556 | CD₃OD | 0.9 (m), 1.4-1.9 (m), 2.00 (m), 2.42 (m), 2.62 (m), 3.0 (m), 3.20 (m), |
| | | | | 3.26 (m), 3.36 (s), 3.58 (t), 3.62 (m), 3.76 (s), 4.04 (t), 6.7-6.9 (m), |
| | | | | 7.24 (d), 7.42 (dd), 7.52 (d), 7.92 (d) |
| I-120 | 1.53 | 562 | CD₃OD | 0.9 (m), 1.22 (s), 1.24 (s), 1.4-1.8 (m), 2.00 (m), 2.42 (m), 2.60 (m), |
| | | | | 3.02 (m), 3.06 (m), 3.36 (s), 3.60 (t), 3.80 (s), 4.02 (t), 6.76 (m), |
| | | | | 6.84 (d), 6.94 (m), 7.14 (m). |

1. A compound of formula I 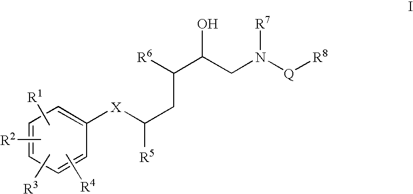
wherein
R¹ is hydrogen, halogen, hydroxy, lower alkoxy, cycloalkoxy, lower alkoxy-lower alkoxy, lower alkylthio-lower alkoxy, cyano-lower alkoxy, hydroxy-lower alkoxy, carboxy-lower alkoxy, lower alkoxycarbonyl-lower alkoxy, carbamoyl-lower alkoxy, N-mono- or N,N-di-lower alkylcarbamoyl-lower alkoxy, or aryl;
R² is hydrogen, halogen, cyano, carbamoyl, lower alkyl, lower haloalkyl, cycloalkyl, halocycloalkyl, cycloalkyl-lower alkyl, halocycloalkyl-loweralkyl, cyano-lower alkyl, hydroxy-lower alkyl, lower alkoxy-lower alkyl, lower haloalkoxy-lower alkyl, lower alkoxy-lower alkyl, lower haloalkoxy-lower alkyl, cycloalkoxy-lower alkyl, halocycloalkoxy-lower alkyl, hydroxy, lower alkanoyloxy-lower alkoxy, hydroxy-lower alkoxy, halo-(hydroxy)-lower alkoxy, lower alkanesulfonyl-(hydroxy)-lower alkoxy, amino-lower alkyl, lower alkylamino-lower alkyl, di-lower alkylamino-lower alkyl, lower alkanoylamino-lower alkyl, lower alkoxycarbonyl-amino-lower alkyl, aminocarbonylamino-lower alkyl, lower alkylaminocarbonylamino-lower alkyl, di(lower alkyl)aminocarbonylamino-lower alkyl, aminosulfonylamino-lower alkyl, lower alkylaminosulfonylamino-lower alkyl, di(lower alkyl)aminosulfonylamino-lower alkyl, amino-lower alkoxy, lower alkylamino-lower alkoxy, di-lower alkylamino-lower alkoxy, lower alkanoylamino-lower alkoxy, lower alkoxycarbonyl-amino-lower alkoxy, aminocarbonylamino-lower alkoxyl, lower alkylaminocarbonylamino-lower alkoxy, di(lower alkyl)aminocarbonylamino-lower alkoxy, aminosulfonylamino-lower alkoxy, lower alkylaminosulfonylamino-lower alkoxy, di(lower alkyl)aminosulfonylamino-lower alkoxy, oxo-lower alkoxy, lower alkoxy, lower haloalkoxy, cycloalkoxy, lower halocycloalkoxy, cycloalkyl-lower alkoxy, halocycloalkyl-lower alkoxy, lower alkenyloxy, cycloalkoxy-lower alkoxy, halocycloalkoxy-lower alkoxy, lower alkoxy-lower alkoxy, lower haloalkoxy-lower alkyl, lower alkoxy-lower alkenyl, lower alkenyloxy-lower alkoxy, lower alkoxy-lower alkenyloxy, lower alkenyloxy-lower alkyl, lower alkanoyl-lower alkoxy, lower alkylthio-lower alkoxy, lower alkanesulfonyl-lower alkoxy, lower alkylthio-(hydroxy)-lower alkoxy, aryl-lower alkoxy, optionally N-oxidized pyridyl-lower alkoxy, thiazolylthio-lower alkoxy or thiazolinylthio-lower alkoxy, imidazolylthio-lower alkoxy, optionally N-oxidized pyridylthio-lower alkoxy, pyrimidinylthio-lower alkoxy, cyano-lower alkoxy, carboxy-lower alkoxy, lower alkoxycarbonyl-lower alkoxy, carbamoyl-lower alkoxy, N-mono- or N,N-di-lower alkylcarbamoyl-lower alkoxy, carboxy-lower alkyl, lower alkoxycarbonyl-lower alkyl, carbamoyl-lower alkyl, or N-mono- or N,N-di-lower alkylcarbamoyl-lower alkyl;
R³ is hydrogen, halogen, cyano, carbamoyl, lower alkyl, lower haloalkyl, lower alkoxy-lower alkyl, cycloalkoxy-lower alkyl, hydroxy-lower alkyl, lower alkylthio-lower alkyl, lower alkanesulfonyl-lower alkyl, optionally partially hydrogenated or N-oxidized pyridyl-lower alkyl, thiazolyl-thio-lower alkyl or thiazolinylthio-lower alkyl, imidazolylthio-lower alkyl, optionally N-oxidized pyridylthio-lower alkyl, pyrimidinylthio-lower alkyl, amino-lower alkyl, lower alkylamino-lower alkyl, di-lower alkylamino-lower alkyl, lower alkanoyl-amino-lower alkyl, lower alkanesulfonylamino-lower alkyl, polyhalo-lower alkane-sulfonylamino-lower alkyl, pyrrolidino-lower alkyl, piperidino-lower alkyl, piperazino-lower alkyl, N′-lower alkylpiperazino-lower alkyl or N′-lower alkanoylpiperazino-lower alkyl, morpholino-lower alkyl, thiomorpholino-lower alkyl, S-oxothiomorpholino-lower alkyl or S,S-dioxothio-morpholino-lower alkyl, cyano-lower alkyl, carboxy-lower alkyl, lower alkoxy-carbonyl-lower alkyl, carbamoyl-lower alkyl, N-mono- or N,N-di-lower alkyl-carbamoyl-lower alkyl, cycloalkyl; phenyl or naphthyl that is unsubstituted or substituted with one to three groups independently selected from lower alkyl, lower alkoxy, hydroxy, lower alkylamino, di-lower alkylamino, halogen, trifluoromethyl, trifluoromethoxy, and cyano; hydroxy, lower alkoxy, cycloalkoxy, lower alkoxy-lower alkoxy, cycloalkoxy-lower alkoxy, hydroxy-lower alkoxy, aryl, lower haloalkoxy, lower alkylthio-lower alkoxy, lower haloalkylthio-lower alkoxy, lower alkanesulfonyl-lower alkoxy, lower haloalkanesulfonyl-lower alkoxy, optionally hydrogenated heteroaryl-lower alkoxy, heterocyclyl-lower alkoxy, optionally partially or fully hydrogenated heteroarylthio-lower alkoxy, such as thiazolylthio-lower alkoxy or thiazolinylthio-lower alkoxy, imidazolylthio-lower alkoxy, optionally N-oxidized pyridylthio-lower alkoxy, pyrimidinylthio-lower alkoxy, amino-lower alkoxy, lower alkylamino-lower alkoxy, di-lower alkylamino-lower alkoxy, lower alkanoylamino-lower alkoxy, lower alkanesulfonylamino-lower alkoxy, polyhalo-lower alkanesulfonylamino-lower alkoxy, pyrrolidino-lower alkoxy, piperidino-lower alkoxy, piperazino-lower alkoxy, N′-lower alkylpiperazino-lower alkoxy or N′-lower alkanoylpiperazino-lower alkoxy, morpholino-lower alkoxy, thiomorpholino-lower alkoxy, S-oxothiomorpholino-lower alkoxy or S,S-dioxothiomorpholino-lower alkoxy, cyano-lower alkoxy, carboxy-lower alkoxy, lower alkoxycarbonyl-lower alkoxy, carbamoyl-lower alkoxy, N-mono- or N,N-di-lower alkylcarbamoyl-lower alkoxy, carboxy-lower alkyl, lower alkoxycarbonyl-lower alkyl, carbamoyl-lower alkyl, or N-mono- or N,N-di-lower alkylcarbamoyl-lower alkyl; or
R² and R³ taken together with the atoms through which they are attached form a fused dioxolane, dioxane, benzene or cyclohexene ring, wherein said ring is substituted with up to 2 substituents independently selected from lower alkyl and lower alkoxy-lower alkyl;
R⁴ is hydrogen, lower alkyl, hydroxy, lower alkoxy, cycloalkoxy, lower alkoxy-lower alkoxy, or cycloalkyl-lower alkoxy; or
R³ and R⁴ taken together with the atoms through which they are attached form a fused dioxolane, dioxane, benzene or cyclohexene ring, wherein said ring is substituted with up to 2 substituents independently selected from lower alkyl and lower alkoxy-lower alkyl; provided that R³ does not form a ring with R²;
X is methylene or hydroxymethylene;
R⁵ is lower alkyl, lower haloalkyl, cycloalkyl, halocycloalkyl, lower alkyl-cycloalkyl, lower haloalkyl-cycloalkyl, cycloalkyl-lower alkyl, aryl, aryl-lower alkyl, heterocyclyl, heterocyclyl-lower alkyl;
R⁶ is amino, lower alkylamino, di-lower alkylamino, or lower alkanoylamino;
R⁷ is hydrogen, lower alkyl, lower haloalkyl, cycloalkyl, lower alkoxy-lower alkyl, or lower haloalkoxy-lower alkyl;
Q is carbonyl, thiocarbonyl, or sulfonyl;
R⁸ is lower alkyl, lower haloalkyl, C₈-C₁₅alkyl, C₈-C₁₅haloalkyl, cycloalkyl, halocycloalkyl, lower alkyl-cycloalkyl, cycloalkyl-lower alkyl, halocycloalkyl-lower alkyl, lower alkoxy-loweralkyl, lower haloalkoxy-lower alkyl, cycloalkoxy-lower alkyl, cycloalkoxy-cycloalkyl, lower alkylthio-lower alkyl, lower haloalkylthio-lower alkyl, lower alkanesulfonyl-lower alkyl, lower haloalkanesulfonyl-lower alkyl, lower alkylthio-cycloalkyl, lower haloalkylthio-cycloalkyl, lower alkanesulfonyl-cycloalkyl, lower haloalkanesulfonyl-cycloalkyl, aryl, aryl-lower alkyl, aryl-lower hydroxyalkyl, arylcycloalkyl, aryloxy-lower alkyl, aryloxy cycloalkyl, arylthio-lower alkyl, arylsulfonyl-lower alkyl, arylthio-cycloalkyl, arylsulfonyl-cycloalkyl, lower alkanoyl-lower alkyl, hydroxy-lower alkyl, amino-lower alkyl, lower alkanoylamino-lower alkyl, N-mono-lower alkylamino-lower alkyl, N,N-di-lower alkylamino-lower alkyl, piperidino-lower alkyl, hydroxypiperidino-lower alkyl, lower alkoxypiperidino-lower alkyl, morpholino-lower alkyl, dimethylmorpholino-lower alkyl, thiomorpholino-lower alkyl, S,S-dioxothiomorpholino-lower alkyl, carboxy-lower alkyl, lower alkoxycarbonyl-lower alkyl, carbamoyl-lower alkyl, N-mono-lower alkylcarbamoyl-lower alkyl, N,N-di-lower alkylcarbamoyl-lower alkyl, carboxy-(hydroxy)-lower alkyl, lower alkoxycarbonyl-(hydroxy)-lower alkyl, carbamoyl-(hydroxy)-lower alkyl, N-mono-lower alkylcarbamoyl-(hydroxy)-lower alkyl, N,N-di-lower alkylcarbamoyl-(hydroxy)-lower alkyl, 5- or 6-membered carboxycycloalkyl-lower alkyl, 5- or 6-membered lower alkoxycarbonyl-cycloalkyl-lower alkyl, 5- or 6-membered carbamoylcycloalkyl-lower alkyl, 5- or 6-membered N-mono-alkylcarbamoylcycloalkyl-lower alkyl, N,N-di-lower alkylcarbamoylcycloalkyl-lower alkyl, cyano-lower alkyl, sulfamoyl-lower alkyl, lower alkylsulfamoyl-lower alkyl, or di-lower alkylsulfamoyl-lower alkyl, imidazolyl-lower alkyl, oxopyrrolidinyl-lower alkyl, benzimidazolyl-lower alkyl, oxadiazolyl-lower alkyl, pyridyl-lower alkyl, oxopiperidinyl-lower alkyl or quinolinyl-lower alkyl, piperidin-4-yl-lower alkyl, or lower alkanoylpiperidin-4-yl-lower alkyl, wherein said aryl, imidazolyl, benzimidazolyl, oxadiazolyl, pyridyl, quinolinyl, aryloxy, arylthio and arylsulfonyl groups are optionally substituted with up to four groups independently selected from halo, cyano, nitro, optionally halogenated lower alkyl, optionally halogenated lower alkoxy, optionally halogenated lower alkylthio, optionally halogenated lower alkanesulfonyl, and lower alkoxycarbonyl;
or R⁸ is OR⁹ or NR⁹R¹⁰
R⁹ is 1) hydrogen, lower alkyl, lower haloalkyl, lower alkenyl, (C₈-C₁₅)alkyl, (C₈-C₁₅)haloalkyl, cycloalkyl, halocycloalkyl, lower alkyl-cycloalkyl, cycloalkyl-lower alkyl, halocycloalkyl-lower alkyl, lower alkoxy-loweralkyl, lower haloalkoxy-lower alkyl, cycloalkoxy-lower alkyl, cycloalkoxy-cycloalkyl, lower alkylthio-lower alkyl, lower haloalkylthio-lower alkyl, lower alkanesulfonyl-lower alkyl, lower haloalkanesulfonyl-lower alkyl, lower alkylthio-cycloalkyl, lower haloalkylthio-cycloalkyl, lower alkanesulfonyl-cycloalkyl, lower haloalkanesulfonyl-cycloalkyl, aminocarbonyl-lower alkyl, lower alkyl-amonocarbonyl-lower alkyl, di(lower alkyl)-amonocarbonyl-lower alkyl, or 2) aryl, aryl-lower alkyl, aryloxy-lower alkyl, arylthio-lower alkyl, or arylsulfonyl-lower alkyl
wherein the aryl groups are optionally substituted with up to four groups independently selected from halo, cyano, optionally halogenated lower alkyl, optionally halogenated lower alkoxy, optionally halogenated lower alkylthio, and optionally halogenated lower alkanesulfonyl;
R¹⁰ is 1) hydrogen, lower alkyl, lower haloalkyl, (C₈-C₁₅)alkyl, (C₈-C₁₅)haloalkyl, cycloalkyl, halocycloalkyl, cycloalkyl-lower alkyl, halocycloalkyl-lower alkyl, lower alkoxy-lower alkyl, lower haloalkoxy-lower alkyl, alkylthio-lower alkyl, lower haloalkylthio-lower alkyl, lower alkanesulfonyl-lower alkyl, lower haloalkanesulfonyl-lower alkyl, or 2) aryl or aryl-lower alkyl
wherein aryl is optionally substituted with up to four groups independently selected from halo, cyano, optionally halogenated lower alkyl, optionally halogenated lower alkoxy, optionally halogenated lower alkylthio, and optionally halogenated lower alkanesulfonyl;
or R⁹ and R¹⁰ taken together with the nitrogen to which they are attached form a 4-, 5-, 6- or 7-membered heterocyclic ring composed of carbon atoms and 0 or 1 N, O, or S atoms in addition to the nitrogen atom to which R⁹ and R¹⁰ are attached, said ring atoms being substituted with the appropriate number of hydrogen atoms and optionally substituted with up to four groups independently selected from halogen, (C₁-C₆)alkyl, halo(C₁-C₆)alkyl, lower alkanoyl, lower alkoxycarbonyl, aryl, aryl-lower alkyl, and oxo, such that substitution of one oxo group on a carbon atom forms a carbonyl group and substitution of one or two oxo groups on sulfur forms sulfoxide or sulfone groups respectively; wherein the aryl and arylalkyl groups are substituted with up to four groups independently selected from halo, cyano, optionally halogenated lower alkyl, optionally halogenated lower alkoxy, optionally halogenated lower alkylthio, and optionally halogenated lower alkanesulfonyl;
and the enantiomers, diastereomers, and salts thereof.

2. A compound of claim 1 of the formula Ia 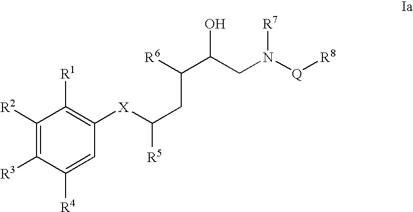
or a salt thereof.

3. A compound of claim 2, wherein
R¹ is hydrogen or aryl;
R² is hydrogen, lower alkyl, cycloalkyl-lower alkyl, lower alkoxy-lower alkyl, lower haloalkoxy-lower alkyl, lower alkoxy-lower alkoxy, lower haloalkoxy-lower alkoxy, lower alkoxy-lower alkoxy-lower alkyl; cycloalkyl-lower alkoxy, phenyl-lower alkoxy that is unsubstituted or substituted by lower alkyl, lower alkoxy, hydroxy, halogen, nitro and/or by amino; optionally N-oxidized pyridyl-lower alkoxy, lower alkylthio-lower alkoxy, lower alkane-sulfonyl-lower alkoxy, lower alkanoyl-lower alkoxy, cyano-lower alkoxy, carboxy-lower alkoxy, lower alkoxycarbonyl-lower alkoxy, carbamoyl-lower alkoxy, lower alkylcarbamoyl-lower alkoxy, or di-lower alkylcarbamoyl-lower alkoxy;
R³ is hydrogen, halogen, cyano, lower alkyl, lower haloalkyl, aryl, hydroxy, lower alkoxy, or polyhalo-lower alkoxy; or
R² and R³ taken together with the atoms through which they are attached form a fused dioxolane ring, wherein said ring is substituted with up to 2 substituents independently selected from lower alkyl and lower alkoxy-lower alkyl;
R⁴ is hydrogen, lower alkoxy-lower alkoxy, lower alkoxy-lower alkyl, or cycloalkyl-lower alkoxy; or
R³ and R⁴ taken together with the atoms through which they are attached form a fused dioxolane ring, wherein said ring is substituted with up to 2 substituents independently selected from lower alkyl and lower alkoxy-lower alkyl; provided that R³ does not form a ring with R²;
X is methylene or hydroxymethylene;
R⁵ is lower alkyl or cycloalkyl;
R⁶ is amino, lower alkylamino, di-lower alkylamino, or lower alkanoylamino;
R⁷ is hydrogen or methyl;
Q is carbonyl, thiocarbonyl, or sulfonyl;
R⁸ is lower alkyl, lower haloalkyl, C₈-C₁₅ alkyl, C₈-C₁₅ haloalkyl, cycloalkyl, halocycloalkyl, lower alkyl-cycloalkyl, cycloalkyl-lower alkyl, halocycloalkyl-lower alkyl, lower alkoxy-lower alkyl, lower haloalkoxy-lower alkyl, cycloalkoxy-lower alkyl, cycloalkoxy-cycloalkyl, lower alkylthio-lower alkyl, lower haloalkylthio-lower alkyl, lower alkanesulfonyl-lower alkyl, lower haloalkanesulfonyl-lower alkyl, lower alkylthio-cycloalkyl, lower haloalkylthio-cycloalkyl, lower alkanesulfonyl-cycloalkyl, lower haloalkanesulfonyl-cycloalkyl, aryl, aryl-lower alkyl, aryl-lower hydroxyalkyl, arylcycloalkyl, aryloxy-lower alkyl, aryloxy cycloalkyl, arylthio-lower alkyl, arylsulfonyl-lower alkyl, arylthio-cycloalkyl, or arylsulfonyl-cycloalkyl wherein said aryl, aryloxy, arylthio and arylsulfonyl groups are optionally substituted with up to four groups independently selected from halo, cyano, nitro, optionally halogenated lower alkyl, optionally halogenated lower alkoxy, optionally halogenated lower alkylthio, optionally halogenated lower alkanesulfonyl, amino, lower alkylamino, di-lower allylamino, and lower alkoxycarbonyl;
or R⁸ is OR⁹ or NR⁹R¹⁰;
R⁹ is selected from 1) hydrogen, lower alkyl, lower haloalkyl, lower alkenyl, (C₈-C₁₅)alkyl, (C₈-C₁₅)haloalkyl, cycloalkyl, halocycloalkyl, lower alkyl-cycloalkyl, cycloalkyl-lower alkyl, halocycloalkyl-lower alkyl, lower alkoxy-loweralkyl, lower haloalkoxy-lower alkyl, cycloalkoxy-lower alkyl, cycloalkoxy-cycloalkyl, lower alkylthio-lower alkyl, lower haloalkylthio-lower alkyl, lower alkanesulfonyl-lower alkyl, lower haloalkanesulfonyl-lower alkyl, lower alkylthio-cycloalkyl, lower haloalkylthio-cycloalkyl, lower alkanesulfonyl-cycloalkyl, lower haloalkanesulfonyl-cycloalkyl, aminocarbonyl-lower alkyl, lower alkyl-amonocarbonyl-lower alkyl, or di(lower alkyl)-amonocarbonyl-lower alkyl, or 2) aryl, aryl-lower alkyl, aryloxy-lower alkyl, arylthio-lower alkyl, or arylsulfonyl-lower alkyl
wherein aryl is optionally substituted with up to four groups independently selected from halo, cyano, optionally halogenated lower alkyl, optionally halogenated lower alkoxy, optionally halogenated lower alkylthio, and optionally halogenated lower alkanesulfonyl;
R¹⁰ is 1) hydrogen, lower alkyl, lower haloalkyl, (C₈-C₁₅)alkyl, (C₈-C₁₅)haloalkyl, cycloalkyl, halocycloalkyl, cycloalkyl-lower alkyl, halocycloalkyl-lower alkyl, lower alkoxy-lower alkyl, lower haloalkoxy-lower alkyl, alkylthio-lower alkyl, lower haloalkylthio-lower alkyl, lower alkanesulfonyl-lower alkyl, or lower haloalkanesulfonyl-lower alkyl, or 2) aryl or aryl-lower alkyl
wherein aryl is optionally substituted with up to four groups independently selected from halo, cyano, optionally halogenated lower alkyl, optionally halogenated lower alkoxy, optionally halogenated lower alkylthio, and optionally halogenated lower alkanesulfonyl;
or R⁹ and R¹⁰ taken together with the nitrogen to which they are attached form a 4-, 5-, 6- or 7-membered heterocyclic ring composed of carbon atoms and 0 or 1 N, O, or S atoms in addition to the nitrogen atom to which R⁹ and R¹⁰ are attached, said ring atoms being substituted with the appropriate number of hydrogen atoms and optionally substituted with up to four groups independently selected from halogen, (C₁-C₆)alkyl, halo(C₁-C₆)alkyl, lower alkanoyl, lower alkoxycarbonyl, aryl, aryl-lower alkyl, and oxo, such that substitution of one oxo group on a carbon atom forms a carbonyl group and substitution of one or two oxo groups on sulfur forms sulfoxide or sulfone groups respectively; wherein the aryl and arylalkyl groups are substituted with up to four groups independently selected from halo, cyano, optionally halogenated lower alkyl, optionally halogenated lower alkoxy, optionally halogenated lower alkylthio, and optionally halogenated lower alkanesulfonyl;
and the enantiomers, diastereomers, and salts thereof.

4. A compound of claim 3, wherein
R¹ is hydrogen;
R² is (C₁-C₄)alkoxy-(C₁-C₄)alkoxy, (C₁-C₄)alkoxy-(C₁-C₄)alkyl, or cycloalkyl-lower alkoxy;
R³ is fluoro, chloro, bromo, cyano, (C₁-C₄)alkyl, (C₁-C₄) haloalkyl, aryl, (C₁-C₄)alkoxy, or (C₁-C₄)haloalkoxy;
R⁴ is hydrogen;
X is methylene;
R⁵ is (C₃-C₅)alkyl;
R⁶ is amino;
R⁷ is hydrogen or methyl;
Q is carbonyl or sulfonyl;
R⁸ is (C₃-C₁₁)alkyl, (C₃-C₁₁)haloalkyl, (C₃-C₇)cycloalkyl, (C₃-C₁₁)cycloalkylalkyl, (C₃-C₁₁)-alkoxyalkyl, aryl, aryl(C₁-C₃)alkyl, aryl(C₃-C₆)cycloalkyl, arylhydroxy(C₁-C₃)alkyl, aryloxy(C₁-C₅)alkyl, or aryloxy(C₃-C₆)cycloalkyl wherein aryl or aryloxy may be unsubstituted or substituted with one to three groups independently selected from halogen, cyano, (C₁-C₃)alkyl, halo(C₁-C₃)alkyl, (C₁-C₃)alkoxy, halo(C₁-C₃)alkoxy;
or R⁸ is NR⁹R¹⁰;
R⁹ is 1) hydrogen, (C₁-C₁₀)alkyl, (C₃-C₇)alkenyl, (C₃-C₇)cycloalkyl, (C₃-C₆)cycloalkyl(C₁-C₅)alkyl, (C₁-C₆)alkoxy(C₁-C₆)alkyl, or aminocarbonyl(C₁-C₅)alkyl, or 2) aryl or aryl(C₁-C₄)alkyl
wherein aryl is optionally substituted with up to 4 groups independently selected from fluorine, chlorine, cyano, (C₁-C₃)alkyl, halo(C₁-C₃)alkyl, (C₁-C₃)alkoxy, halo(C₁-C₃)alkoxy, and (C₁-C₃)alkanesulfonyl;
R¹⁰ is hydrogen, lower alkyl, or lower haloalkyl; or
R⁸ and R⁹ taken together are with the nitrogen to which they are attached form an azetidine, pyrrolidine, piperidine, azepine, piperazine, morpholine, or thiomorpholine ring said ring being optionally substituted with up to two groups independently selected from halogen, (C₁-C₃)alkyl, halo(C₁-C₃)alkyl, and oxo, such that substitution of one oxo group on a carbon atom forms a carbonyl group and substitution of one or two oxo groups on sulfur forms sulfoxide or sulfone groups respectively;
and the enantiomers, diastereomers, and salts thereof.

5. A compound of claim 3, wherein:
R¹ is hydrogen;
R² is 3-methoxypropoxy, 3-ethoxypropoxy, 4-methoxybutyl, or 2-(cyclopropyl)ethoxy;
R³ is fluoro, chloro, bromo, cyano, methyl, ethyl, isopropyl or tert-butyl, trifluoromethyl, pentafluoroethyl, phenyl, methoxy, difluoromethoxy, or trifluoromethoxy;
R⁴ is hydrogen;
X is methylene;
R⁵ is branched (C₃-C₅)alkyl;
R⁶ is amino;
R⁷ is hydrogen;
Q is carbonyl or sulfonyl; and
R⁸ is propyl, 2,2-dimethylpropyl, butyl, tert-butyl, n-pentyl, 2-methyl-2-butyl, hexyl, 2-hexyl, 2-methyl-2-pentyl, 2,2-dimethylpentyl, 3-heptyl, 2-methyl-2-hexyl, 2,4,4-trimethylpentyl, 2,2,2-trifluoroethyl, 3,3,3-trifluoropropyl, 4,4,4-trifluorobutyl, 1,1,1,3,3,3-hexafluoro-2-methyl-2-propyl, cyclohexyl, 1-methylcyclohexyl, 4-methylcyclohexyl, cyclopropylmethyl, cyclopentylmethyl, 1-cyclopentyl-1-pentyl, cyclohexylmethyl, 2-cyclohexyl-2-propyl, 2-cyclopropyl-1,1-dimethylethyl, 3-cyclopropyl-2-methyl-2-butyl, 3-methoxypropyl, 2-propoxy-2-propyl, phenyl, benzyl, 3-methyl-benzyl, 2-fluorobenzyl, 3-fluorobenzyl, 4-fluorobenzyl, 2,4-difluorobenzyl, 2,3-difluorobenzyl, 3,4-difluorobenzyl, 4-cyanobenzyl, 2-(trifluoromethyl)benzyl, 3-(trifluoromethyl)benzyl, 4-(trifluoro-methyl)benzyl, 4-(trifluoromethoxy)benzyl, phenethyl, 3-phenylpropyl, 2-phenyl-2-propyl, 3-(4-fluorophenyl)-3-pentyl, 1-phenyl-1-cyclopropyl, 1-(4-methylphenyl)-1-cyclopropyl, 1-(4-fluoro-phenyl)-1-cyclopropyl, 1-(4-methoxyphenyl)-1-cyclopropyl, 1-(2,4-dichlorophenyl)-1-cyclopropyl, 1-phenyl-1-cyclopentyl, 1-phenyl-1-cyclohexyl, 1-(4-fluorophenyl)-1-cyclohexyl, 3-hydroxy-2-methyl-3-phenyl-2-propyl, 2-(4-cyanophenoxy)-2-propyl, or 2-(4-chlorophenoxy)-2-propyl;
or R⁸ is NR⁹R¹⁰;
R⁹ is hydrogen, butyl, isobutyl, t-butyl, pentyl, hexyl, 2,2-dimethyl-1-pentyl, 2-methyl-2-hexyl, 2,4,4-trimethyl-2-pentyl, allyl, 2-(cyclopropyl)ethyl, cyclohexylmethyl, 2-(cyclohexyl)methyl, cyclohexyl, 2-methoxyethyl, benzyl, 2-phenylethyl, 3-phenylpropyl, 3-(4-fluorophenyl)-2-methyl-2-propyl, 3-fluorophenyl, 3-(trifluoromethyl)phenyl, or 2-(aminocarbonyl)-2-methyl-1-propyl,
R¹⁰ is hydrogen, methyl, or isobutyl;
or R⁹-R¹⁰ is —(CH₂)₅— or —(CH₂)₂O(CH₂)₂—;
and the enantiomers, diastereomers, and salts thereof.

6. A compound of claim 3, wherein at least one, two, or preferably all three of the asymmetric carbon atoms of the main chain have the stereochemical configuration shown in formula Ib 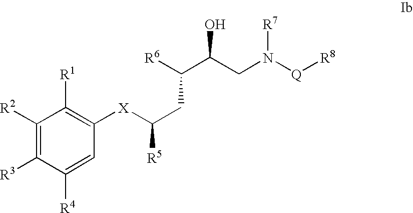
and the pharmaceutically acceptable salts thereof.

7. A compound of claim 1, wherein X is methylene and R⁵ is isopropyl.

8. A compound of claim 3, wherein X is methylene and R⁵ is isopropyl.

9. A compound of claim 6, wherein R⁵ is isopropyl.

10. A compound of claim 1 which is:

| Cpd. No. | Name |
|---|---|
| I-1 | N-((2S,3S,5S)-5-(3-(3-methoxypropoxy)-4-methoxybenzyl)-3-amino-2-hydroxy-6- |
| | methylheptyl)butyramide |
| I-2 | N-((2S,3S,5S)-5-(3-(3-methoxypropoxy)-4-methoxybenzyl)-3-amino-2-hydroxy-6- |
| | methylheptyl)-2-cyclopropylacetamide |
| I-3 | N-((2S,3S,5S)-5-(3-(3-methoxypropoxy)-4-methoxybenzyl)-3-amino-2-hydroxy-6- |
| | methylheptyl)pentanamide |
| I-4 | N-((2S,3S,5S)-5-(3-(3-methoxypropoxy)-4-methoxybenzyl)-3-amino-2-hydroxy-6- |
| | methylheptyl)pivalamide |
| I-5 | N-((2S,3S,5S)-5-(3-(2-cyclopropylethoxy)benzyl)-3-amino-2-hydroxy-6- |
| | methylheptyl)-2,2-dimethylhexanamide |
| I-6 | N-((2S,3S,5S)-5-(3-(3-methoxypropoxy)-4-methoxybenzyl)-3-amino-2-hydroxy-6- |
| | methylheptyl)hexanamide |
| I-7 | N-((2S,3S,5S)-5-(3-(3-methoxypropoxy)-4-methoxybenzyl)-3-amino-2-hydroxy-6- |
| | methylheptyl)-2,2-dimethylbutanamide |
| I-8 | N-((2S,3S,5S)-5-(3-(3-methoxypropoxy)-4-methoxybenzyl)-3-amino-2-hydroxy-6- |
| | methylheptyl)-3,3-dimethylbutanamide |
| I-9 | N-((2S,3S,5S)-5-(3-(3-methoxypropoxy)-4-methoxybenzyl)-3-amino-2-hydroxy-6- |
| | methylheptyl)-4-methoxybutanamide |
| I-10 | N-((2S,3S,5S)-5-(3-(3-methoxypropoxy)-4-methoxybenzyl)-3-amino-2-hydroxy-6- |
| | methylheptyl)benzamide |
| I-11 | N-((2S,3S,5S)-5-(3-(3-methoxypropoxy)-4-methoxybenzyl)-3-amino-2-hydroxy-6- |
| | methylheptyl)-3,3,3-trifluoropropanamide |
| I-12 | N-((2S,3S,5S)-5-(3-(3-methoxypropoxy)-4-methoxybenzyl)-3-amino-2-hydroxy-6- |
| | methylheptyl)-2-cyclopentylacetamide |
| I-13 | N-((2S,3S,5S)-5-(3-(3-methoxypropoxy)-4-methoxybenzyl)-3-amino-2-hydroxy-6- |
| | methylheptyl)cyclohexanecarboxamide |
| I-14 | N-((2S,3S,5S)-5-(3-(3-ethoxypropoxy)benzyl)-3-amino-2-hydroxy-6-methylheptyl)- |
| | 2,2-dimethylhexanamide |
| I-15 | N-((2S,3S,5S)-5-(3-(3-methoxypropoxy)-4-methoxybenzyl)-3-amino-2-hydroxy-6- |
| | methylheptyl)heptanamide |
| I-16 | N-((2S,3S,5S)-5-(3-(3-methoxypropoxy)-4-methoxybenzyl)-3-amino-2-hydroxy-6- |
| | methylheptyl)-2,2-dimethylpentanamide |
| I-17 | N-((2S,3S,5S)-5-(3-(3-methoxypropoxy)-4-methoxybenzyl)-3-amino-2-hydroxy-6- |
| | methylheptyl)-2-methylhexanamide |
| I-18 | N-((2S,3S,5S)-5-(3-(3-methoxypropoxy)-4-methoxybenzyl)-3-amino-2-hydroxy-6- |
| | methylheptyl)-2-phenylacetamide |
| I-19 | (2S,3S,5S)-5-(3-(3-methoxypropoxy)-4-methoxybenzyl)-3-amino-6-methyl-1- |
| | (butanesulfonylamino)heptan-2-ol |
| I-20 | N-((2S,3S,5S)-5-(3-(3-methoxypropoxy)-4-methoxybenzyl)-3-amino-2- |
| | hydroxyheptyl)-4,4,4-trifluorobutanamide |
| I-21 | N-((2S,3S,5S)-5-(3-(3-methoxypropoxy)-4-methoxybenzyl)-3-amino-2-hydroxy-6- |
| | methylheptyl)-2-cyclohexylacetamide |
| I-22 | 1-((2S,3S,5S)-5-(3-(3-methoxypropoxy)-4-methoxybenzyl)-3-amino-2-hydroxy-6- |
| | methylheptyl)-3-(1-(4-fluorophenyl)-2-methylpropan-2-yl)urea |
| I-23 | N-((2S,3S,5S)-5-(3-(3-methoxypropoxy)-4-methoxybenzyl)-3-amino-2-hydroxy-6- |
| | methylheptyl)-1-methylcyclohexanecarboxamide |
| I-24 | N-((2S,3S,5S)-5-(3-(3-methoxypropoxy)-4-methoxybenzyl)-3-amino-2-hydroxy-6- |
| | methylheptyl)-4-methylcyclohexanecarboxamide |
| I-25 | N-((2S,3S,5S)-5-(3-(3-methoxypropoxy)-4-ethylbenzyl)-3-amino-2-hydroxy-6- |
| | methylheptyl)-2,2-dimethylhexanamide |
| I-26 | N-((2R,3S,5S)-5-(3-(3-methoxypropoxy)-4-ethylbenzyl)-3-amino-2-hydroxy-6- |
| | methylheptyl)-2,2-dimethylhexanamide |
| I-27 | N-((2S,3S,5S)-5-(3-(3-methoxypropoxy)-4-methoxybenzyl)-3-amino-2-hydroxy-6- |
| | methylheptyl)-N-isopropylpentanamide |
| I-28 | N-((2S,3S,5S)-5-(3-(3-methoxypropoxy)-4-methoxybenzyl)-3-amino-2-hydroxy-6- |
| | methylheptyl)-2,2-dimethylhexanamide |
| I-29 | N-((2R,3R,5S)-5-(3-(3-methoxypropoxy)-4-methoxybenzyl)-3-amino-2-hydroxy-6- |
| | methylheptyl)-2,2-dimethylhexanamide |
| I-30 | N-((2S,3S,5S)-5-(3-(3-methoxypropoxy)-4-methoxybenzyl)-3-amino-2-hydroxy-6- |
| | methylheptyl)-3,3-dimethylhexanamide |
| I-31 | N-((2S,3S,5S)-5-(3-(3-methoxypropoxy)-4-methoxybenzyl)-3-amino-2-hydroxy-6- |
| | methylheptyl)-2-ethylhexanamide |
| I-32 | N-((2S,3S,5S)-5-(3-(3-methoxypropoxy)-4-methoxybenzyl)-3-amino-2-hydroxy-6- |
| | methylheptyl)-2-methyl-2-propoxypropanamide |
| I-33 | N-((2S,3S,5S)-5-(3-(3-methoxypropoxy)-4-methoxybenzyl)-3-amino-2-hydroxy-6- |
| | methylheptyl)-3-ethoxy-2,2-dimethylpropanamide |
| I-34 | N-((2S,3S,5S)-5-(3-(3-methoxypropoxy)-4-methoxybenzyl)-3-amino-2-hydroxy-6- |
| | methylheptyl)-3-phenylpropanamide |
| I-35 | N-((2S,3S,5S)-5-(3-(3-methoxypropoxy)-4-methoxybenzyl)-3-amino-2-hydroxy-6- |
| | methylheptyl)-2-m-tolylacetamide |
| I-36 | (2S,3S,5S)-5-(3-(3-methoxypropoxy)-4-methoxybenzyl)-3-amino-6-methyl-1- |
| | (pentanesulfonylamino)heptan-2-ol |
| I-37 | N-((2S,3S,5S)-5-(3-(3-methoxypropoxy)-4-methoxybenzyl)-3-amino-2-hydroxy-6- |
| | methylheptyl)-2-(2-fluorophenyl)acetamide |
| I-38 | N-((2S,3S,5S)-5-(3-(3-methoxypropoxy)-4-methoxybenzyl)-3-amino-2-hydroxy-6- |
| | methylheptyl)-2-(3-fluorophenyl)acetamide |
| I-39 | N-((2S,3S,5S)-5-(3-(3-methoxypropoxy)-4-methoxybenzyl)-3-amino-2-hydroxy-6- |
| | methylheptyl)-2-(4-fluorophenyl)acetamide |
| I-40 | N-((2S,3S,5S)-5-(3-(3-methoxypropoxy)-4-methoxybenzyl)-3-amino-2-hydroxy-6- |
| | methylheptyl)-2-(4-fluorophenyl)acetamide |
| I-41 | N-((2S,3S,5S)-5-(3-(3-methoxypropoxy)-4-methoxybenzyl)-3-amino-2-hydroxy-6- |
| | methylheptyl)-5,5,5-trifluoropentanamide |
| I-42 | N-((2S,3S,5S)-5-(3-(3-methoxypropoxy)-4-methoxybenzyl)-3-amino-2-hydroxy-6- |
| | methylheptyl)-4-cyclopropyl-2,2-dimethylbutanamide |
| I-43 | N-((2R,3S,5S)-5-(3-(3-methoxypropoxy)-4-methoxybenzyl)-3-amino-2-hydroxy-6- |
| | methylheptyl)-4-cyclopropyl-2,2-dimethylbutanamide |
| I-44 | (2S,3S,5S)-5-(3-(3-methoxypropoxy)-4-methoxybenzyl)-3-amino-6-methyl-1- |
| | (benzenesulfonylamino)heptan-2-ol |
| I-45 | N-((2S,3S,5S)-5-(3-(3-methoxypropoxy)-4-methoxybenzyl)-3-amino-2-hydroxy-6- |
| | methylheptyl)-3,5,5-trimethylhexanamide |
| I-46 | N-((2S,3S,5S)-5-(3-(3-methoxypropoxy)-4-methoxybenzyl)-3-amino-2-hydroxy-6- |
| | methylheptyl)-N,2,2-trimethylhexanamide |
| I-47 | N-((2S,3S,5S)-5-(3-(3-methoxypropoxy)-4-methoxybenzyl)-3-amino-2-hydroxy-6- |
| | methylheptyl)-2-(4-cyanophenyl)acetamide |
| I-48 | N-((2S,3S,5S)-5-(3-(3-methoxypropoxy)-4-methoxybenzyl)-3-amino-2-hydroxy-6- |
| | methylheptyl)-1-phenylcyclopropanecarboxamide |
| I-49 | N-((2S,3S,5S)-5-(3-(3-methoxypropoxy)-4-methoxybenzyl)-3-amino-2-hydroxy-6- |
| | methylheptyl)-4-phenylbutanamide |
| I-50 | N-((2S,3S,5S)-5-(3-(3-methoxypropoxy)-4-methoxybenzyl)-3-amino-2-hydroxy-6- |
| | methylheptyl)-2-methyl-2-phenylpropanamide |
| I-51 | N-((2R,3S,5S)-5-(3-(3-methoxypropoxy)-4-methoxybenzyl)-3-amino-2-hydroxy-6- |
| | methylheptyl)-2-methyl-2-phenylpropanamide |
| I-52 | N-((2S,3S,5S)-5-(3-(3-methoxypropoxy)-4-methoxybenzyl)-3-amino-2-hydroxy-6- |
| | methylheptyl)-2-cyclohexyl-2-methylpropanamide |
| I-53 | N-((2S,3S,5S)-5-(3-(3-methoxypropoxy)-4-methoxybenzyl)-3-amino-2-hydroxy-6- |
| | methylheptyl)-2-(3,4-difluorophenyl)acetamide |
| I-54 | N-((2S,3S,5S)-5-(3-(3-methoxypropoxy)-4-methoxybenzyl)-3-amino-2-hydroxy-6- |
| | methylheptyl)-2-(2,4-difluorophenyl)acetamide |
| I-55 | N-((2S,3S,5S)-5-(3-(3-methoxypropoxy)-4-methoxybenzyl)-3-amino-2-hydroxy-6- |
| | methylheptyl)-2-(2,3-difluorophenyl)acetamide |
| I-56 | (2S,3S,5S)-5-(3-(3-methoxypropoxy)-4-methoxybenzyl)-3-amino-6-methyl-1- |
| | (benzylsulfonylamino)heptan-2-ol |
| I-57 | N-((2S,3S,5S)-5-(3-(3-methoxypropoxy)-4-methoxybenzyl)-3-amino-2-hydroxy-6- |
| | methylheptyl)-1-p-tolylcyclopropanecarboxamide |
| I-58 | (2S,3S,5S)-5-(3-(3-methoxypropoxy)-4-methoxybenzyl)-3-amino-1-(N-isopropyl-N- |
| | butanesulfonylamino)-6-methylheptan-2-ol |
| I-59 | (2R,3S,5S)-5-(3-(3-methoxypropoxy)-4-methoxybenzyl)-3-amino-1-(N-isopropyl-N- |
| | butanesulfonylamino)-6-methylheptan-2-ol |
| I-60 | N-((2S,3S,5S)-5-(3-(3-methoxypropoxy)-4-methoxybenzyl)-3-amino-2-hydroxy-6- |
| | methylheptyl)-2-(4-fluorophenyl)-2-methylpropanamide |
| I-61 | N-((2R,3S,5S)-5-(3-(3-methoxypropoxy)-4-methoxybenzyl)-3-amino-2-hydroxy-6- |
| | methylheptyl)-2-(4-fluorophenyl)-2-methylpropanamide |
| I-62 | N-((2S,3S,5S)-5-(3-(3-methoxypropoxy)-4-methoxybenzyl)-3-amino-2-hydroxy-6- |
| | methylheptyl)-2-(4-fluorophenyl)-2-methylpropanamide |
| I-63 | N-((2S,3S,5S)-5-(3-(3-methoxypropoxy)-4-methoxybenzyl)-3-amino-2-hydroxy-6- |
| | methylheptyl)-2-cyclopentylhexanamide |
| I-64 | N-((2S,3S,5S)-5-(3-(3-methoxypropoxy)-4-methoxybenzyl)-3-amino-2-hydroxy-6- |
| | methylheptyl)-1-phenylcyclopentanecarboxamide |
| I-65 | N-((2S,3S,5S)-5-(3-(3-methoxypropoxy)-4-phenylbenzyl)-3-amino-2-hydroxy-6- |
| | methylheptyl)-2,2-dimethylhexanamide |
| I-66 | N-((2S,3S,5S)-5-(5-(3-methoxypropoxy)-2-phenylbenzyl)-3-amino-2-hydroxy-6- |
| | methylheptyl)-2,2-dimethylhexanamide |
| I-67 | N-((2S,3S,5S)-5-(3-(3-methoxypropoxy)-4-methoxybenzyl)-3-amino-2-hydroxy-6- |
| | methylheptyl)-1-(4-methoxyphenyl)cyclopropanecarboxamide |
| I-68 | N-((2S,3S,5S)-5-(3-(3-methoxypropoxy)-4-bromobenzyl)-3-amino-2-hydroxy-6- |
| | methylheptyl)-2,2-dimethylhexanamide |
| I-69 | N-((2S,3S,5S)-5-(3-(3-methoxypropoxy)-4-methoxybenzyl)-3-amino-2-hydroxy-6- |
| | methylheptyl)-3-hydroxy-2,2-dimethyl-3-phenylpropanamide |
| I-70 | N-((2S,3S,5S)-5-(3-(3-methoxypropoxy)-4-methoxybenzyl)-3-amino-2-hydroxy-6- |
| | methylheptyl)-1-(4-chlorophenyl)cyclopropanecarboxamide |
| I-71 | (2S,3S,5S)-5-(3-(3-methoxypropoxy)-4-methoxybenzyl)-3-amino-1-(N-isopropyl-N- |
| | benzenesulfonylamino)-6-methylheptan-2-ol |
| I-72 | N-((2S,3S,5S)-5-(3-(3-methoxypropoxy)-4-methoxybenzyl)-3-amino-2-hydroxy-6- |
| | methylheptyl)-2-(2-(trifluoromethyl)phenyl)acetamide |
| I-73 | N-((2S,3S,5S)-5-(3-(3-methoxypropoxy)-4-methoxybenzyl)-3-amino-2-hydroxy-6- |
| | methylheptyl)-2-(3-(trifluoromethyl)phenyl)acetamide |
| I-74 | N-((2S,3S,5S)-5-(3-(3-methoxypropoxy)-4-methoxybenzyl)-3-amino-2-hydroxy-6- |
| | methylheptyl)-2-(4-(trifluoromethyl)phenyl)acetamide |
| I-75 | N-((2S,3S,5S)-5-(3-(3-methoxypropoxy)-4-methoxybenzyl)-3-amino-2-hydroxy-6- |
| | methylheptyl)-1-phenylcyclohexanecarboxamide |
| I-76 | N-((2S,3S,5S)-5-(3-(3-methoxypropoxy)-4-methoxybenzyl)-3-amino-2-hydroxy-6- |
| | methylheptyl)-2-(4-cyanophenoxy)-2-methylpropanamide |
| I-77 | N-((2S,3S,5S)-5-(3-(3-methoxypropoxy)-4-methoxybenzyl)-3-amino-2-hydroxy-6- |
| | methylheptyl)-2,2-bis(trifluoromethyl)propanamide |
| I-78 | N-((2S,3S,5S)-5-(3-(3-methoxypropoxy)-4-methoxybenzyl)-3-amino-2-hydroxy-6- |
| | methylheptyl)-2-ethyl-2-(4-fluorophenyl)butanamide |
| I-79 | (2S,3S,5S)-5-(3-(3-methoxypropoxy)-4-methoxybenzyl)-3-amino-1-(N-isopropyl-N- |
| | benzylsulfonylamino)-6-methylheptan-2-ol |
| I-80 | N-((2S,3S,5S)-5-(3-(3-methoxypropoxy)-4-methoxybenzyl)-3-amino-2-hydroxy-6- |
| | methylheptyl)-2-(4-chlorophenoxy)-2-methylpropanamide |
| I-81 | N-((2S,3S,5S)-5-(3-(3-methoxypropoxy)-4-methoxybenzyl)-3-amino-2-hydroxy-6- |
| | methylheptyl)-2-(4-(trifluoromethoxy)phenyl)acetamide |
| I-82 | N-((2S,3S,5S)-5-(3-(3-methoxypropoxy)-4-methoxybenzyl)-3-amino-2-hydroxy-6- |
| | methylheptyl)-1-(4-fluorophenyl)cyclohexanecarboxamide |
| I-83 | N-((2S,3S,5S)-5-(3-(3-methoxypropoxy)-4-methoxybenzyl)-3-amino-2-hydroxy-6- |
| | methylheptyl)-1-(2,4-dichlorophenyl)cyclopropanecarboxamide |
| I-84 | 1-((2S,3S,5S)-5-(3-(3-methoxypropoxy)-4-methoxybenzyl)-3-amino-2-hydroxy-6- |
| | methylheptyl)urea |
| I-85 | 1-((2S,3S,5S)-5-(3-(3-methoxypropoxy)-4-methoxybenzyl)-3-amino-2-hydroxy-6- |
| | methylheptyl)-3-butylurea |
| I-86 | 1-((2S,3S,5S)-5-(3-(3-methoxypropoxy)-4-methoxybenzyl)-3-amino-2-hydroxy-6- |
| | methylheptyl)-3-tert-butylurea |
| I-87 | isobutyl (2S,3S,5S)-5-(3-(3-methoxypropoxy)-4-methoxybenzyl)-3-amino-2-hydroxy- |
| | 6-methylheptylcarbamate |
| I-88 | N-((2S,3S,5S)-5-(3-(3-methoxypropoxy)-4-methoxybenzyl)-3-amino-2-hydroxy-6- |
| | methylheptyl)piperidine-1-carboxamide |
| I-89 | 1-((2S,3S,5S)-5-(3-(3-methoxypropoxy)-4-methoxybenzyl)-3-amino-2-hydroxy-6- |
| | methylheptyl)-3-(2-cyclopropylethyl)urea |
| I-90 | N-((2S,3S,5S)-5-(3-(3-methoxypropoxy)-4-methoxybenzyl)-3-amino-2-hydroxy-6- |
| | methylheptyl)morpholine-4-carboxamide |
| I-91 | 1-((2S,3S,5S)-5-(3-(3-methoxypropoxy)-4-methoxybenzyl)-3-amino-2-hydroxy-6- |
| | methylheptyl)-3-pentylurea |
| I-92 | 1-((2S,3S,5S)-5-(3-(3-methoxypropoxy)-4-methoxybenzyl)-3-amino-2-hydroxy-6- |
| | methylheptyl)-3-pentylurea |
| I-93 | 1-((2R,3S,5S)-5-(3-(3-methoxypropoxy)-4-methoxybenzyl)-3-amino-2-hydroxy-6- |
| | methylheptyl)-3-pentylurea |
| I-94 | pentyl (2S,3S,5S)-5-(3-(3-methoxypropoxy)-4-methoxybenzyl)-3-amino-2-hydroxy-6- |
| | methylheptylcarbamate |
| I-95 | 1-((2S,3S,5S)-5-(3-(3-methoxypropoxy)-4-methoxybenzyl)-3-amino-2-hydroxy-6- |
| | methylheptyl)-3-(3-methoxypropyl)urea |
| I-96 | 1-((2S,3S,5S)-5-(3-(3-methoxypropoxy)-4-methoxybenzyl)-3-amino-2-hydroxy-6- |
| | methylheptyl)-3-(2-ethoxyethyl)urea |
| I-97 | 1-((2S,3S,5S)-5-(3-(3-methoxypropoxy)-4-methoxybenzyl)-3-amino-2-hydroxy-6- |
| | methylheptyl)-3-cyclohexylurea |
| I-98 | 1-((2S,3S,5S)-5-(3-(3-methoxypropoxy)-4-methoxybenzyl)-3-amino-2-hydroxy-6- |
| | methylheptyl)-3-hexylurea |
| I-99 | 3-((2S,3S,5S)-5-(3-(3-methoxypropoxy)-4-methoxybenzyl)-3-amino-2-hydroxy-6- |
| | methylheptyl)-1-methyl-1-pentylurea |
| I-100 | 1-((2S,3S,5S)-5-(3-(3-methoxypropoxy)-4-methoxybenzyl)-3-amino-2-hydroxy-6- |
| | methylheptyl)-1-methyl-3-pentylurea |
| I-101 | 1-((2S,3S,5S)-5-(3-(3-methoxypropoxy)-4-methoxybenzyl)-3-amino-2-hydroxy-6- |
| | methylheptyl)-3-pentylthiourea |
| I-102 | 1-((2S,3S,5S)-5-(3-(3-methoxypropoxy)-4-methoxybenzyl)-3-amino-2-hydroxy-6- |
| | methylheptyl)-3-benzylurea |
| I-103 | benzyl (2S,3S,5S)-5-(3-(3-methoxypropoxy)-4-methoxybenzyl)-3-amino-2-hydroxy- |
| | 6-methylheptylcarbamate |
| I-104 | (2S,3S,5S)-5-(3-(3-methoxypropoxy)-4-methoxybenzyl)-3-amino-1- |
| | (butylaminosulfonylamino)-6-methylheptan-2-ol |
| I-105 | 1-((2S,3S,5S)-5-(3-(3-methoxypropoxy)-4-methoxybenzyl)-3-amino-2-hydroxy-6- |
| | methylheptyl)-3-(3-fluorophenyl)urea |
| I-106 | 1-((2S,3S,5S)-5-(3-(3-methoxypropoxy)-4-methoxybenzyl)-3-amino-2-hydroxy-6- |
| | methylheptyl)-3-(cyclohexylmethyl)urea |
| I-107 | 3-((2S,3S,5S)-5-(3-(3-methoxypropoxy)-4-methoxybenzyl)-3-amino-2-hydroxy-6- |
| | methylheptyl)-1-cyclohexyl-1-methylurea |
| I-108 | (2S,3S,5S)-5-(3-(3-methoxypropoxy)-4-methoxybenzyl)-3-amino-1-(N- |
| | (butylaminosulfonyl)-N-isopropylamino)-6-methylheptan-2-ol |
| I-109 | 1-((2S,3S,5S)-5-(3-(3-methoxypropoxy)-4-methoxybenzyl)-3-amino-2-hydroxy-6- |
| | methylheptyl)-3-(2,2-dimethylpentyl)urea |
| I-110 | 1-((2S,3S,5S)-5-(3-(3-methoxypropoxy)-4-methoxybenzyl)-3-amino-2-hydroxy-6- |
| | methylheptyl)-3-(2-methylhexan-2-yl)urea |
| I-111 | 1-((2S,3S,5S)-5-(3-(3-methoxypropoxy)-4-methoxybenzyl)-3-amino-2-hydroxy-6- |
| | methylheptyl)-3-(2-carbamoyl-2-methylpropyl)urea |
| I-112 | 1-((2S,3S,5S)-5-(3-(3-methoxypropoxy)-4-methoxybenzyl)-3-amino-2-hydroxy-6- |
| | methylheptyl)-3-phenethylurea |
| I-113 | (2S,3S,5S)-5-(3-(3-methoxypropoxy)-4-methoxybenzyl)-3-amino-1- |
| | (pentylaminosulfonylamino)-6-methylheptan-2-ol |
| I-114 | 1-((2S,3S,5S)-5-(3-(3-methoxypropoxy)-4-methoxybenzyl)-3-amino-2-hydroxy-6- |
| | methylheptyl)-3-(2-cyclohexylethyl)urea |
| I-115 | 1-((2S,3S,5S)-5-(3-(3-methoxypropoxy)-4-methoxybenzyl)-3-amino-2-hydroxy-6- |
| | methylheptyl)-3-(2,4,4-trimethylpentan-2-yl)urea |
| I-116 | 3-((2S,3S,5S)-5-(3-(3-methoxypropoxy)-4-methoxybenzyl)-3-amino-2-hydroxy-6- |
| | methylheptyl)-1,1-diisobutylurea |
| I-117 | 1-((2S,3S,5S)-5-(3-(3-methoxypropoxy)-4-methoxybenzyl)-3-amino-2-hydroxy-6- |
| | methylheptyl)-3-(3-phenylpropyl)urea |
| I-118 | (2S,3S,5S)-5-(3-(3-methoxypropoxy)-4-methoxybenzyl)-3-amino-1-(N- |
| | (allylaminosulfonyl)-N-isopropylamino)-6-methylheptan-2-ol |
| I-119 | 1-((2S,3S,5S)-5-(3-(3-methoxypropoxy)-4-methoxybenzyl)-3-amino-2-hydroxy-6- |
| | methylheptyl)-3-(3-(trifluoromethyl)phenyl)urea or |
| I-120 | 1-((2S,3S,5S)-5-(3-(3-methoxypropoxy)-4-methoxybenzyl)-3-amino-2-hydroxy-6- |
| | methylheptyl)-3-(1-(4-fluorophenyl)-2-methylpropan-2-yl)urea |

and their enantiomers, diastereomers and salts.

11. A compound of claim 1 which is:

| Cpd. No. | Name |
|---|---|
| I-28 | N-((2S,3S,5S)-5-(3-(3-methoxypropoxy)-4-methoxybenzyl)-3-amino-2-hydroxy-6- |
| | methylheptyl)-2,2-dimethylhexanamide |
| I-42 | N-((2S,3S,5S)-5-(3-(3-methoxypropoxy)-4-methoxybenzyl)-3-amino-2-hydroxy-6- |
| | methylheptyl)-4-cyclopropyl-2,2-dimethylbutanamide |
| I-50 | N-((2S,3S,5S)-5-(3-(3-methoxypropoxy)-4-methoxybenzyl)-3-amino-2-hydroxy-6- |
| | methylheptyl)-2-methyl-2-phenylpropanamide |
| I-52 | N-((2S,3S,5S)-5-(3-(3-methoxypropoxy)-4-methoxybenzyl)-3-amino-2-hydroxy-6- |
| | methylheptyl)-2-cyclohexyl-2-methylpropanamide or |
| I-62 | N-((2S,3S,5S)-5-(3-(3-methoxypropoxy)-4-methoxybenzyl)-3-amino-2-hydroxy-6- |
| | methylheptyl)-2-(4-fluorophenyl)-2-methylpropanamide |

and their enantiomers, diastereomers and salts.

12. A compound of claim 1 which is:

| Cpd. No. | Name |
|---|---|
| I-6 | N-((2S,3S,5S)-5-(3-(3-methoxypropoxy)-4-methoxybenzyl)-3-amino-2-hydroxy-6- |
| | methylheptyl)hexanamide |
| I-17 | N-((2S,3S,5S)-5-(3-(3-methoxypropoxy)-4-methoxybenzyl)-3-amino-2-hydroxy-6- |
| | methylheptyl)-2-methylhexanamide |
| I-21 | N-((2S,3S,5S)-5-(3-(3-methoxypropoxy)-4-methoxybenzyl)-3-amino-2-hydroxy-6- |
| | methylheptyl)-2-cyclohexylacetamide |
| I-26 | N-((2R,3S,5S)-5-(3-(3-methoxypropoxy)-4-ethylbenzyl)-3-amino-2-hydroxy-6- |
| | methylheptyl)-2,2-dimethylhexanamide |
| I-31 | N-((2S,3S,5S)-5-(3-(3-methoxypropoxy)-4-methoxybenzyl)-3-amino-2-hydroxy-6- |
| | methylheptyl)-2-ethylhexanamide |
| I-33 | N-((2S,3S,5S)-5-(3-(3-methoxypropoxy)-4-methoxybenzyl)-3-amino-2-hydroxy-6- |
| | methylheptyl)-3-ethoxy-2,2-dimethylpropanamide |
| I-39 | N-((2S,3S,5S)-5-(3-(3-methoxypropoxy)-4-methoxybenzyl)-3-amino-2-hydroxy-6- |
| | methylheptyl)-2-(4-fluorophenyl)acetamide |
| I-40 | N-((2S,3S,5S)-5-(3-(3-methoxypropoxy)-4-methoxybenzyl)-3-amino-2-hydroxy-6- |
| | methylheptyl)-2-(4-fluorophenyl)acetamide |
| I-42 | N-((2S,3S,5S)-5-(3-(3-methoxypropoxy)-4-methoxybenzyl)-3-amino-2-hydroxy-6- |
| | methylheptyl)-4-cyclopropyl-2,2-dimethylbutanamide |
| I-74 | N-((2S,3S,5S)-5-(3-(3-methoxypropoxy)-4-methoxybenzyl)-3-amino-2-hydroxy-6- |
| | methylheptyl)-2-(4-(trifluoromethyl)phenyl)acetamide |
| I-75 | N-((2S,3S,5S)-5-(3-(3-methoxypropoxy)-4-methoxybenzyl)-3-amino-2-hydroxy-6- |
| | methylheptyl)-1-phenylcyclohexanecarboxamide |
| I-78 | N-((2S,3S,5S)-5-(3-(3-methoxypropoxy)-4-methoxybenzyl)-3-amino-2-hydroxy-6- |
| | methylheptyl)-2-ethyl-2-(4-fluorophenyl)butanamide |
| I-82 | N-((2S,3S,5S)-5-(3-(3-methoxypropoxy)-4-methoxybenzyl)-3-amino-2-hydroxy-6- |
| | methylheptyl)-1-(4-fluorophenyl)cyclohexanecarboxamide |
| I-85 | 1-((2S,3S,5S)-5-(3-(3-methoxypropoxy)-4-methoxybenzyl)-3-amino-2-hydroxy-6- |
| | methylheptyl)-3-butylurea |
| I-91 | 1-((2S,3S,5S)-5-(3-(3-methoxypropoxy)-4-methoxybenzyl)-3-amino-2-hydroxy-6- |
| | methylheptyl)-3-pentylurea |
| I-92 | 1-((2S,3S,5S)-5-(3-(3-methoxypropoxy)-4-methoxybenzyl)-3-amino-2-hydroxy-6- |
| | methylheptyl)-3-pentylurea |
| I-98 | 1-((2S,3S,5S)-5-(3-(3-methoxypropoxy)-4-methoxybenzyl)-3-amino-2-hydroxy-6- |
| | methylheptyl)-3-hexylurea |
| I-99 | 3-((2S,3S,5S)-5-(3-(3-methoxypropoxy)-4-methoxybenzyl)-3-amino-2-hydroxy-6- |
| | methylheptyl)-1-methyl-1-pentylurea |
| I-109 | 1-((2S,3S,5S)-5-(3-(3-methoxypropoxy)-4-methoxybenzyl)-3-amino-2-hydroxy-6- |
| | methylheptyl)-3-(2,2-dimethylpentyl)urea |
| I-115 | 1-((2S,3S,5S)-5-(3-(3-methoxypropoxy)-4-methoxybenzyl)-3-amino-2-hydroxy-6- |
| | methylheptyl)-3-(2,4,4-trimethylpentan-2-yl)urea or |
| I-116 | 3-((2S,3S,5S)-5-(3-(3-methoxypropoxy)-4-methoxybenzyl)-3-amino-2-hydroxy-6- |
| | methylheptyl)-1,1-diisobutylurea |

and their enantiomers, diastereomers and salts.

13. A compound of claim 1 which is:
N-((2S,3S,5S)-5-(3-(3-methoxypropoxy)-4-methoxybenzyl)-3-amino-2-hydroxy-6-methylheptyl)-4-cyclopropyl-2,2-dimethylbutanamide and its enantiomers, diastereomers and salts.

14. A compound of formula II: 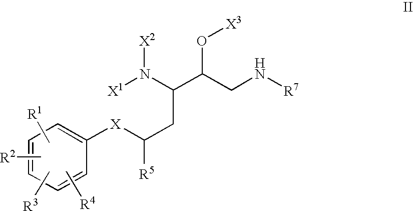
wherein
R¹ is hydrogen, halogen, hydroxy, lower alkoxy, cycloalkoxy, lower alkoxy-lower alkoxy, lower alkylthio-lower alkoxy, cyano-lower alkoxy, hydroxy-lower alkoxy, carboxy-lower alkoxy, lower alkoxycarbonyl-lower alkoxy, carbamoyl-lower alkoxy, N-mono- or N,N-di-lower alkylcarbamoyl-lower alkoxy, or aryl;
R² is hydrogen, halogen, cyano, carbamoyl, lower alkyl, lower haloalkyl, cycloalkyl, halocycloalkyl, cycloalkyl-lower alkyl, halocycloalkyl-loweralkyl, cyano-lower alkyl, hydroxy-lower alkyl, lower alkoxy-lower alkyl, lower haloalkoxy-lower alkyl, lower alkoxy-lower alkyl, lower haloalkoxy-lower alkyl, cycloalkoxy-lower alkyl, halocycloalkoxy-lower alkyl, hydroxy, lower alkanoyloxy-lower alkoxy, hydroxy-lower alkoxy, halo-(hydroxy)-lower alkoxy, lower alkanesulfonyl-(hydroxy)-lower alkoxy, amino-lower alkyl, lower alkylamino-lower alkyl, di-lower alkylamino-lower alkyl, lower alkanoylamino-lower alkyl, lower alkoxycarbonyl-amino-lower alkyl, aminocarbonylamino-lower alkyl, lower alkylaminocarbonylamino-lower alkyl, di(lower alkyl)aminocarbonylamino-lower alkyl, aminosulfonylamino-lower alkyl, lower alkylaminosulfonylamino-lower alkyl, di(lower alkyl)aminosulfonylamino-lower alkyl, amino-lower alkoxy, lower alkylamino-lower alkoxy, di-lower alkylamino-lower alkoxy, lower alkanoylamino-lower alkoxy, lower alkoxycarbonyl-amino-lower alkoxy, aminocarbonylamino-lower alkoxyl, lower alkylaminocarbonylamino-lower alkoxy, di(lower alkyl)aminocarbonylamino-lower alkoxy, aminosulfonylamino-lower alkoxy, lower alkylaminosulfonylamino-lower alkoxy, di(lower alkyl)aminosulfonylamino-lower alkoxy, oxo-lower alkoxy, lower alkoxy, lower haloalkoxy, cycloalkoxy, lower halocycloalkoxy, cycloalkyl-lower alkoxy, halocycloalkyl-lower alkoxy, lower alkenyloxy, cycloalkoxy-lower alkoxy, halocycloalkoxy-lower alkoxy, lower alkoxy-lower alkoxy, lower haloalkoxy-lower alkyl, lower alkoxy-lower alkenyl, lower alkenyloxy-lower alkoxy, lower alkoxy-lower alkenyloxy, lower alkenyloxy-lower alkyl, lower alkanoyl-lower alkoxy, lower alkylthio-lower alkoxy, lower alkanesulfonyl-lower alkoxy, lower alkylthio-(hydroxy)-lower alkoxy, aryl-lower alkoxy, optionally N-oxidized pyridyl-lower alkoxy, thiazolylthio-lower alkoxy or thiazolinylthio-lower alkoxy, imidazolylthio-lower alkoxy, optionally N-oxidized pyridylthio-lower alkoxy, pyrimidinylthio-lower alkoxy, cyano-lower alkoxy, carboxy-lower alkoxy, lower alkoxycarbonyl-lower alkoxy, carbamoyl-lower alkoxy, N-mono- or N,N-di-lower alkylcarbamoyl-lower alkoxy, carboxy-lower alkyl, lower alkoxycarbonyl-lower alkyl, carbamoyl-lower alkyl, or N-mono- or N,N-di-lower alkylcarbamoyl-lower alkyl;
R³ is hydrogen, halogen, cyano, carbamoyl, lower alkyl, lower haloalkyl, lower alkoxy-lower alkyl, cycloalkoxy-lower alkyl, hydroxy-lower alkyl, lower alkylthio-lower alkyl, lower alkanesulfonyl-lower alkyl, optionally partially hydrogenated or N-oxidized pyridyl-lower alkyl, thiazolyl-thio-lower alkyl or thiazolinylthio-lower alkyl, imidazolylthio-lower alkyl, optionally N-oxidized pyridylthio-lower alkyl, pyrimidinylthio-lower alkyl, amino-lower alkyl, lower alkylamino-lower alkyl, di-lower alkylamino-lower alkyl, lower alkanoyl-amino-lower alkyl, lower alkanesulfonylamino-lower alkyl, polyhalo-lower alkane-sulfonylamino-lower alkyl, pyrrolidino-lower alkyl, piperidino-lower alkyl, piperazino-lower alkyl, N′-lower alkylpiperazino-lower alkyl or N′-lower alkanoylpiperazino-lower alkyl, morpholino-lower alkyl, thiomorpholino-lower alkyl, S-oxothiomorpholino-lower alkyl or S,S-dioxothio-morpholino-lower alkyl, cyano-lower alkyl, carboxy-lower alkyl, lower alkoxy-carbonyl-lower alkyl, carbamoyl-lower alkyl, N-mono- or N,N-di-lower alkyl-carbamoyl-lower alkyl, cycloalkyl; phenyl or naphthyl that is unsubstituted or substituted with one to three groups independently selected from lower alkyl, lower alkoxy, hydroxy, lower alkylamino, di-lower alkylamino, halogen, trifluoromethyl, trifluoromethoxy, and cyano; hydroxy, lower alkoxy, cycloalkoxy, lower alkoxy-lower alkoxy, cycloalkoxy-lower alkoxy, hydroxy-lower alkoxy, aryl, lower haloalkoxy, lower alkylthio-lower alkoxy, lower haloalkylthio-lower alkoxy, lower alkanesulfonyl-lower alkoxy, lower haloalkanesulfonyl-lower alkoxy, optionally hydrogenated heteroaryl-lower alkoxy, heterocyclyl-lower alkoxy, optionally partially or fully hydrogenated heteroarylthio-lower alkoxy, such as thiazolylthio-lower alkoxy or thiazolinylthio-lower alkoxy, imidazolylthio-lower alkoxy, optionally N-oxidized pyridylthio-lower alkoxy, pyrimidinylthio-lower alkoxy, amino-lower alkoxy, lower alkylamino-lower alkoxy, di-lower alkylamino-lower alkoxy, lower alkanoylamino-lower alkoxy, lower alkanesulfonylamino-lower alkoxy, polyhalo-lower alkanesulfonylamino-lower alkoxy, pyrrolidino-lower alkoxy, piperidino-lower alkoxy, piperazino-lower alkoxy, N′-lower alkylpiperazino-lower alkoxy or N′-lower alkanoylpiperazino-lower alkoxy, morpholino-lower alkoxy, thiomorpholino-lower alkoxy, S-oxothiomorpholino-lower alkoxy or S,S-dioxothiomorpholino-lower alkoxy, cyano-lower alkoxy, carboxy-lower alkoxy, lower alkoxycarbonyl-lower alkoxy, carbamoyl-lower alkoxy, N-mono- or N,N-di-lower alkylcarbamoyl-lower alkoxy, carboxy-lower alkyl, lower alkoxycarbonyl-lower alkyl, carbamoyl-lower alkyl, or N-mono- or N,N-di-lower alkylcarbamoyl-lower alkyl; or
R² and R³ taken together with the atoms through which they are attached form a fused dioxolane, dioxane, benzene or cyclohexene ring, wherein said ring is substituted with up to 2 substituents independently selected from lower alkyl and lower alkoxy-lower alkyl;
R⁴ is hydrogen, lower alkyl, hydroxy, lower alkoxy, cycloalkoxy, lower alkoxy-lower alkoxy, or cycloalkyl-lower alkoxy; or
R³ and R⁴ taken together with the atoms through which they are attached form a fused dioxolane, dioxane, benzene or cyclohexene ring, wherein said ring is substituted with up to 2 substituents independently selected from lower alkyl and lower alkoxy-lower alkyl; provided that R³ does not form a ring with R²;
X is methylene or hydroxymethylene;
R⁵ is lower alkyl, lower haloalkyl, cycloalkyl, halocycloalkyl, lower alkyl-cycloalkyl, lower haloalkyl-cycloalkyl, cycloalkyl-lower alkyl, aryl, aryl-lower alkyl, heterocyclyl, heterocyclyl-lower alkyl;
R⁶ is amino, lower alkylamino, di-lower alkylamino, or lower alkanoylamino;
R⁷ is hydrogen, lower alkyl, lower haloalkyl, cycloalkyl, lower alkoxy-lower alkyl, or lower haloalkoxy-lower alkyl;
X¹ is an amino-protecting group;
X is hydrogen or together with X³ is a bivalent protecting group;
X³ is hydrogen or a hydroxy-protecting group;
and the enantiomers, diastereomers, and salts thereof.

15. A compound of claim 14 of the formula IIa 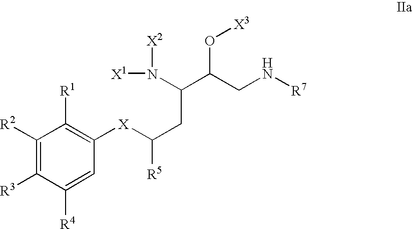
or a salt thereof
wherein:
R¹ is hydrogen or aryl;
R² is hydrogen, lower alkyl, cycloalkyl-lower alkyl, lower alkoxy-lower alkyl, lower haloalkoxy-lower alkyl, lower alkoxy-lower alkoxy, lower haloalkoxy-lower alkoxy, lower alkoxy-lower alkoxy-lower alkyl; cycloalkyl-lower alkoxy, phenyl-lower alkoxy that is unsubstituted or substituted by lower alkyl, lower alkoxy, hydroxy, halogen, nitro and/or by amino; optionally N-oxidized pyridyl-lower alkoxy, lower alkylthio-lower alkoxy, lower alkane-sulfonyl-lower alkoxy, lower alkanoyl-lower alkoxy, cyano-lower alkoxy, carboxy-lower alkoxy, lower alkoxycarbonyl-lower alkoxy, carbamoyl-lower alkoxy, lower alkylcarbamoyl-lower alkoxy, or di-lower alkylcarbamoyl-lower alkoxy;
R³ is hydrogen, halogen, cyano, lower alkyl, lower haloalkyl, aryl, hydroxy, lower alkoxy, or polyhalo-lower alkoxy; or
R² and R³ taken together with the atoms through which they are attached form a fused dioxolane ring, which is substituted with up to 2 substituents independently selected from lower alkyl and lower alkoxy-lower alkyl;
R⁴ is hydrogen, lower alkoxy-lower alkoxy, lower alkoxy-lower alkyl, or cycloalkyl-lower alkoxy; or
R³ and R⁴ taken together with the atoms through which they are attached form a fused dioxolane ring which is substituted with up to 2 substituents independently selected from lower alkyl and lower alkoxy-lower alkyl; provided that R³ does not form a ring with R²;
X is methylene or hydroxymethylene;
R⁵ is lower alkyl or cycloalkyl;
R⁷ is hydrogen or methyl;
X¹ is lower alkoxycarbonyl, 2-(trialkylsily)ethoxycarbonyl, or α-phenyl- or α,α-diphenyl-lower alkoxycarbonyl that is unsubstituted or substituted by lower alkyl, lower alkoxy, nitro and/or by halogen, or is 2-halo-lower alkoxycarbonyl;
X² is hydrogen or together with X³ is carbonyl or lower alkylidene;
X³ is hydrogen, tri-lower alkylsilyl.

16. (canceled)

17. A compound of claim 15, wherein
R¹ is hydrogen;
R² is (C₁-C₄)alkoxy-(C₁-C₄)alkoxy, (C₁-C₄)alkoxy-(C₁-C₄)alkyl, or cycloalkyl-lower alkoxy;
R³ is fluoro, chloro, bromo, cyano, (C₁-C₄)alkyl, (C₁-C₄) haloalkyl, aryl, (C₁-C₄)alkoxy, or (C₁-C₄)haloalkoxy;
R⁴ is hydrogen;
X is methylene;
R⁵ is (C₃-C₅)alkyl;
R⁷ is hydrogen;
X¹ is lower alkoxycarbonyl, or α-phenyl-lower alkoxycarbonyl that is unsubstituted or substituted by lower alkyl, lower alkoxy, nitro, and/or by halogen;
X² and X³ are both hydrogen, or taken together are lower alkylidene;
and the enantiomers, diastereomers, and salts thereof.

18. A compound of claim 17, wherein
R¹ is hydrogen;
R² is 3-methoxypropoxy, 3-ethoxypropoxy, 4-methoxybutyl, or 2-(cyclopropyl)ethoxy;
R³ is fluoro, chloro, bromo, cyano, methyl, ethyl, isopropyl or tert-butyl, trifluoromethyl, pentafluoroethyl, phenyl, methoxy, difluoromethoxy, or trifluoromethoxy;
R⁴ is hydrogen;
X is methylene;
R⁵ is branched (C₃-C₅)alkyl;
R⁷ is hydrogen;
X¹ is lower alkoxycarbonyl, or α-phenyl-lower alkoxycarbonyl that is unsubstituted or substituted by lower alkyl, lower alkoxy, nitro, and/or by halogen;
X² and X³ are both hydrogen, or taken together are lower alkylidene;
and the enantiomers, diastereomers, and salts thereof.

19. A compound of claim 15, wherein at least one, preferably all, of the asymmetric carbon atoms of the main chain have the stereochemical configuration shown in formula IIb 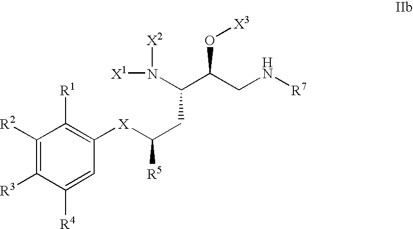
and the salts thereof.

20. A compound of claim 19, wherein
R¹ and R⁴ is each hydrogen;
R² is 3-methoxypropoxy, 3-ethoxypropoxy, 4-methoxybutyl, or 2-(cyclopropyl)ethoxy;
R³ is fluoro, chloro, bromo, cyano, methyl, ethyl, isopropyl or tert-butyl, trifluoromethyl, pentafluoroethyl, phenyl, methoxy, difluoromethoxy, or trifluoromethoxy;
X is methylene;
R⁵ is isopropyl;
R⁷ is hydrogen;
X¹ is tert-butoxycarbonyl; and
X² and X³ are both hydrogen, or taken together are isopropylidene
and the salts thereof.

21. A compound of formula III 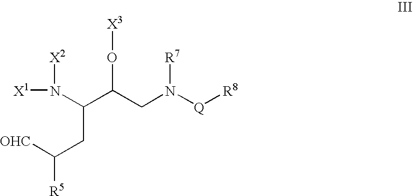
wherein
R⁵ is lower alkyl or cycloalkyl;
R⁷ is hydrogen, lower alkyl, lower haloalkyl, cycloalkyl, lower alkoxy-lower alkyl, or lower haloalkoxy-lower alkyl
Q is carbonyl, thiocarbonyl, or sulfonyl;
R⁸ is lower alkyl, lower haloalkyl, C₈-C₁₅alkyl, C₈-C₁₅haloalkyl, cycloalkyl, halocycloalkyl, lower alkyl-cycloalkyl, cycloalkyl-lower alkyl, halocycloalkyl-lower alkyl, lower alkoxy-loweralkyl, lower haloalkoxy-lower alkyl, cycloalkoxy-lower alkyl, cycloalkoxy-cycloalkyl, lower alkylthio-lower alkyl, lower haloalkylthio-lower alkyl, lower alkanesulfonyl-lower alkyl, lower haloalkanesulfonyl-lower alkyl, lower alkylthio-cycloalkyl, lower haloalkylthio-cycloalkyl, lower alkanesulfonyl-cycloalkyl, lower haloalkanesulfonyl-cycloalkyl, aryl, aryl-lower alkyl, aryl-lower hydroxyalkyl, arylcycloalkyl, aryloxy-lower alkyl, aryloxy cycloalkyl, arylthio-lower alkyl, arylsulfonyl-lower alkyl, arylthio-cycloalkyl, arylsulfonyl-cycloalkyl, lower alkanoyl-lower alkyl, hydroxy-lower alkyl, amino-lower alkyl, lower alkanoylamino-lower alkyl, N-mono-lower alkylamino-lower alkyl, N,N-di-lower alkylamino-lower alkyl, piperidino-lower alkyl, hydroxypiperidino-lower alkyl, lower alkoxypiperidino-lower alkyl, morpholino-lower alkyl, dimethylmorpholino-lower alkyl, thiomorpholino-lower alkyl, S,S-dioxothiomorpholino-lower alkyl, carboxy-lower alkyl, lower alkoxycarbonyl-lower alkyl, carbamoyl-lower alkyl, N-mono-lower alkylcarbamoyl-lower alkyl, N,N-di-lower alkylcarbamoyl-lower alkyl, carboxy-(hydroxy)-lower alkyl, lower alkoxycarbonyl-(hydroxy)-lower alkyl, carbamoyl-(hydroxy)-lower alkyl, N-mono-lower alkylcarbamoyl-(hydroxy)-lower alkyl, N,N-di-lower alkylcarbamoyl-(hydroxy)-lower alkyl, 5- or 6-membered carboxycycloalkyl-lower alkyl, 5- or 6-membered lower alkoxycarbonyl-cycloalkyl-lower alkyl, 5- or 6-membered carbamoylcycloalkyl-lower alkyl, 5- or 6-membered N-mono-alkylcarbamoylcycloalkyl-lower alkyl, N,N-di-lower alkylcarbamoylcycloalkyl-lower alkyl, cyano-lower alkyl, sulfamoyl-lower alkyl, lower alkylsulfamoyl-lower alkyl, or di-lower alkylsulfamoyl-lower alkyl, imidazolyl-lower alkyl, oxopyrrolidinyl-lower alkyl, benzimidazolyl-lower alkyl, oxadiazolyl-lower alkyl, pyridyl-lower alkyl, oxopiperidinyl-lower alkyl or quinolinyl-lower alkyl, piperidin-4-yl-lower alkyl, or lower alkanoylpiperidin-4-yl-lower alkyl, wherein said aryl, imidazolyl, benzimidazolyl, oxadiazolyl, pyridyl, quinolinyl, aryloxy, arylthio and arylsulfonyl groups are optionally substituted with up to four groups independently selected from halo, cyano, nitro, optionally halogenated lower alkyl, optionally halogenated lower alkoxy, optionally halogenated lower alkylthio, optionally halogenated lower alkanesulfonyl, and lower alkoxycarbonyl;
or R⁸ is OR⁹ or NR⁹R¹⁰
R⁹ is 1) hydrogen, lower alkyl, lower haloalkyl, lower alkenyl, (C₈-C₁₅)alkyl, (C₈-C₁₅)haloalkyl, cycloalkyl, halocycloalkyl, lower alkyl-cycloalkyl, cycloalkyl-lower alkyl, halocycloalkyl-lower alkyl, lower alkoxy-loweralkyl, lower haloalkoxy-lower alkyl, cycloalkoxy-lower alkyl, cycloalkoxy-cycloalkyl, lower alkylthio-lower alkyl, lower haloalkylthio-lower alkyl, lower alkanesulfonyl-lower alkyl, lower haloalkanesulfonyl-lower alkyl, lower alkylthio-cycloalkyl, lower haloalkylthio-cycloalkyl, lower alkanesulfonyl-cycloalkyl, lower haloalkanesulfonyl-cycloalkyl, aminocarbonyl-lower alkyl, lower alkyl-amonocarbonyl-lower alkyl, or di(lower alkyl)-amonocarbonyl-lower alkyl, or 2) aryl, aryl-lower alkyl, aryloxy-lower alkyl, arylthio-lower alkyl, or arylsulfonyl-lower alkyl
wherein aryl is optionally substituted with up to four groups independently selected from halo, cyano, optionally halogenated lower alkyl, optionally halogenated lower alkoxy, optionally halogenated lower alkylthio, and optionally halogenated lower alkanesulfonyl;
R¹⁰ is 1) hydrogen, lower alkyl, lower haloalkyl, (C₈-C₁₅)alkyl, (C₈-C₁₅)haloalkyl, cycloalkyl, halocycloalkyl, cycloalkyl-lower alkyl, halocycloalkyl-lower alkyl, lower alkoxy-lower alkyl, lower haloalkoxy-lower alkyl, alkylthio-lower alkyl, lower haloalkylthio-lower alkyl, lower alkanesulfonyl-lower alkyl, or lower haloalkanesulfonyl-lower alkyl, or 2) aryl or aryl-lower alkyl
wherein aryl is optionally substituted with up to four groups independently selected from halo, cyano, optionally halogenated lower alkyl, optionally halogenated lower alkoxy, optionally halogenated lower alkylthio, and optionally halogenated lower alkanesulfonyl;
or R⁹ and R¹⁰ taken together with the nitrogen to which they are attached form a 4-, 5-, 6- or 7-membered heterocyclic ring composed of carbon atoms and 0 or 1 N, O, or S atoms in addition to the nitrogen atom to which R⁹ and R¹⁰ are attached, said ring atoms being substituted with the appropriate number of hydrogen atoms and optionally substituted with up to four groups independently selected from halogen, (C₁-C₆)alkyl, halo(C₁-C₆)alkyl, lower alkanoyl, lower alkoxycarbonyl, aryl, aryl-lower alkyl, and oxo, such that substitution of one oxo group on a carbon atom forms a carbonyl group and substitution of one or two oxo groups on sulfur forms sulfoxide or sulfone groups respectively; wherein the aryl and arylalkyl groups are substituted with up to four groups independently selected from halo, cyano, optionally halogenated lower alkyl, optionally halogenated lower alkoxy, optionally halogenated lower alkylthio, and optionally halogenated lower alkanesulfonyl;
X¹ is an amino-protecting group;
X² is hydrogen or together with X³ is a bivalent protecting group;
X³ is hydrogen or a hydroxy-protecting group;
and the enantiomers, diastereomers, and salts thereof;

22. A compound of claim 21 in which at least one, preferably all three, of the asymmetric carbon atoms of the main chain have the stereochemical configuration shown in formula IIIa 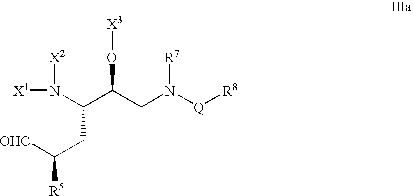
and the salts thereof.

23. (canceled)

24. A compound of claim 22, wherein
R⁵ is (C₃-C₅)alkyl;
R⁷ is hydrogen;
Q is carbonyl or sulfonyl;
R⁸ is (C₃-C₁₁)alkyl, (C₃-C₁₁)haloalkyl, (C₃-C₇)cycloalkyl, (C₃-C₁₁)cycloalkylalkyl, (C₃-C₁₁)alkoxy-alkyl, aryl, aryl(C₁-C₃)alkyl, aryl(C₃-C₆)cycloalkyl, arylhydroxy(C₁-C₃)alkyl, aryloxy(C₁-C₅)alkyl, or aryloxy(C₃-C₆)cycloalkyl wherein aryl or aryloxy may be unsubstituted or substituted with one to three groups independently selected from halogen, cyano, (C₁-C₃)alkyl, halo(C₁-C₃)alkyl, (C₁-C₃)alkoxy, and halo(C₁-C₃)alkoxy;
or R⁸ is NR⁹R¹⁰;
R⁹ is 1) hydrogen, (C₁-C₁₀)alkyl, (C₃-C₇)alkenyl, (C₃-C₇)cycloalkyl, (C₃-C₆)cycloalkyl(C₁-C₅)alkyl, (C₁-C₆)alkoxy(C₁-C₆)alkyl, or aminocarbonyl(C₁-C₅)alkyl, or 2) aryl or aryl(C₁-C₄)alkyl
wherein aryl is optionally substituted with up to 4 groups independently selected from fluorine, chlorine, cyano, (C₁-C₃)alkyl, halo(C₁-C₃)alkyl, (C₁-C₃)alkoxy, halo(C₁-C₃)alkoxy, and (C₁-C₃)alkanesulfonyl;
R¹⁰ is hydrogen, lower alkyl, or lower haloalkyl; or
R⁸ and R⁹ taken together are with the nitrogen to which they are attached form an azetidine, pyrrolidine, piperidine, azepine, piperazine, morpholine, or thiomorpholine ring said ring being optionally substituted with up to two groups independently selected from halogen, (C₁-C₃)alkyl, halo(C₁-C₃)alkyl, and oxo, such that substitution of one oxo group on a carbon atom forms a carbonyl group and substitution of one or two oxo groups on sulfur forms sulfoxide or sulfone groups respectively;
X¹ is tert-butoxycarbonyl;
X² together with X³ is isopropylidene;
and the salts thereof.

25. A compound of claim 22, wherein
R⁵ is branched (C₃-C₅)alkyl;
R⁷ is hydrogen;
Q is carbonyl or sulfonyl;
R⁸ is propyl, 2,2-dimethylpropyl, butyl, tert-butyl, n-pentyl, 2-methyl-2-butyl, hexyl, 2-hexyl, 2-methyl-2-pentyl, 2,2-dimethylpentyl, 3-heptyl, 2-methyl-2-hexyl, 2,4,4-trimethylpentyl, 2,2,2-trifluoroethyl, 3,3,3-trifluoropropyl, 4,4,4-trifluorobutyl, 1,1,1,3,3,3-hexafluoro-2-methyl-2-propyl, cyclohexyl, 1-methylcyclohexyl, 4-methylcyclohexyl, cyclopropylmethyl, cyclopentylmethyl, 1-cyclopentyl-1-pentyl, cyclohexylmethyl, 2-cyclohexyl-2-propyl, 2-cyclopropyl-1,1-dimethylethyl, 3-cyclopropyl-2-methyl-2-butyl, 3-methoxypropyl, 2-propoxy-2-propyl, phenyl, benzyl, 3-methylbenzyl, 2-fluorobenzyl, 3-fluorobenzyl, 4-fluorobenzyl, 2,4-difluorobenzyl, 2,3-difluorobenzyl, 3,4-difluorobenzyl, 4-cyanobenzyl, 2-(trifluoromethyl)benzyl, 3-(trifluoromethyl)-benzyl, 4-(trifluoromethyl)benzyl, 4-(trifluoromethoxy)benzyl, phenethyl, 3-phenylpropyl, 2-phenyl-2-propyl, 3-(4-fluorophenyl)-3-pentyl, 1-phenyl-1-cyclopropyl, 1-(4-methylphenyl)-1-cyclopropyl, 1-(4-fluorophenyl)-1-cyclopropyl, 1-(4-methoxyphenyl)-1-cyclopropyl, 1-(2,4-dichlorophenyl)-1-cyclopropyl, 1-phenyl-1-cyclopentyl, 1-phenyl-1-cyclohexyl, 1-(4-fluorophenyl)-1-cyclohexyl, 3-hydroxy-2-methyl-3-phenyl-2-propyl, 2-(4-cyanophenoxy)-2-propyl or 2-(4-chlorophenoxy)-2-propyl;
or R⁸ is NR⁹R¹⁰;
R⁹ is hydrogen, butyl, isobutyl, t-butyl, pentyl, hexyl, 2,2-dimethyl-1-pentyl, 2-methyl-2-hexyl, 2,4,4-trimethyl-2-pentyl, allyl, 2-(cyclopropyl)ethyl, cyclohexylmethyl, 2-(cyclohexyl)methyl, cyclohexyl, 2-methoxyethyl, benzyl, 2-phenylethyl, 3-phenylpropyl, 3-(4-fluorophenyl)-2-methyl-2-propyl, 3-fluorophenyl, 3-(trifluoromethyl)phenyl, or 2-(aminocarbonyl)-2-methyl-1-propyl,
R¹⁰ is hydrogen, methyl, or isobutyl;
or R⁹-R¹⁰ is —(CH₂)₅— or —(CH₂)₂O(CH₂)₂—;
X¹ is tert-butoxycarbonyl;
X² together with X³ is isopropylidene;
and the salts thereof;

26. A composition comprising an effective amount of a compound of claim 1 or enantiomer, diastereomer, or salt thereof, and a pharmaceutically acceptable carrier therefor.

27. A composition of claim 26 further comprising α-blockers, β-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, or endothelin receptor antagonists.

28. (canceled)

29. A method of inhibiting renin which comprises administering to a subject in need thereof a therapeutically effective amount of a compound of claim 1 or enantiomer, diastereomer, or salt thereof.

30. (canceled)

31. A method for treating or ameliorating an renin mediated disorder in a subject in need thereof comprising administering to said subject a therapeutically effective amount of a compound of claim 1, or enantiomer, diastereomer, or salt thereof or composition thereof.

32. A method of claim 31, wherein said disorder is hypertension, congestive heart failure, cardiac hypertrophy, cardiac fibrosis, cardiomyopathy post-infarction, nephropathy, vasculopathy and neuropathy, diseases of the coronary vessels, post-surgical hypertension, restenosis following angioplasty, raised intra-ocular pressure, glaucoma, abnormal vascular growth, hyperaldosteronism, anxiety states, or cognitive disorders.

33. A method of claim 31 further comprising administering said compound of claim 1 or enantiomer, diastereomer, or salt thereof or composition thereof in combination with one or more additional agents selected from the group consisting of α-blockers, β-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, and endothelin receptor antagonist.

34. A method of claim 33 wherein:
α-blockers include doxazosin, prazosin, tamsulosin, and terazosin;
β-blockers include atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts;
calcium channel blockers include dihydropyridines (DHPs) and non-DHPs, wherein the DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts and the non-DHPs are selected from the group consisting of flunarizine, prenylamine, diltiazem, fendiline, gallopamil, mibefradil, anipamil, tiapamil, and verampimil, and their pharmaceutically acceptable salts;
the diuretics include a thiazide derivative selected from amiloride, chlorothiazide, hydrochlorothiazide, methylchlorothiazide, and chlorothalidon;
ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril;
dual ACE/NEP inhibitors include omapatrilat, fasidotril, and fasidotrilat;
ARBs include candesartan, eprosartan, irbesartan, losartan, olmesartan, tasosartan, telmisartan, and valsartan;
aldosterone synthase inhibitors include anastrozole, fadrozole, and exemestane;
aldosterone-receptor antagonists include spironolactone and eplerenone; and
endothelin antagonists include bosentan, enrasentan, atrasentan, darusentan, sitaxentan, and tezosentan, and their pharmaceutically acceptable salts.
